SEQUENCE LISTING (33 sequences)

SEQ ID NO: 1 - DNA, 413 bp, Homo sapiens
  Genbank Accession No. AI968491
tataacatttaaaactacttatttttatttatatcagagtaacaaatacttatgaagtcctttgaagttcagcgcacaaatttctcgtgtggggcggtgggtgtggccatgttcttgctcttccttctttacacatttgagttgtgccttctgttcttaaagagattttcctttgttcaaaggatttattcctaccatttcacaaatccgaaaataattgaggaaacaggttacatcattccaattttgccttgggtttgaagagtctctcatggtggcacagtcctccagggtagctatgttgttgggctcccctacatcccagaagctcagagactttgtcaaaggtgtgccgtccacccattgccactgaccctcgacaacctggtctgacagtccaataaaaaactctc

SEQ ID NO: 2 - DNA, 375 bp, Homo sapiens
  Genbank Accession No. AI198352
gtcatttttccccactagacttcaagttccatgaaagagatttattttgtcctctgttctgtggtgtgcttggcataaagtagataagcaagcatctgctgaaggaatgaattaaatgccagctgcaatccccaaattacagtagatacacgggaaaattttctttaggaggctgaattctaacagcatgatatcaaacaaggatagggtcaatacagaggccagtgctgtttgaaagtcatcatttcaatggtaactccttcacttgtgctggactcggtctcccctcaggagactcctttcctgttcaaagtcagtattttatatactcaaaaccaacaacaaactccaggtgtaactntattccaccttctc

SEQ ID NO: 3 - DNA, 591 bp, Homo sapiens
  Genbank Accession No. AI925439
aaaagtgttctgtttaatacgctttgtctggtagtgcttgggtgcctgtggttggtttctctcactggaaccagtcctgggccccactcgcctggcttcctccagtcgccccaggtcctggggctggtgtgtcagaagctgcctttgtcctctccattcatccatccttgggcctgtctggcctatgatgccctcattcagctctcagggtccagaggtgactggtgtggatcctgctcgctgtgccaagatggccctccagatgcggcataccatccctcccctgatgcgggtgtagatgaggtcatctcgagtggcataggtgagcagagtgtggagggtgaagctatggttcaacagcatttggatggtgcctgaatccacattcagttcctgtagccactgcaccaggccctggtccgttgaaagagcagttggaggctctggggccaggacataggtccgggcttnctcattcagccgctgtagagcccgctgcaccagggcctggtactcccgttccttccccgccaggatttcgcgcagccgatcagtctctgccctcaagaagctcagctgcaccatcagagg

SEQ ID NO: 4 - DNA, 3060 bp, Homo sapiens
  Genbank Accession No. AF026031
ctacggagtgcatcggacgtcgaagcctagagtctctgcgtctttccctcttccgctgcctcattcctttccttcctagccttggtcgtcgccgccaccatgaacaagaagaagaaaccgttcctagggatgcccgcgcccctcggctacgtgccgtggctgggccggggcgccactggcttcaccacgcggtcagacattgggcccgcccgtgatgcaaatgaccctgtggatgatcgccatgcacccccaggcaagagaaccgttggggaccagatgaagaaaaatcaggctgctgacgatgacgacgaggatctaaatgacaccaattacgatgagtttaatggctatgctgggagcctcttctcaagtggaccctacgagaaagatgatgaggaagcagatgctatctatgcagccctggataaaaggatggatgaaagaagaaaagaaagacgggagcaaagggagaaagaagaaatagagaaatatcgtatggaacgccccaaaatccaacagcagttctcagacctcaagaggaagttggcagaagtcacagaagaagagtggctgagcatccccgaggttggcgatgccagaaataaacgtcagcggaacccacgctatgagaagctgacccctgttcctgacagtttctttgccaaacatttacagaccggagagaaccatacctcagtggatccccgacaaactcaatttggaggtcttaacacaccctatccaggtggactaaacactccataccgaggtggaatgacgccaggactgatgacacctggcacaggtgagctggacatgaggaagattggccaagcgaggaacactctgatggacatgaggctgagccaggtgtctgactccgtgagtggacagaccgtcgttgaccccaaaggctacctgacggatttaaattccatgatcccgacacacggaggagacatcaatgatatcaagaaggcgcgactgctcctcaagtctgttcgggagacgaaccctcatcacccgccagcctggattgcatcagcccgcctggaagaagtcactgggaagctacaagtagctcggaaccttatcatgaaggggacggagatgtgccccaagagtgaagatgtctggctggaagcagccaggttgcagcctggggacacagccaaggccgtggtagcccaagctgtccgtcatctcccacagtctgtcaggatttacatcagagccgcagagctggaaacggacattcgtgcaaagaagcgggttcttcggaaagccctcgagcatgttccaaactcggttcgcttgtggaaagcagccgttgagctggaagaacctgaagatgctagaatcatgctgagccgagctgtggagtgctgccccaccagcgtggagctctggcttgctctggcaaggctggagacctatgaaaatgcccgcaaggtcttgaacaaggcgcgggagaacattcctacagaccgacatatctggatcacggctgctaagctggaggaagccaatgggaacacgcagatggtggagaagatcatcgaccgagccatcacctcgctgcgggccaacggtgtggagatcaaccgtgagcagtggatccaggatgccgaggaatgtgacagggctgggagtgtggccacctgccaggccgtcatgcgtgccgtgattgggattgggattgaggaggaagatcggaagcatacctggatggaggatgctgacagttgtgtagcccacaatgccctggagtgtgcacgagccatctacgcctacgccctgcaggtgttccccagcaagaagagtgtgtggctgcgcgccgcgtacttcgagaagaaccatggcactcgggagtccctggaagcactcctgcagagggctgtggcccactgccccaaagcagaggtgctgtggctcatgggcgccaagtccaagtggctggcaggggatgtgcctgcagcaaggagcatcctggccctggccttccaggccaaccccaacagtgaggagatctggctggcagccgtgaagctggagtccgagaatgatgagtacgagcgggcccggaggctgctggccaaggcgcggagcagtgcccccaccgcccgggtgttcatgaagtctgtgaagctggagtgggtgcaagacaacatcagggcagcccaagatctgtgcgaggaggccctgcggcactatgaggacttccccaagctgtggatgatgaaggggcagatcgaggagcagaaggagatgatggagaaggcgcgggaagcctataaccaggggttgaagaagtgtccccactccacacccctgtggcttttgctctctcggctggaggagaagattgggcagcttactcgagcacgggccattttggaaaagtctcgtctgaagaacccaaagaaccctgggctgtggttggagtccgtgcggctggagtaccgtgcggggctgaagaacatcgcaaatacactcatggccaaggcgctgcaggagtgccccaactccggtatcctgtggtctgaggccatcttcctcgaggcaaggccccagaggaggaccaagagcgtggatgccctgaagaagtgtgagcatgacccccatgtgctcctggccgtggccaagctgttttggagtcagcggaagatcaccaaggccagggagtggttccaccgcactgtgaagattgactcggacctgggggatgcctgggccttcttctacaagtttgagctgcagcatggcactgaggagcagcaggaggaggtgaggaagcgctgtgagagtgcagagcctcggcatggggagctgtggtgcgccgtgtccaaggacatcgccaactggcagaagaagatcggggacatccttaggctggtggccggccgcatcaagaacaccttctgattgagcggttgccatggccggtctccgtggggcagggttgggccgcatgtggaagggctctgagctgtgtcctccttcattaaaagtttttatgtctcgtgtcaaaaaaaaaaaaaaaaaaaaagaaaaaaaaaa

SEQ ID NO: 5 - DNA, 646 bp, Homo sapiens
  Genbank Accession No. AA524258
tttcctaatcattacaaaatattctttattacttttaaattaaaaattagaaaaaatgaaaacaaggtatctcaaactctgaaagacactgagacttcagaaaatcctaacatttgagatctcaaacgaaactaacaaatattgactaatttaccattactaggtgccgagatgttttatccctccaggagactatgagcctcctaaatatccctgacttattatgacagagcaattccaacagtcatgaaggttccctggaaatcacttagagttgatcttgattgaattagtccagccttcagagaaaccagggctgtcacttgcccccgtgaaccaccattgtgctgtatcggaactggttccaatcctcttcttgtgtctggacctccaagcacctcaggttttccctgctcctcatagaaaaattcttcctctgcatttgcaaacattaacgcagctttctttttgttgctaggtttctttttggaattgttttttcctgcttccacaaatgtcttactaatcagaaggtaaaagtagcacttcccacatggcttattgggtctgtgtgccccgccagttcttctgaagctgctggtacatggcagacgatctgtgtgagggacataatgatctttcactt

SEQ ID NO: 6 - DNA, 2285 bp, Homo sapiens
  Genbank Accession No. AF004430
acgcggcgagcttctcccggcgccgcccgctcggctcccatagcgcccgcgacagcggtccggacgccgcccgaacatggactccgccggccaagatatcaacctgaattctcctaacaaaggtctgctgtctgactccatgacggatgttcctgtcgacacaggtgtggctgcccggactcctgctgttgagggtctgacagaggctgaggaggaggagctcagggctgagcttaccaaggtggaagaggaaattgtcactctgcgccaggtcctggcagccaaggagaagcactgtggagagctcaagaggaggctgggcctctccaccctgggggagctgaaacagaacctgtccaggagctggcatgacgtgcaggtctctagcgcctatgtgaaaacttctgagaaacttggagagtggaatgagaaagtgacccagtcagacctctacaagaagactcaggaaactctttcacaggcaggacagaagacttcagctgccctgtccacagtgggctctgccatcagcaggaagcttggagacatgaggaactctgcgaccttcaagtcgtttgaggaccgagttgggaccataaagtctaaggttgtgggtgacagagagaacggcagtgacaacctcccttcctcagcggggagtggtgacaagcccctgtcggatcccgcacctttctaagcctgtggttgcttcacccgctgcagagcacacgcaacccagcctcagcatcacagccgcagctctgttcagcggagcagccagccagggcggatgagcagagccggccctgaggacagtcctgcccatccacgcggagatgtggctgccgcgtttgcatgaatttgaagaacacaggcttgtacacagatgttttacactcacgtttgtagatgaaacagatcactgtgctgtccttcctaggggtgcaggaagtggacagggcggagggtttgaaagaatattgagccaaagcccaggctccctttgggaatcatgttagcccatcagaatgttgaaggattgaagagttctaagcataaaataagtggcattttctgacttcttcctcctcctccttccctgactcacagaaggaatgcaatcacccagcaagtcctacctgttacgcaattttttatctcaaaatgccgaacgagaaaactgtccattttctgagacccccagaaaggaaactgaccctcagcagctgcctgattgttacgcgaatctagctttaacggaagcaaattcattattttttaaatgcagtggacttttcaaaaagtttaaattaggcaaagcagctttagcctcatagaatattatttctttggactcaagctgaaatacaagccttacattgccttatgctttatttctttctaatttttatatgtatatagatgagggttccttaatggttgtgagcattgtgtggaattttacacctggcctgcgtggcagcctcttccagttgaggtgttttatgtcacgcacactccatcccagtgtacaaaacctgcttctcttctcaaccgtggcagctcccgctggctcctatgccctgccctaaagggctcttgagcctctgggaatgggaggggccaagagaaggaaaaccctgtctttagcaccctttaaaagaactgtgccccccttctcagtgctgcctttgcatgggcctggcccggctcacattcgtcagtgactccaaccctcctgcttgctgtacttgggatgaaacgaccccacaggtcaggtggagggtggggcgtgggcatcagccaggattgccgttacagtctttttctcaggagctacaaagatctcttcctgttactaaatagtcgcaccccagcagcctctctcgcacaccggggccctgcatgtcagatggcgtggtctgcagggggagctctgtgccttagtggctcttggcaggacactgagggcctgcctgtggtgtgcccggctctgccactcccgggaggggaagggctgctcagctcaaggtgtcctgttcggtagagcaagtgtcctctgacagccgtgtccccggacagttcagacacccttggggatggcactccacacacgacagagatgcaggggccagggaagcccagcgctcggtgcccttcgtccagggttaaaatcggcctgtggggtgtggtgagaaggcaggttgtgcgggtgttgaccgatgtatcttttccttaaagttattataataatgggtaatttgtcaataaagcattcctttggg

SEQ ID NO: 7 - DNA, 821 bp, Homo sapiens
  Genbank Accession No. AI805297
tttgtatcaattaatttttttattcagaacctggatctttaagactggtataaacaaaaataaaatacaaacaacttgttattaacataatagctgcagttattaactgcaagaatacagtaaaaaggagtcatactaaatgttaatgttcttgcaacagaattttcccaatcaccttaaggtagcaaattctggcaagaaatgataattcatttcttattaaaaatacttaaagaatttccaagattgatcagttgtgccacaaataaaacagcagtttattcaaaagagcccttacctttctcttattttcctaaaagatctgccatttacaggtaactactaatttcagatcagttattcaaaccagggttcttcctgtaaatccacatcttagctttatatatagtaaacttacactcatattccctttccagtcttgaagctaaacacttttgtaaaaggaacaatctaatatcacttcatatgaatcttgttcttactttcttctatctcatttntattttcttctattatttgtcttcctcctcaccacccaaaagctgtttcttgcatgtggaaaaggagaagactagcagccacttctgcagcaaggcccagagtgaaggtaaaatctgtgacagctggaccgtgcagcctctgctaatggaggctgtatgagtccctggagtgaagggcaggcagaccactcctttggttgggtatctcactctaacattgtacacatcttcttctgctctttggggagattatttgagaatatttaatttactttttagagttcacaataaactagccta

SEQ ID NO: 8 - DNA, 5803 bp, Homo sapiens
  Genbank Accession No. D80000
agattgagaactttaagtcgtacaagggtcgacagattatcggaccatttcagaggttcaccgccatcattggacccaatggctctggtaagtcaaatctcatggatgccatcagctttgtgctaggtgaaaaaaccagcaacctgcgggtaaagaccctgcgggacctgatccatggagctcctgtgggcaagccagctgccaaccgggcctttgtcagcatggtctactctgaggagggtgctgaggaccgtacctttgcccgtgtcattgtaggaggttcttctgagtacaagatcaacaacaaagtggtccaactacatgagtacagtgaggaattagagaagttgggcattctcatcaaagctcgtaacttcctcgttttccagggtgctgtggaatctattgccatgaagaaccccaaagagaggacagctctatttgaagagattagtcgttctggggagctggcgcaggagtatgacaagcgaaagaaggaaatggtgaaggctgaagaggacacacagtttaattaccatcgcaagaaaaatattgcggctgaacgcaaggaagcaaagcaggagaaagaagaggctgaccggtaccagcgcctgaaggatgaggtagtacgggctcaggtacagctgcagctctttaagctttaccataatgaagtggaaattgagaagctcaacaaggaactggcctcaaagaacaaggagatcgagaaggacaagaagcgtatggacaaggtggaggatgaactgaaggagaagaagaaggagctgggcaaaatgatgcgggagcagcagcagattgagaaggagatcaaggagaaggactcagaattgaaccagaagcggcctcagtacatcaaagccaaggagaacacctcccacaaaatcaagaagctggaagcagccaagaagtctctgcagaatgctcagaagcactacaagaagcgtaaaggtgacatggatgagctggagaaggagatgctgtcagtggagaaggctcggcaggagtttgaagaacggatggaagaagagagtcagagtcagggcagagatttgacgttggaggagaatcaggtgaagaaataccaccggttgaaagaagaagccagcaagagagcagctaccctggcccaggagctggagaaattcaatcgagaccagaaagctgaccaggaccgtctggatctggaagaacggaagaaagtagagacagaggccaagatcaagcaaaagctgcgggaaattgaagagaatcagaagcggattgagaaactggaggaatacatcaccactagcaagcagtccctagaagagcagaagaagctagagggggagctgacagaggaggtggagatggccaagcggcgtattgatgaaatcaataaggagctgaaccaggtgatggagcagctaggggatgcccgcatcgaccgccaggagagcagccgccagcagcgaaaggcagagataatggaaagcatcaagcgcctttaccctggctctgtgtacggccgcctcattgacctatgccagcccacacaaaagaagtatcagattgctgtaaccaaggttttgggcaagaacatggatgccattattgtggactcggagaagacaggccgggactgtattcagtatatcaaggagcagcgtggggagcctgagaccttcttgcctcttgactacctggaggtgaagcctacagatgagaaactccgggagctgaagggggccaagctagtgattgatgtgattcgctatgagccacctcatatcaaaaaggccctgcagtatgcttgtggcaatgcccttgtctgtgacaacgtggaagatgcccgccgcattgcctttggaggccaccagcgccacaagacagtggcactggatggaaccctattccagaagtcaggagtgatctctggtggggccagtgacctgaaggccaaggcacggcgctgggatgagaaagcagtagacaagttgaaagagaagaaggagcgcttgacagaggagctgaaagagcagatgaaggcaaaacggaaagaggcagagctgcgtcaggtgcagtctcaggcccatggactgcagatgcggctcaagtactcccagagtgacctagaacagaccaagacacgacatctagccctgaatctgcaggaaaaatccaagctggagagtgagctagccaactttgggcctcgcattaatgatatcaagaggatcattcagagccgagagagggaaatgaaagacttgaaggagaagatgaaccaggtagaggatgaggtgtttgaagagttttgtcgggagattggtgtgcgcaacatccgggagtttgaggaagaaaaggtgaaacggcagaatgaaatcgccaagaagcgtttggagtttgagaatcagaagactcgcttgggcattcagttggattttgaaaagaaccaactgaaggaggaccaagataaagtacacatgtgggagcagacagtgaaaaaagatgaaaatgagatagaaaagctcaaaaaggaggaacaaagacacatgaagatcatagatgagaccatggctcagctacaagacctgaagaatcagcatctggccaagaagtcggaagtgaatgacaagaatcatgagatggaggagattcgtaagaaactcgggggcgccaacaaggaaatgacccatttacagaaggaggtgacagccattgagaccaagcttgaacagaagcgcagtgaccgtcacaacttgctacaggcctgtaagatgcaggacattaagttgccactgtcaaaaggcaccatggatgatattagtcaggaagagggtagctcccagggggaggactcagtgagtggttcacagagaatttccagtatctatgcacgagaggccctcattgagattgactacggtgatctgtgtgaggatctgaaggatgcccaggctgaggaagagatcaagcaagagatgaacacactgcagcagaagctgaatgagcagcagagtgtgcttcagcgtattgccgcccccaacatgaaggccatggaaaagctggaaagtgtccgagacaagttccaggagacctcagatgagtttgaagcagcccgaaagcgagcaaagaaggccaagcaggcattcgaacagatcaagaaggagcgctttgaccgcttcaatgcttgttttgaatctgtggctaccaacattgatgagatctataaggccctgtcccgcaatagcagtgcccaggcattcctgggccctgagaaccctgaagagccctacttggatggcatcaactacaactgtgtggctcctgggaaacgcttccggcctatggacaacttgtcaggcggggagaagacagtggcagctctggccctgctctttgccatccacagctacaagccagcccccttcttcgtcctggatgagattgatgctgccttggataacaccaacattggcaaggtggcaaattacatcaaggagcagtcgacttgcaacttccaggccatcgtcatctctctcaaggaggagttctacaccaaggccgagagcctcattggagtctatcctgagcaaggggactgtgtgatcagcaaagtcctgaccttcgacctcaccaagtacccagatgccaaccccaaccccaatgagcagtagcagtatttttgccctcccgccctgtctggatccctaagctgtccctctcccaatctctggatatttgactcccaaccttccccctacctcctggccctttttggtgtagtcatgggatttaggcactgctaatcaagcatgaagaggaacagaggtgatgttaggtctggagcaaaaattcctgaacgacagggagtattctggcctctgaaaggaggtgctgagctgaacagggccatctgttcatcacacacacccccttcctccccctcatcacccataatcgtgggccccttgggcctcttgcccactgtgtgtgtgggtatgtatgtgtgtatgtatgtatccgcatgtgtgcatgtgagtatgtttgcaaaataataaaggatattggagacctgttttagaaggagcctaggctgaatttgattccaagagagcttaggatgacagcacccctgagctgggcaaaggtactcaggacctcataggagtcttaggcagttacctgaaactgccttcattcactcatttgtgtattcattcatttatgtattcatcagacacataccgaacaccctctatttgtcaggctctgtgcttggaatacagagttgaatcagacatgatctctaccctcctagtaaggagatacagtgggttcatgaatgactatagttagctgaatgtcatatgtactttgaatttgagaagtgggtgatcccctctaggcttcctggaggtcacatttaagctagaccttgacaaattggtaggatttggtcaggcactaggagtggagcatgagctctggggacagacagttatgggttctggtcccactttttatcacttactagttgtttgaccttgggcaagtcatttgaccttctgtgcctcagtttcctcatctgtaaaatggggctaacaatattacctacctcataggatttaatgatgtcaagctcctcactggaggccttatcccttcgtggagcccactaggtgccgacccctcagaatataaccctcatgcctggacccctgagagcttctgatcccagctattagggacagaagaagcctccaaatctggaaggtgctgaatgccctgctgactgggaaagtttcagggcactgatggggtctacctggtaagcggagggcctgaggaaacctgtagcttcaatcatgtctggtaaccgggtgcctgagccccaatctgggttgtgaggaaataggggagaggtatcctgggccacatcccagcctaacacctgtgaggttcattttaggaactaacctcattagctataaggatcatgcagaggcagcaaagccgggtgcgatgagctcagcctttactcattcacatacaccatcacactttaattccaatctgtatattgctttttaaaagttaagtccattctaattacccaaatatgcatgaattcattctccttttgagaagttagattgttaaagatagtctcattcagctaccaaccactccttgatccttcccttcttagtggctgttgtttgttgtacttccgtttagactttgttttaatgcttgtacgtacatatgtgaactcattggaaatattgtgtgtttaatgcaaatgatatattgaattgtttagcaatttgttttctttgcttaacgatgtttttgagatctgtgcatgttacttaatgtagctcaatccatcttctgtaattgctgtatagattgtcatcatatgattaccacattttacttacgcatttcttttgtgatggacattaagactgtttttaggttttgctattacaaaatactacacaggagcatcactatgcctgtgtgaaagtatatgtatgaaagtttacctagggttgattcctagaagtggaattgcaaagtcataggatatttatatattggtttttaataatacttccaaattgccctcctgtactatttactcagtatttttcttgaggttgatctgaggtctaacattgttatcctatatcattttcatcccaagtagtgatatctgtgaaatcacaggtttgatgtgtgctaattatgtattcttctaatacatattaaaagacataactatcaaaac

SEQ ID NO: 9 - DNA, 672 bp, Homo sapiens
  Genbank Accession No. AI984100
tttttttttagactgtaaatgtattttattttgtaagtgacaatcaaaatatatttgtgctactgattttttcaaatattaaatgggatcttaacaaaatttgtccactttgatgtattcatgtataattttagaatctgcaaagaccctgactaaagaaacagaagtaattgcacaactgagactgaaggtgttacagtgctgcaaatgatgccagatgtcttacatttagccaaggtccccttttcgaaggtcccttgattttgagcattaggaacaacagaggaatatacactgaaggcatcattaatagcgtttttcgttgctttatttaaatacgacagtatccaacatagctgctaaacactggacaatatttgaggtcataagtacatccacatgttcctccaaatgacgtttggggtcctgcttgccaacattctttattgccagctgttcaggtgtcatcttatcttcttcttctacagccttattgtaattcttggctaattccaacatctcttttaccactgattcattgtgtttacaatgttcactgtagtcctgaagtgtcaaacctttcatccaactcttnctatgcaaatttagcaacatctttctgtccagttcatttttccgatagntaatagtaatggagtcataatgtctgtt

SEQ ID NO: 10 - DNA, 675 bp, Homo sapiens
  Genbank Accession No. AF035940
acgaggcagacggcggcagtgcggcttgctcttggaagttcaggctcggttgtcttttgggagccatggagagtgacttttatctgcgttactacgtggggcacaagggcaagttcggccacgagttcctggagtttgagtttcgaccggacgggaagttaagatatgccaacaacagcaattacaagaatgatgtcatgatcagaaaagaggcttatgtacataaaagcgtgatggaggaactgaagagaataattgacgacagtgaaattaccaaagaggatgatgcattgtggcctcctcctgaccgagtgggccggcaggagcttgaaatcgtcattggagatgaacacatttcttttacaacatcaaaaattggttcccttattgatgtcaatcaatccaaggatccagaaggcttacgagtattttattatcttgtccaggacctgaagtgtttggtcttcagtcttattggattacacttcaagattaaaccaatctagactgaatattggtgtggacatggggggtgggtgggagtagaaaattttgtgtatatcagggcagtatttttttatgaactataaatgattgtctttaataaatatgtgataaaatccaatttttattattttataaagacctgaacatgtgaaaaaaaaaaaaaaaaaa

SEQ ID NO: 11 - DNA, 489 bp, Homo sapiens
  Genbank Accession No. AI951998
ctaatccaaacgcacttctctttattcaaaccagggtcaaactggtcaatgggaaacgccctgaagccacgtgcctggggagaaaggcttcctactcggttcggttcagcgctgcgtgggatccacgcggctggctgtgcgcaacccccacagttcacctcagacactaccaagcaggtcagtcgacaaaagcaaggaattaaacaaaaaacagaaatacactcagtagatttcttctagaagctcccagagtttctggaccaccaagtcccaacccccaaagccaggagcgaggggactaacagcgcaccccctccaccagtgccgacggaaaccccgttttaaattaaaaaataagccagtatacatcgtagaaaatttctcttaaaaatctcacaatttgtaaatgtatattttttctttaacataaaagtttacaatataccgtagaacaaaaggctcaggaaaataatttccaaaaaaaaggaa

SEQ ID NO: 12 - DNA, 1806 bp, Homo sapiens
  Genbank Accession No. U12707
agcctcgccagagaagacaagggcagaaagcaccatgagtgggggcccaatgggaggaaggcccgggggccgaggagcaccagcggttcagcagaacataccctccaccctcctccaggaccacgagaaccagcgactctttgagatgcttggacgaaaatgcttgacgctggccactgcagttgttcagctgtacctggcgctgccccctggagctgagcactggaccaaggagcattgtggggctgtgtgcttcgtgaaggataacccccagaagtcctacttcatccgcctttacggccttcaggctggtcggctgctctgggaacaggagctgtactcacagcttgtctactccacccccacccccttcttccacaccttcgctggagatgactgccaagcggggctgaactttgcagacgaggacgaggcccaggccttccgggccctcgtgcaggagaagatacaaaaaaggaatcagaggcaaagtggagacagacgccagctacccccaccaccaacaccagccaatgaagagagaagaggagggctcccacccctgcccctgcatccaggtggagaccaaggaggccctccagtgggtccgctctccctggggctggcgacagtggacatccagaaccctgacatcacgagttcacgataccgtgggctcccagcacctggacctagcccagctgataagaaacgctcagggaagaagaagatcagcaaagctgatattggtgcacccagtggattcaagcatgtcagccacgtggggtgggacccccagaatggatttgacgtgaacaacctcgacccagatctgcggagtctgttctccagggcaggaatcagcgaggcccagctcaccgacgccgagacctctaaacttatctacgacttcattgaggaccagggtgggctggaggctgtgcggcaggagatgaggcgccaggagccacttccgccgcccccaccgccatctcgaggagggaaccagctcccccggccccctattgtggggggtaacaagggtcgttctggtccactgccccctgtacctttggggattgccccacccccaccaacaccccggggacccccacccccaggccgagggggccctccaccaccaccccctccagctactggacgttctggaccactgccccctccaccccctggagctggtgggccacccatgccaccaccaccgccaccaccgccaccgccgcccagctccgggaatggaccagcccctcccccactccctcctgctctggtgcctgccgggggcctggcccctggtgggggtcggggagcgcttttggatcaaatccggcagggaattcagctgaacaagacccctggggccccagagagctcagcgctgcagccaccacctcagagctcagagggactggtgggggccctgatgcacgtgatgcagaagagaagcagagccatccactcctccgacgaaggggaggaccaggctggcgatgaagatgaagatgatgaatgggatgactgagtggctgagttacttgctgccctgtgctcctccccgcaggacatggctccccctccacctgctctgtgcccaccctccactctcctcttccagggcccccaaccccccatttcttccccaccaacccctccaatgctgttatccctgcctggtcctcacactcacccaacaatcccaaggccctttttatacaaaaattctcagttctcttcactcaaggatttttaaagaaaaataaaagaattgtctttctgtctctctat

SEQ ID NO: 13 - DNA, 1932 bp, Homo sapiens
  Genbank Accession No. X92106
cagcgagccgcagcgcaatcccggcgctcgcccaaggaccctggaagctaccgttaccccgccggcagcgtgggcgccatgagcagctcgggactgaattcggagaaggtagctgctctgatacagaaactgaattccgacccccagttcgtacttgcccagaatgtcgggaccacccacgacctgctggacatctgtctgaagcgggccacggtgcagcgcgcgcagcatgtgttccagcacgccgtgccccaggagggcaagccaatcaccaaccagaagagctcagggcgatgctggatcttttcttgtctgaatgttatgaggcttccattcatgaaaaagttaaatattgaagaatttgagtttagccaatcttacctgtttttttgggacaaggttgaacgctgttatttcttcttgagtgcttttgtggacacagcccagagaaaggagcctgaggatgggaggctggtgcagtttttgcttatgaaccctgcaaatgatggtggccaatgggatatgcttgttaatattgttgaaaaatatggtgttatccctaagaaatgcttccctgaatcttatacaacagaggcaaccagaaggatgaatgatattctgaatcacaagatgagagaattctgtatacgactgcggaacctggtacacagtggagcaaccaaaggagaaatctcggccacacaggacgtcatgatggaggagatattccgagtggtgtgcatctgtttgggtaatccaccagagacattcacctgggaatatcgagacaaagataaaaattatcagaaaattggccccataacacccttggagttttacagggaacatgtcaagccactcttcaatatggaagataagatttgtttagtgaatgaccctaggccccagcacaagtacaacaaactttacacagtggaatacttaagcaatatggttggtgggagaaaaactctatacaacaaccagcccattgacttcctgaaaaagatggttgctgcctccatcaaagatggagaggctgtgtggtttggctgtgatgttggaaaacacttcaatagcaagctgggcctcagtgacatgaatctctatgaccatgagttagtgtttggtgtctccttgaagaacatgaataaagcggagaggctgacttttggtgagtcacttatgacccacgccatgaccttcactgctgtctcagagaaggatgatcaggatggtgctttcacaaaatggagagtggagaattcatggggtgaagaccatggccacaaaggttacctgtgcatgacagatgagtggttctctgagtatgtctacgaagtggtggtggacaggaagcatgtccctgaagaggtgctagctgtgttagagcaggaacccattatcctgccagcatgggaccccatgggagctttggctgagtgatactgccctccagctctttcctccttccatggaacctgacgtagctgcaaaggacagatccagggactgaagccaaagttatgcaagggactgtgtgttgccacaggacacagtcagatttccagtctccaccaggaacctcttcagaaagtgtgctttatgctgaaacagaatactgttaaaggaaaaaaaagaggggggaagatcaggtcatactatctactctcctcatctctaacagctcaggatctcttagcattttaattagatgtaattgtttgtctttaactgtcaaaaagtttggttctgtgtctgtgttttaataagacgagaggacgagcgattgaggtgtatggagagaaaacagacctaatgctccttgttcctagagtagagtggagggagggtggcctaagagttgagctctcggaactgcatgctgctggacagtatcactgtctttcctagatggcagtcactgaattc

SEQ ID NO: 14 - DNA, 2428 bp, Homo sapiens
  Genbank Accession No. AL050353
cagacgcgcgggcgggaagatggcggctgggttcaaaaccgtggaacctctggagtattacaggagatttctgaaagagaactgccgtcctgatggaagagaacttggtgaattcagaaccacaactgtcaacatcggttcaattagtaccgcagatggttctgctttagtgaagttgggaaatactacagtaatctgtggagttaaagcagtaagtctaaatatgtcctattgagatttgagttacaaagttagctttatttattagattgtaatatacacctgtaattctactcttgatttggtagttgggtgtcaatttttaatgtttaatttcccaagagctttcactatgagtgttagataatatgacccaagtttaatgactaagtgaacaattgtcaaatgtagcaaaatgaaacaacttttaaaatctgagtcaccattttaaaaaaattgttatatcatttgagacattaacagatttttggtgacatccttttagccagtgatgacatgtaatctgtgtaaatgtgtagaaaattgaaattgttgaaaagaaaaatttgagggaatactggctaccatttactgagtacttgctatgttccagacacctttttaggtgctttacagaattatccactttaatgcctataacaatcatttgttacttgtcttcacctcctgttttacagatgactttcagtaacctgtccaggtacataagactcataagtaaaacttagacataaatctgactccaaagcctgtgcttctttgactacagtctaccctgtgttatttaacctcagttgagattcttctttcgtaaacctggagtgatgtataacagtaagatttgagaacttgctccttgtcaggttttatgcctgggatttaacaggtctatctcatttggcccttattaacagtccctaggagtagacactattttcttcccatttgacaagatgaggagtttaaggtaagaggttaaataacttgcctgaagtctctcagccagtaagtatggctctgacatttgaactggcttagtaattgcagagcatttgcttttttttttttttgagacggagtcttgctctgtcgcccaggctggagtgcagtggcacgatgtcagctcactgcaacttccgtctcctgggttgaagtgattctcctgcctcggcctcccaagtagctgggattacaggcgcccgccaccatgcctggctaatttttgtatttttagtagagacggggttttgccatcttggccaggctggtctcaaactcctgacctcaggtgatccgcccgcctctgtgtcccaaagtgctgggattaccggtgtgagccaccgcgcccagctgagcttttgctctttaccactaaagcagcagttcctaaatcttgctctatcatctggagaacaaattaagattcttggctagtagttgggggcacaaacccagttactattttttaaaagatctccaggtaatcttgccgagcagcactgtagtgtagtccactgtactgtagtgccttcttatcttgtagagctattgtgaggatcagcaccaatgtccgtagaggccctgacacaattccctaagctgatatttgatagagccttccataactgattgctaaattgtcttcataatttttcagtagcttctttcaataggaaacatttctttgctactataggaatttgcagcaccatcaacagatgcccctgataaaggatacgttgattccggtctggacctcctggagaagaggcccaagtggctagccagttcattgcagatgtcattgaaaattcacagataattcagaaagaggacttatgcatttctccaggaaagcttgtctgggttctatactgtgatctcatttgcctcgactacgatggaaacattttggatgcctgcacatttgctttgctagcggctttaaaaaatgtacagttgcctgaagttactataaatgaagaaactgctttagcagaagttaatttaaagaagaaaagttatttgaatattagaactcatccagttgcaacttcctttgctgtgtttgatgacactttgcttatagttgaccctactggagaggaggaacatctggcaacaggaaccttaacgatagtaatggatgaggaaggcaaactctgttgtcttcacaaaccaggtggaagtgggctaactggagctaaacttcaggactgtatgagccgagcagttacaagacacaaagaagttaaaaaactgatggatgaagtaattaagagtatgaaacccaaataaacagccaccacattttcaaaacagatttgtaaaaattgtatttgttaacactgtgcacaaacgttttatactaaataaatatcaaactacattcttctgaaaaaaaaaa

SEQ ID NO: 15 - DNA, 3131 bp, Homo sapiens
  Genbank Accession No. U69127
gcacgaggcggacgggaggccggaccggggagccgagcgcgcgtcggcggcgtcggcggcggcggcgacggcggcgggggcggtaatggcggacgtggtgcaggggcagagcgctcctgtggggatgaaggccgagggttcgtggatgccctgcaccgggtccggcagattgctgctaaaattgattcaattcctcacttgaataattccacacctctagtggacccctcagtatatggatacggagtacaaaaacggcccttggatgatggagtaggtaaccagttaggggccttggatcatcaaaggacggtaataacggaagaattcaaagtgcctgacaaaatggttggatttattatcggcaggggaggtgagcagatttcacggattcaagcagaatctggttgcaaaattcagattgcttcagagagttctgggattccagagaggccctgtgtacttaccggaaccccagaaagtattgaacaagccaaacggctcctgggacagattgtggaccgctgtcgaaatggacctggctttcataatgacatagacagcaacagcacaatccaggagattctcattcccgcatctaaagtgggtctggtcatcggcagaggaggggaaacaatcaagcagttgcaggagcggacaggggtgaagatggtcatgatccaggatggcccattgcccacgggagcagacaagcctcttcgtatcactggagatgcatttaaagtacagcaagcaagagaaatggtactagagattatccgagaaaaagaccaagctgactttcggggtgtacgcggcgatttcaactctcgaatgggaggaggcagtatagaggtatctgtgcctaggtttgctgtggggattgtaataggaagaaacggggaaatgatcaaaaagatccagaatgatgctggtgtgaggattcagtttaaaccagatgatgggattagtccagaaagagctgcccaggtcatgggccctccggatcggtgtcagcatgcagcgcatatcatcagcgagctgattcttacagcccaggaaagagacggctttggaggcctggcagcagccagaggaagaggtcgtggccgtggcgactggagcgtgggagcccctggtggcgtccaggagataacatacacggtgccagccgataagtgtggcctcgtcataggcaaagggggtgagaacatcaaaagcatcaaccagcagtcaggggcgcacgtggagcttcagaggaacccccctcccaacagcgaccccaacctgcggagattcaccatcaggggggttccccagcagatcgaggtggccaggcagctcatagatgagaaagttggcgggaccaatctcggagcacctggagccttcggacagagtccattcagccagccacctgccccacctcatcaaaatacctttcctccaaggagctccgggtgcttcccaaacatggctgccaaggtgaatgggaacccccacagcacccctgtgagtggtcctccggcctttctgacccagggctggggcagcacctaccaggcgtggcagcagcccacacagcaggtcccaagccagcagagccagccgcagagcagccagcccaactacagcaaggcctgggaagactattacaaaaaacagagtcacgccgccagcgctgctcctcaggccagctccccaccggactacacaatggcctgggcagaatattacagacagcaggtcgctttctacggacagacgttagggcaggcgcaggcccacagccaggagcagtaggacagcgtcctcgtggccaactctcccctcaaaatcattgtcaaagaaaacctatttgtaacagaggtttttacatttgcaaaccttttgatgaagaactgttgttttgttctgtgaaacactatatttagattaacggaatttttctaaaaatcaggaaaattagttactaaaaattgctgatcatttttgtttcattatttttgttatttcaaatgtataagctctgggattctttttggagcaatacctacaaagtcaggcaccagaatgtgcctcagagctgtgacatttcaacatgatggttttggtttggtttggttttgtgtcctttttatttgcagttttttctgttgcaacagaaagtggcttggaagtcttaggtggtatgtaacaaattctttttaaaaattttaaagcagtatttaagtattcttaaatgtgtaaattcatttaatgttttacttctaatttcttgtatcttggctgtctggttttattgcatttttaaaaaaactgaactattatgtattgtaatgaatgatgtgaattctgaactgagacagataattgtattgctgtccaacaggggttgggaggggcattttgtagggtttgtataatttctagagttcgaaaggggtaatataaaaatgtgctcgtgtgtttagcctacgtttttctcatgcctccattaccagttgcaattgtgtatctaagacctccaagcttgtttttaaaacaaaacaaaaaacctttctctattatctcagaaananaaatactgnwnttttaatattaagaanttcaatataactttnaacacacactgtggaacattaamcgtataaaaatgtagttataactatttttaattccaaggtgttgtttgcttttttcttttaaatattttcttaatattttgtcagattaactagatgaataaataaatctagtattaaccgcattatgaattaaataattttgatttaatgaaagggataatatgatttccagtgtttactgtagtgtatcttgtacagataacatgtatttttaaaggaaaaaaaacggaattgaagctattttttcttgcatttctaattgacctgagggacattccgtttgaaatgtactgaagttacagtttctggttttttctccttatttttcttataatgcttgaaatgtctaactattaaaaaagacaattggaaaatgttatgcatggggtttttaagaaaacaaagtgttctttttatttgactgacaattcattttacactctatataataaaatctccacaaggcatcttgtgggcaaagtc

SEQ ID NO: 16 - DNA, 1793 bp, Homo sapiens
  Genbank Accession No. AF035309
aagcgatgggcgcgggaatggccggcccacgggtcgcaggagacgggacgccagtcctttggctccgttccgctggctccttcgtcagtactgacacctcgggcttgtagagcacttcacgcagtgcaaagcgccccccgtctatatcatatcgcctctcggtcctcctaaaagtcgtatgagatggagctggaggaggggaaggcaggcagcggactccgccaatattatctgtccaagattgaagaactccagctgattgtgaatgataagagccaaaacctccggaggctgcaggcacagaggaacgaactaaatgctaaagttcgcctattgcgggaggagctacagctgctgcaggagcagggctcctatgtgggggaagtagtccgggccatggataagaagaaagtgttggtcaaggtacatcctgaaggtaaatttgttgtagacgtggacaaaaacattgacatcaatgatgtgacacccaattgccgggtggctctaaggaatgacagctacactctgcacaagatcctgcccaacaaggtagacccattagtgtcactgatgatggtggagaaagtaccagattcaacttatgagatgattggtggactggacaaacagatcaaggagatcaaagaagtgatcgagctgcctgttaagcatcctgagctcttcgaagcactgggcattgctcagcccaagggagtgctgctgtatggacctccaggcactgggaagacactgttggcccgggctgtggctcatcatacggactgtacctttattcgtgtctctggctctgaactggtacagaaattcataggggaaggggcaagaatggtgagggagctgtttgtcatggcacgggaacatgctcaacaaggtagacccattagtgtcactgatgatggtggagaaagtaccagattcaacttatgagatgattggtggactggacaaacagatcaaggagatcaaagaagtgatcgagctgcctgttaagcatcctgagctcttcgaagcactgggcattgctcagcccaagggagtgctgctgtatggacctccaggcactgggaagacactgttggcccgggctgtggctcatcatacggactgtacctttattcgtgtctctggctctgaactggtacagaaattcataggggaaggggcaagaatggtgagggagctgtttgtcatggcacgggaacatgctccatctatcatcttcatggacgaaatcgactccatcggctcctcgcggctggaggggggttctggaggggacagtgaagtgcagcgcacgatgctggagttgctcaaccagctcgacggctttgaggccaccaagaacatcaaggttatcatggctactaataggattgatatcctggactcggcactgcttcgcccagggcgcattgacagaaaaattgaattcccaccccccaatgaggaggcccggctggacattttgaagattcattctcggaagatgaacctgacccgggggatcaacctgagaaaaattgctgagctcatgccaggagcatcaggggctgaagtgaagggcgtgtgcacagaagctggcatgtatgccctgcgagaacggcgagtccatgtcactcaggaggactttgagatggcagtagccaaggtcatgcagaaggacagtgagaaaaacatgtccatcaagaaattatggaagtgagtggacagcctttgtgtgtatctctccaataaagctctgtgggccaagtcaaaaaaaaaaaaaaaaaaaa

SEQ ID NO: 17 - DNA, 611 bp, Homo sapiens
  Genbank Accession No. AI126004
ttttttttttgttaacattaagcccatttattaacaagtatttggcccaataattatagttaggtaaataaaatacttcagaactattttataggctgccttacaaataagataactgtcaaagatgcattctctttatgtgaaaagttacacatgttctaagtttaacactccacaatgaactgtgaaatttccctaagctaatacttaatattggaaaacttcaacagaatattggaaaacttcaacatcatctcttttctcaagcaccttcatattgctcccatagttcttataaattgtccaataacccagacaaatttccttgtggacaagaatttttaaaatttaaagtattatatattaatacaatgactttagttaaaagtaaaaatttattgatccaaaactgccaaaaaccttatgctaagcaaaaacttcatttaagcttaatgctctttttaactcctgcacgaatgccagataattcaccactataacgttgctcttctttacgaacttcacgaacctggcctcttcttctaattttggctcttctgaacttctctctgtgtttcactctgggattgcgatcaatcttctttctccta

SEQ ID NO: 18 - DNA, 633 bp, Homo sapiens
  Genbank Accession No. AA149864
gtcatcatctgctctgagtggaaggccgttcagagaggctagaggttcttattctggctataaattatgtgagtaaaattgtgctaaccagttaaaagtactgtacacccatgctcaatatatagtcctggaaatagcaattgaaacatgtcttctcacaagagaaaatgacagttttaatgatgtatttgatgaatttaaactttaagtcaggtgctgcaaattggaaagaagacttgtggtgttttaagttgctgtggacacttttaagaaacttagaacccatggaacccttgtttatcgccatgcaaattacaatcttgaatgagtgttttttaaaaataaagtattagaaaaatgtgtagtaaagatgtaaaattaaaaaatggaattctccattaactgtggattttactaaatagaattactggtgaagcagatttatccatcgagactatctggtatgcgttatgtatgtagtctgttgctgctgaaagatgtctgtgtgcctgtatcaacatgtgacttcatgtaaagtttctttgtgttcacagttcttagcaaatgcagttacaatccatagatagccagcagtggatgttactccaggaaaatgcaggattaaaatgtcct

SEQ ID NO: 19 - DNA, 3064 bp, Homo sapiens
  Genbank Accession No. L27213
cgagtcggtgacaagttggtgccctgccatgccagctggcctggcccccgtccttcgcaggaagaagaagaagaaaaagctggaccggaggcctcatgaggtgttcgtggagctgaacgagctgatgctggaccgcagccaggagccccactggcgggagacggccccgtggatcaagtttgaggaggacgtggaggaggagacggagcgctgggggaagccccatgttgcctcgctctccttccgtagccttctggagctcaggaggaccatcgcccatggagctgccctcctggacctggagcaaaccaccctgccaggcattgcacacctcgtggtggagaccatgattgtgtctgaccagatccggccggaggacagggccagcgtcctacgtaccctgctactgaagcacagccatcccaacgatgacaaggacagtggcttctttccccgaaacccatcgagctccagcatgaactcggttctggggaatcatcacccaactcccagccatggccctgatggggcggtgtctaccatggctgatgacctgggggagccagccccactctggccacatgaccctgacgccaaggagaagcccctccacatgcctgggggagatggtcaccgggggaaaagcctgaagctgctggagaagatccctgaagatgctgaggccacggttgtgcttgtgggttgtgtgcctttcttggagcagcctgcagcagccttcgtgcgtctgaatgaggctgtactcctggagtctgtgcttgaggtccctgtcccggttcgcttcctcttcgtgatgctggggcccagccacaccagcactgactatcacgagcttgggcgctccattgccacccttatgtctgacaagctgtttcatgaggctgcctaccaggcagatgaccggcaagacctcctaagtgccatcagcgagttcctggatggcggcattgtgatccccccgtccgaggtggagggccgtgacctgctgcgctccgtggctgctttccagcgagagctgcttaggaagcggcgagagcgtgaacagaccaaagtcgagatgaccacacggggtggctacacggcccctgggaaagaactgtctttggagttggggggctctgaggcgacccctgaagatgaccccttgctgcggacgggctcggtatttggggggcttgtgcgggatgtgaggcgccggtacccccactaccccagtgacctgcgagctgcgctgcactcccagtgtgtggccgctgtgctcttcatctacttcgcagccctcagccctgccatcaccttcggggggctgctgggagagaagaccgaggggctgatgggcgtgtccgagctgatcgtgtccaccgctgtgctcggcgtcctcttctctctgctgggagctcagccgctgcttgtggttggcttctctgggccgctgcttgtgtttgaggaagccttcttcaagttctgccgagcccaggacctggagtacctcactggccgggtgtgggttggtctctggctggtggtcttcgtccttgccctggtggcggctgaattcatcttcctggtccgctacatctcgcctttaacccaggagatctttgcctttctcatctcactcattttcatctacgagaccttctacaagctctacaaggtgttcacagagcacccactgctgccgttctacccccctgaggggtccctggatgctggtctggagccaaatggctgtgccctgccccccaccgagggccccccaggccccgagaaccagcccaatacggcactgctctcactcatccccatgctcggcaccttcttcatagccttcttcctgcgcaagttcaggaacagccgcttcctggggggcaaggctcgtcgcatcatcggggactttggcatccccatctccatcctggtgatggtcctggtggattactccatcacagacacctacacgcagaagctgacggtgcctacagggctctcagtgacctctcccgataaacgctcgtggttcatcccacccctgggcagtgcccgtcctttcccgccgtggatgatggtggcagccgctgttcccgccctcctcgtcctcatcctgatcttcatggagacacagatcacggcgcttatcgtcagccagaaggcccggaggctgctcaagggctccggtttccacctggacctgctcctcattggctccctggggggcctctgtgggctgtttgggttgccctggctcacggctgccacggtccgctccgtcacccatgtcaatgcgttgacagtgatgcgtactgccatcgcgcctggtgacaagccccagatccaggaggtgcgggagcagcgggtcactggtgtgctcatcgccagcctcgtgggcctgtccatggtcatgggggctgtgctgcgtcggatcccattggctgtgctctttgggatcttcctgtacatgggggtcacgtccctgtctggtatccagctgtcccagcgtctgttgctcatcctcatgccggcaaaacaccatcctgagcagccctatgtgaccaaggtgaagacgtggcggatgcatctgttcacctgcatccagctgggctgcatcgcactgctctgggtggtcaagtccacggcggcctcactcgcctttcccttcctgctgctgctcacggtgcctctgaggcattgccttctgccccggctctttcaggacagggagctgcaggcgctggactcggaagatgctgaaccaaacttcgatgaggatggccaggatgagtacaatgagctgcacatgccagtgtgaccctgaagacagtgcccctcagagaccccaagaccttagggattgacacctgggcctgggcctcagcagagagccctgggctgggggctcctcaggacccagagatgtgcctggaaccactcctgatgccatggctagagtggccccccctgacttctgcccggggtgttgacctcgcctcacctttcacagaccagaccggcacaggctttgagctcattataaccacactccttg

SEQ ID NO: 20 - DNA, 83120 bp, Homo sapiens
  Genbank Accession No. AL035398
agagaggtcccttgtcccttctactgtgtgaggactcagaaggtggtgtctatgaagaagcaggccctcaccagacaccaacatgtctgctgccccttgatctgggaccttgcagcctctagaactctgaaaaatcgatgtttgttgttttataagccactcagttggtggcattttgttagagtagcctgaacacggactaagtcaaacagaagaacccacaaaccagctacagagttgggcatttggagaaattcaaaaatgagtcagacataactccttattcttgaggtgccctaagagatgggacacagcagctgcccaggtgcattagtttgttctcacattgctataaagaaatacctgagactgggtaactcataaagaaagaggttgaattggctcacagttgcacaggctggacaggaagcatggtgctggcatctgctcagcttctggggaggcctcaggaaacttacaatcatggcagaaggtgaacgggaagcatgcacatcccatgactggagcaggagtgagagagagagggaaatagagggaaggtgccatacacttttaaacaaccagatctcatgagaacacattcactatcaagagaacagcaccagtggggaaatccgcccccatgatccaatcacctcccatcaggctccgcctccaacactgggaattacaatttgacatgagatgtgggcagggacacagatccaaaccatatgaccagattaatacgatttgaggcatcacgaggtcattaaagagagggaataaaagactggggctccaggaagaaggctctggaatccagcagagggtcaaggaccagcttgtaaagctggtggtgcctgagaagtacctaggagaacatagatgctgtgacgtttgatgtagctgttttttgttttgtgttttggtttttgagacagagtctcactctgttgcccaggctggagtgtgcagtggcgtgatcttggctcactggagcctccatctcccaggttcaaatgatcctcatgcctcagcctcctgagttgctgggattacaggtgcacaccaccacgcctggctaatttttgtgttttcagtagagacagggtttcaccatgttggccaggctggtcttgaactcctgacctcaagtgatccaacaacttcagcctcccaaagtgctgggatgacaggcatgagccaccatgcccagcctgatgtagctgtttctgtgcacattatttgctgtggggtatattcagatttcttaatacaagatgattctttgcctcatgacttacacaccattttctatttaatttcagctatgatattggaaatggacatgtcttttcaaggaaaataaaagcaggctttctggaatggcgacttccaaacatatttgtcaatttaaaggagctgggagtggggaccctatgctccgtaagcactctcttagctgttcttggctgtgctccccgcttcagcttcacactgcccttgctgtgaagggagcagcctgggccgggcgcggtggcttacacctgtaatcctagcactttgggaggccgaggtgggtggatcacctgaggtcaggagttcaagaccagcctggccaacatggtgaaactccatctctactaaaaatacaaaaaattagctgggcatggtggcaggtgcctgtaatcccagctacttgggaggctgaggcagaagaatcgcttgaacccaggaggcggaggttgcagtgagccgagattgcgccattgcactccagcctgggggcaacaagagcaaaactctgtctggaaaaaaaagaaaggagcagcttggcaaaccccaccttgtcgcttttgtgagtgcctctgaccctttggctgccaggacgggcgtattttatggaaatgctaagcaccaacagagtaaagtggtttggtttttcacagtggtgggagataatagctccaaattgtctttttcagcactgagtgaagaaatgaaagacaaaggtggatacatgagcaagatttgcaacttgctacccattaggataatgtcttatgtaatgctgccctgtaccctgcctgtggaatctgccattgcgattgtccagaggtgagcattttaggtggctccgtgtcttcctcacagggttgatatgaggatgaaacaagatgatagatcatggtggcatgtagtctgggacctggattgtcgtgccacagatcacagctcacagtctatgtgcaatgcccctgaatgttgcccacctgtcctcaagccacacatgcacctgtaactcagtgcaagcccagaaactccccgtggggactcctagagctgtcagtggcctcacatagcagctggtccagtctcttgtgattgcccaaggaaactgaggcctggagagcttggggtcactgctctgaggccatagagatgcctagtagaagggccaggcctagaagcaggatccttgctgcccctctgagctgtttccatttaaaatcacatgaaggccggcgccgtggctcacggctgtaatcccagcattttgggaggccaaggtgggtggatcatgtgaggtcaggagtttgagaccagcttggccaacatggtgaaatgccatctgtactaaaaatacaaaaattagtggagcatggtggcacgtgcctgtactcccagctacttggaaggctggggcagaagaatcgcttgagcctgggaggcagaggttgtagtgagccaagattgtaccactgcactccagcctgggtgacaggagagaaaccctatctcaaaataaaatgaaaggtaatgaaatgaataaaataataaatcaagtcacggccgggcacggtggctcacacctgtaatcccagcgctttgggaggccgaggtgggtggataatgaggtcaggagttcaagaccagcctggccaacatggtgaaaccatgtctctactaaaaatacaaaaattagctgggcatggtggtgcatgcctgtaatcccagctactccggaggctaaggcaggagaattgcttgaagcaggacctaggaggcggaggttggttgcagtgagccgagatcatgccactgcactctagcctgggctacagagcgaaactccgactcaaaaaaaaaaaaaaaaaaaaatcaaatcacatgaaagtagaacatagggaattccatctttcgttctaggcatagtttgttaatatgattcagagccagcagttaggagaacacagtgtgactctcctagaacttcttgattgggcttcctctgattgggtttcctctgattgggcttcctctgaaagtgggggggatggggggtggggagcagaatggtcagagcttggctcagcagtcagactgctcttcttcaaatcctggctgcattgcttactacagctgtgtgactccagatgactgaatccacctctctgtgctgcagcttcccgtctagagagatcacctggagcagagggtggtcaggagactcaatctggttactgactcacagtgcaggagtactcatcccatagtaagcatccagctagagatgttgatttctattttcaggtaataatgatgatcgtaaaattagagacagataaaaggtatgggcattaggccagggcactgcaatttctaagctgtgtgacctcaggcaagttactcgacttctctgagcctcagcggtttcatccgcaatatatggataggaaaaccgacctcagtgggttgtctgacagtggagggcacttgattaaaaaaaaaaaaattaccctggtctgaatattaccctggactgaaagaaaaatattgagctaatacaggcatcaggaatggggctgcagggagtccagggaagggagaacgaagagcctgaaggtgtgaggaggtgcgagtgctgatctgtctgctacaaagaggctgctgagcctcctgtggatgtggccctggacttggcagtttaatacctgagctgttaaaataacctcagatgctgtgttctttaaggggtaggattcagattcctgctgaaatgcttctgaaagggagggaatgagccagcccatccccagttgctttttaagatcattgggaagttctggtcttgccatttgtccctggaccactcttaggtcctcctgccccacttccatctgggtgtgtgccctgggctgtccaccacacagctacatcctgccatcttccctcctggagccactgtgccatgcatggatctgtagcttcatttttcttggcttttccctggtttttctggagcagagtctctagtaaactcccaaggaagaaaacgtttgactttatgtgtgttgggaaacgtgctttttttctattacatctcagtgataggttggccatgtctagaattgcaggttgaaaatcatttcctctcagtatattggttagtgagaagcctgggactgagacagtcacattctcacttctttgcaggtgagtgctcttaggactgtctttttatcccttatactctgaaatgtcatatgtcttggtgtaagtccttatttcagttattgagctggacaagtactggagaccccttcagtcaaagccttctgtcattctccagctctaggaaattatcttctattgttatttctgttattccttcccttccattttcttttttctttttttttttttttttttgagacagggtcttactctggtgcccaggctggaatgcagtgacctgatcatggtacactgcagcctgaacctcccagactcaagtgatcctcccacctcaacctcctaagtagctgggactgcaagcacacatcaccacacccaacaaatattttttaaaaattttgtaagatgggatcttactatgttgcccagactttttcttcctcttcctggggctcttattaggaagatgtttgacttcctgggttggattcctgtctccgtgtctgactttctctctttgtcatatttttcatcactcgttgtctttttgcgtctgctctgacagatttcctcaaattttgtcttctagtcctatcctacagtttttactttcagcaaatataatttaatctccaagagtactctcttgttcttttttcttagcattctgttcttgttttatggatgtaacattctcttggaatatttgctgtcctctagatcatcccttctccatttcttcttgggctagtttttctgtttcttcatctttctcttttatgctacttattctgggcgtgttcttggtgggttttttcccatatagcaacagaggacttggagctcagggagaaaagggtaggtgcatcacctggcagagctcccagacagtgacaggcaggctgcgggaaggatgtctacttggcggtgctaccgctttcctagaaaccctttccctggagctggttgaactgttgggttttgccctggtggtgaacgctggctccccgtgctctgcctgtttcatcaccagccccctccccttctgcctggggtccagtaatctgttgaaatatatatcttgctcattggtgagctcctgctccttcctcgttgctcttgcagatttatcacttctcgtaaggctgcgcttgtacttcgggattttctctgtgccacactgggaaacatagggtggttgcatgctgcagtcctgagcacttatttcactcacatctttacacgaagatttggtgggtgtttactttgtttttagtaagttagtctgtcatgtcctttgatcctttttttttgttttttgagatggagtctctctgtgtcctccaggctggagtgcaatgtcgcgatctcagctcactgcaacctccacctcctgggctcaagagattctcctgcttcagtctcctgagtagctgggattacaggcatgtgccaccacacctggctaatttttgtatttttagtagaggtggggtttggcatgttggccagcctggtctcaaactcctgacctcctgacctgcctgccttggcctcccaaagtgctgggattacaggtgtgagccaccacacctggccctgattaatcttttaatgcccagtctctccttcaaaagccggctcctttctctccctcgccttcctagattccttctccactccccaggatcagcctcctcctccccaccccaccactgccggggggatgtctgtggtcaggcatttatcagagaccctgaggtgggggtcctttatgtgtctgggggatggagagtctagaggaggtagcgttcagacctctccatggtgcctctgctgggctcacatgtgaccaagcacagcaaaccatgaggcaggggatggtcttgaccatgagagcccttgcagcagctgccatgggcctcagctcctctccaagctgggaagagccctgaaaagccaaggtgtttttttttccctctttatttcagtgtaagtcccttgagctttcttgaaccagaagtgggctcattttgctttagagatttcaggtgggcttgtccttgtcctagcatcccagatccaccttctgggaagtcatcagattggaggtgatgttggcagcttttgtaaacaaagggtagtgttgtaagctgttgtgtctgcctatgtgtgtgtttgtgtacttggtctcatctctgcagactggtgacatggcttccagatatgcccgacgatgtcctgtggttgcagtgggtgacctcacaggtgttcactcgagtgctgatgtgtctgctccccgcctccaggtaaatactttggctgtgggtgtgtgggccggacgggcacctctctcatctgatgaggcctcacacgacattctagaaacagctggctgaacaccaagcaaggagcttgcccttgggtgtggggaccctgtctcatgggaggcagctgagtcagtcagaggtcctggcacacctgctgagagctgccacccaggccaacctgaaccggagcctgggaagacttcccgtcggatgagtctctttgagtgcagcattgatggtggaagagcagagaggccccagataagcagggaaaggtgcttcagacagagtggctgggatgaggactggggagtgtcagatagcgctggcgtgtctgagcgaaggagctctggcacccatggcacaggaaggaggtgggaccctggaggggcagggctagcagagctcctcggagcgtgtggctaggtgcctggtaatgcaagccccctgtcctccaccctctgttgtactgagtcacagtctccggggtgaagcctagcagtctgcgttgacaggccccaggggatgccgctacttcctgaattctgaattctggaaactgagccggagttcagggcctggctcccattaccagggttgggcgttatcctgaaaatcataggccttggtttcctcacttggctaacaggggtgatccccatcccctcaatgggtttccgtgagctcctgagagcccgtagcatggtacttggcacatgctgggcatcaggaggtatggcctctcttgctattgttgttattggtagacacagaaggatttaaaagtaggggaatgcaaagatccgatttgctagggaagagggcagtagtggccaagtagagggtggatcctgggccctggctggcagcaggcagcaaggggggctgccagggcccaggcagggacgatctgtagaccgagaggcttcctaaggctcttggacaggaggaggtgtcggttccaagcctaaggagtggggcagccctggtgactggtggtcagtggtgccaggcggtgggtggtaggacaccctggcaggcaagtaggtttgtgtgggggaaactgataggcccctccagggattcgttggtggacaacacctgtgatgtccagtgggaggtgtccaggtagctgggagggccacaggcttggaagacctaggtggtgacatcagcccagcactgagggctagaagaagctgtgtctctggctgtgacggcaccctagagtgtgtgtggtgccctctactggccggcaatgtgggtccaccgtagctcagactgcacactgcagcagcgggaacggcctctaagccaacttcctccatgtgtttcaggtcccaaatgccagtgagcagccaacaggcctccccatgcacacctgagcaggactggccctgctggactccctgctcccccaagggctgtccagcagagaccaaagcagaggccaccccgcggtccatcctcaggtccagcctgaacttcttcttgggcaataaagtacctgctggtgctgaggggctctccacctttcccagtttttcactagagaagagtctgtgagtcacttgaggaggcgagtctagcagattctttcagaggtgctaaagtttcccatctttgtgcagctacctccgcattgctgtgtagtgacccctgcctgtgacgtggaggatcccagcctctgagctgagttggttttatgaaaagctaggaagcaacctttcgcctgtgcagcggtccagcacttaactctaatacatcagcatgcgttaattcagctggttgggaaatgacaccaggaagcccagtgcagagggtcccttactgactgtttcgtggccctattaatggtcagactgttccagcatgaggttcttagaatgacaggtgtttggatgggtgggggccttgtgatggggggtaggctggcccatgtgtgatcttgtggggtggagggaagagaatagcatgatcccacttccccatgctgtgggaaggggtgcagttcgtccccaagaacgacactgcctgtcaggtggtctgcaaagatgataaccttgactactaaaaacgtctccatggcgggggtaacaagatgataatctacttaattttagaacacctttttcacctaactaaaataatgtttaaagagttttgtataaaaatgtaaggaagcgttgttacctgttgaattttgtattatgtgaatcagtgagatgttagtagaataagccttaaaaaaaaaaaaatcggttgggtgcagtggcacacggctgtaatcccagcactttgggaggccaaggttggcagatcacctgaggtcaggagttcaagaccagtctggccaacatagcaaaaccctgtctctactaaaaatacaaaaattatctgggcatggtggtgcatgcctgtaatcccagctattcggaaggctgaggcaggagaatcacttgaacccaggaggcggaggttgcggtgagctgagattgcaccatttcattccagcctgggcaacatgagtgaaagtctgactcaaaaaaaaaaaatttaaaaaacaaaataatctagtgtgcagggcattcacctcagccccccaggcaggagccaagcacagcaggagcttccgcctcctctccactggagcacacaacttgaacctggcttattttctgcagggaccagccccacatggtcagtgagtttctccccatgtgtggcgatgagagagtgtagaaataaagacacaagacaaagagataaaagaaaagacagctgggcccgggggaccactaccaccaatgcgcggagaccggtagtggccctgaatgtctggctgcgctgttatttattggatacaaagcaaaaggggcagggtaaagagtgtgactcatctccaatgataggtaaggtcacgtgggtcacgtgtccactggacaggggtcccttccctgcctggcagctgaggcagagagagagaggagacaaagagaaagacagcttacgccattatttctgcatatcagagacttttagtactttcaataatttactactgctatctagaaggcagagccaggtgtacaggatggaacatgaaggtggactaggagcgtgaccactgaagcacagcatcacagggagacagttaggcctccagataactgtgggcgagcctgactgatgccaggccctccacaagaggtggaagagcagagtcttctctaaactcccccagggaaagggagactctctttcctcgtctgctaagtagcgggtgttgttccttgacactttttgctaccgctagaccacggtccgcctggcaatgggcgtcttcccagatgttggtgtcaccgctagaccaaggagccctctgatggccctgtccaggcataacagaaggctcgcactcctgtcttctggtcacttctcgctacgtcccctcagctcctatctctgtatggcctggtttttcctaggttatgattgtagagtgaggattattataatattggaataaagagtaattgctacaaataatgattaatgatattcatatataatcatatctaagatctatatctggtataactattcttgttttatattttattatactggaacagctcgtgtcctcagtctcttgcctcagcacctgggtggtttgccacccacaattttccacatgcttctggtctgcgttctttttttctccttgcatcttctcattctctgatcaccccaacctctctctgtcttccctactctgccagccttgatggagacaagcccttgagaccagaactcaccttttccccaggtgtgataatctattagcaaggtcagtgtaatatgactgacggtgaaacgtgtatttttttctatttatgcatttgagtacagtacgtacaagaaaaataatggtgtgctcaaactgttaaatgttggaaaagaaagatacaacccttacccatattgtgtaagtgccctggagtagaagaaccagcaagctcagacaaagcacttgactgagaagacagaccctttaaaggaaacgggttctagggacaaactctacgtgggcctgttctcttgataagaccgtgaactctttgagaaaagaggctacttgtgaaaataatgagcccccttcggggcaggagttccggggtttgaacctgccttcttacatcttgagggctaagtgagttcccaaggcctctgttcagtggttgctccgtcagtgagctcaggtctggtgagtggcagggtcttccacccccaaccccaccgggtgtcagagcaagacactgtcccccatggagctggaatggggtggaggagcccacatctggcacccacgtggcctccttgtgacggacccacccttgcaatgttggaaaggaaagttacaagtttcttttcccaagtttcccagtaggctttgttctgttagcttcacgccttcggtcattagcagacataaataaactttaaccattgttttttatcttttttttttttttttgagatggagtttcactcttgttgcccaggctggagtacaatggcacaatctcggctcaccacaacctccgcctcccgggttcaaacgtttctcctgcctcagcctcccaagtagatgccaagcctcccaagtagatacaggcatgtgccaccacgcccagctaattttttgtatttttagtagagatggggtttctccaggttggtcaggctgatctcgaattcccgacctcagatgatccacccgcctcggtctcccaaagtgttgggattccaggtgtgagccaccacgcccggccaacagtattttctaataaccagtatatttccatatacatgtgtacatgggtattgtgattgttatcaggaaaaaatatattaaatggctgataggagaccatgggacgtattttctttctgcttttaaaaattattcaggccgggtgcggtggctcatgcctgtaatcccagccctttgggaggccaacgtggacagatcacctgaggtcaggagttcgggacaagcctggccaacatggtgaaaccctgtctctactaaaaacacaaaaattagccaggtgtggtggcaggcgcctgtaatcccaattagtgggttggctgaggcaggagaatcacttgaacccaggaggtggtggttgcagtgagctgagatcgtgccactgcaccccagcccgggtgacagagtgagactccatctcaaaaaaataaaaaataaaaaataaaaaataaattattcaatcttctttatttttattgttttaatgactaaagttatttttagtagaaacagggtcttgctatgttacccacgctagtcttgaactcattggcttaagcagtcttcctgccttagcctcccaaagtgctgggattacaggcatgcctggctcctccaccttcttaaaataagcatttgttttataattttttccaagtgtgtatcaagataaggaaatcaggaagtgtaatattcttatagaaatggccaaggcctcccccttcacctgtgcctcagatgctacccaatcccgccttctctgtccctccagaaggcaccctttgcttaggcctccctctcttcctgaaccacctctggaagtttcttattggcctatgaatgcattcttatttcttcttatcaaataaagccttccctttaatttatggcacatttatgctttggaatccactctcaggaataatcagtatgtagcatattacacgtcaggcggcaacattctttttttttttttttttttttgaggcagggtcccgctctgtcatccaggctagagtacggtggtgcaatcatagttcactgcagcctcaacttcctggggtcaagccatcttcccacctcagtctcccaagtagctggaaccacatgtgtgcactaccacacccagctagtttttttttgtagagacagggccttgccttgttgcccaggctggtctccaagtcctgggctcaagtgatgctcctgccttggccttccaaagtgctaggattacaggtgtgagctaccatgcctggtccaacattcttcatttggtaaatggctaaacttagtgcagagtatgagcctgattttgtttaaaaaaaaaatgtgtgtgggtgtgtatatgtatctttgagtgtatataaaaagactgaaagagggctgggcacggtggctcatgcctgtaatcccagcactttgggaggctgagggaggcggatcacttaagtcaggagtttgagaccagactgggtgaaaccccatctctactaaaaaatgcaaaaattagctgggcatggtggcgggcacctgtaatcccagcaaccagggaggctgaggcaggagaatcacttgaacccaggaggtggaggttgcagtgacccgagatgcagtgagactccacctcaaaaaaaaaaaaaaaaaaaaaaaaaaagactaaggatatatatcaaaacccttatggcagactgttatttgtaattgtattttatttgtcgtgcttatatgtgttgcccaagtttctatggtgaacggtatgcatcactttcagcatgagaaaataactcctaataaacgtgattcttaaaggactctccctgtgtacatcctttccaaggagccccgatgtaccctgcttccctcacagccaagctcctctaggacagtcctccctagggggttacggccttgctcctttgatggccctgccacagcccaggggctctttcctgggcactgtggaccagaccctcctagaatccctctccttcctcaaccccagactctcaggcaccccatctctatcctggacaccgagcttgtcaccaaggcccagctgcagctgctgtgctgagtgtgcacagccacctctgggaaggctgtgctccccctgaggacctgggtcctcagtctctgactctggggggattcggggccacctacccaccttctggctctactcaacaccagcagcccctcaacatcaagccctccagctcacagcacctcagctctgcagctccaaagccaccccagcctggaactggggactcagggagactccctcgctccaactccagctggtcccagcactgcctctgctgtcatctctggaatctggtctcttgtccccagcccactgctgccagggtcagccctggtactttctacctgggttgctgcaacagcttccctctgggggccgcctgttttcagccttgggcccatctgccctccacgttgctgctgggcaagctgaggccccaccacacccccagcaccttcctgtgcctccaggccttccctgccagtacaccccctccagcccctgaagccctgtcagcagctcagagcctttccccggagtcaccctgcttcccgaggggacccacgttgctgctgggcaagctgaggccccgccacaaccccagcgccttcctctgcctccagactccctcctagcacaccctctccagctcctgcagtcccaccagcagctcagagcctcttccctgagtcaccctgcttcccgaggggacctccacccagggaatgcccttccaggctcctctcactgattcaccttcactcctcagaggtcaggtgctggctgatagaggcaatgaggtgagcccctcacctcagggcgctcagcccctgggaaggtgatgaaaaagggcaccccaaggggtgtcccaaactaataggtggatgactcccgtgggaagatccagtctcccctccccatggccaattaaatttggggtgtttttctttacttttttgaggcaaggtcttgctttgttgctcaggctggagtgcagtggcacaatcacagctcactgcagccttgacctcctgggctcaagcaatcctcctccttcagcttcctgaggaggttttaattaaataaattaaaagctaattaaaaacatttttttttttttttggtagagatggggtctcactgttttccaggctgatcttgaactcctggactcaagcaatcctcttgcttcagcctctggagtagctgggactacaggtgcttgctactatgcccagctattttttttattttttatttttattttgtagtgatggggtgtgtttcctaggctgatcttgaactcctgggctcaagcaatcctcctgcctcggcctcccaaagtgctgagattataggtatgagccattgcactcagcctggattgtattaaggaaactagcttagtcactaaactgcacttctacttctgattggacaagattggcactttgtaaaccaggttttctgggatgctggtttccttgaagatgtttctagctggtattatcatataggataggctgagttatgctgcattaacaagcaaaacccaaaaggtttatttctcattcgtgctgtatgtccagcaaatgtggctgggcttcattttaagtcgtcctcactcccagacctagcctgtaggtgccccaccattgggggatcgctgattgccatggcatggttagagaaatatggttcttaaaggttctcatccagagtggcacatctctcttctgctcacaatgtgttgaccaaagcaagtcacatggatgggagcagggagatgcaatcctctcttgtgcccaaaagaggagcacaagaaatactgttgaatagcattgccagaggccacagaaaaatctccaaaaccaaaaatctctatgacccaataaattcaagaaatactgggttaaagagaaggaaaccagtatatgcactgtgagacttctcagtctttattatatacattgtaaccatcacagacagcctttaatatatacatcttaactatcacagacagccttcagtatattatatacattataaccatcacagacagccttcaatatattatatacattataaccatcacagacagcctttaatatatacattataactatcacagcctttaatatattatgtacattgtaactatcacagcctttaatatattatatacattgtaactaccaccgcctttaatatagtatacccattgtaactatcacagcctttaatatacacattgttaactaccacagacagcctttaatatattatatacattgtaactatcacagatagcctttaatatatacattgtaactatcgcagacagccctttgtttgagggtaaagcatgtgactttgttttcctcctgttccctctgccatcctccagggcctcacacacaataggctctcaaatattgcaaccatcttgcactctgcagggcaacctcatggcttatcctttacaagtggcatactcagaagaataacgaggaagaaagtgtaagaaaagagggctgtttgggcaaagaaaatctggttgattcgatgtttacagagtgccaggccccatgcttgatctcatgaggtcctcagaccttatgccttaggctatcatttccattttacagataaggaattgaggctcagggaactgagagtgagggaagggcaggggcctggctggtgctgggttctgtctctttccaggcaagaagggacatgtggacttgaatatttagctcagggatgttggaggcctagggtgctgctatttaaatggaaagggtgtcagggtttctgggagggagtaaggggacagaagaaaagatgcttctgttatcagaagggagtggcaacgtgacaggatactgaagcttcctcttttcttattctgtctcgtttctttcaatatacttatcacaatgtgtggctgatatttattatgcctttttcttttctattctcttttcttttttggggcaggggagctgttttgccaccaaaacctaagcccatggccgtcttcttcaccgatatgactggttctcatgcaagcacctagatggccctcgcccataattactggctgcagttgagtgaatagcggtcattatctagcgcccaccgtgtgcccggcccggtgccgggagctttgctttccgtctctcacattaggcccttttaatccaacccccaagaaggagacacgaccatcgtcttcattttgacgattttgagtgggggttccaatgacttgaacgaaaggagccagggcaccaatccgggtttgtgtagcagatgccggtgagacccgactactgcagctcgggggtggccggacccggggtctccccgctgagtctcggggcttcaccggcgcgtgggcacaagcacagcaccctcgggtcgggggtgctaggagggctaagagcagcaggacttgagtgacacccaacagctggtctgatctctttcaggcgccaacgccagacgccagcctcccagcggaccctctcgcagacccaccttcaccccacgcgccctccccacacagtccacgcgccagcagcttcgcggatgtcagtgaacgctcttcgcttgcgcagtcagaacgtcctccgcggccgtgtgaacatccgggtcacaggcgcataaggaccaatgagcgaaggcatcgcgagccagggggcgcggagaaggcggggaatcatggccgcccccagtgttccgcgtccgggggtttgtgggagttgccttgacctgcagctccgccaccgcggacccgccttctgccctcagcagcagacgctctgtcccgcccgggcagctctgcgaggcagcggctggagagggaaccatggggactgtgcacgcccgggtaagaggcccagcgacccgcgggctgcgaggcctctgggccagcagggcagacgggcgccgcggggagacgctctcgtggctgcgcccagggttcaccgggagagagggtgccaagggtgccgggctgggtggaggaggccgaaaagaggtcgcctcgggccagcgagtcttgcagaccttcgagcaggaacctgataggagggacgagacttggctaaaggtcactcggcgagtccgtggcccaaaggacttgccaactttctgtgatgtcttagaccgcagtgaatgtgctctttctttgcatgattttaagttttctctctttactggtcacttccagcctttccccgctcagccgctgctggagccattgtaggctcctagagcaacaagaatcctttgaagtcttctaactcaaacctatctaggggtcctcttttctcctgtctaccgtgcaaaagacggaaccgctctctgccttacttcctgttttaaagtggaggtagtaatagtacctacctcgcctggcttaaaagagaattaaaagacaatgcatgtgaatacttgggtgcctggcgcctgactaactgtactggttgccaaactcctctcgtttatcctaatcttaaataagggccagataatgagttacttctccaaggtcacatagttactggttccaaatggcctaaaacctcagatttcctgagtctcagccccaacactacttttctactgcaactttgattgcttcacttgacctgcatagacgttttcagtagttccccattaacttccaaaaaaatttagcacagaagtcgcagcctgacatcgataattggacttaacttccctttatatgtctttcattcagatcaaacaccgtttctcttctgacatctctttgtgtaactgtaataggtctgttgctcagtgtgctcgataagtcaatacactcagacagtgggttgcagcagagaaagagtttcagttgcagggccgctgaacgaggagatgggaaaagagaggaaacctcaaatccatctccttcaggagtttggacctcatgtttttgctgaagtgtggagatccttgattggtggaagagtgaagggtgaagttatgggacagggagatgaggaaacttctcatgctgattccgttcctctgttgcggggaggagtcttcaaactggttggcagcatcagttttgctggaattcgagatctgaaaaacatctaaagcaactcttaagcaaaagccttatgattctaattttagaaatcctatctgtaaaatcaatggggatgcaaatagtatctgtgacttttggttactagcaagtaggtcagagtgcaacctgagctcttcctgccatcttggctcctgtggcggcctgccgggagcaggacttctgaaaggaaatatgtctggaaggctgtggtccaaggccatttttgctggctataagtggagtctccggaaccaaagggagcacacagctcttcttaaaattgaaggtgtttatgcccgagatgaaacagaattctatttgggctcatgtatacaaagcaaagaacaacatagtgactcctggcggcaaatcaaacaaagccagagtaatctggggaaaggtaactcgggcccacagaaacaatggcatgattcgtgccaaattctgaagcaatcttcctgctaaggctgttggacacagaatccgagtaatgctgtacccctcaaggatttaaactaatgaaaagtcaataaatcaatgtggatttgtgctcttgaaaaaaaaaaggcaacttgattaatggttaattgtaactacatttcttttgagatggagtctcactgtgttgcccaggctggagtgcaatggtgcaatctccactcactgcaacctccgcctcccaggttcaagcgattctcctgcctcagcctcccatgtagctgggattataggcgcacgccaccacacccagccaatttttgtatttttagtagagacgggggtttcatcatgttggccaggctggtcttgaactcctgacctcaagtgatcatcccgcctcggcctcccaaagtgctgagattacaggtgtgagccactgcgcccagccaattataactacatttctgtccagatgtccagaataattttttttgttgttgttgagacagagtcttgctctgtgcccaggctggagtgcagtggcacggtcttggctcactgcaagctccacctcccagattcacaccattctcctgcttcagcctcctgaatagctgggaccacaggcgcctgccaccatgttcggctaattttttgtattagtagagacggggtttcactgttagctaggatggtctcgatctcctgaccttgtgatccgcctgcctcggccttccaaagtgctgagattacaggcatgagtcaccacgcctggcctttgtccagaattcttgttagccctgtgaggatggctttatttggaccgcactattgcacttcattccccctcccatcatccatctttagcatcaaaatattcccatcttttagaactccgttctgaaagttctttatcccctgcatttggatgggcttcgcctttccgtgccatttcctttggtgccatttacattttggcttgttttgggtctgtttgcattttcgtcttatcctttgatcctaaatttctagatgtaatggtaggaactgcatcttcttggcttgcatcccctgtggcatcttgaacaaagtaatcactcattatttattcagctcatatttattgatggtctgctatgtgcctagcactgtgataggtgccagggatacagggaaaaaagtatctgcctttatggagctcttttttttttttttttttttgaggcggagtctttctctgtcacccaggctggagtgcagtggcgccatctcggctcactgcaacctctgcctcccgggttcaagtgattctcctgcctcagcctcccgagtagctgggactacaggcgtgtgccatcacacctggctaatttttgtatttttagtagagacagggttttgccatgttggccaggatggtctcaatctcctgaccttgtgatccacccgcctcagcctcccaaagtgcggggattacaggcatgagccattgcgcctagcctgtggagctctttttacattaaaattgctgctgctttgatgagtgttttgagaaaaaaacgttttcagttcttttatttgactttttcatcccactagccagtgtctttcagactttttgatcaagactcaaacagtaagaaaacatttgccatttaccacctagtactcatgcttatgtcaaacaaaattttgtgcaataatacttaatatgtgttatgtattggtatattttcagttttcttttttttttttttttttgagatggagtcttgctctgtctcccaggctggagtgcagtggcgtgatcttggctcactgcaacttctgcctcctgggttcaagcgattctcctacctcagccccctgagtggctgggattacagacaggtgccaccacacccagctaacttttgtatttttacctgagatggggtttcaccatgttgtccaggctggtcctgaactcctgatctcaggtgatctgcctgcctcagcctcctgaagtgttgggattacaggtgtgagccactgtgcccagcctgttgttatattttctcttggattctagtctatctcactttaaagatgctgttcataactcattcatttcatgtcccatacccgctgccttgcacatggtgctcaatacacacttattgactgactggtagaccgatctgcttaaaggttaaaggctacatccaaatatttattttatttttttatattacacacacacacacacacacacacacacacacacactttttaatagaaacagggtctcactatgttgcccaggctggtctcaaactcctgggctcaagtgatcctcctgccttggcctctcaaagtgccagtgctaggtacagacatgagctactgtgcctagcccaaatatatattttagcgcattttaccaaacactcaccccatgcctattattctaggcatagaatttatgggatgtacattctatgagggtgaggttcttataagtgcttgtcacacagtaggatcttaatatttggtgaatgaatgaatattagtaacacagttactaatattttgattaaagtatatttacagggtattaatcctataatgactttagggtttagagataatagaaaaatagatgtaagatgtatgaataggtataagaggttgtaactgtgaaacttcaatggtgtcatctcttgaagtctggattttagattaaagaatacggatggacctggaagatctttggtgaaccttgtttcatgcttggagattttagaaagtgcagtggaattttttcagcgtaatgggatataaacctttgcctagataaagcatttttgctaggcattcagattaagatgagtatgtgcacataatcttactttgttcatatttttgagaaatataataattccattttgaaaatactgcacaacaaaattgaaaggatagagatgggaaattatccattctttgcctaacatgatcttccttacccacaaggacccggtcttggtcactgctgtgaacccagcacccagcacacagtggactttcagtacttatttgttaaatgaaagaacggaaaagtcgttcattccccattccacaaatatttattgagtacttccgaggtgccagggcagataaaaataagaaacaggtcaggcgcagtggttcacgcctgtaatcccagcactctgggaggctgaggtgggtggatcatctgaggtcaggagttcaagaccagcctgaccaacatggcgaaaccccgtctctactaaaaatacaataattagccaggtatagtggcacgcacctgtaatcccagctagctgggaggctgagacatgagaatggctcgaacccgggaggtggaggttgcagtgagccgagattgcgccattacactcaagcctgggccacagagcaagactctgtctcaaaagaaaaaaagaaacagaaggggtacctgtgaaggtcacagacttcaagtatcaagaggtgtccctgtgagagaggaaccgattctccctaaagaagccaggaatggcttgcaggagatgacattaaggtggatcttgaagcttcataggggcaagttattctggaagagggggcaaggtatgcaaaagcctgaatgtgcgggtggagcgggtggtggagatggaaactggagtagcagatgcaggccttacggcagagttccttgtaccctgctaaggaacctagatggatgctcttcctttaggtacttctgaggatcatcagaggcttttagggaagggcctggaaagggaatatgatactggcaggtcagttacctttccggggaaaagtggatagtagcccagaggccccactgctccatatttgcagatgttaaacacacactttaaaagtgcctaattgtagagaattcttggagcaaggaaatagtcagatattggttcacatagttttctccaaacttgctgatttgtaagaatcgcttggtgccacactagaccttctgtgtccatctctaagaggtgactgggaatcctcgtgtggttaagcatgtgtcccagagacttatggtcaagcaaacataggaacaaataagaattgaataaatagtatatgttaaatatgtaagtatatataatacaatgtatacataagtatatttatttttatttttgagatggagtcttgctctgtcacctaggctagagtgcagtggagtgatcttagctcactgcaacctctgtctcctgggttcaagcgattctcctgccttacccttccaagtagttgggactacaggcacacaccaccacacctggctaagttttgtatttgtttagtagagatggagtttcaccatgttggccaggctggtctcaaactactgacctaaagtgatctgacctccttggcctcccaaagttctgggattacaggtgtgagtcattgtgcctggcctattattttaaaatagagacgagacttcattatgttgcctagcatgatcttgaactcttgggctcaagcaatcctcccatctgggcctctcagagtgctgggattacaggtgtgacccaccatacttgacctttctttatgttttatttttactattattactatttttagtagagatgaagtctcctatattgcccaggctggtctcaaactcctaggctcaagtgattatgtgtgtgtgtgtgtgtctgtgtgtgtgtgtgtatgtgatattatgtttatgtagatgtttctcaactataacccaataaactcatcgtaagttgaaatgtaagttgaaaatgcatctaatacacatatccgacagaacatcatagcttagcctagcctactttaaacgtgctcacgacacttacattagcctacagttgggcaaaatcatctaacacaaagcctattttagaataaagtgttgaagatctcatgtagtttcttgagtactgtaagtgaaaatcagaatggctgtgtgggtgctcggagtgcagttcctcctgaacgcatatcactttggcatcattgtaaagtcaaaaaactgtaagttgaagcattgtaagtcagagactgtctgtcttaactatatgtagatatatatagagagatagataaatagatagatacaatgtcttcctggaaatattagatgagacacagcctacagaatgctcattcagaagttgctttaatattttgaggtaatatcaattcagattatagaaagagatgctttcgagtccattttgtgagcatatgcagttccctttaatgtgaatgagactttctgaaatgaaaaaacctgtcagggtctacataaaatgttttcttagtaatctggtttgtcaaggtagctatcctaacatgtgtacaattttaattctaaaaattaggatgctttctgtaacaacaggagagcaaattattttccttttttcccctgataaatgcctttgtggcattttatttttaaatgattttttaatttccatcgggcctcagatgtccagaattaggttggtcataatccttcatccatgacatgtcatcatggatttgtgcgtgacccttttggttaatttttttttttttttttttttttttttttttttagagatggggtcttgccgtgttgtgcaggctagtctcaagctgctagcctcaagtgatcctttctccttggcctcccagagtgctgggattacaggcatgagccaccacactccccctgcccttccctgttaattagtttttatattttatttagtgaccaccagtgagttaaccaccaagccaagaacttgctcattagcaatagctgacggcacctttttactccctggtccctattcctgcccctacctctacttgaagggaacaactactgaattttctgcagcaccatctaaagacaaaactcaaaggtaagtgtgtgtatatatgtgacaaagtcatttatctgtggtcgctccctccctttcagagtttggagcctcttccatcaagtggacctgattttggaggattaggagaagaagctgaatttgttgaagttgagcctgaagctaaacaggaaattcttgaaaacaaagatgtgagttttctgttgaaggtcttgatagaattgttagtggaaacattcacttccatacaccactagggagatgtaaaggaattaacctgtttccgtgcctttatcaaagaagagtttgtaaccacctacaactagaaaaactgatgactcttagaaaaacagtttctatttttaaagaagtcgctcttccttggtgatttttatgtgggaggtttagacaaacacacttttgtatttatgacaaaaaccaaaaatatatcaaggacaaactcaaactatacttaaatttatatttccattccctactcccagaaaaaagactgtgggtctcaaatgaaacttttcctaaatttattacctttgaaatttgaatatgcttttccagttcttgaagagtttactctctagcgagactgagaggtgagggccagtttgagtcagcatttattgtgccacctgaaatccctgcggggtctttagaggatgcatacagttttgttttgttttttttatttttaaaattttttttgagatgaagtctcactctgtcacccaggctggagtgcagcggcacaatcttggctcattgcaacctctgtctcttgggttcaagtgtttctcctgcctcagcctccaaagtaactgggactataggcgtgcaccaccacacctggctaatttttctgcacatagttattaagtcaggggtcatggccagcattcattcacaggccagtgctcagcatgcatgttaggaaggacagaggggttgtctgtggcctgctcgggtttagggctcacctggtcctgggtcatctggctggagaggatttgtagctcttgttcacagaaagtacataacgagcaaaggggaagtcacagtgactggttttttgaaaaaatgtgaaagatagtaaagtccagaggtaagaagtgtggattattaaacttgaagaaaaccttgacattaattaaaccttgacattgtctttagtcttgacacctaatctgtttgcctcatgtcatccctaatactgtccctgtgtgacttttaggtggttgttcaacatgttcattttgatggacttggaaggactaaagatgatatcatcatttgtgaaattggagatgttttcaaggccaaaaacctaattgaggtaggtgtggtctctacatggtgtgctttcccagtctcttctgaagaactcgtaaatgtccccatgtgaaaccacagagcaccctgcgcccggcctctgtgccggagatctgcctcctaccccacagggagccttggcacccccaagctgaaccccccgaattcctgtggtgcctcctacaaactccatgtattcgcatttgccttttcctcctgtctcttatcctggagaaagaagtgtctgtcctcttgcgttcctcataactctcccagcgccgtaatgcgagccagtggggttagtgcacagccgttcccaggcaccgcgctgagttcctgagagtgtactctcccttgccaaccccgtggggtgggcgcttatctgggacacactccctgtctcctcagggccctggctctgtctgtggagcagtctccttcctgcactgcaccccgtttctcatctccactgtgggtggtttcctctgcctttctgcccgtgggaccatattgaacatgcccagacagaggtcaccagcaacttctttgtggccgggtccagctggctcctttgtcttcatttcacttgaccttgcagcagcagcagtgtttggcactgtggaccattcccacttaaaaaaaaaaaaaaaaaagaatcttttttcaattttttttagatagggtcttgctctgttgcccaggctggagtgcagtgacacaatctcagctcactgcaacctctgcctcccaagttcaaaggattctcacacctcagcctcccaagtagctgggattacagatgtgcaccaccacacctggctaattttgttgtatttttagtagagatggggctttgtcatgttggccaggctggtcttcaactcttggcctcaagtgatccacctgcctcggcctgccaaaatgctgagattacagccatgagccaccgtgcctgcccaaatttgtatttttaaaaaattaaagtaacatgtaagcacagtaagaaggaagcatagaaaatcaaatggtacagaagggtattgaaatgaaaaggacaattttccctaccccggccaatcccagtctgctctccagaggtaacgtggctgacagtttcctagatatgttgtacgagtgtataaacatgtatgttccttaaacagtaatgccattctctacatttttttttcttttctgacataaattatggagatcttttcacatcagtacataccagtctgttggctgtgtggtacctcgttgtacacactttctaacttttttaaatttttttttctgagatagggtcttgctctggtgcaagtcctgctcaggctggagtccagtggcgtgaacacagctcactgcagcctaaacctcttgggttcaagcaatcctcccacctcagcctcctgagtagccaggactgcaggcacacatcaccatgcctagctaatttttaaagtttttctgtagagacggggttttgccatgttgtccaggctgatctggaactcctggcacaaggggtcctcccaacttggccttccaaagtgctgggattacagcatgagccacctcacctggccctctacattttattataaacagttctccattgatgggcatttaaattaattttcactcttacaaacaatgctgtactaaacatcttagtgtgtctttgtgtacattgactgtgttttaggataaattcctggaagtgggattcctgggtcaaagtgtgcgcactctgcagtgttcactgatgcagccgtattgctctgcaaagagattgcacaagtccactgtcttttttttttttgagacggagtttcgctcttgttgcccaggctggagtgcaatggcgcgatcttggctcactgcaacctccgcctcctgggttcaagtgattctcctgcttcagcctcctgagtagctgggattacaggtgcacaccaccacacccagctaattttttgtattgttagtagagatggggtttcaccatggccaggctcgatttgaactcctgacctcaggtaatccacctgcctcggcctgccagagtgctgggattacaggtgtgagccactgcacccggcccacaagtccactctcttacagcagtccatgagacggtctctttcccaaaattgggttttatcacaatttttaaattcttgccatcttgttagaagaaaaatgccatctcattgttttaaattgtatttatttaattatgaatgagatccagcatttttttttttctgtttagtgttcattcatatctctcttgtgaattaacttggtgtcttctgtcttccattgggtcatttggcttttgttgatttgcaatagctctttgcatattgaagaaatgatcgctttgtagcctcagctgaatgcacctgtcctgctttgttatttgcctttgaatgtgtctgtccctcttcttgcgcttgcctcttccctgggctttggtgaccattttcctgggttgctttttctgtctcatcacagtcattctcaggctctgttgtcagctccttttccctgtgagcatccttaagtgttgcttttccacaaggatcagtctcctgggtcatctcatccacatcccaggtttcagttacagcctgaatgcggagggctccccatgctgtctttctagccagctcctctgtgttgagctgtagccctgactatgcaactgccgactcatcgtttcctcatggcattgccacagccgtctccagctccatggggctgagacctgaagtcatcccttttccatctgtctgctctgcctgtcttcccatctctattcctgacttctctaatggtgttcagcgctagcccagtgctaagcgccgagctgcagtttcagtagcttctttcttagcctctttcttcttctcatgcactctacctaggcagtggcagtgtcctctgacccctgcctgtgaaatgtctccagggtctgtcctcccctccaataccatggcctgcactgggcctgccttcaccctagcccctgaggctgttgccagagcaccccctgcatccttcacctgcaggcttctcccatctgggcagaataccgcattcgtctgtgtgatccgttctttctcacaaggaaatgtgatcctttctgtctcctgctgacagttctacagtgactccctttcacctgcagggtggaatctcaactttccttatcacacgagggcctttccttctctgtgtcctcagcacttgctgccttaccttccttctacctgtccttccaataagaatggactccgtgcatcagaaggaactgctgttgacaacaaagaaaggacacaaagttgaacatttctaacagtggttagagatggttagaaaggccgggcgtggtggctcatgcctgtagtcccagcactttgggaggctgaggcaggtggatcacctgaggtcaggagttcgagaccagcctggccaacatggtgaaatcctatgtctgctaaacatacaaaaaaaaaattcgcttggcgtggtggtctgtgcctgcaatcccagctactcagaaggccgaggcaggagaatggcttgaacctgggaggcagaggttgcagtgagctgagattgcaccgttgcactccagcctgggcagcagagcgaaactctgtctcaaaaaaaaaaaaaaaaaaaaagaaaaaagaaaaaaagaaaaaaaaagaaagtaaagaaatgaatagaaaattgaaacaccacaagaagatgtgcatttcttcctcaaagggagagctgaaaaaaaaaatgaaaaggtgtgcagacacacgtgtctttcttttgccagacataaccactgttcacgattgtgatctcttccagcacaacgttctagactgtgtgttaggtttgtgtatctgcgttagtcttgttgtttatacgaaggggattatatgttgcacactcttgatgccaaggcttggttttgcacttgtgaatgggcatttttgttgcccctgtggtgcttttcccttctgtcatgtggctggtcttggtagtgctctctgctcagtgatctgacttagcaccgaatggtaaactaccagcctcgccgtgtggctgccatttgctgtttctgtatcgtagaagaatatattccttgtttttcttcccccattttataggtaatgcggaaatctcatgaagcccgtgaaaaattgctccgtcttggaatttttagacaagtggatgttttgattgacacatgtcaaggtacatattgtggtgtagtatctttataattccctttctgtccatcagagaaaagctgtttttatggccagatgcagatctgggcagcagagcactgctccctgctgtgtagaacctgaaagttgatcaaacaggtagtccttgagttagcagtctcagggaagggattggattttttgtttgtttttggagaggggtcttgctgtattgcccaggctggtctggaactcctgtgctcaagcgatctacctgcctcagcctcctgagtagctgggcttacagttgcgggcctggatttttgtatgaggccggggaggagtggtgaactttgaagatactgttgtgtgaagccctggactccacgggggcctgagagtgactctgaaggagggggccaggcttcgttcacatagcccagcccagcactgtgcagacccccatctcagtgtatgctttaaaaagaggagggaatccaatttccattttagtttttgcccattttcttgttgctgtgtgatactgggcacgtctctgaacatttattctttggctagtgagcacctgcttcgaggccagccctgtgcaggccccagagggagacacagggcgtggtctcctcctgatgggcttcggtcatagcagcacacagggcgtggtctcctcctgaaggggcttcggtcagtggggctgggttgggggcagaccagtaagtgatggcagcacagtcgctatgttgtgctgtaagaggctgagggaggagacagttcacctggccagggcatgagaaggggatcagagagggtgtccctgaaggggcgatgtctgagctgagacctagaagatgcatctgagcgagtatggggagaagggctggtagtgaggtggggtgaagaatgttctggggacagagatagcacttatcaggcccacgccaggggagagtggctcgctgggtggaccgcaagtgtaggaagagctggagtgcaggttgggggaatggagtgctggaagacaaggaagaaaagccgtcaggagcccagaccgtggtgggcctaacatggcgcggtagagttcccactttagtcagaattcagcagacaccattgaaaaataaaatttggaatgaatctgtgaatgagtaactcttcagaattgaatggggggtaattttgagaaggcctagataagacaatataggaaaatgcttgcaaaaaataaggtgctagataaactaaggacattttcattatttggagtatagtttcagccaacactaacagacctaaaagaacagtggcttagacaagagagaagtctccatctctccacacagctgggagactgccaagggctggtgtggtgctgcgttccctgaggtcaccttgggctggcgtggtttcctgtggttccctggggtcaccttgggctagtgtggtgccttgttctctgaagtcaccttgggctagtgtggtttcctgaggtcaccttgggctggtgtggtgcttcgttccctgaagtcaccctgggctggtgtagtttccccccgtggtgtcttgttgctttgtcatccctagggtccctgagtccctctgatggctgagcaccgtgtcctcattccagtattggaggagcacaggagattgagagggcttacccctccctttaagcacctgtcctaggtgttgtacacggcactgctctcatcccattggccaggacttaatcacttggccacacctaggtgcaggggaggctgggaagtcactggacttgagttaataattggtgtatgggtttatcagttgtgacaaatgtaccacacatgcgaggtgttagtaagtgtgtgtcggggtaaggagaggttcatggaaactctttgtacttttgtagtttgtacaaaaactttgtactttctgctttatcttttttttttacccgcaacccactgttgctggcttcaattctttatctttctgtgcgcctaaagctgctctaagaaataaagtccactaaataaaaaataaagtaaaataaaaaataaaaaaagaaaaagaaagaaaagatggtgccaggcggggtggctcacgcctgtaatcccagcactttgggaagccagggcggctggatcatgaggtcaggagttcgagaccagcctggtcaacatggtgaaaccccgtctctactaaaaatacaaaaaatattggcagagtgtggcggcgggtgcctgtcatcccagctactcggaaggctgaggcaggagaattgcttgaacccgggaggtggaggttgtggtgagctgagatcatccgactgcactccagcctgggtgacagaatgaggctccatctcagaaaaaaagaaaagacagtactgaggctccaccctagaactgttggacctgagttcctagagcggtgcctgagcatcagcatattttgacagttgcccagatgcgtctagatgtccagccagggctgagcacccctgtgtacaaggcaggagtcgcctttccttcctgtgagaggccactcattcctacagagcccagcccgctctgcacaaggcaccccagggccatggtgggtcacgaggtggctgcttccagctcagacccaaatcatacctggtgcgggggaggacagacatgcctcttacacacagtaacacagtaaacaatctcacacttatcttcgagggttactgacacccctattgtcatgtgagttccccattttcatgtcggttgaattgagaacccaaatattttcaaggacctctctaataggagaacagattcctttagcaaatattgtttgcttaagtttgcttaagtgaataatgctgcttatagttatctccttgtagcaaaggtagattgtttttgttttttggtttttggttttttttttagagacagggtcttgctctgtcgtccgggctggaatgcagtggcacagtcatagctcactgcagcctccaactgctgggctcaagtgatcttctcacctcagcctcccgaatagttgagactataggtgctcgccaccacatctggctaatttttaaaatttttgtagctatgttgctggggctggtcttgaactcttggcctgaaagtgatcctcccaccttgtcctcccaaagcactgtgattacaggcctgagtcactgtgctcagccctgtttattgatatattctaccagagaaactagaaacattattttttggtgtagaggtgtgcaatattttgattttgtgtgggagtgttcttaaacgtttctcatgattatattattttctaaaggagattgtaaaatagaatgaaccttcagattgctttttgtcttttttagcaatgggaacccaaagtaaaataacatcctggctgtcttattgtttaatatttttttatttaggtgatgacgcacttccaaatgggttagacgttacctttgaagtaactgaattgaggagattaacgggcagttataacaccatggttggaaacaatgaaggcagtatggtatgctacaggctttttactttcttatattggaacttagagttgggaacttttacactatgactgcattctaaaagctgatgatagtttcaaaattcggtcatttcatttaccttaatctgttgttatttgtggtttctctttaatcaccgtagaattggtgataatatgttttgggttgtttgataacaatgttaacgttagtaattaggaatgatttggagaatgttcatattaatagtctcttgttcaaattcctttaaactctaaatctgtaacattcttgagcctcacgggtggcaaggttagcttgtcccttgcattaatcaggtttgaatgcaggatgtgagcactcagcagcggcatgttttaaagaatgaaagtgattacaaatgaaagactagatctgtagttagcatcttccttctgtttttatgaaagtcccttagctagcatagtctttatccaatccaaagttctcattccttcaatgtagaccactgctattttttttttttcccctcctaggtacttggcctcaagcttcctaatcttcttggtcgtgcagaaaaggtgacctttcagttttcctatggaacaaaagaaacttcgtatggcctgtccttcttcaaaccacggcccggaaacttcgaaagaaagtaggaagcccaacagatcattgagtacactggcctgatagaaaagttaaaataggtggcttatttgtgaaaagaaccatgacagaatcagctgccacttctgcagtgaccacatgctgtagatttcctcttgaagatgagccctacgggcgtagtgttaagtctgttgatttaaatgtgtgcaagttctaatcactgtttcagcttttaaagctctatcccattgtctcctagtttctctgtaaacttatataaagttactggacagttcccttggagctcactgcgggagacggacagaggaatgtcagctgagtacagtgtgagtagcatttcagtccttccctctgggcttgggggaaaacagggtggctttttcactgattcccaaaggcagcaagagggcagcgtggaaagcacaggccctctgccctccgcaccaggtacctcctggccaggtgcctcttctctagccgagtacaggctacagggacccgagagtggctgatgcccatgttagcagctccttaacctggtcctgactacccgaggcaccattggttgtccctccccatatgtgtggagactgacagtttctcatagtcagcagagttggtcagatcactggaaggtttcttttttgttgttgttgagacagaggtttgctcttgtcgcccaggctggagtacaatggcacgatctcggctcactgcaacctccgcctccccggttcaagtgattctcctgcctcagcctccgtcaccgtgcccagctaatttttgttatttttagtagagatggcggtttccttatgttggccaggctggtctcgaactcccgacctcaagtgatctgcccgtctcagcctcccaaagtgctgggattacaggtgtgagccactgcacctggtccggaaggtttcatttttggttcacacctaagtttacacctagaagggtgtgattaaatgccaccattggaattcgtatcatttggcctttttggggctttatgataacctgctttttttttttctccttcctttcgccactgttcctgactcctcttgttctgggctttagttcttgcggagagccctgagatagaatactatgtaagagtcgttccccacatgtggtcttcagaccagcagcatcgacctcagctgtgggcttgttagaaatgcacattcgcgggctccaccttgagctgccaagtccgactcacagagagtggggcccaggaagctttaagtgtgctctccctccaagaacccagcagaagccctggaggcgaccacctgggcttgactcccccgtctcccttagggctgtgcgacctcaggcaaatgactcatctgtgaaatgaaggacaggagcgcggggtctcccagggccgttgtgagggttggatgagaggcgtggtaaagctcctgatgcggcgcgtggcgcagtttaggtgcttgaggtgcgttagtgccccaggcctctggtccttgcagaccaaggccatgagaactcagagcacacagagctgggccaggtagactccaggttcaagatggagataggagaatccaggcacatttgcactttcatttccttttctccaaattttcttgtctggaagggagcagccctgagacacctttctgctttgaaaccgtcctcggatgccctctggtggggccgtggaggggaaacctgggccacatgctgctctctccagcctgctgtcctccagcaggtctccaggtgcctgtgtgcacccacagtggtcgggctcccgggagcttagccacccttctccccagtgtcatgcttcatttctttctaagcctttttaaagagcagatttttaggagagtagaaataccttattttttcacaaggaatagcagagatgaaagacaccaaagtgtagactcggccctcgggtgggtgcaggtagaggggaggaggaaaggggagggcagacaccttgtcgccggcagccctgaagctgagctcctagaggaacggaccagatcatactcagtggtatccttggggcccatgtcaggccttctaaggaatgaagaaatgatttaggaagctctacacctcccgtaggaatttgggaaagtgcctgaatcccaggtggccctaagtgtagagatggtggctgtgcaggctgcccctcctgtaccatctccccagtgcctggcatggtgccctgtgggtgctgaggtgggtgcctctcaccatgaaggcgggatggagtgttctgggcctgctggtgacccacttaccccatggcctggagttcaattgcaagcctgagggctagagagggcccagcatgggacaccccctggggagtatggggtgacgtggcttccttgtgagagttaaatcactgtcctaccacttctcaaagccaccaaacatcgttcactgttcagaaaacatttctctgagatagaaatctaagatgagctgtcctggcagcaggaaagagagcgtcaggctgggaagcaggtgccacctgcttggaccccagaattcacccggctcaggcctctgctgcaggaaggatccctcgcctcccacataactctcgtctctgccaccttctctctgggccggtgcaagtcgctgggttgccaggcctggcttcagacagggtcgggtggaccaggtggggtttgcgccatcagatcatcttaggtgtcttacccattacaaagaagtgggaaagagatagcacctgtcgatgatagagctgcctgcagccctcagaccagccctgtccctcacctggggtgcagtcctgctacttagagcagtactgcctttgattcccttcacagctttccccctttcttcctttgtccttcttccctctggttttctcttccccttcttctcttcttccccttctccctctctctcatcattaaaaaaaattagatatttagttcattgtttcatgatgcttcattccaaaaaatgatttgcaacaagttcctaagtatgtgtggtccggaaagatgtctgatctccatgttgcagtttcccatatggaagaccagccacactgtcaagtgggaaggcgtatggcgagaactgggctgcctctcaaggacggcgtcatttgctgttcgaaaagaaagcggacattcactgaaatcatctctttcggtaacggtttctcttagttggagtaaataattttgttgatggaaccatgctatgcattgggaaaacagtctttgccaatatcagggctgactgagaagcgtcacccagatggggtgtccacagtggcggcccccactccccgagggcttgtggtgtcctgcctcgcgtggtcctttccgaataatgtcagtggtaacaggaagaggtgtgcttcagagctgactgtggaatttcagctttcaaaaagaaggaggaaccaggctgagccgactgaggccagggcgtcgagctccttgagagaacgaacccggtggtgggtgttcaactcccacgtagtgagcaggaatcgtttattcagttgaagctcaagtctcagtgtggacgtctcagtgtcctcctatgtaagatagggacagtcaccctcacttcctccaagggctctgtgaacacgaaatgacatgatagttgtagagtgctagcgtggtgtggcccacgtgctgtgagtgctcatggttatctaatagaacttcttaaagtgaaaatatccccattcaaatttctttcagcacgccatggtcatcgattctcggaattcttccatcttaccaaggagaggtgctttgctgaaagttaaccaggtagtgttgtttcacctgtgacccctgcagggtgaggggagccaaactttggacagtagataaagtcccggtgggctgggtgggcctgggggtgggcagtcccacagagtaatcgcacatgcagtatcgcttttgaaggtcgccagctccactctcagggcttccagcccggaaattgctccctaccccctgcagttcagtcttgtgccttcacagaacccaaggtgggggtaaggctgtggcatccttgatgagggcctgtaataggccaggaggagggctgtgacttggtgttacctgggagagagtgtccgaaggcaggggtcacccctcaatggctgcagggcaggtgcatcgtggcagcaggaggtgctggaggaaggtcttcaccaccttagaggagcgtgcttctctgctggtgttggaggcgggctgttagtctgtctttgggttgccctcttgttggaggcagggggatagttctttactgtaaggtacctacaaggtgcaggcacagtgaggagcccaccttggtggcttgagagtgagggcagcaggggctgcactgaaccttgaggcttccccgagcagctaccctcgctctggccggtggtacttaggtgttgtcaagcagggcaaaagcccgggggtgcctgaccttcagtgtcagaggagcaattagaaatcaggtttttatacaaaatagcccacattgtgaaaggtcagctgagtttttccccaaatgccgatgaagccaacgagccaagccgtctgtgctgagctctgtgttcagacactcaggctgccagtgctccctgcacacagccagagggcagcctctgcacctcttggatctgctgatcctccatcccagtccgtccactgttgctgtccgcaacaattaggatgtggcttctaggagacagaattttctgtagcctttaaaaaaatacaaaacggcaaaattaaatcctgatgcaaaaacatgattctgtagcccctgtaataagtctgtttgctccctttgacctgagtgctccttccctgcaggaactggcaggctacactggcggggatgtgagcttcatcaaagaagattttgaacttcagttgaacaagcaactcatatttgattcagtgagtatctaacggatgctggcacctgcactgtcagcccttactttggggatcttaccacagaatcttagctcctgaatatctaaagcacagagtggaagcctgggcggtaatgcttaacctttgatgtttccagtttagccagatttgcaggtagtctggggagaagccataaatattttcttttaaaagtaacctctgatggccgggcgcagtggttcacgcctgtaatcccagcactttgggaggctgaggtgggtggatcacaaggtcaggagatcgagaccatcctggctaacacggtgaaaccctgtctactaaaaaaaattagccgggcgtggtggtgggcgcctgaagtcccagctactcgggaggctgagacaggagaatggcatgaactccggaggcagagcttgcagtgagccaagatcacaccactgcactccagcctgggtgacagagagactccttctcaaaaaaaaaaaaaaaaaaaaagtaacctctgctaatcgttacacataggaccatgagagagcccctccccgtttatcatctcagggccttggaggaggggtggctggaggacagaggggtgtctcctgcccttgaaatgctgttccgtggaccttctgagtcttcactttgctgggtctacagggctctgggtgctttcagaaatttttttttttttttaatctttagtcctgttgggtcagtttctcctgattcactcaggagtgaggagaaagtgactggattctggtctcgccagaggagccccaggctgggagggcacctggcatatttctcacgtcttcccagcacccagtggctgggggcagctctgccctgcctttgctgcctaggtccttatctaccttaaaacctgggggcaaggattgagtgtaatgaaatggtgcatgcgactgtttctagagagctcattttaaaataatttgttactgcccctattcagcccttgactctgcactttgaacttggttcctgccttcctctgcccctgccaccacaggcctccttgttgtccggcacgtgagcccatcctgatcccttctgcagcactggatgttgtcgcctgctcgcctgttctcactttatctctcttgttttcttgactcatcattgctgcgctcttccactcctcagttttctcttctcagggccctttctgcctcataggcatggcttgcccctagatctctgttgccatctccccaccctgcttctctctgggacagtctcctacagccctgtggctgcagctgctgcctgtgtgtagcaactctggttctcctgcctccttcctgggcccagattcaagtgctcctggctgtctcagcccctaccttacagacacctcatatcaacagagctcattgccttccccacaaatctgtacccacgcctgggctccccatccactgacctgcacagtgcacccggctgcccttgaacttaactccctggctgctctcagtcctcccctcagccacctgccacccactgggcgctgcccttcctcctctcagcactgctcatccgcactgaaccaatgtcgcgttctgagtcgtctctgctcctctcctactccctgccttccccttcccccctctacactatggacatattagtctttttgagtcgtctttactccccttccactccctgccttccccttccccttctccacactgcagatgtgttagtgtttctgaaacacaagccttaccatatcactttcctacacaaactgcttgtgctgttagggtgcctgtagcccttgatccttcatcactgtgcctgtctcatgcttcggctctgtggaaatactttttgtttcccagaaggcattggctcaccctgactttcaggcactcgtattcactctgtctgccgctggctgtaactccctttttttcagttgtttccttggcctcccactccctgcctttaagactttggtataggtgtgtctcctgcaagccttcttcaacctcctagatggaagtgggggctcctccactgtgcccgtagctcctgtgcttatcctgtggtagtgcccatccatctacccacccatttacccatccatccatccatccgtccgtccatccatccacccatccacccatccacccacccacctacccacccacacatccatccatccatccatccatccattcacccacccacccacccacccatccatccaccagtctaacacatgtatgctggctgccaggcactgtccaggcactaagaatatatcagtgaacaaagcccaattcctgctctcaaggaacttccattcttgaggagggaaacctataagctagtatctttatatatcattgaggaggcggtgacaagtgttgtgaagaaaaataaagcagaattaaggggtagaaagtgaccagaaggggatgccatctagttaggatggtcagggagggtggctagtaaggcaacccccgagcacagcccagagcacggtgagggagcaagggtaggacagcgggacacctcgggcgggagcatgaattccaagagcagtagtggcaggagggccttggaggccggatgtgggctcttgcttccactctacatgagagggaagccctggagccttctgagcagagtccggacacctggctgagaagaaaatgccagctgctgtggggacagaagcttgtccttcctacagaagcttctgagaaagccatttctgagcagcttggagggacataggcatttgcaaggcagagaacgtgtgaggctcggccagccgcccgaaaacatggggtgcaggggagcaggagagcctctacctggttgaggacattgttgagaaggttttgtggcatgaagatgggctcactgagccaagactggctactgcctcctgccctgtaagagacaaggctctgtccttgagagtggggttgggagtgcgtctctgcattgggggagccttgcagccggtaccttctttcagcctaaaatgcgtgtgccctgaatccttccacattttgaggccgcggcaaagaggcctgagcaggtgcagggggcctggctccctcctctgtgaggccttggctcctgctgctgggcgtcacttcccgttgcagagcaggggcctgccctgtgtccgaatggaagccgggccctgctcgccttcggtgtgtcttgatttggactttggaattgcgtgttttaatagaatccacgtggcccttcgaagggtagctctgcaacgggacaagccaggcattctcctgcggcccttgcaccccatcgcggagctctgcacgccacgggcattcagtgtgtggccagttgctagttgctgatgttcctggagtgaaaggaatgctgggagaaaacaaccatttgtttctatttgaacaggttttttcagcgtctttctggggcggaatgttggtacccattggtgataagccgtcaagcattgctgataggtaagtactaatcaatgaatggataatttgcacatattttcctttggatcttttcagttgttttttataagatattttagagaagtttatttaggagagcatagtatttcaaatattcctttagtatttatgtttaagctaatttctaaatatcgggtatacctcacacttaatattttttgcttttgttggggaagttactttattatatttaaaatgtattgttcccttttgattatgaaggaatatagttcatactcattgcagaaatgcaaaaatggagaaacacttaaagaaattagagaaagcactcactaagtcatgagaaattctttataagcagcattgtagtggcatatgtttcattatttgcatataccataactttttagcctaccgtgtattttttgatatttcattggtttctaatttgtgatttttgtaaataacattgtaaataataacttttaagataacttgttgtttattttattttctgtggtattactctctcaaagtaaaactcatagtattgctgggtgctgcttttaggaagcttacagcaatttatacccctaccagcagggtatgaacttgtacttgttgcttcgaaatactaatcattgtcctctagggcaaaaaaacttttttttcaaccctgcattagttaaaatatactggtacagcttttttggatggtggtttggaagtagaacaattttaggtttataaactctggatcctaacagtttcacaaaactccatgaatctaatttacaaaagtacttttctaagtgtgtaaagatttatgtgtagaaggatgtccattgtagcattgctgatgaatagcaaagaatttgaaactgccttatgtaagttccgcagtagggaaatggtgaaattctagcacaaccattccagatctatgtaattattaaaaataaggagatatgtgttcataagttaacattttgcacatgaatatgtttagtggatccctttccagcgtgcatgcaggtgtagcaccgagggcctgggaagccagccaccaaaacagtgacggtgggcaccccagagcctgggatgcagcaggcagaaccatttcactcgttatgttatatgcatcctggatcatttgaattcaaatgtgtgatttatcgtttcttttttattttatttatgtatgttttttgagacagagtcttactctgtcacccaggctggagtgcagtggcacgatctcgacttactacaacctctacctcccgggttcaagctattctcctgccccagcctcccaagtagctgggactacaggcacccggcaccacgcccagctaatttttgtatttttagtagaggcgaggttttaccatgttggctaggctggtctctaactcctgacctcaggtgatgcgcccacctcagcctcccaaaatgctgggattacaggcgcgagctaccgcacgtggcctgattgatcatttctttaaaaacgaagcttgctgtttttgcaattcatgttaatggggtattcagaaaagccttgcttagaaatcccaagtagtaaaaacatatttataggttttaaaactcaaattaaaatgaaaaaattcataaaggcttttatttggtaggaaaaccagaaaaagccgcgccctgttccctttgtgctgttgcagacctgcagggtgttcttagcctcccgcggcctcaaaggtcagagggctctgagggcacctgccggggttgtgtccttggctgcctcttggctggaaagattacacagaatttctcttgtcatttaaattcattttttagtaattatcataatctaaaatcgggttctaatatgctgttcaatttttcagaaagtggatgggtttttatgtaattaatttttaattcacgcttttcatattattcagatatggttggtgagagtgtgacaatccagttcattagggaacaagaggagctcatatttaaaaattataaacctaacaagaattcttttttggtctgtttctagtattatagcccccctttttgtaatattgggattatactattaaataatttgtatcctgctcttcttcccctaatatcgcactgtgagccagtaggaggcccgccatgaaaaggaactgcgtcatgaattctcacagacagctgatagttcctttgtgacagacagtttgccctgcccctctctcctagtgtgtttcttggcatcttgtgtctcacctgcatctccaaagtttgcagtgtctccttagatgttgagatttttatgaaattcccctgtagtgtgcaccctcttggtctttctcaaaggttttgccttgtaaaaatgctgtcgataatctagtatcgaatgtgagtctgacatgtgtttcctacgcgttccgtgtgtcatgtcgtttctcacctcctgactttcctctgacctgtgtgctgttttgtcctaggttttaccttgggggacccacaagcatccgcggattcagcatgcacagcatcgggccacagagcgaaggtctgtcctttcccctcacggcgccaagtctagaaggctcgcgtctcactttggaaaccatgtacctgttattagcttacagcgataatagacattcctctttcacggtggtggaattagcacctaacacagaacttaccatgtagcaggtgtagaacatttattgagagaattgtaggaaaatgagaaaatatatctgagcaaaaactctgtgaaaatgacccgcagtcctacccagagagaacatgtgtaaacgtgttgttgtctgttccctgcaggtttgacatgtgcttcctactcttcctttgtttacatattattattttttttgagatggagtctcgccctgttgcctaggctggagtgcaatggcgcgatctcggctcaccgcaacctccgcctcctgggttcaaatgattctcctgcctcagcctcccaagtagctgggattacaggcacccgccaccatgcctagctaatttttgtatttttagtagagatggggttttaccatgttggccaggctggtctcaaactcctgacctcgtgatccgcctgcctcagcctcccaaagtgctgggattacaggtgtgagccactgggcccgacccctatgtattattattaatattatcattattattttgagacagagtctcgctctgtcgcccaggctggagtgcagtggcacgatctcggctcactgcaacctccgcctcccgggttcacacgattctcctgcctcagccccctgagtagctgggattacaggcacacaccaccacgcctggctaactttttgtgtatttttagtagagacggggtttcattatgttggccagactggtcttgaactcctgacctcgtgatccaccggccttggccttctaaagtgctgggattacagatgtgagccactgcgcctggcccctacgtattatttttaaacccatttctctcatcacctcccataaaaatgtaacagtatatttttatgcgagtaaatgccatttttaacaggtacatatttcatattattgacatcctatattttagttaataggcttaatttgttagagcagttttaggtttacagaaaagttgggcaaaaagtacagtgttcccgtataacccccatagcttccttattattagcaccttgtgttaggatgcttcattggttaggattggtgaagcaatatgataaccttattaactcaaggccatagtttacatgagggttcactcttagtgttgtaccttctgtgggctttgacaaatgcatgatgtcctgcattcaccattccaggatcacacaaagtggcttcactgcgctaaaaatgctgggctcccttcatccctcccaccagaccactggaaacgacaggtctttgaactatccatggagttttgccttttccagacagtttgggagttgggatcatacagtttgtagccttttcaagttgtcttcttttccgtagtaatatgcacttaaggtccctccatgtctttctgggacccaataacttgtttcttcttattgctgaataatattcctttgtctggaagtaccacagtttgtttgtccattcagctgccgaaggacagcttggttgcttctagtgtttggcagtgatgagtaaagcttctatcaacattcacgcgcaggttgttgtggacagcttctgggacataagttgtcacatctgctgagtcagtgccggggagcgcagttgtttggtcacgtgttaagagcacgcctcccctttgtaaggagctgccaggcgagcttccggggtggccgcgtccttttgcatttccccagtgaggaatgagagagagaaagtttatcatcttaatggctgcgtgttttgtcttatgacacactattatctttttaaagaccaaatcccaagctttggacattgagtttgtgtttttttttttcttttatgattataaaacaatgtattcaccttcgaactgaatgttggtactcatctgtaagttttatttatttatttattttttctttctttctttttttttttttttttgagatggagtcttgctctgttgcccagactggagtgcattggcgcaatctcagctcactgcaacctccacctcccaggttcaagtgattctcctgcctcatcctcctgagtagcatgattacaggtgcccgccaccatgcccggctaatttttctatttttattagagacggggtttctccatgttggccaggctgatctcaaactcctgacctcgtgatttgcccgccttggcctcccaaagtgctgggattacaggtgtgagccagcacgcctggccaagttttactttaatatgaaattctagatgtgaaatcgccagatcttttagggctttatcgtactttctgggaaggttgtctcatttatactcctagaaccgtctctgacatgcaacatccgtcagaggtatctccacatgctatggaactttaaaacatcatttaagtggatgcatattattccattattaaacattctcccctgattttcttactttttttttttttgagacggagtctcgctctgttgcccaggctggagtgcagtggcgtgatctcagctgactgcaacctctgcctcccaggttcaagcgattccccctgcctcaacctcctgaatagctgggattacaggtgcccgccatcacgcccagctaatttttgtattttttaatagagatggggtttcgccgtgttggccaggctggtcttgaactcctgacttcaggtgatctgcccgccttggccccccaaagtgctcatattacaggcgtgagtcactgcacccaggatttcttaaaatcttcagcacctttgttgcttccatttttatgtcattataagcaacaactacactctgcataaatttttgtcgaaagtatagattatttcttttcactcgtatttagagtgcttcattacagtgtaatttttaagaaaagtaacttaagtttttgatggttttatttcatctgtttattataagcaagtgtgatatgccgagtctatgagagctgagcctttacctacctgagaagtaaggcatgctggtgttccactcaccacctccgtgcggttggccagactctgtgggcaagaatgtaaattgctataatttttttgtgaaagtggtttgatgtatcaagagccttaaaatgtgtatgttcaagaattgttaataatactattaatgatagccccaagctggaaactggaaaccgaaatgcccatcaacagtaggaaatggtcagataattatggtatcttctactgtaatgaggaagaacaagttacagccactggctgaatgatggcatgagtaaatctcagaaacatgtcgggaggaaaagctagacccagaggaatgcctcctggaagatcccatttataaagcttggaagcaggtgaagtggcctgtgctgtgagagggtggggcagtggctgcatgtggtatgggcaccaggtggtggttagaagggaaggcaaggtctgatgtggggtgtgcttggtttcttggtcctggtgctctctgggcagatgtgtttcagatatagaaattcattgagctgtacacttaggatgtgtatactttcctatgggtatgcacaataaaagcttaaaaaaatacatgtttgtgccaggaaactcatcataggttaatgtccatgataaaggacttagaaaccagtatgacaatagaagagaaatagatcattaaatattggtccacctatgttgtttttcaggcataaaaaccccattttcaaggaagataaagcgttaggagaaagtgtttacaatatattaaaactgaaaaaaagtaggactggaaactgtgtatatatatatatatacacacacagagagagagagagcaagcaagcaccacattttattggcttgcttatttggttttacatttacattataatccactggaagaaaggacatcaggatgttagctgtttatttctgaatggtagaaattaaaatattttcatttttttctctttatgttgatcagatgttctacaatgagtatatttaacttttatgttcagaataaaatcagttatttaagaagagagaggaggagtggtttctgggaaacaaaaaacaaggttgttctcctgcaatttgttcattctctgttcccatcagagctctcgtgttgaaagggattaaggagatgttggtgtcttttttttccttcctctggattgtgaggaactgaagtctttaaatgaatcagcagttcattccttgaagttagtcttgaagacatcagtattttcccatttcatggtctgtcattttgtattagaggagagtaagacactgtataaatggtattttgcaacaaagtataaacctttgggttgtatgttttctgttgctttatagtttaaaatggaatggacaggaacgtttttagaaatatgcaaatacatgctctcagtggataggcttacactttggcaaaagtaacctaaatccaagcggtcatgaaccgttgagaattgtctcttctctggagacactgagctggaacctggtctcgctgtgcagtgggtggcaggcagcctctgccttttgattaatcatgtgcagctgtctccacacactgcagagacgctttctgcattttgtctctattgcgctctcgaaaatttggcaaaataatgcatttcatttgcaggtggaagtgagttggtcatctacatttgtggataaagttattgtcatgagactcatttcttcaaagcatttcacagatacgatgaatgacagagtgcattccttcctcaacgacattggctttgtttgcctcctcagttaaatcaaggtgtgaaacaaaccaggagaaaaagaaagattatttaaaatgaggccatcagtatcaggaatgagaagaacagctgcttgcaaactccagcactgtgtggcgttgtttacaggacagaaatcttgcttctgtaagttgtggaaagttaacgtgatgttaaccttgtcggaccttgtttttgttctgcacccctcctttgcttaggagactacctaggtggagaagcgtactgggccggcggcctgcacctctacaccccattacctttccggccaggccagggtggctttggagaacttttccgaacacacttctttctcaacgcaggaaacctctgcaacctcaactatggtaaaacttgcgctattcaagaaaccattgtagtacagttgttttcatgtttaaaagcacagtacacaaagaggtgtctgtcttttttttttttttttgagatggagtctcgctctattgccaggctggagtgcagtggcgcaatctcggctcactgcaacctccaccttctgggttcaagtgactctcttgcctcagcctcccgagtagctgggactacaggcgcgtgccatcacgcccggctaattttttgtatttttagtagagacgagatttcaccatgttggccaggatggtctcgatctcttgacctcgtgatccacaccccccttggattcccaaagtgctgggattacaggtgtgagccaccgtgccaggccatgtctgtctttttagattacatagtcattaaagttagagactgtgccttacttgaaaaaaattcttacaacagctaacataatgcccagcattaagtggttggttgagcgaatgaagcatacaagaaaaataatcatctaatgcccttacgaagaaaaatctgtttgtctatttaataaacaagtcatgatggccacagagctgagtaggattgggtgccttcctcagggagttcaggtccagaagagactcccagcagatgcctgggtgctaggggtgcctggaaagcttcctagaaggcatggaagtctccaagctgagactcagagtggggcttcccaggtggtcaggggatggggaaaagtcattctaagcagagggaacagaatatgccaggcccaaagaattgagaaggcgtggctcattctggatgtgagtaagtgtggctccaagtcctctcttgacctgggtcctaatctcacttcatgcatcctccctaatctctaggcctggctttaaaaacaactgcagggcccagccagggggagccagggcttgtctgggagagctgggagagcttggaaggacgggcacgcactgttgctcacaggtctttctcttttcaggggagggccccaaagctcatattcgtaagctggctgagtgcatccgctggtcgtacggggccgggattgtcctcaggcttggcaacatcgctcggttggaacttaattactgcgtccccatgggagtacagacaggcgacaggtacgtgttgggaattattttccacaatcacatcccactctccagtaattttattttgttttggacgtggttaatttcattagtggcttttaagcttgaagattttaagagaattaaatagttgcaaaataatgaaaacacatgtattttcctcctttgcgtttttctccatctagctgggaggagcaggtgtgaagcgttcccaccccctccctcaggtgctttgtgcttattgttcccagggtagtgggagcccctgccagggtctggctggaggaattgacaggtgatttggttcctgtgtgctgaaaacagaaggcagtgttttgagcagttgactttgcaggccagggtctctaggaaagttggggtgagtgaagctttgttcctgaccacagaggacacgatcttgagaacccctctctctacagcagaggaacccaccttcttggcttttcttcaccccttattttattgaggaaagattccctaaatgttacatgggtgaatatggtatagggctagttacacagttatataaacaaattctatttcttacatgcattgtgtaaaattttttttttttttgagacagactctcattctgtcgcccaggctggtgtgcaatagcacgatcttggctcactgcaacctctgcctcccaggttcaagagattctcctgcctcagcctcctgagtagttgggattacaggcatgcgtcaccacacccggctaattttttgtatatttagtagagacagggtttcaccatgttggtcagggtggtgtcaaactcctgacctcgtgatctgcctgcctcagcctcccaaagtgctgggattataggcatgagccaccacgcccggcatttttttttttttttttttttgagactcggtctttctctgtcacccagctggagtacaatggcctgaccatggctcactgcagccttgacctcctgggctcaagtgatccccctgcctcagcctcccaagtagctgggactacacaccaggctaatttttgtattttttgtagagacagggttttaccacattgcccacccaggctggtcttgaattcctgggctcaagccatctgcccgcattggcctcccaaagtactgggattacaggtatgagccaccgcacccagctgcgttgtgtaaattttaagagattcagacgtgttttcattatagtgtatctcggtcagctcaggctgccatcacaaaataccatggagtatttaagagtgacttaaacaacagatgttcatactctgccagttctggaggttggaaagtccaagatcagggtgctggcagatttggttgctgttgggttctcttcttggctcgcagatggcagccttgccactgtgtcctcacttggaggaggaagagtgcaggccagtgccccgatgcctcttcttatgcggcccctaattccatcacgagggccccatcttcgttacctcgtctgcacctcagcacctcccaaaggcctgcctccaggtatcaacaccatcctgtcagggttagggcttcagcatgtgaatcttgtggggacatgaatgttcagtccacaacagtgttcatactgtctcttatttttgtatatgcattcacagcaccccagcctgttttttctcttcatttttgtgtctacctttatgttgccaccaaatggtggtgctgttggaggcttaacccggtttcaaataggttgtagtttgtgccagttgaaggaaatgctgtatcatttttactctctccgtcttccgcatcagcatgtaggggatggtgctttgtaaaggggagccgcattcgcaagtgtttctgagcatcgcctctcaggtgcagtctgcgtctgtcattcaggtcctttacggttattgcttcagcggaatctgctccttacactcttgccagaaggcccttcagcatctgctccgcgtctggggacacggcaggggctgccaggctgctgcggctccctactgatgacagggccttcagagatggcggcggctgctcccacaaccgccagctcccattcccctccacgcctctcctgttctccacacaaagcccaagctggaaagggtgtagtcacgcaggctgcatgcatgtgtgcctgggggcccagctacccgggcttggggcccagcttggccactctgtgtgactgtgtggcccggggtgagtcacaaaacctctctgggtgtccattttcatgcccagaggatggacgatcatgatggtgactgttgcagtttggagaactcagtgagttactgcatgcagagcccttggcgcaccgcctggcctggggttgggaagtggttatttttcctgggctgctctgctgctgatacacccggcgtggccagcccctcacacaagggaacaggttcctgtgggaggtgttgcccctccccctccacatcatctcagctaacagtttgtgacaagccatagatgggatgatgcatcctgattttggagataataaagtgaaaaagtgggcacctttttccagagcgagactgcatcagataactccacgccgttactgtcttcagcagaccaggctggttttgcaagtttctttctatgaagcccttattccctctgcagttgggagtgttgggctccctggcctaacagccaggttctcatttgaatccttgcaggtagccccagaggcgctgtgacgctgctgcaccaacacctagcttaagtgggtggttttgagtggttgactgcaggcccggggctggaggggcgttggagcgagggaagctttagataccgctctctgacacagtccctgctgctctgggacccgccactgtgcacgtctcgggcagggagggtctgggcagcccacgctgccatcaccaccattgcagtgctctttgtagccactgggtgtcagtgtgccctgagaagtcaacgcggcttttaggagctctgttgaattgaccctttctgaaataattttcatatgaagtggttacatttacctttcagctttacttccgtctcttcaggttaaatctaaaaaacacgtttcagagattaatttcaaaatatggtttattccgggaggaagcagcatcctaagcacgtgacatttaaagaccaggctataaggaagtgcctctgcccccaggccaggtggcagctgttcagatgtttattatggacagtgagctctgaacggggtcagcctggcaccccgagtgtggaagacattttcgctcagtgtgaggccttgtttgaggttggtcatcaatattggaatttcgtgaagttggagtgaggttgccagatttaatcttcatttctaaaatttggtagctggcaggatggggtatcgtgtgtgtagaaattatccacaggtttcccccataactgaggcaggcacactgtaaataggacttcagacattcacaaagaaggaaacagttttgagatgtttgcttactgttatgtcgcaagtgatttgtggcaccactgtctctgggatctaacagcattctgtcagtttgtgtcttaggagtccggtctctggagacacagggctgaatcaggcaggctcgcttgggagagcagctcacagttagcagcaggaagacaagaaagtggatcatcttggttgttggggagggtgctgagagggccccctggagcaggtccctgagctgaatcttcctagaggacagacagccaggtgcttgcagaagacacgcagggacagtggtcctggctaacaaaggcaggagcaaagctgtgcaggtgtgcgctgtcggcgggcaccgggcagaaccgcgtcctacaggaacagaagggggagtggggaggtccaggccctgagctcccacgcctttgccttccagccccgctgacctttttccccttgggtatatgccagggtctttgagctcaggacttcatctgccttgttcaccgctgaggtccccatgactacaactgcacctggtgttggaagtgagagccaggtggagaggctcctggcgtgtggtgggaggtggggtgcaaggcgccaagggtgctgttggcatgaccttcctaaagcaccccatgctgggtgcttcctggcctccagcctcagagtccaagttcgtcagaagcctttgaacatcagactccaagaccctgtgccggcagtggcagtgctgggtgagaagaaggtgggagatgaccaggagccctgcaccaagacagcggccgtgagggagggagagagcgtggggtgcacagcagaaggtggatgtttggggctgtctggaggatgccaaggctggcttgcccctggtctggtggaacttcgcagcgctgctttgaatgtttgcagtgggtattttgttctgtgacatgtttatgtggtctctgagcataaacctatgcttgtgaagttgtttaatctgtttgtttgtacttagagtgacaggcctttattagaatgcttgcttgttttctgaattacatatgccaagagcttgacttcctttttagctcctagcttatgttcaggcatttttctaagtagcgaatgtaggtatagactagtttgaaggagctgagagtgtacaatctaaaaacagatctgaacacaactaaatggtacaaatgcagcccgggttttgatgtggattctggtgttttaaggccatggatgtggcttactgtaatcttgaaggggctgcagtcctggcttctggtgagaggactgcagtgccggggctggttaataagcacccttcatcctgcaggaggccggcgcagcatttgtgagtatctgtgttgaatctcttcgtggatcagatattgtgtcttcttgctcagagtcaggttggaaaaggaaaacttgccgccggtgtgcatgtgctccaaatcctcagcttgggcaagggcacgggcgtcgtgaataaaggagccattcttgctggccttttctagaaattgcccacagcttgcaaaaaggctgtgttccctggccccggctgcggctgtgtaggagtctgaatatcattttccccagaagttgaggtccctaggttaggcccaccttgtcccaaatgggcagcattggccttgccccatgcacaggctccaggcggacagagctgctgcaggcatgctgtcagggggacaggctgccccccagctgtgcatggcagtgtgtcggaaagaacaaggcctgtgggtgcccctgagccgggtctggagtcctgtcctgccacttctcagccgtgtgactggagcctctttgctcctctctgaaaatgggtctggtggtttgttcccaggttcttaaccactgtgtggagtcacacctgcagaaggtcagctcataacagatatggcaaccaatgtgacctttgcatccttccttcctggggtcaggagcaggtctaagaggtggtcaggctaaacccctgtagggctgtgggtactgctggtttcctaagccccgggaccttctgggggccgggcggaccttaagttctgtccacctgcctctcctccatcctcactacccaccttgtccttccggctccttccctccctgctccgcctctcatcggccctctgtcctctccgtccggagaggggaacgtgaaggaggtgaggagggagtagtgcaggaggatttgggtctctccttctttcccttttccattctccgagggcttaaccagctggtgaaggttcttaaccagcaaaggaggaagcagccggggccggtgagggtgaggccggcagccaggcaggaaggcagcaggaggaggaggaagcggaggcggcaccttctgagaggcgcatgctcagtgagtcgtgaagatggcagggctggcggagcggccgccgcatctgatctctcccctttttttaggatatgtgatggcgtccagtttggagctgggataaggttcctgtagccgacacccctacaggagaagctctgggactggggcagcagcaaggcgcccatgccacacaccgtctctcgaggaaacgcggttcagcgattctttgactgcggaccctgtgggaaaccccgtcaataaatgttaaagacacactccgaggcagcgtggatgtggtttgctttcactcaacagtatggcccgcgtggcctttccccgggaccacccgtgttctcacacacccgcccagtgtctgcgagtctccgtgtttggttcccgtgtaccagacatgatgtattcagagccaggcagcaggagggagctttgccccagaggaagctcagccatgctgttacggagagggcgcgctcccctcgatgcaccagccgttgtcagaggaggcccacggcagcgggcgcttggagccacagtgctcgagccatttgagtgtgtgagtggcaagggagaaatacccaaaccgtgtaacattagggacactgaagcaaagacggagcctggtgtctacagacgaatggcagcatgaatgagtgtgcgttttctggggctgctctaagtcaccacaaacgtgggggcttaaaacaacacacattaatcctcttgtagtctggagatcagagtccagaatgggtcttgctgggctgcgtcctcctggaggcgctaaggggaaatccccctcctttgccagcagtgagaggcaccacattccttggccgacgtcacctgcatctttaaagccagccatgtggtgtcccccatctctccctcttgcctcctctgcgtctctgctgtctttgaaggacccctgtggtgacctcggatctgcccggggaatccaggggtcttatttctaggtcagcagatgagcgtccttaattccgtctgcagacttagcgcccccttgtcatgttatcgtcacgtttacacgatccagggattaggaggtgtgcgtcttcggctgggtgggagtgcgggacattattctgtctcacacaactagtatctctctaatatttgggggataatcttaaattggtcacacataaaaaaagtttttgaagaacgtgaaacctcctttgaattgaaaaagtggtgccatatggaaacgtctagtgttgcaggtgatgccccgcagggcttcgcacatgcattactctccgattttttctctctctcagctggggctgagtccccgtcactttcagtcctccccgtaacttgttgtttatgtctttgccaaatgacctgaagatttgggcctcagtctggggacccatgttatccctgggccacactctcaggtgccccgggactgtctcgccctcattacctgcagaagctgcagggattttacagagaatatataaaaagagacagttttggttgggcgtggtggcttgcacctgtaatcacagcagtttgggaggccgaggtgggtggatcatttgaggccagtagttcaagaccagcctggccaacatggtgaaaccccacttctactaaaaatagaaaaattagctgggcgtggtgacagatgcctgtagtcccagctactcgggaggctgaggcaggagaatcgcttgaacccatgaggcagaggttgcagtgagctgagatcccgccactgcgctccaacctgggcgacagagtgagactctgtctcaaaaaaaagagttttgccttccttataatagttaggaacatggaaaaatgtgctttttccccctaaatgatttattctttacagtttcacgcttatgcctttgagaaaaccagaaacccatgtttcaacactgtcatgatcagaccatcaacttagaccagtgaaatctgaataatacagactcagcactatgatctttgagaaatcatctactacatcagtggagaacccagcgaaagggtcacctgagaaaagttacctgtttaatcaagcaagtctggggtaccccaggtcgctacagccagctccttaatgaaagctcaccagggccagctgtctctcgtgcggctgcagcgtgtttaatttgcaggtggactccgcgggctgctcctccacaggtgaagtgaaaacagtggaggtcctgggtgaaaatgcgggttctgttcatcctcagaaggtggtaagtgggcagactagagcgtgaagccagaggcacttcatcaagtgctttccacaaaagaccaaaaataaggcacgttcattgtgggaaggaatttcatagctgctattttagagagctcagagagcaaagaaagatggaaagcctcacaaaatcccccatgaaatcctcccttggctcagtgattctttgcatcttaatgttactccgtttcttctctaaatacctcaattttctggactggaagagagctgggtatttgtttaattaacctcttgctttactcggcgagggatgggccatgaaaccagtgggctgcttccgctgaagaatgctgccggctccgtgccggaagcctgctggctcggtcatcttccatttgtaacaagaagattgattttagtgaacctaatcttcacaatgcttctggaagaacagctgtgatggatcactcctctgttatcgtgaatctgcggtgctctcggtacacacactgtgacctttaacaaacggtttcatcagtggtgtgcctgccacactgtgccacaaggctcttttgtacctggctgctgtctacaagtcttatgacccagtgagggagcagactgttctgccaaatgccatcgctactaggacaaaggacagtcacctggtgaggatctgccatcaggccacattttcccattgggtgactcctgcaaaggccctacactttgacgtcctccccatctccggcagctccttaaactaagaactcattttaatttctgaattctccctttttaaagaccagataaatttacctttgggacagttagatgtgcacaaatgcaccatgtgtttaaagatcaccaaagaggagagaaaaggggattccttagtccagagaacaaaaactggtgagctgtgatttaaacaaaacgaatgttgttaaaatcaggttgtactcagaagtccaaatttctagcttctcttgagaaactggatccgacaaacaaaaaatgttcattcctacctgcagcaggaggctggagctgagacgtgggtgttcctgcagctggggcctgtgctcccaggcactcatggcttctctggctggttcacttctccctggctcagacagatcaggagtggaaacatctgaatatgctcaaggaggaggtaagttttgggggaaaaactgtcagaaagtggatgagggctgggtgcagtggctcatgcctgtaaccctagcactttgggaggctgaggagggaggattgttgcttgagcccaggaatttgagactactctgcacaacatagtgaaaacccatctatatgaaaaaaaaaaaaaaaaaaaaaaaacagctgggtgtggtggcatgcatctgtggtctcatctacttgggagacttaggtgggaggatcacttgagcccaggaggttaaggctgcagtgagccgtgattgcaccactgcactccagctggggtgacagagcgagaccctgtcacaaaaaaaaagaaagaaagtggactagcagtgagcccagggtcaggggccctggctcttgccctgttggggctgtttaccaaatccactcactgctttcctcaggtttttcttctaccaaaggagaagatcttggtgtattcagcaatagattccactcttagtggatgccactcatgagctttctagggctttctacccccaaggtaaaggctagatgatttttaacaccagagattgattgttttgcctgtctgaggacattatgtacaaggagttgaacagtggctctttgattgtgattcagactggaggtgaatcccagctctgtggttgaccagccgtatgaccttgagcatgttctcatgtgggttcaaatactgggccactgtgaatatgaggcacagtcccctgtttcccagtgaagaaaagccaggtggaggagagtggccaacctgagtaaataacttttagaaatacagatgaaataggccgggcgtggtggctcacgcctgtaaaattttgcagatcttttaatatctgtcttcaaaaccagagggattcttgcatctggttctgcattcagtttgttgcatttgaagttgttcgattgacgcatatgaagaaaatgtgttttcacatagatacgtatttggaaaagagagtattttagtagccttttcagataactgtgaacattctttgatacttcaccaaaactcaactaatgaacggttttgaaaggttagttgcagtgtggaatctgaaaccccgtctatgtctttttcacactctgttccattaagatcccctggtctacctggcactgtgattggattgtttacccaggcatgcttttgtaacatcatatgctagtcatttgcaaaatactggttccctgaattaggcacatcttccaaatgtcaacatgtcttcattatgcaaaacagaagatcacatttggccaggtgcggtggcccatgcctgtaatcccagcactttgggaggcccaggtgggcggatcacctgaggtcaggagttcgagacaagcctggctaacacagtgaaaccctgtctctactgaaaatacaaaaaattagctgggcgtggtggtgggcgcctgtagtcccagctactcaggaggctgaggcaggagaatggcgtgaacccgggaggcggagcttgcagtgagccgagatcacgccactgcactccagcctgggtgacagagcgagactccatctcagaaaaatcaatcaatcaatcaataaaagatcacattcattaatatcagcacccgttggatcaggaaaatctaagtacagatgctcgttgacttacatttgataaacccgttgtaattcaaaaatattgtaaattgaaaatgcattaagtacacctaacctactggacatcgtggcttagcccagcctccctggaacatgctcagaacacttatattagcctacagttgggaaaaatctaaaacaaagcttattttataagaaagtattgaagatcttgtgcagtttgttgaataccgtacatgacatagaaattgcaaccatttcacgctgttgtaaaagtcagtacatcggaagtgggaccattagaagttagggaggtattgggaagctgccaagctcatggtgctggatccaagtttccaaaaattccagttttcacttgaagcccgcattttaccattggcagcaaacacttgaagctcactttgtccatttccaaggaaatgtctgccagatacccacgtctgaatcaccatagtttgttagtcgctcttccagggaaaaacaatgttccatgaacacagcggcaattcaggcctggacccaatcacccagggctttccttgagacatatgagcccctgtgagcagcagaagtgttctctgcttaggatgtttgaacacagggtaaaagcgctgagatgtaatgacagggagcattttgctgcttcattcatgagcgaaactggcatttgtgtttccctccccaagcacagtggcgaagaatacaatggccataggggtgtgttgcctctgccattctctgccagggtgcaggcactttcacccacagctgcttttgcatcgtgagtgcaaatgtcaacccagtgaaccaagtaactaagtaatgtctttgcattattataaatatagttttaacccaatggactccctggaggagtctcaggatctccagggggccgtggaccacgcttcgggaaccactgatctatcgttgtccaaagctaattttttcagctttcggggtcctccccaggtgacttgcaatagagtaattgtgagaatgcccataagggctaagctgggacttaagcaccctccttttattttcagatgtaaatcatattcacctccctgccgcagatcctttgtgaaatggggtagtcgagcctatcagtatcgagagaaacaaaagcgaagtgcctggcacgtggtaggaactcagaagtggcaggagggggcagtgggtgttggtggaggggcagaggcgggtgcgtggtctctgcgctccgtggggtgggtaagaacctgccaatcccagtgggttctagtaggagggatgtctttcctgcgtggctgtacacaggagaacactgtaaagagcaaggactggcattaaattctgcattaaacgtgaggcctgtttttacatttacacatacagatttcagtttgcgtagtgaaccgtgggaggctgtctcagtcgtgccttcctggggtcaaaccagttggcctgttttcaagaatgaaaaatgctttgcccgaaagtggggcgaatggtaatcacagacacacacgtctcggtgccagagttgtagctttatacatagtgccttcctgacaagtaaattactggattcctttgccaaatcattagataacttttaagagcaacaaacattcatgcaaaaggagattcagcattatcttgttagaaaaatagtcatgatttttgaaaagcaaggacattctgtgaagtttgaccaactgtgaaaattacagcctttcttccccgtacttcccccgtgcttttcaatcaatcagtttctaatggatgaagcattccctacatttgtcactagatggatgtgccggtcaattaaaatattctgacttcgcagggttgagataatagccagcctggatccatccgttactgtgaaatagaaggtgaccttcctgtattgagtcagagacccaaatacaattgctttgatcttattatcatagccagaaattaccgttcatctaaacgtaatttccctgaaatactgagcgggcagagctactgctggaaataccagaacatcataaagagactgtgatgtggccttggggatgaaaggtgacatcttgacttgacatccattccacctgactgcagaaatcaagtggaatggaatttcctgggatgttctgtatctagtaaaggctaagtaggattcatcaccaaaccttaacccagagagattaagccctgttataatgtgctgcggcagccacgccaggactggactccctggaatctgaccctttctttccattcccacactcccaggccagggtcctagcctttcctgtctgtctgaaaactgctcttccaggggtggggagcaggaaaccttgtgttggggtggaaagagcctgtgtttaggagttggagctggctttggattctcacttcctttgttcgcttattcgttcctccaacatggacattgggccaggtgcagggtgttgtacaggtggctcttctggcaactgccgtcaggtttaccctcctctttagccccagaggtcccacagcctcctgtgtctgcccatcccttacctccgatgtccccttccctgaagttgttccaaatgacctgtagttgccgctgctgggcctggcttctgcagggctggccactccccttctccagggagcccctagttctgtttggagaactttgccaggtgctggcccttccccactcttgggatggaagcctctagaacaggatggagaggggtgtatccatctgcatgtcattctctggtcagagaaaataagttccaccttttctgagcttgagtgagaaaaaaacaatcaaactagatgtttgccaaagacatcacagcagacacgaagtagcagagccatgggcacagaaaagggagcctgggtaccccctgctggcagaggtgctggggcaggagcaggcgctccaggtcgcgcgcgaagtcagtgaggacccttttgagctccccgcaactgaagaaggaattaataaaatcaactataacctaacagtagtagtaatagaaattttaaaatcctctcaaagttgctgcaaagtgtgaccccccccttactctcaagttaaaagagaatattaacagtctgtcttctctctgtggacagtggaccttatctatactccccaactccacattcctcaaagtttgttacaggcccagcgagttcctgcttacctccctagcatggctgcagggtcacaagaccaataagtttaggtggcaagacatgtttctctcaagatataagaaatgtaatgctgtctttgtttcttgcttctgtaacttgcttcccgcctcacgtagttcctgccttaagatgtttacaagtagaaaaagccctttgttcggggctcaaactttctggacctatgtccggctgagctggtgatcaccttaatttaataaactctcctgaaccttttccgtctctccagtctttgattgtcctgcaacacaacgtccggtttgtgagcatgcactgcttttgtcttctgggctaagccagaacaggcaatagggttaagcacctgaagtacattttaaggagaattaggagccattgttccaaaaagattctaaaaatggatgattgatttaaaaggttctcagggacagtgctgtgtactcaacagactcctgaccgacctgcttatgagccttgaaaacagaaaatctttattttgcaataaaggtgggaaaaatacaacagctctttaaaaatcaatgattaaaaaaattttatagttttatttatttatttttgagagagggtttcactctgtcacccaggctggagtgcagtggcacaatcacggctcactgcagcttcaacctcttggacttaagcaatcatcctgcttcagcctcccaagtaactgagactataggcgtgtgccaccgtgcccagctaattttttctattttttttttttttttctgtagagacagggtgttgctatattgcctaggctggtgtcaaactcctgggctcaagcagtcctcctgccttgaactcccaaagtgctgagattacatgtatgaggtaccacgcctggccgtttttttttttttctttttacacaaataatagcatactgtagttcctgttacatgcttttctctttcttttctttaattttttcagagacaaggtcttgctctgtcacctaggctggagtgcagtggtgctgaggcaggagaatgggttctggaggcagggtacctaaggctgatttgcactgatttcctagagctgaatcaaaaggaaaaccccacctctccacacccaagtaacgaaaggatcagaggctactcaaccctcccacctttccactgcatggcagatgaaaaatgggaagttcctctgattggtcccctcccacaaccaatcagactggtcgcaggccacttcttcatttacatcggttgtaatcaagtaacaacctagagggtatttaaaccccagacaattctgtaaccagggctctcagccactgctagagcctgctcccactttgaggagtgtacttctgtttcaatatatctgtgcttccgttgcttcattctattgtttctttgtttgtgcgttttgcccaattctttgttcaaaacgccaagactcataatcaagaccctccactggtaacagcgctatcatagctcactgcagtctcaactccctggctcaagggatcctcccacctcagtctctggaatagctgggaccacaggcgcatgccaccatgcccagctattttaaaagtattttttatagaggcagggtcttgctatgtttcccaggatggtcttgaacttctgggctcaagtgatgctcctgcctcagcctcagcctcccaaccctctgggattacaggcacaaggcaccacaccaggcccatggaatgctttcaatagtgtatatcctagcatcttacgccactccaagtgtagtgcaggaatctgacgatacattagaaatgcagagtcctggccagatgccgtgtctcatgcctgtaatcccagcactttgggaggccaaggtgggcagatcacggggtcaggagattgagaccatcctaaccaaaatagtgaaacgctgtctctaactaaaatacagaaaatcagtcgggcgtggtggcatgcgcctgtagtcccagctacttgggaggctgaggcaggggaatcacttgagcccagaaggcagaggttgcagtgagccgagatcgtgccactgcactacagcctggtgacagagcgagactacatctcggaaaagaaaaaaagaaaaagaaatgcagagttccaggccctgcccagcccaccctgtggttttgcaagattcccaggtggcctgtgtgtgcagtcaggattgaggagcaccctctaggtttgaactcagcacacttccataccttcagccctctcctctcataggtaactcagctcccacctgccacgtggcaggatctcctggacaggcagccagaaagctagtaacagcaaagctttgttacctaacatttccttctatttgtgtgcagtacctccccctgtcatgccccgtgtgagggcaaatcaccttcatcgctgaatctttcacctgggcaagtgcaggaggggagccccagatcagctgctcccatccatgtgagttctgctgcgcggatgcaaaaggcacaaagatcccagggacccataggaagcttcacaatggttaccatccacctactgtcagtggccactaccttccgttcattcaaccgtttgggggacagcgatcaagctcttagtgtgtgcgaggctgcgtttgcgggtacagcgggcggagatgacacttcaggtctagtggggaagaacaggtcagcagggggtacccaggaaagatggggagggggtgcccagctccgcctatgggaggaaggtgggctggggggagtggagtctcctccgagaagcccatcctttcccgtgctgctttgcagtttagcatggggcctgcatcaggctctgcactcccacgctggaggccagagattcatgtgaatttccagtcgtgggtcatggccagagcatcctttattctctttcccaaggtcgggctctctgttcaccctcttctgcctctgggagcctctggactcatttcttccctcctgcgctcctcagcgggaagtctccatctccttccttcctgtagattcctcttctccacgagaaagttttagaggcttttccaatcccccaaggcaagactttgaattacctttagttttaggccctgatttatgtgggtttgacaacaggctttaaaaaagtttttttttggtaaaatgcacataaaatgtaccatttgaggtctacagttccgtggcttaaatccatttacactgttgagcaaccatcaccaccacccatctccagaactctttcattccgaagctgaacctgcacccattaggcactcactcccaattcggtccttcccagcccctgcagccactgttctacttcctgtcgctattaatttgactcctctaggtgcctcatctaagcaggatcatacagtatgtgtccttttgtgactggcatatcacactagcatcatgtcctcaaagttcatctgcgttgtaggctgagtcagaatttcatgtatatacgcatgcatacaccacattttctttatcccttcatgtgtcagtggacgtttgggttgtttctacctttagagacaggttttttttgttgttgttgttgttttgtttttgagacagagtcttgctctgtcacccaggctggagcacagttgcatgatctcggctcactgcaacctccgcctcccaggttcaagcaattctcctgccttagcacccccgcccgcctccgagtagctgggattacaggcgcgcatcaccacgcccaactgatttttgtattttagtagagatggggtttcacatgttggccaggttggtctcaaactcttgacctcaagtgatccacccacctcaacctcccagcatgctgggatttcaggcgtgagccaccacgcgcggtctagagacaggattttatgattgagtgaatatacctggatgcctactatgtgcaaggaaagcatgcttctgagaaaaaaagattcattttgcatttttcttttagattttgagaaaaccataccatttagttatagaaaagtatttatcttttgcagcatactttggctgactttgataacactcagactgtattagagatagatgcttgggtctgccactctgaggacacggatccaagagtgctgccacaacctgcctgtctttcttgcttgcttataaaaatagagatgacaggctgggcatggtggcttatgcctgtaatcccagcgctttgagaggccgaggcaggcggatcacgaggtcaggaaatcgagatcatcatggctaacacgaggaaaccccatctctactaaaagtacaaaaaaaaaattagctgggtgtgggggtgggtgcctgtagtcccagctactcgggaggctgaggcaggagaatggcgtgaacccgggaggcagagcttgcagtgagccgagatcgcaccactgtgctccagcctgggcgacagaacgagactccatcttaaaaataaataagtaaataaataaataaagctccatattttaaaagctcattatagtatttaagtagcagtgatgtttgaatagtcttttcatgccttcccttttactgatttccttgcgaatttgaaattttgactgtcttaagataatttagcacacagaattgagctttacattttaatattttcctgatttaagaggataatttgcttacagctgttcagacctctgatctcaattggtaatgagctacagtaggtatgctatgtggatgagcatgggacagattttaacatacaagttgcaaacccaaacccatgaaagtttctatcacttttatgtcagactgagtttttaactgatgataatgcctgtctgagtattcattttctcaatcaaaatgtattctaatcagttttgaaatcagttttgaaatcagttctatttgctctaaataacatcaacttgttatttttttgtacggacagatacaaaggcagagttcaaatcagcaattccccattgacaaagtattaaggtaaagtggcattctgccataaaggtttatatatatatgtaaaaacctatatatatacacacatatgtatgtaatatataatatatgtgatatatgtaatatgtttattacatatatataaaataggttttaaaacctattatataaactctcttgttaatatataaaccattatttaaccctttaaaaaccctcttttatataataaacattatatatgtaaaaacctatatatacacatatatatgctcatatatgcatacacatgtgtgcatatgaatctttttttttttttttttgagacggagtttttctctgtcactgaggctggagtgcggcggcgtgatctcagctcactgcaacctctgcctcctgggttcaagcaattctctgcctcagcctctcgagtagctgggattacaggtgcccacctccacgcccagctaattttttgtatttttagtagagacagggtttcaccatcttggccaggctgatcttgaacttctgacctcgtgatccaccctcctcggcctcccaatgtgctggaattacaggcatgagccactgcgcccgtccccatatgaatctttaaaattgcaaatgtaattcccattagaatgtaagctccgcagaggaggtcttgttcattttgttcacgagtgcaccccacggcctggaatcacacataaactggcactcagtatttgtggaatgaatgaatccatgagtgtgcaaaggagaaagcacaagttgcttttctgcttaaccccatgtgatgaatggcaccacgtcccactaagatcactctgccctttcccctgccagggtgtctcataatgttccctggggcggctgcactgtcctataatttgtgtaatcaaacaggcacatttaggtgatccccattcccctatatgacaagcagtcctgcagtgtgctcatacctttgtataattgtgcagaaactttcatggaataaatgatgagaagtggagttgttactgacgcacttttggaagtactgtgctgatgcattcctaatgaagctcccttagagttcagaacaacagaatctgaatcactgttgttttcaaacatcggtttttggtggtaccatcagcatcaccactattaggactttcaggtaaaatgtgcagtaaatgaattgaattttggttgctctctgctcccgaatctgcatagtatctgattttgtatggcctgcgagtgaaggatgagtttacgttttaaatgtttgggaccaaaaaaatcaaaaattttacgacatatgaaaattatgtgaaattcacatttctgtgttcttaaaaaaaagttttattggaattaggcaccatgcctattttttttttttttttttttgagatggagtctcgctctgtcgcccaggctagagtgcagtggcgcaatctcagctcactgtcatctctgcctcctgggttcacgtcattctcctgcctcagagacagggtttcactgtgttagccaggatggtctcgatctcctgacctcgtgatcctcccgcctcggcctcccaaagtgctgcgattacaggcgtgagccactgcgcccggccatgcctgactaattttttaaaaatatttttcatagagatggactcttgctgtgttgcccaggttgttctcgaactccagggctcaagtgatcctcctgcctcagcctcccaaagccctgggatgaaggtgggagctgcacgcacctggcctgcttacacatcatctgtggctgctttcaagctacaatagcaaaattgggtccttgtagccaggcacggcggctcacgcctataatcccagcactttgggaggctgaggcaggtggatcacttgaggtcaggcgtttgagaccagcctggccaacatgatgaaaccctgtctctactttatacaaaagttagccaggcgtggtggtgcatgcctgtaatcccagctgctcgggaggctgaggcaagaacattgtaagcctgctggtgcttgaacctgggaggcagaggttgcagtgagccaagatcacaccactgccctccagcctgggtgacagagtgagatgctgtctcagtaaataaataaataaataaaatttaaaaaattgagtccttgcatcagagtctgtctggccccttacacacccctgccgtagaccactgtgtcttccctggccacagcaatgttctcatgatgtgctatggccgccacatccttagctccagcatcgtagagcaagttccctcccacaaagccctacacttcagttctcccctacccccgtcatcattctctccttcatccaaaacaccactctcatcccctcactgctctcaaattgggtgggaggagaaaataaaaacctttcattttgcaagcaggtgatacctggatggcaaatgaatggcaaaagaaattcagactaaggtgtgaatgtctaatagacttggcctttgttctcccacccacctaagaggtcctggcatgtttcaaggagcttgctcatcccccattttctcatagctggtcagaggggagtctttttttaaagggggcataatctttgggggactttcccagttttcccccatggaagactaggaaccaataacttggtaccatgttatttattccttcttcatctataaaatatttaagggaacaaaacgcatatgtggtttactctgcctggtatgcaaaaagagaactcggaggaattgctaataaatgaaaatccaacttcccttgtctcagagtttgctgatgcttccaaaatcatttgagtgaaagacggttgatcagtgtagttggtgattcatgttctgctcacacgtggcatgggcagaagtgtttccatcacagttttaaaattctaggcaagtctggtcatctcctcctgctcttagtatttactgtgaacatcgcaggttactctcttgccagatcactaaattagcagactgattaaataacaccatttcaaaatgctagtttagatattatggttacagcgtgcttgaaaaccctaccccaaagtgcagtaagattgcctcaaaagtataacctgggggaaataaggcattttctccatcgcacatcaattaaatatttccatcacttctctacacacttctaactcgcctaaaattgcttggaggtctgaaaatgaacaacacaagaagacatttcacagtcttaagtattccctggaatgagttcccttttctctcccaaagagctacaggcggtttttatttatttatttattttgagaaatatcagcttctttttagttctattaaagagaagtgaaagaaagctgacaagctttatttatttaactttttttttttttaacttttttgagacagtctcgctctgtcacccaggctggagtgcagttgcgcgatgtcagcttactgcaacctccacctcccgggttcaggcgattctcgtgcctcagcctcccaagtagctaagactacaggcacgcaccaccacgcccggttaatttttttgtagttttagtagataaaggtttcaccacgttggccaggctgatctcaaactcttgacttcaggtgatccgtctgcctcggcctcccaaagtgctgggattacaggcgtgagtcaccgtgcctgaccgcttgcagcttttaagaccctctttaagcacttagagtgctgccatgttatccagaaggaaactcacacatgtaaagaatccaaattatcccatccaggggtatcacgtattgactttcttttccccaagaatttattgtggtctaatttatatagagggaaatgcacacgtttcagtggaacgtctgttgacttttcacgtttgtgtacactgtgtgactaccaccagatcaggctgcacatcacccagccagcctgctggtgcccatatgcaggggagatcctcagtgcccctcaccacgccggcgaagccactatcaacacagaacagaggtcgcctgctcttgaacttcaaataagtggattcacgcagtctgtgctcttttgcatctgtctggcctctttcattcaacataatgttttggagatacactacattgttctttttttttctggatggagtctcactctgtctcccaggctggagtgcagtggtgcgatctcggctcactgcaacctccacctcctgggttcaagcgattctcctgcctcagcctcctgagtagctgggattacaggtgcccgccaccatgctcagctaatttttgtatttttggtagagacagggtttcgccatgttggccaggctgctctcgaactcctgacctcaagtgatctgcccacctcggcctcccaacgtgctgggattacaggtgtaagccaccatgcctggctcggtgtaacttctattgaaaaagatttttctcagcactctgggaggctaatgtgggtggatagcttgaggccagcagtttaagaccaacctgggcaacatagcaagacctccatctctgtttgaaaaagaaaagaagagaaaaagatccacatgtaagtggacctgcacagttccagcatgtattgttcaagggtcctgtgtttgggtactatacacagaggaacaatgtttccgtttgttcaaacacactggactcaggtccctctttctgcaggcccagattctccaaggcaagagtgtgggtaagggaggttgactgagatcacacagtgcagcccccagcatggggcacagagtgcatgcagcaccagctcaggaaatgtttcctccttgtccttaattaagattatatggaactggcctcttcagaagccacagttcaaaagcaaaccaaatggttttctttaataggggcagctgcatgtaatctgattttcataatcattctccaagtgtacaatgtatgcatagccctggcacgaggcatatgtggggagcatattaagaagcaaagacggcattgatggagaaggagctaagttcatatttctgcatctcattaaattctctctttttattagaaagtggcactttccaatcttgaagttaatatttatgtttgctgctctgcaaagcaatttggaaagtagttttagtgagattaccccagagagaaagctagttatgtaaactccacaggagccaggccaggaaggaggaaagtttttccggtttgatcattgtccttccttcctctcaccttgaaaagaaatacaacttagtccacagccatcaagccagttcttgattgtcatctgtagcacatacaagacaaaaaaatcattacaacagacacaacaattttttttttttttgagacggaatctcactctgttgcacaggctggagtgaagtggtgcaatctcagctcactgcaacctccgcaccctgggttcaagcaactctcctccctcagcattctgagtagctgggattgtaggtgcccgccaccatgcctggctaatttttgtatttttagtagagatggggtttcaccatgttggccaggctggtctgaaactcctggcctcaaatgattggcctgccttggcctcccaaactgctgggattacaggcgcgagccactgcgcctggcccagacacagcaattaaaaaaaaaaaaaagttctaacctgcatttaataatgatacaggagatagaaagaaattatttagacagatagtgagggtgaaagagtccttgacaagatctctcttctaataaaaagcagcccccaaaattattttttttctaacaaagagcagcctgaaaaatcaagctgcaaacatagataaacatgctaaaaacttacatgggtgaatgcagacagctgtgccaatagaaaagggctacctggggccaggcgtacccaacatgaagactccatctttccttttctgttgttaccacgtatacaataaagaaacgaacaacatgacactggccagacagagaatccatctacataatacataggattggggctgggcgcggtgactcatgcctgtaatcccagcactttgggaggctggggcgggcagatcacctgaggtcaggagtttgagaccagcctggccaacatggtgaaaccccatctctactaaaaatacagaaattagctgtgggtgatgatgcacacctgtagtcccagctacttgggaggttgaagcaggataatcatttgaacccaggagacggaggtttcagtgagccaagattgtgccactgcactctagtctgggcaacaagagcaaaactctgtctcacaaaaacaaacaaacaaaaaaagattagggtgcagacagccaacttttaacaccttatacaaatgatacacctagctctaaccagtttttcacgccttatacaaatggcacacctggcccaaccaatcgtttgtgccctatgcaaatcagacaccaccttctcaaactaatctgtaaaatcccctgacatttcaccatggaaccaacaacgcatttttttgggatttctctctgggcagagagctcttctctgtctttttctttttctttctttctttctttttttttttttttgagatggagtctcgctctgtcgcccaggctggagtgcagtggcgtgatctcggctcactgcaagctccacctcccaggtttacgcaattctcctgcctcagcctcccgagtagctgggactacaggcacctgccaccaggcccagctaattttttgtatttttagtagagacggggtttcaccgtgttagccaggatggtctcgatctcctgacttcgtgatccgcccgcctcagcctcccaaagtgctgggattacaggcatgagccactgtgcctggcctagctcttctctttcacctattaacttccactctaaacctcactctctgtgtgtccatgtcttagttttctgtggctgtgagacaatgaatctcgggtatcaccccagacaaatgaggccacttcaataacacatcatcctatccaagtgtaaacatgaaattcgcttatgtttcatatacaccttgtacacagagtctgaaggtaattttatacaatattttaaataattttgtgcgtgaaacaaagtttatgtacaagagatattacagctgaagggggctggaagggtcttttttcccctggggctgctgaataaactgtgtgttatgtgcctggtgcttcaactgtaacctgtcatgtgaggtcaggtgtggaattttccacttttggcatcacgtcagtgctcagaaatgttcagattttgaaccatttcattagggatgttcgacctgtggctgtttttacttccccattttattttattttattttttttgagatggagtttcactcttgttgcccaggctggagtacaatggcatgatcttagctcactgcaacctctgcctcctgggttcaagtgattcttctgcctcagcctcccgagtagctgggattacaggtgtgcactgccatgtccagctaattttgtatttttagtagagatggggtttcgccctgttggccaggctggtctcgaactcctgacctctggtgatctgcctgcctcagcctcccaaagtgctgggattacaagtgtgagccaccgtgcctggccaccttccccatttgagatgtgagaggtaatccaccctaaccataaattagtttttcatagtgttaaataagaaataaatcatcttagtctctagacaaagaggtgcaaggaaaattgagtgaatgaatgcagtgctttgggtctgagaatacaaaaggtgaacgatgataaactttaccaagaaggccctatttacatgggatttcttagtagcgagtaccacgcttgcatttaacataggattccatctgtatacaaattaggtctataaaatgccaggaaccctccagtaacgcaaagcccaagacagttcctcttacaattaagcaccacttggttgtcactagttctgcttgaaatgtggggccactgttctttcacactgtaatggcatattcaaactgtctctgccatgttcaaattagcaataaccaaatgaaaattgtaaattctgtgtgttcactggcaagccggaaccactgacgaaagcttcaaaaatccattatttcctgggcactcaccagaccccccacagatgcatctctgtgtcttcagcctctggacctcggcttttgcccattcacatcccccatgaggatggctcttgtccattttctctcccagttggtgtttcttctccccttgctctctcctggttagaggctcttcactgggggtccgtggggtgccaagggcagtaggggtgtcaggggggccttccaaagcaggctattgtttgagaatgatgttgcatgtcacgtgtcacacatgtgacattgtgtgtgcttctccagactagagggtctgtcccatgcaccagattctcaaagaggccagagacccctgccctagatgacagtctcaccccaggatacagagagtacccggatctctgttcagccctgctatgtcacaatcccactgagatgtcaataaccacatctcggccaggcacagtggctcacacctatactcccagcattttgggaggctgaggcgggtggatcacctgaggtcaggagtttgagaccagcctggccaacatggtgaaaccctgtctctactaaaattacaaaaattagctgggtttggtggtgggcatctgtaatcccagctacttgggaggctgaggcaggagaatcacctgaacccaggaggcggaggttgcagtgagctgaggtcttgccattgcactccagcctgggcgacaagaatgaaactccatctcaaacaaacaaacaaaaaactaaccacatgctcgatgtttctgtgtcttccctgctggttagtgacatccaaggcaggccacatgagtaagaaccctctgagatcttccacccttcacttccctaacctggaatccagtcattctccaggttttggggctcccaactccaaatgttcattcctctttgtcccccacttccatgtgacactgcctgggtgtgtagcctcagcaccccttttgtcgaattggtctctggctctggggtttcctcgcttggtcctccacactgggtcagttctctcaatggccacctccttccttgcacaagatcaagtgcagactcctgacctgggtgctggagggcagtcctttccatcgcccccgttctcctcacccagattctcaggccacatgggcgggcttgccatggttatggttgactcgtgcgctgcccccatcttggtccatttgcattgctataaaggaacgcctggagctggttaattttttatttttattatttatttatttagagacaaagtctcgctcttgtcccccaggctggagtgcaatggcatgatcttggctcactgcaacctccacctccaggttcaagcgattctcctgcctcagcctccccagtagctgggattataggcgcctgccaccatacctggctaatttttgtatttttagtagagacagggttccaccatgttggccaggctggtctcgaacttgtgacctcaggtgatctgcccgcctcggcctcccaaagtgctgggattacaggcgtgagccaccacgcctggcctatttttatttttttatttttgagatggagtctttctctgttgcccaggctggagtgcaatggcatgatcttggctaactgcaacctccgcctgccgggttcaggtgattctcctgcctcagcctcccgagtagctgggattacaggcatgcatcatgacacccagataatttttgtatttttagtagagacagggtttcaccatgttggccagtctagcctcaaactgctgacatcaggtggactgctggcctcggcctctcaaagtgctgggattgcaggcatgagccaccgtgcccggctgaagctggtaattaataaagaaaagaggttttgtcgctcacggctctgcaggctgcacaagcagcatggcacagcatctgcacgggtgagggccgcaggctgcttctcctggcaaaaggagcaatggagccacatgcacagagattacagggcaagagaggaagcaagaaagtgcgggggacggccagctctcaagggagctaacagagtgcaaactcactcattactatgaggatggcacaaaaccatgagggatcctccccaggacccacacaccccccatcaggcccgactcccaacacgggattgaatttcaatatggggtttagatatttggagtcaaatatccaatacatagcactcccaaaagatgctgaaatcctaatccccagtactgtgactgggacattatttggaggtagggttgtatggaaaaatcaaactgcctttcctcttctctcacaccacaagtgtcaacactcctgtgactcctgtgatgaacgggctgaggatttctccccgccagcaagtcggcactcagttctgcagcggcgccagctgggtgtcccccagtccagttcaattccagcatggtccatctgcagacagcacaagatcccacagggtgagggctcagtcccacaagacagtcaccacttttttttttttttttttgaggcagggtctggctctgttgcccaggctggagtacagtggcatgatcatggctcactgcaacctcgacctcctgggctcaagtgatcctcctagctcagcctcctgagtagctgggaccccaggcatatgccattacacccagctaattttttattttgtacagagggggtcttgccatattgcccaggctggtctcgaattcttagcctcaagtgattctctggcctcagtctcccatagtgttgggctctcaggcatgagccattgcatccagccacctgccaccacttctgatgccagtcacaagccccagctagttctgcctgtgcttctgactggctataaattgagattcttacccccctcaggtttgattagctggagtggctcacaaaactcagggcaacactgacattttctggatgattacaaagggtgttacaaaggcttctgatgaacaccagaggaagagactggtagggcaaggatggaggaggggcattaagcttccttgccctccccaggtgcaccaccctcaggaacctccatgtgtttagctatttggaagcttctccaactctgtcctttcttttttttttttgagactgagtctcactctgtcgaccaggctggagtgcagtggcgtgatctcggctcactgcaacctccacctcctgggttcaagcgattctcctgcctcagcctcccaagtagctgggattacagacatgcaccaccatacctggctaatttttgtatttttagtagtgacggggtttcaccatgttggccaggctgactcctccaactcctgacctcaagcgatccacccaccttggcctcccaaactgctgggattatagacgtgagccaccacgcctggccccccttttgggtttttatggagccttcattggctaagcacaa

SEQ ID NO: 21 - DNA, 107818 bp, Homo sapiens
  Genbank Accession No. AL023553
gatcttggctcactgcaacctctgccccctgggttcaagcaattctcctgcctcagcctcctgagtagctgggattataggtgcccgccaccatgcccagctaatttttgtgtttttagtagagacagggtttcaccacgttggccaggctggtttcaaattgctgacctcaggcgatccacctgcctcagcctcccaaagtgctgggattacaggtgtgaaccactgcacccggcctcttgctgtcatttctttggattcatttattcttctctggtaacaaggtacccttgaaggttgttattcatagtttaagattaactaatacaggcccggtacagcggctcatgcctgtaatctctgctttgggaggctgaggcgggaggattgcttgaggccaggagtttgaaatcagcctgggcaatgtagtgagaccctatcactacaaaaaataaacctaaaagaattagggtgggcgcagtggctcacgcctgtaatcctagcactttgggaggccgaggcaggtggatcacctgaggtcaggagttcaagactagcctgaccaatatgtagaaactgcatctctactaaaaatacaaaaattagcaaagtgtggcggcgtgtgcctgtagtcccagctacttgggaggctgagacaggagagttacttgaacccgtgaggtggaggttgcagtgagccaagatcatgccactgcacttcaacctgggcaacagagcaagactccctttaaaaaaaaataaaaaaaatattgggaggccgaggcaggcggatcacgagttcaagagatcgagaacatcctggccaacatggtgaaaccctgtctctattaaaaatacaaaaattagctgggcatggtggtgggtgcctgtagtcccagctacgcgggaggctgaggcaggagaagtgcttgaacccgggaggcggaggttgcagtgagctgagatcgctccactgcattctagcctggcgacagagcaagaatccatctcaaaaataagtaaataaacaaataattgggtgtagtagtgtgcgcctgtagtcccagctaccgaggaggctaagatgggaggatcatttgaagtcaggagtttcagactgcagggaagtatgatcttgcccctgcactccagcctggacaacagaacaagacactatctcttaaaaaaaaaaaattaactaatacagatcccaccataacaatcatttatttattgctgtgtaacaaattgcccccaaacttaaaggcttaaaacaacaacaagcatttatctcatatttttgtgggccaggaatttgggagtggcttggctgggtggttctggcttggggcctctccagaagttgcagtgaagatgtcagccggggctagccatctgacgggttgaaccggggctggagcattcgcttgcaagatggctcactcacacctggcaaggtggtgctggttattggcaagaggcccctgttcctgtctaggtgggcctctcccaggattgcttaacaatatggggactgtcttcccccagagcaatccaagagagagagcaagccaggcagaagctgttcttcttatgacctagtctcaaagtccaatagcatcccttgtcacattctataaagtagaagtaagtccgccacacattcaaagggagggtaatggctgggcacggtggctcatgcctgtaatcccagcactttgggaggccgaggcagatggatcacttgaggtcaggagtttgagaccagcctggccaacgtggtgaaaccccgtctctactaaaaatacaaaaattagacaggcatggtggtgcgtgcctgtaatcccagctactgggaagctgaggcaggagaaattgaagcttgaacccaggaggcggcggtatcagtgagccgagatcccaccactgcactcccacctgggcggcaagactccgtctcaaaaaaaaaaaaaaaaggaagggaattaggcaccaccttttgaagggatgaggaaacatccaaagctcacgcacagggttttcctcattcattcttccctctgacccatggatatttgttgaatctccagtatgtgccaggtagtccagacatagagatgagtaagataaataaggcttgatctcaaccctcaagaagcttatagtgcaggtagactaatagctatggtttggatagtgttttacagcttaatgagcactttcacttatggtcttatttaaccttcccagtaccccgtgtgatcaccagagctggttatatccctattttctatctcctctgggttttagtcaatgagggcttttcttacattgatacaccttacatgatagaacttgactttgatcccagcgatttttgtctataaatcccttgcacttttttcctaccccaggctgtgttccttgtggctgctttgtcatccctgtcctgcagatgaggaagctgaggctgcagttactgaacagctcttgacactttaattcctggatttttttcaccatactgagctgccctcggggatggactctcctaagtgctccattgacagtggctgtcatgtttcttcttgcagaaagctctgggtgtgcggcagtaccatgtggcctcagtcctgtgccaacgggccaaggtggcgatgagccactttgagcccaacgagtacatccattatgacctgctagagaagaacattaacattgttcgcaaacggtaaggctgcagatgggaggctgtgactgtcaagggcattgcgtctgctgcctgcccgtcaggcagagaaggaggtgttttgtgaaggatgcttggtgatagtggcagggtcaaggtattcaaggtacaaaggtttctgctgaggaaaggcattccagatcagtggtgtaacttctgtcctctctgccaagggagaatctgtgttatcaagaagcctctagatgtgaaccacagccaggtctctgagacactttggcactataacctgaacctcccaaccttttcagggcaggctgctgaccctcggcctgccctctggaagccaccaggcccagtgcctggagaatcatcttctatttcctcttcagtgcacaggcacagaggagagctcaggtctgagacccagaagataaactaagatgggacctttttaatttaattttattttattttattttattttatttatatatatttgaggcagagtatatatatatttatatatataaaataaaaatatatataaatatatatatatttgaggcatagtctcgccctgttgcccagactggattgcagtggtgcaatcttggctcactgcaacctctgtctccccagttggaacgattcttgtgcctcagcttcctgagtagctgcaattacaggcacctgccaccacacccggctgatttttgtagttttagtagagatggggttttgccatgttgcccaggctggtctcgaattcctgagctcaagcaatcagcttgcctcggcttcccaaagtcctgggattatagacgtaagccactgcgcctggcctattttattttatgattttatgttatgttattttattttttgagacagtctcactctggtccaggctggagtacagtggtgcagtctcggctcactgcaacctccgccccccaggttccagcaattctcatgcctcagcctcccgagtagctgggactacagggacgcaccacgatgcctggctaattttttgtatttttgtggagatggggtttcatcatgttggccaggctggcctcaaactcctgacctcaggtgatccacctgcctcagcttcccaaagtgctgggattacaagtgtgagccactgtgcccagctgggactatttttttttttgagacagggtctcactctcttgcccacactggagtgtagtggcacgatcacagctcactgcaacctcaatctcttgggtttgagtgatcctcccacctcagcctcctgagtacctgggaccacaggcgtgagccaccatgctcacctactttttttattttttgtagagatggggtatcgccacattgccactgatctcgaactcctgggctcaaaactgttctcccaccgtggtctcccaaagtgctgggattacaggcgcgagccatggtgcctggcctaaggtgggatcttgagcatgtcttttttctggacctcattttcctcacttggtaaaatggcagtcagctctgccctgctgtcctgtgtgccaggtcctgggttagatgctgccaccaaaaggatgaataaggtaccacagaggatgaggagtcggggctggcaatgcacaccagcctgcactgccacacttccaccttgctcttcatcaggcatgaaggagagctgtcatagaagtttgctttgattgattggaacctgaacaactgcagatggggctgtgcccttgcctgcctggtggcttctttgatactgacagtggtgatcctgggagagaacaaatctgagtctacccgctgggtagctgaggctcagctggagaaggaacctgcccacaaccatgcagtgcatgagtggtggtgtctcgtctttgtaccctctagtactctaaccactaccccatactccctttctcatgccactttgctttctatctactggtatccccacttcggttttaagggtagggacttccttaatctggatctccagcctccagcacagcatcatctaattatagtaaaggacctattgtttgtcaaggacggctgtgacaggagtacccaccttgttgtccagtctggcctgggtacttgacacacattatttctgttctcaacaacccctgtaggttgttgctttccatctctgccccccaccattctgcaggtaacaaaattgaaagtcaggttggggaaataccctaaggccatacatgtattgagtgactgaccccacactcaagcctgggtctctgtgactccagggcttgttcttgtgggtttcttctgggctccatgttagaatcacctgggaagctttaaaaatgacggacacctgggccccactcccagcaattctgattcaagtgatctaggcattgctattcaaaatgtggtccatgggccagcagcattggcatcacctaggagcttgttagaattccagcatcttagaccccactccagacccactgagtcagaaccggccatttaacaggatccccaagggacttacgtgcctgtcaaaatgtgagaagcactggcccgttccccagcaggtcccatagtgcagtcaggacgaagaaccactgtccctgtcaatataggacgttgtatttattcttggagctctcacaccgctgctcagtggaaaagtgatagatttttctttactctgtaaaacacttccaccttgctttcatcaggtatgaaggagagctgtcatagagatttgttttgttgttgtttgtgtttgtttctgttttagactgagcctcactctgtcacccaggctggagtgcagttgtgcaatctcggctcactgcaacctccacctcccaggctcaagcgattgtcctgcctcagcctcctgagtagctgggattacaggcgtgcatcaccatgcccaggtaatttttgtatttttagtaaagatgggggtttcaccatgttggccaggctggtctcgaactcctgacctcaagtgatccacctgtctcagcctcccaaagtgctgggattacaggtgtgagccactgcgcctggcctttttttttttaattaataaaaaaattttaaagatgggctgcccaagctggtcttgaactcctaggctcaagtgatccccctgcctttggcctcccaaagtgttgggattacaggcctgagccactgtgcccagcaagaggttcgctttgagataacttgacaacagagtattattcactatttagtttttgcattaaacaattaataacaacaacaaaataccctttaaggcactgggagttttttagttttaattttatcattaaaattgacaactacaacaatatttttccttgcttacttttattccaaccttgctttgcaaccagaaaagtccaataaatttttttctttttttttttaaagtccaataactttgaacattttccagattagtacagacgaggggattgttctctgttttctttttttttgaaacggagtctcgctctgtcgcccaggctggctggagtacagtggaacgatctcggctcactgcaacctctgcctcccaagttcaagtgattctcctgcctcagcctcccgagtagctgggattacaggcgcctgccaccacgcccagctaatttttgtatttttagtagagaaggggtttcaccatgttagccgggatggtctcaatctcctgaccttgtgatccacccgcctcagtctcccaaagtgctgaggtgacaggtgtgagccaccacacctggtctgttctctgttttcattgttgccacagagcaggtctcaacaaggtggggggtggtcagttttgtccctagggcccatttggtgatgtctagagatatttttggttgtcataacttggctggggttggggcttactgttacccaatgggtaaccaggaatactaccaagcatcctacagcgcacagggcagccccacaacaaaagaattctgcggcccaaataggttcatagtgccctggctgagaaaccctgacacggagaaagagggcagaggtgaggttatgctaaaaagaattatattctagccgggtatggtagcccacgcctgtaatcccggcactttgggaggccgaggcaggcggatcacttaaggccaggagtttgagaccagcctggccaacatggtgaaacactgtgtctactaaaaatacaaaaaaattagtcaggcatggtggcgagcgcctgtaatcccggccactcgggaggctgaggcaggagagccgcttgaacccaggaggtggatgttgcagtgagctgagaccacaccacagcactccagccagagtggtgtctctggtgacagaggaagagactctggctcaaaaaataaaaataaagcgttctattctaagaattttttagaaggccaggggacttgctgggcgacttctcttccaccttgtgtctccaacagaaggggtctaagtctgttcttgagaactggatctgtgtcatctggagagcagagggacagatggatatgaagacccattttgtcatcctcagttaatcctcagggacattcaatgatgaacccatcctaaagcttcagcattgctgagcttttttatatagcttcaatcatttgcttccaaggtcttgggtttttaagcccatgtgctattttttgtctccctcggggcccagaaggaggggctgtgagtcattgcccgtacaccagccttggcggtgctgccgcaatcggcgcaggctgtacctcaggctagtgctgtttttattcatagagatgctcagagttgccagttggtctgctgttgccagcagatccagcaccttagggacttttttttttttttttttttttttttttttgaggtagggtcttgctctgttacccaggctggagtgcagtggttcgatcatggcttactgcagccttgaccttctggggtcaatccatcctcccacctcagccttcccagtatctgggactataggcacatgccaccatgcctggcttctttttatgttttttgtacaggtggagttgcccaggctagtctcaaaacgcctggactcaagtgatctgtctgcctcggcctcctaaagtgctaggatgacaatcgtgagccaccacacccagctgggaactcttactttgaagaggttgggtaaacttgggggtgagggactggaacccagcttgcagaaggaagtttctctgccatctcactatgcatattgatgataacagtaatgataactgagaaaggatgaggtggctcacacctgtaatcccagctcttcgggaagatcatgtgagcctaggagttcaagaccagcctggtcaatacagtgaaaacccatttctacaaaaataaaataaataaataaataaaattagccaatgtggtggtgcgtgcctataatcccagttactcagaggctgaggtgggaggatcgcttgagcctgaaaggttgaggctgcagtgagctacaattgtgccactacactccagcctgggtgacagagcaagaccctgtctcaaaataataacaataataataataataataataataatgaaaatggaacattcaccatgtgctggccctattccagattccaggtgtggagagaccgggtagattagcgtcaggagcacagccgtagggccacactgctgcctcttcgtggtccagctgtacccctcactggcttcgtggtctggagcaaagctactctgtccctctgtgctgcagcttcctcatctgtgctgtgaggataaaatgagttttatacatgaaaatacttaagataatgctgagctgaaagtaaatgctacgataacgctggtctctagtgacagaaagccaatcagtagtggcaaaggggcgggagggagttaaccaaggggtggggaaacttgaaggagagatggtcagtatcttgattgtggtgctggtttcacaggtgtatacagtgtcaaaacttagcaaattgtgtaccttaaatatgcagtttattgtatgccaattatacctctgtttaaagctgtttaaaaatgaaaggctatagaagtggaagctgttatctcatcccagtgcaagccctggtatttagagatgggctggctgagctgaggagagcagttgcttatcttgttaaatagtggtttcagtaataggtcaggtttgggcttttttgttttttttttgtttgtttgtttttattagagacagcatgtcactctgttgctcaagctggagtgcagtgatgtgatcagggctcactgcagccttgatctcctgggcttaagtgatcctcccgcctcagcctcctgcgtagttgggactacaggcacgcatcaccctgtctggctcattctttttttttttttgagatagagtcttgctctattgcccaggctggagtgcagtggcgctatctcagctcactgcaagctccgcctcccgggttcacgccattctcctgcctcagcctcccgagtagctgggactacaggcgcccgccaccacgcctggctaatttttttgtatttttagtagagacagtgtttcaccatgttagccaggatggtctccatctcctgacctcatgatccgcctgtctcggcctcccaaagtgctgagattacaggcatgagccaccgcacccggcctcattctgttttttatagagacaaggtctcactctgttgtccaggctgatcttgaactggcctcaagcgatcctcctgctttggtctcccaaaatgctaggattacaggcgtgagccaccacaccaggcccccaggtcagggttaaactcagagcttctgactcccagtctgaaacttttttcttggcctcaaacaataggacagagcctgtcaggtgtgggttgaactctgctcctccttaggaaggaagtctcaagcttggcctaagctttgagaattctggggggcagagcgtgaggtctgaaaggggctgttgtggcaggcagggcggccacaacagcccctttgctctgttgtgtgggtgcagccgggagtcagccggaacgttgagcctggcagggagagtggacccgcgcagagctcagtctagatggctgggaggcttgtggggagaagacggttgagtcctgacagggccggaccccagggcaggtttggactgccattttggctctgatgaggaagggaggggtggggcctccggggaaatggggtctaggaggaggaggagctgtgagaggccagtggcagtgacccggtctctgttcttgaaaactgacagatactcctgtctatgtctggagctaatgggcaaagtcttagtgtcatagttgggtagccaggcatcccaggcctgaggtgtggagggttgtagatggcggacagggtggacaggtgaaggggatgctggtcctgattccctgtgccttagtggggctgagggcacagagggaagtagcgaaatctagctgtgaacttggtcactctcctgagcctgtttcttggttgttgttaaatggctttcttggttgttgttgttgttaaatggcaggaatgatgagatatgcatgttgcggggacttagtaggaattacgatgctgggaaagccaggatcctttgaaaagaaccagcgctgccctcttgtgatgttgtcattcacttactcatcaagtaattgtggagcacctaacaagtgccaggtgctgcaggaggtgaggaggtggtgcagtgaacaggacccagcagtccccacccaagtggaacgttgaggtcctgagttcagactccctgtccctggccactgttgaggttgccacatggactgagagggagaggcagggcgggaggaggccgtgcagctagcaccaggccacccttctgctcttctccccacagactgaaccggccgctgacactctcggagaagattgtgtatggacacctggatgaccccgccagccaggaaattgagcgaggcaagtcgtacctgcggctgcggccggaccgtgtggccatgcaggatgcgacggcccagatggccatgctccagttcatcagcagcgggctgtccaaggtggctgtgccatccaccatccactgtgaccatctgattgaagcccaggttgggggcgagaaagacctgcgccgggccaaggtgagcagaaggtggctttgggggtgggcaagtgggcaagactgggcgagaggcctcaccctcacactggagcaaaccagggcattgcctccaaattctcacacctctttggattggtctgcttttaaaacttgattttacttgtttctctttctaaaaatagcacttgctcattatgttaaaaaatacagaggaagcctaaagaaagaaaaaatcatccttgaccttaccacactgcaggtcctttttctgtcctgttggaggatgatgtggatcctgccaggtgctctggaaggggccatgcaggcctccctgtctgcctgagcagtgaggggtgcatcttagcatagtttgccagcatcccatttgcacccactcccgctctcctctgaccctccaaggaccctctccgggaggtaggcgtgctagggtcactatttgtaaatttgaggcggcagtggcatcatacttgagttctgtaactacatgagggcacgattagggctcaatgctgatcctggctcccactctggtgctctgtcactgaggagcctcctttgggggccattcttctgtgagttttgcttaactacatgggggtggctgtatgccatgctgaaggcttttcccccacccttctatgaaatcgagttcctgctccttctcccaggctcggcttgtgggacctttgtgtcagcctgctcttggcaacagcgggcctctctctcccagctctgggcctccccactctccagcccctgcagtgggagtgggtccccctcacggggaggggcgttggcaggagctgaatgctggcagggctgcggcctgtgggcatgtgattgaaagtgctccagctcccagggtcagcatggctcaaagcctcccagccccatgggaaacactcggggcggcgctctcactcagaccctctcttccaccctggccccctggtggtatcctggctccacagtggctctttcccctcagctgaaaggacagtcctgcctgccggccggcggtttccacattagcaacaacactggtgggggtaataatgttacaggagctcccacagtgccgggaactgcacatggccgggaacgtgggtcctctgcagccctggggacagctgcccacaacagggctgctaccttctggggctggctttctgcttgctttatgggctcagagtgaatcttctggagatgagatgcacactcatagcattcagagctcattccctcatgcgttcaacaaattgaacattctttatgtgccaggcactgggctaggtactgaggttcagtgtgaacaagtcaggcaggggccctgccttcagggatctgcttgtgggtgtggacactaaacaatggccgaggtaggtaactgcagatctgggtatgtgttaggaagggaaagtgcgggacttggtcagtataaaaaagaggtctaatttagatgctgggggaaagagagagcttctttgaggatgtgacagataacctgaggactgaacatatgagttgcattgactggaggaagaacatgctagaaagaacagcatttgcaaagaccctgagccaggaagaaacttgatgcttttgtggaagtgagagggggcctgtactgctggagttctctgtgtttggaagagcgggtggtatgaggtgggcaggggcagccaggaacgcgagggctgtgggaacgctatggtcaccatcctcagagtcctagaacagtttaagcggagaaagaatatggtcttttttttttttttttttttgagacagagtcttgctctatcccccaggctggagtgcagtgacacacaatctcggctcactgcaatctcggctcactgcaacctccgcctcctgggttcaagcgattctcatgcctcagcctcccgagtagctgggattacaggcacccaccaccatacctggctaatttttgtatttttactagagacagggtttcaccacgttggccaggctggtttcaaactcctgacctcaggtgatccacccacctcggcctcccaaagtgctgggattataggtgtgagccaccacgcccggcctagaatgtggtcattattacgtgtatttgtaaagtgtccctctggctagggctcagaggcagtgacctgtctagggtcacagaaccagagatggatccaggctgagtccaggccgagactctggcaccagtctggggtcctcccacaccccttcctcacacttaacacagacgtgtagtcacatgcgcccagacacgaccacagcctcctgtcctggataccaatttcataagtgaccgatttgtaaatgaaaaagctgatgtattaattattaattggaaagagactgctctggaaacccacgttctttgccattctcttacctgggggaacccagtgggccttcagagggcacacactggtctggagtttcagtgaaggggtgtgtggtttgagtgtatctcatcaagggtccgagggcactggctgaggtcccttctctgaagcatcgctctcccatgccggaggctggcaggtcccatttcctagttcctcctttgagactcagtagagtccactagaatcccaccgttttgttactggggtcagtctcactgaccccacacagggacctcccatgtcttccagataaaccctggcctcacatctgctgtgcagtgggcccttccctgtccctcaaggaagcttccttcagattataggcaggatggctacagcccgtgtcatcatttcgtttggctgaaacctccttttcagaacagtggtcccttcgacagccttccatttgggcccatcaaacacattaccaagaattcagcacttccgacagtcagacaccagtccagggggctctctcaggtgtcgtctaagcctgggatctccagcaagttatttcaaaaaaagcctctggggtttttgttttgttttgaggcagggtcttgccctgtcactcaggctggagtgtagtggtgcgatctcggctcattgcagccttgaactcctgggctcaagtgatcttcctgcctcggcctgcaagtagctgggaccgcaggtgcacagctaatttttatttattttattcttatttttattttttgagacagtctcgctctgtcttgtaggctggagtgcagtggcacgatctcggctcattgcatccttgtctcccaggttcaaccaattcttgtgcctcagcctcccgagtaactggaattacaggtgtgcactaccatgcctggctaatttttgtatttttagtagagacagggtttcgccatgttggccaggctggtcttgaactcctgacctcaagtgatctgcctgcctcagcctcccaaagtgttgggattacaggcgtgagccactgcacccagccactgcgcctaggccagggcctgcctctgcactgctggcctcatctggggacttgagtgccacaggcactcttggcaattatttagaccagtcccttctccagataaggaaactgagacccagaccagggcagggacttgttccagacccaccaccctggccaggtgtcacagaactggggctccatctctgctcccctgtccttctctctgacactgctagtggagactgggccaacatccaagctctttggaattatttttagaaggtctctaagagagggcctgtctccagtgccagtatttcagagtcagaagggaggctgatggggtggggagggcttggtgagggtcacctggacacaaaccatgttgctaacgcccaattattgatttgtctcaataggacatcaaccaggaagtttataatttcctggcaactgcaggtgccaaatatggcgtgggcttctggaagcctggatctggaatcattcaccaggtaaagctgggctcagtctgccgtcccaagggcccaagccagagaagtatgttccaggcctatggggggagggggtttgaggcccaggggggctgaggggaactggatgacatatccatcagctcttatgctcttcctcctttgttttgaaactgattttcattatgtcattatacgtgctcattttctgggtgagaagttaaaataatacctcctaaccacccaggtaaccagcattttaagtgcgacatgtttctttccacattgtcttctgggtgtgtactcaagcatctctattcccataaactctcatacagaacatggtattattttgttttttttcacaagtgtcaccccttgtggcatgcatcactgacatttgatgagcccatactgtgtctcaggtactaggctgagcacttgaaagatatttgccatgccctgtgaggtaggtgcttttactatccacaccctacagaggaggcagctgaggcacagagatgttaaccttcttgcccagggacacctagctggcttactgtggagccaggcctgtgggcaggagggctggtctggagcgagtgccccatgccaccgtgctgctctgccgcctccctcgcccccagagctctttctgcttctgcacgtggcgctcctcggatttctttttggctccttcatggtgttctgtagggtgaaggtaccataggtcatgtggccatgtcagccgggggctcctctgaggtgtcccaaggatggatcttcttttggtggacagttggacagttttgtctctggaagtgcaagtgtgaacgtgtatatcctggggaggtgggtgcttccctttcttccaagggcactgttagggaccactgacctagtttgtgtccatttacatccaccctgtatgtatgaaacctgcctcttcgcacctttgcaaggtggcccttccccccacctccatcccagacctcactgtcctggcctcctcaggatgtgctgagtcaggctgtaatctgacgccgcatgggctcccagcaccgccccgctctgaaaccagagccagcaggggatcactcctctgccctgcccaaccatcactcttgccaggaaggggctgcgtggaatggccgtttgcagcggcctgtggggactagaaagcccctctctgcagcagggttagggagtcaggtggacccagtcttggccttctgttacaaagggcactgcaatgcaggtgtctccctttctacctcagtacaaatgcccaggcctttatggcagctcctgggccaaagtttggagccgtgagtgatggcagctcacatctgccaccggctagctgctaagcatacagcagctcgtgtgtttgtttctttaaaaagaaaatgcccaggggccatgggagtactttgcagctttactttcttcccgccttgatgtcacgtcttcagcagcctcagtccattaggtccaacccaaacctgaacccctttccctctgggaagtgccctggccccacttggcccgcagaccttcctccacatcacagctttctggttcctctgtggagcctccccggccctccccgtgcctggcacagtggcttccagtggatcctctgtgggatctcttcacattatgttccagcatctgccaagtgctttctgcactggatcatgtgctctgtggggacagggactgtggcttatttattcctctctagctctgggtttgggaccaggtcagtacagagcaggtccttaatgtatgtgtgtgttgtgtggatggatggctgaatttccaggtgtagttctaggcattagtccttcttcccagccctcacccccatctgtccagccacacccacagaccggggagctcactgtcacggccagcgtcatcgtgacatcttctgcagtcctcatgtctgcctgacctctccccctggatgtggaccacaagtgtctacaccgtgtggaccacaagtgtctacaccgtgtagaccaccccgaattctgtgtgcccagccttcgtggatccaacttttacaagcttgaggctctttagggatgcactagtcttaaatccttgtacgtggcagcattggccagttcttcgcagccaggtcttccctctgcatccccaagagatgagacctttggaccttagagtctgctaggcagatgcttgtcctgaggcagaggccctggggtttctgtaagatttctgggagcactggtgggtcctgagagttcacagagcctgtcacacctctcatgtgttcttccccacatccccgggtggggcactaactccatctgactgatgagcggcagaacctcaggtgaaggctcttgctgcaaaacaataaataaaaatgttgcttcccgcaggcactattctgtgcctcatggacaggatctcatccagtcatcagtgaaccaatgatgtgagcacgattgttagctatttcttgatagggaaactgagctccgagagaagtgacttgtccatggtcccacaacaactagtagatggtggggccagacctcagaccccccgatacaggacgcctggctcagggcttgccaagcagtgtcctgcagcttcagggggccagtgcctcagaggtgcctggtgcctgggccacacttgggtgttgagcctatacagcacccaaggtgttgagcacttgggtgttgaggtgttgctgcctttcttaccaaatgttttgggggaagtgctccccacggctgggaggagcgcctcagtgaagggagtactgtgaaatgggacaggtgatgaggcagggtggcatcgcgccttgagcaagtcacatgggttctcagagccacctccagatgctgaagtggacgaggcatcacctattctttgcagttgaggaaaagttcatagctttgtgtacccactggggcctcccaggacttcttcctcaggggccttgtggggctggagccagctcctggaagaaccaccaaccccgatgagaagccaggtggggatgctccacacccaggatgaggccgggcctgggcccatcacaggacagtttctctggttcttgtcacatcctggggtcatggtggtatgggtgtttggttactgtgctacaaatgaggaaactgaggcatggggcagcattcaaaggcccaagacatatagcaggaaatatcagagctattcaagagcccaggccacctgtctcccaaggctctggctcttcttggccaccctggagacggcctggattaaatggggctgctcctaccagttccatcctgggagagtggctggacgtggttgggaggccccgggtcagtggggccattttttggtattctcggctgagggcttctaaatataacatcttggattatttttcagattattctggaaaactatgcgtaccctggtgttcttctgattggcactgactcccacacccccaatggtggcggccttgggggcatctgcattggagttgggggtgccgatgctgtggatgtcatggctgggatcccctgggagctgaagtgccccaaggtgagggtggggagggactcattctgggctggctgtggggtggtggttggtggggatgaacgggagacggtgggacccaggagggaaaagggaacaagttagactcgaatcttctgggagggaggtagagaccaataagcagcaatgtgaatggcagcagggccatcctgacttcgtggctggcacaggcacacacggcctctcacagccgcctcgccccctcctgtccaggtgattggcgtgaagctgacgggctctctctccggttggtcctcacccaaagatgtgatcctgaaggtggcaggcatcctcacggtgaaaggtggcacaggtgcaatcgtggaataccacgggcctggtgtagactccatctcctgcactggtgaggaaggcggccaggcgacgtggcccctaccctgtgctgggcctgatgggtctccagttgggagtagaagcggtgaatggccttcacttgagaatctgtctgttccatttggggacttgggatagacagaggtagaaggaaaattggagacagcattagagattgtctagtccacatcctcattaaacagatgaagagattgagatacctgtcgaggctgccggtgacttcctaactgtgtggccacacacggttaggagccaagtcagcttctgctgctgggcccaggccatagcactaggccaccttgggtgatctagacgaatccagagaggacagaggcagtggtgggaatgggcagagccagggcttcattcatgaagcagctgcaggcccacatcctcccttcgctgaagttcgagcctcactcacttcccttggattagagatcggtgcctgtccgcctccaagaccaggtaggagccagctgctccctgcctgtcactcctggtgacctggcctggatgcctggtcctcagaaatccagggagatttgtaacagtctagacttcccaggtgcctgcctgggctccctggcagccctgcagaactctggttcccctgcagtgatgggggctcactcctgccacgggactgctcagtcaggacgttggtaacaggcccatacctccgtcctagtggctcctgaggcaggtggcctggtagcagctgcagtacctgtgagccccaccagttctgtgacgctttgtggtccagacagtgtgtatcatttcagtttttgtttttgttcttgttttttgagacagagtctaactctgtcgcccaggctggaatgcagtggtgcaatctcggctcactacaacctccgcctcctgggttcaagcaattctccggtctcagcctcctgagtagctgggattacaggtgtgcaccatgatgcctggctaattttttttgtatttttttttagtagagatgggttttcaccatgttggccaggctggtctcaaactcctgacctcaggtgatccacccacctcggcctccatcactggaaaacttgagacacagaaaagtgtctccaccatggtatggctgaaggagggcaggccaccaggcagacagaccaagatccgaatgtcagcatctacctttatgtgctttatggcatgaccttgaatctctctgagccagttcctcccctgccaaaaagaatagcaactgggcagtgtctggtcaggtcagcctacagtacgtggtgcctgttcaatctcttgggcgacagtcaggccccaagctcgcagctcagtgtgtgggggctgaatcttcacctgagtcctcttcattctacccagcagccaccctgagctggggccctgagctctgatccccatggcctcccctcaccggcccaggtttttacccagggcctcaagggatgagtcggcccgctgtcaccacatctctctgagtgggaggagaagcaatgcagctccctggtgtgaagatgcaggtggccgcgtagcaggtgtgtgggaccctggcccggccaccagccaatgcccggggctctgttgctccacaggcatggcgacaatctgcaacatgggtgcagaaattggggccaccacttccgtgttcccttacaaccacaggatgaagaagtacctgagcaagaccggccgggaaggtgagctggcaggggcaggcccgtgtgggtggaacagtcacatgcccgctcctccccaagacagagctatgcctttccctgtagggcaggggttcttaacccattgtctccagacaggcgtccgaggctctgagaaagccctgaagaggtgcaatatgtttctgagtagattgtctggagctctctccagcttctcaaagggcctggcatctgagttcaagatagccccacctcaggatgggcatttcattttccctggaaggtggctgaatgcccgcccttctcggcctggtccctgctgccccctacaggaaactccggaaatgacatggatcgcttctgttgataattcctgacctagcaggggaacatttggaaggagaaccactttcccagcctcccctgacaacttccctgaggctctagcctggcttctttggagctcagagggcattggcacctgtgaagggtggtgcgttgtgggagtgaacgaggagccagagaccaggggccatggggagagccgtaagcacgtggaatccttggaaggctcctccctttccttattgagtgactgcttcttcaaaacccagctcttcacttccaggacaccctccccggctctgaaccttagctggcgctcctcctctgtgtctccgtgtccctgacagctcctgcactagggtgtgagctctgtaagggcagagacagcagccttggtttcttcattggcctagtcagtgcccagggtgggcctggtgtttggcaggtgctcaggtgggtggtgagtgaactctcaagaacagtttatgtttcacgtgctccatccccgtcccttgttgatttcagacattgccaatctagctgatgaattcaaggatcacttggtgcctgaccctggctgccattatgaccaactaattgaaattaacctcagtgaggtgaggagacaattaactgggttcaagaagtttctgagagtagtggggagcagggcgggtcctgcctaaatactcactgagggccgggtggtggctcacagctgtaatcccagcactttgggaggccaaggcaggtgggtcagttgaggtcagcagttcaagaccagcctgtccaacatagtgaaaccccgtctctactaaaaaaaaaaaaaaaaatacaaaaattagctcggcatagtgccatgtgcctataatcccagctattcaggaggctgaggcaagataatcgcttgaacccgggacgcagaggttgcagtcagccgagatcgcaccactgcactccagcctgggcgacagagcgacactgtctcaaaaacaaaacaagaaaaacctcagggtgactcgtgggcccctgggtccccagtgttcctgcaaactgcttcctgaagcagcctggagcagtgggtggggaggggggagacacagccacggggagagcagaggtagctctggctgctgaaccagaagtggatgtcagtttgccaccagctcagctttaggagaagccaaggtttcaccctgaagggtttttgttatctgagttcagagggcaaatcccagcagggttaggcaccctgagacgcacagatcagagcaggtgtttgcagagccagaggaagggcaggctagaggatcccgtctgcaggagttcacacctcggctctgccacttaagttgtgtggccttggtcacgcacttagcctctgtaggccccttgctccatcacgtgtggaacctacagcagtcgtgaggattgaatgaattcatgtaactaacactgacggaggtttactgcatgctgcgcacatagctgagtgcctcgcatttactgatgattatgatatgtaaagcgtctggccttaagtccatttttgtcatgattccaaaagcggccgggcatggtggctgacacctgtaatcccagcactttgggaggccgaggcgggcagatcatgaggtcaggagattgagaccatcctggctaatgtgatgaaaccccgtctctactaaaaaaatacaaaaaaattagccagacgtggtggcgggcgcctgtagtcccagctactcgggaggctgaggcagaagaatggtgtgaacccgagaggcggaggctgccgtgagccaagatcgtgccactgcactccagcctgggcagcagagcgagactccatctcaaaaaaaaaaaaaagattccaaaagcaacatcgaatgggagataatgaacttaggaagctgacagccttgatccattccatgcctctgctgtctcctagctatgtgacttcaagcacagcatgtcacttctgagcttcagttccttcatctgcagagctgagcccagccttgcagggctggagggagcccgggagcgggtgtgtgcagagccaactgctcagttcttccctgtgatgaggtttcagtcaagagaaagtcgttggcctctctgtggggacgtggtgaggcagtgaaagaggctgtccccgcttcaaggtttcttccctcctctctttcttctccttgcatgtttgtttcttcagctgaagccacacatcaatgggcccttcacccctgacctggctcaccctgtggcagaagtgggcaaggtggcagagaaggaaggatggcctctggacatccgagtgggtgagcaccttccaccccatctgtttagcaggtctcagggccagtggctctgcccagggctgtagacaatcacctatgcctactgtgtggcactttctaaggtgggcagtttctgaaatgggtttattattgaaaaattactttttcggccagaagtggtggctcatgcttgaaatcccagcatttcaggaggccaatgcaggtggattacttgagtctaggggttcaagaccagcctgggcaacatggaaaaaccccgtctcttaaaagaaaatgcaaaaattggccaggggcagtggctcacacctgtaatcccagcactttgggaggccgagatgggtggatcatgaggtcaggagatcgagaccatcctggctaacacggtgaaaccccatctctactaaaaatacaaaaaattagccaggggtggtggcaggcacctgtagccccagctacttgggaggctgaggcaggagaatcgcttgaacccaggaggcggaggttgcaatgaaccgagattgtgtcactgcactccagcctgggtgacagagcgaggctccgtctcagaaaaaaaaaaaaaaattggccaggtgtggaggcgcatgcctatagtcctagctactcaggaggctaaggtgggaggatcacttgagtcctggaggtcaaggctacagtgagccgagatggtaccactgcattccagcctgggcaacaaaggcagactcaaaactgcagactcaagcaacagagcctgcctccaaaaaaaaaaaaaaaaaaaaaaaagaaagaaaagaaaaattactttttcttggcagaaaagaaaaatatgtttattgtagagaatattcacaatcaagaagaatttttaaaactcctaatctcaacccccagccaggagcaagctcttagagctcttggagaggtgtgctgtgtttctgtgtattgtgtgcatagattttcccgttttacattttatatcacccatgctgctttatgtgacttttcatgctttacacatgcagacgccctggcatgccagtcactggcatctgctgctgctgggtacagtgtggctgtgaagagcatggagcctggagccagatggcctgggcttgaatcctggctctgccacctaacagccactaacagctgagtgaccctgagcaaactacttcactgtctatgtgtgggttccccatctgtaaaacaggagtcatgatcgtactgacacctttgaaggtggtgttgggcctactgctgctatcaccaatgagaaaaataaataaaaaataaagttgtctggagaagtgacaatatgtgaaaagagatttctataccgtctggccgaaaataagggcaatataatgtgttcgcccttattcattgaagtaattttaagaaaaaaaatatttggtggtttaataattagaaagtaaattactaaattaataaaagaaactatcttggctggttatatgggcttttgccagtaatcccaacccttttttttttttttgagacagagtcttgctctgtcacccaggctggagtgcagtggcacaatctcggctcactgcagcctccgcctcccaggttcaagcagttctgcctcagcctcccgagtagctgggactacaggcgcccgccaccatgcctggctaatttttgtatttttagtagagatgggggtttcactgtgttagccaggctagtctggaactcctgacctcaagtgatctgcctgcctcggcctctcaaagttgtgggattacaggtgtgagccaccgcgcctggccagtcccaacactttgagaggctgaggcaggaggatcatttgatcccaggagttcaagagcagcctgggcaacatggtgagactttgtctctacaaaaacaatttattgaattagccaggtgtggtggtactcacctgtgggtcccagctactcaggaagttgaggcaggaggagttgcttgagccccaggagttagagactgcagtgagcaagcctcgactgtgcccttgcactccagcctgggcgacagagcaagaccccatctcaacaacaacaaaaagagactgtctagacagaaagtgcgatgttattcaggttttatagactttttaaaaagaactcttattgccctgattataaaacaatctttaaaacccagaaaatactagaaagtataaaagaagaaaattagcatctcctgtcagccccagcccatcaaggatggctgttcgtatgagccctctgacgtgcctacacgtgtacatgatattgctgtaaaaggggtcatagtcctaatgtgttcagcatcatggccaatggctacagcatctttcttcaaatgacgccacgtcatgagttagcctgtcctttattgttggatatttcggttgcttgcaactggtctctgttaaaaaagtctctggcccagcccagatggtgaactcttttggccaagtctggataatttgaagtcaggtggagacctctgctcactgtctcctcctgacccttaaccccaccacccacaatgcaccaggtctaattggtagctgcaccaattcaagctatgaagatatggggcgctcagcagctgtggccaagcaggcactggcccatggcctcaagtgcaagtcccagttcaccatcactccaggttccgagcagatccgcgccaccattgagcgggacggctatgtgagtgcccatatccccctgcccatctcccccaccccatgctgagtaatgcctccaggcggcacaagcccagaggcctgttgggccggggctggggcaggtcccagtggctgccctggggttccagtacagcacttcgtcctgcagccaccacatcaccccttcccatcagactctcacccacccttgacattctgtcttcctctctccctggcaggcacagatcttgagggatctgggtggcattgtcctggccaatgcttgtggcccctgcattggccagtgggacaggtaagaggcgtatcttttgacaagacagccccttgtgcacagggtacagagccccagaagttggagggggaattattggggtggagagaagagactccagccaaggtctctagctccagggactcttgcccattagaagcctctggcagacatgcctgggaagaggggctgggtgaggaagggccctgcagaggcactggggggcaacttggctgaagcctgggctggcaggaggaagccagcgcggcctcaccctgacggacacagcaggagccgtttgggaggaggcagtgccccaggtcaggaaactcaggccttgaccaaggccttgcctctggcttacgtgtggttgcaggctgggcccagtgatcctgaggttcccttctgctctgcgcgtggccccaggaggaggcaaccctggcagggcctcttcattttccctcggtaggagctaggctgggctgcgccacaggaacccagcttatctgtcctcgggacaggccaggtgacaaggccagatatccctaaccctgatccctctgacctggcaggaaggacatcaagaagggggagaagaacacaatcgtcacctcctacaacaggaacttcacgggccgcaacgacgcaaaccccgagacccatgcctttgtcacgtccccagaggtgagactgcccagctgcgcacaagcctgggatggcctctgggggtccctggcgggtcagaggaggaggcagaaggagatggggactggggtcatccaagtggtagccaggagctacaggccttcccagcctcaggcgcatgcttggtgcttcctgcctggggctccctgggtcatgggattatgagatatttatacagtggtttgtgcttatgagcatggaatttggaatctcactcactgcctaggacacatcccagccctgcctctaactcgctgtatgagtgtggccaagtcacttaccttctctgtgtccccgtaatcctcgggattacctgggaaagtgcgtgggacacatgcagtttccaaccccaggtaggagttccacaaaagcgaggcctttgccattgttcccgtggccagagtgagccttgcccttggcggtattcagccctggccgtgactggcacatggccaggctctcttgacttggttgtttatctgcgtgttctacaaatatgtcttgctaaatgggtgaaggaatggctccttccatgtttttagtgtcctgcgcactgagcaggtttgcatcataccagcatttctgcgccagggcccccactccctccaggatagctgtgaggagagagaacagcactgccggcccggccctgcagggatgggagaggccgcactgcccttggcttcctgagtcctctgcaggcccaaggccccgtggtcccaaagatcgtcctgcagctctggcctctgaggaacacaggggtctgggaagaacatggaaatttcctgggttcctttctcagttttggcctaggcttttggtaggtgcaggagacaggagtggcaattggtgctgaccaacaaactggccacctccatttcagattgtcacagccctggccattgcgggaaccctcaagttcaacccagagaccgactacctgacgggcacggatggcaagaagttcaggctggaggctccggatgcagatgagcttcccaaaggggtgagcgcccacgccccctgcttgctggttgctgtgtggccacgtcacttccttctcaacctcacagcacctcctgtgcaggcagggagggcgctgctagtgagaaggaagcagctctgttccctgggaggggaggtggggcccgaggaaacttgccttctgagagtctgtccttgtgggaactgaggactcagcaccccacgcatccccattccctgctgcaggagtttgacccagggcaggacacctaccagcacccacccaaggacagcagcgggcagcatgtggacgtgagccccaccagccagcgcctgcagctcctggagccttttgacaagtgggatggcaaggacctggaggacctgcagatcctcatcaaggtcagcagcatggggacggcaggacagccccaccctgccagggccccccgtcccctgagcatcgggaagggccatgaacctggaggaagtgagcacagtcaagacgcaggtgggagatggaagggaggtttggctgcagagcagagagggtatcgcaacgcagtccagcgtcccccttctctgtggccccgaactgggcagagctagatctggccagcctccgtctggggccctcagccatccagcagcagctgatcagagccacctccagtgggtgtgggcaggtgagtacagctcaggctgggctgggacagtgtgtgtgattgcacagcaggctccacacctggcacgtccacacaggctctggaagccatggacaactcctgcccccacagttgggcacccgtggatatgggatggcttgtgtttggcaccgaccaaaaacaagcctttttggtgtggccagaggcctccaatctgtgtcagatatttatttatgctgcttattaaggggtctccaggcacccctgtgacagaagagactaatcagtcatcagccaggacccaggcatgtcctgggctcctgtgtccagcatgaggtctgtggctgatcttgcagctgaggcctgaagggtgagcgaacattgacctgtcccaactttgggcggcctctgccccataagggagactgagcagccagaggcctttgaggggatgaaggcctggcctgagcccatgtggccttagggtggaagcaccaggaccacagaacacgtgtctgaagacttgcctgcctctcacccctctgtcacccctcctgggccccggggcctgctgcctgcctctggagggcttgtcatccacccctccagggccatgccctgacctctgtcctctctacttaccacccaaggtcaaagggaagtgtaccactgaccacatctcagctgctggcccctggctcaagttccgtgggcacttggataacatctccaacaacctgctcattggtgccatcaacattgaaaacggcaaggccaactccgtgcgcaatgccgtcactcaggagtttggccccgtccctgacactgcccgctactacaaggtgggtcagagttgataggggcaatgccagtggtcactcctgaaggggcctgcaaggcaggtgcagggaggacattaggggagtggaaactgggaaggaggccgaccaagcccaaaggggactgctgtggaagggaggagaggcctgcagcccctccctgtggctgagaaggcatgaggcccaggtccggtggtacagccgggtccctgcaccaggaggagttagtgagagatatcttaggatatctggccctagacaaagacaaggaagggggccgactcaggaagtcagaggccaaaagctcagagagggggctacacggggcctcacagtgagcaggcagagagggtctgaggtgattggactttttctgctttgagaaacaaacagaaccagggctgaacccaagtcctggcccagccgggtgaaaggactctggcaccccctggtggctgggtggggcagagggtgctcccaggaagggggcgccttgagcttcacagatgcatcttgtgtggggcccggaggccgtccctgtctcacccaacctccctccacacacacctgcctctgccaagcaccaatgggtggcttctgtcttctttgccactgcaaacaaccacgtgcctctgtcccctcggggcctcgtttgggtctcattcacgcaggcttcacttgcccttaggcagcaggcgaggaagggcccctccagcccctttaccgggagcctcaggatgcccaggcgccaggtgggtgaggccaggcaggtagggccagacaggtgaggacggtgccctcctctgccttataaccttacccccgcttgcctgacagaaacatggcatcaggtgggtggtgatcggagacgagaactacggcgagggctcgagccgggagcatgcagctctggagcctcgccaccttgggggccgggccatcatcaccaagagctttgccaggatccacggtgagctggagtctgtacccaggccatcctcatcccatccctagtgatcaaggtcactctccctgcccgtggctgagttgggcctggttctaggctgtgtccactgcagcccacaggcccgtcagcctcttgccccttcttaggctcacacagtgcacatccgacgctcagcttcccggcttcccgcaggccctgcttccaggcttgtagatctgagccgctgagatctaggacatgtgccagggggttctttctgatcatgaatgtgcagcaggaaggcctcgcagacctcagcaccagcgcacacttgctaggggcacccctagtgaaagggagcagaccagggccccatagtcactgcccgggcattgtcccaggcagcaggattaggggcatctcccagagccccagatgggttcagaaaatgaagctctccaggctagtcaggcccccgatgaccgaatgccgcctgctttccagagaccaacctgaagaaacagggcctgctgcctctgaccttcgctgacccggctgactacaacaagattcaccctgtggacaagctgaccattcagggcctgaaggacttcacccctggcaaggttaggggcccgggtccccctgaggtggtggggtgaggggcagccaccttgtttcccctcctgcactggccccagggtagcttctcccaggaggcttcattccagctggaaaggcccccagttctccaggtggccccacagagaaagcaaagtggcttctcagagttgggggttggagtcaacccggggccctcacacctccccaacctccctttactcaccaggacctggcactcaggggacagcccacccactgcaggaccctctgggccccaggaatcccctgtaggtgccacctgggtctgacctgggccatcaggcacagactggcctaggatttggtttgcctgctgacctcttaggtccccaggcagtgccctgtctccctgacccccctgcggggccaagggcacacagtacccaccacttccacccacacccaccttctccttgcagcccctgaagtgcatcatcaagcaccccaacgggacccaggagaccatcctcctgaaccacaccttcaacgagacgcagattgagtggttccgcgctggcagtgccctcaacagaatgaaggaactgcaacagtgagggcagtgcctccccgccccgccgctggcgtcaagttcagctccacgtgtgccatcagtggatccgatccgtccagccatggcttcctattccaagatggtgtgaccagacatgcttcctgctccccgcttagcccacggagtgactgtggttgtggtgggggggttcttaaaataactttttagcccccgtcttcctattttgagtttggttcagatcttaagcagctccatgcaactgtatttatttttgatgacaagactcccatctaaagtttttctcctgcctgatcatttcattggtggctgaaggattctagagaaccttttgttcttgcaaggaaaacaagaatccaaaaccagtgactgttctgtgagtgattggtgtctgtgccgtttgttgtcaagtccagggtccaggaagggtcttttcagtcaaggtcagtggaggcccaacagccacagccacagatggatccatcactgcagtgaaggaggagcagggcctagatggtggaggagcggggcaagctggtgggcactcctggccttgcctcactgtccagctcgagacactatctccttagcatcggcctcagctgctgttgtcttcacagccggccataccaccttcccgcaggctggtagggtccactgtccagccccagggctagttgctggtccaccaggagagaaggcccaggcctggctcactgatggatccctgccagggataaagaacacgcacatttcactgacatgctggcttgaaccaacctggacaggaggtgaagggagcccagcaggcccagcacaggcaaagaagaggcggggcacacggcagccaagaggctctggagagatgtgggagccacagcagtgctcccaacgggcacgcagcacgcagggcgcagacccttctccagggactccaggccctgatagggtcagggacccttcctcacccttaggcctgccctccagggccacctaggttctaatcgtggcaggaaggctgagcaaacacaaggcccatgctccagacgcatccaacagggagcagccttgaaagccactcttccccggagccctcccttttatttcactgctgcaatgctgctcctacagggagccctaccttttttttgttgttttttttttttgagacagtctcgcactgtcacccgggctggagtggagtggcacgatcttggctcactgcaacctccacctcccgggttcacaccattctcctgcctcagcctcccaagtagctgggactacaggcgcccgccaccacacccggctaattttttgtatttttagtagagatggggtttcaccgtgttagccaggatggtctcaatctcctgacctcgtgatccgcccaccttggcctcccaaagtgctgggattacaggcgtgagccactgtgccccagcagccctacccttaatctcagggctttgatctctcccaagccccttccttgatttctttctccctcaaacccctccctaccccccaattttttaaagatagcatttcattgtgttgcccaagctgtaatgcagtggcacaaccttgaactcctgggcttaagggatactcccatctcagcctcccaagtagctgggaccacagggcacgccaccatgccctgctaattgttttttatagagacaacttctcacttttttagccaggctggtctcaaactcctggcctcacacgatcctcccactttggcctcccaaagtgctgggattatagatgtgagccactgcacttggcctcaaagccgctttatcagtggtcttgtccaccgtgagctctgtcccctttgcaagctgactcgcccaccccaccagcagccatgtacctttactgcctcacagccctggcctacagaaactcaggctctccagggagaagtgaccagccaagaccccccaaacaggatccctgacctgctgggggagatcccagagcagaagccccaggacacagcttccagccctgcactccggctctcccgcctcatgggggcttcagcaccatcagagccactcaccacaagcgtgtacggagcctccttctttggcagctcctcactggaagtggtggcatctgctgagctgggccctgtgggggacgtgtcaacaaagctctcgtccaccacccggaagcggatctcctcgccggtgtccatgtagaggtcgtgtgctccttcctccgtctcgtactcccacacccacacctgctccgcttcgtcgctgagggggtccagggtcaaggcagcaaccagatagtgggaggggcttccctcagcacccactccccgaccccgcaggaaaaaccaatctcagggtgagatcactgtggctgggaagtcacaggaaaagcaggtcccagccccatgcacaaggcctgggccaaacagagaggaacaagcgctcttggaggccaggacccaggaaggggaacatggggtccccagccaggcaccacggccagacgcacatggatctcagagcaccagagcaggaggggacgagacagggtccgagtcccacctctcaagacaagagcaacctgggcctcctgccacggagccccacacacccttttcatcaaagccttgtgacaggctgatgggcacccaaggacttgccgttttcaagaggacatagtgggatgggaggtcacctgcccacttggatgacaggtggtgagagaaccagccgggctgcgccaggttttctaacacatcctggagtcacatgcagacccagagccgtgctgccacccagccagggcagtcccccttggctctccatcgtccctggcagagcccacacaggggagccatgggttctctggtggcagtcccactgttgggtcctgcctctgtgccaggtcccgtgctgtgcaccgtgggtacagctgtgacatgtcagagtccttcccagtgaggagcctcagatttgtggatagccccacagaaacagacaaccccagtacagcaaggtgaggggtgggacagggacaacggaggcccaggaggaacacatcttagcccacgacccggcacacaggcaaggcctcacgtcccagggaccagtgcatgcctccagggcaagtgatctttaagatgattcctgcagggacaacctaagtgaagtgaaccggaagtgaatggtgtcccccaggacaccacagctctaatggtgtgcagtgtacaacccagacagccaagcctgatggccccgacaaagacaggcatgtgggcatcacatgactccctgcatgggaggcacagacaggctggtgcctctggagcaaaagcacaggcgaggggcaggcacagaggccagtgacatcaggttacaggccccaaaggccaccctaaggagtggggaccttcctttggagaggcctgagctccctgtgacaaaccactttaacccttggaggatacaacttggctggctgctgcagtgactctggggggatgagaatgtcatcgaagaagcctagagagactggaaggaaggaagcaggtgacggcctgagatgggggtcccagtggacgccaaacactgcggggtcttatgcttgacaggcaagccctgcctggggctgcccacgaggcggatgtcgctgtggaaacctaggccccttccccacctaatgcctctgcgtacaccacccacatagcggagcagccctgggcaaagcccggagcacctagtccccaaaacctctgcacacaacaggggccagtgggcgctaaagactcccagtcacttccgccagcttcagtgcccaccctgaagtcctgagtgacggcctcacagtgaaaccgcactgctgatggaggtcctcacacaacacataaaagggagggtgggcaagcttctctgacaccacggggaaggttcggcctcgacacctgagccctcccctgaccatgtgggtggacacagacctcctgcccccacagtgttcccaagtcttgtctgaggcgtcagggccactgttaccgtgcactccttctgggctgcagcctttgatcttcccaatgagaatctcatctaggaatggatgaaacaccacgcagcgaaaatggactggaaatgaagacagcccatcagtgatgctgaccggggccccagtgcgctcccatgtcaccctcaggtgccagcacacctgggggcctgtgtggctgctccatgccacatccccctgcaagcctagaccctccctttgtctcctgcaagttcacagcatggcccccacaggggggcagcagcagctgtcctgtaactgggcactcccaaaactgccaggttactcacacctgagcccagattctgagccatcagtggctccaaagtcaatttcaacctccctccaggctctccttcagctcctaaaaagggcagaagcaactggtaaagaacgcagggtttggaaactcccacctcctgctgagcagcaaagtcggccccaggcacagctctgacctggagaggaggctaaaaatgccctggtgttgtgaggcgcaggccagcagcccagcaaaaatgcctctgtgggttctcaggccatgctgcaggtggtccccactcacctgactgcaaggaaaacaccaggtggggtctgtgcccacatcgcacactctgaccccaggccctggcattagctgtatgtcaccttgtcactcaggcccttgctcgaagccccaaagtaggagtccaacgctgacctgagaatcagcacgcggctgccccctgctggttaccaacagagttcccaccgtgaggataaggcagtggctcagagccagggtgacccactcacagccacatagccatggggagacacctggtctttctgtacccaccagacagggccaccgggtcgaatcttgccttcctcagacaggtttaatcccaaacacaggcttccacgatgccgtcaaccatttagtcggccaacatgccccccaggacggcagggaccctggcatcagcagggcatgaggagaggcagccgatggacctctcaccctgggaagacacaaacgtgagccatcacagccaccatgaagatgactgggccttgtgctgacctcccagagagcagatgagggcggccaagggccacttacgcacagatggaaaaaccctgtcccccacatggcccaacattcagtttggtttaataaatcctcacagaggccgggcggggtggctcaagtctgtaatctcggcactttgggaggccgaggtgggcagatcacctgaggtcaggagtttgagaccagcctggccaacatgataaaacaccgtctctaccaaaatacaaaaattagccgggtgtggtaccaggtgcctatgatcccagccactcaggaggctgaggcaggagaatcacttgaaccaggaggtggaggttgcagtacgccaagatcatgccactgcgctccagcctgggcaacagagcaagactccgtctcaaaaaaaaaaaaaacttcacagagcctgcaccaggtttgtgccaggcccagtctgggcactgggctcacgggggtgggacagcacatgatcctgcgcccagggagccccaggctggaacaaccacatccaaaagcaaatgctgggcacccggagtcccccatggatggatgagtaaacaaaacatgctggagacacacaatggaatattatgcagccttacaaaggaaggaattctgacatgcactacagtgtggctcaacctcgggctgaaccttcacatggggccaagtgaaagcagccagacacaaaagccacacagtgtgcgactccattcatgtacagtgtccagaaccacatccatagagacagaaagcagttaagtggctgccagaggctgggtgaaggacaggattagcagtgactgctactgggcggggtttctttgtagggtgataaaaacgctgtggaatggccccgcgcacggtggctcaagtctgtaatcccagcactttgggaggtcgaggcgggcggatcacctgaggtcagcagttcaagaccatcctggccgacatagtgaaaccccgtctctactaaaaaaatacaaaaattagccaggcgtggtattgggtgcctataatcccagctacttgggaggctgaggcatgagaattgcttgaaaccaggaggcagaggttacagtgagctgagactgcgccattgcactccagcctgggcaacaagagtgaaactctgtcaaaaaaaaaaaaaaaaaaaaattgtggaattagatagtggtgatggcttagctgatgagggtgttgttcctggtgggagggggccctctcctgccctgctcatatctgcctacttaccataacccctggaggcctgtggtgccagtcactgtattcctccattcctatagttcctccaaactggaacttgcatctagaatttttaacagattcaactcctagcgtttagcaacagaactgcctcataatcggtgttgagttcttccaccaggaactatataatgcttgtttgtctctctttgggggaagctaaaaagttaatgatcattgatgagatctgtcttcataaggagttgcaaaatggtgataatgtaattaaaaaaaaaagatagtggtgatggttgcacaactcttaatatacaaagccactaaattgtatgctttgaaagggtgaattttgtggtatgtgaattatatctcagtgaaaaaacagaaggacacagagcaacaggctggtcagccaggagagtgctggatctgcagttcatctcacaagagccccacctggaggtctagggatgtgtagcctgcagaggggatgcctggaaagtcagaaaggatgagtctaaaggaccatcaagaagaggaaggaagctgggcgcagtggctcacgcctgtaatcccagaactttgggaggccgaggcgggcggatcacttgaagccaggagtttgagaccagcctggccaacatggcaaaacccagtctctactaaaaatacaaaaattattagctgagcaggccgggcgctgtggctcacgcctgtaatcccagcactttgggaggccaaggcaggatgatcccctgaggtcgggagttcaggacaagcctgatcaacatggagaaaccccatctctactaaaaatacaaaattagccgagcatggtggtgcatgcctgtaatcccagctactcgggaggctgaggcagaattgcttgaacccgggaggcagaggttgcagtgagccgagactgcaccactgcactgcagcgtgggcgacagagtaaaactcttgcattttttagagacagggttttagcatgttgcccaggctggtctcgaactcctgacctcgtgatccgcccgcctcagactcccaaagtgctgggactacaggcatgagccaccatatctgccccgtgaaaaagttctggaggtgggccgggcgcggtggctcacgcctgtaatcccagcactttgggagcccaaggcgggcggatcacgaggtcaggagatcgagaccatcctggctaacatgatgaaaccccgtctcaactaaagatacaaaaaaaaattagccgggcgtggtggcgggcacctgtagtcccagctactcgggaggctgaggcaggagaatggcgtgaacccgggcggcggagcttgcagtgagccgagatcacgccactgcactccagcctgggcgacagagcgagactccatcttaaaaaaaatgaaagttctggaggtggatggcggtgaaggttgcataagaatatgaatgtgcctaatgccactgacttacacacttaaaatgtttaaattggtaaatttcatgttatgtatattttaccacaatagaaaaaaatgaaataaatgataaacatgtcaaaaaaggtttaaaaaaaaaaaaaaggccaggcgcagtggctcatgcctataatcccagcactttgggaggccaaggcgggcggatcgcctgaggtcaggagttcaagactattctggccaacatggcaaaaccccatttctactaaaaatacaaaaattagccaggcgtggtggtgggcacctgtaatctcagctactcgggaagctgagacaggagaatggcttcaacccgggaggcagagaggttgcagtgagccaagatggtgccactgcactccagcctgggtgacagagaagactctgtctcaaaaaaaaaaaaaaaaaaaggctttaaaaaaaaaaaggtggggggtgtctctgcaccaactctagctgaggaggctgcccgatttgcaaatcaatataacatttaaaaatgttaaaaaataaataactaaaggtggtcacaaaggagcgctgctctagactgagagcagggcccccgatcagtatggggcaggcgggggcagggtacagcaggtcccaggcttggaggatgtggggaacacgggctggcaccaaatgtcgagggataaacttggccagtgggggtcagggtcccatgccacaaaggccatcatctgtgagcactccccacgtgcggggcgctagaccacagcttcactcagacttgctcgttgactcctcacagatatttgatgaaggaggaaggctctattactgttattatactgtgtgcacagatagggaaaccgagtttcagagaggtcaagcaactgctcccaaggcccaggcaaataaatattcctctgagcaaaggtgaaggcacagcatgacctggtgctgagcccttcagcactggctcagtgacagccctgagtgaggctgtggggttgcaggagagggagcagtgggaacagggcctggggtcaggcatcagcctcaaagcatggcaattggttcccaccacaaaatcacagttcaccctgcatggtgctcaacaggacttttatagctgcagggttaaccccattgtacagaggaggaaaccaaggccgagagaagcagaggtacccgcccaaggtccccatccaggagaccagagccagaattaattcaaatctaggtccttgtgatcactccatgcctcccacctggttcccaagaaaacagagactcaggacagatgagggtgggggctaataaaagccagcagtagaaatcttccaattgaatagacagaatttcctccaactttttttttttgagacggggtctctctctgtcacccagctggagtgcagcggcgtgttcttggcctcctgggttcaagcgattctcctgcctccacctccacctcccagggagctgagattacaggtgtgtacaccaccagacctggctaattttttttttttttttttgagatacagtctcaccctgtcgcctagggtggagtgcagtggcacaatctcagctcactgcaacctctgcctcctgggttcaagcgattctcctgcctcagcctccggagtagctgggattacagacatgtgccaccacacccaaataatttttttttttttttttttgagacggagtttcactctgttgcccaggctggagtgcagtggtgcgatctcggttcactgcaagctccgcctcctgggttcactccattctcctgcctcagcctcctgagtagctgggactacaggtgcctgccgccacacctggctaatgttttgtatttttagtagagacagggtttcaccgtgttagccaggagatctcctgacctcatgatctgcccgcctcggcctcccaaagtgctgggattacagaagtgagccatcgtgcctggctttttttgtagttttagtagagatgaggttttgccatgttggccaggctggtctagaactcctgacctcaggtgatgcacccgcctttacctcccaaagttctgggattacaagcgtgagccaccacacccggccgcaacttgacttttttctccaagtttttctggaagttctagtacctattaccacttgtctcctggacaaccaaggcccctgtccaaatgcggagactgggctcagctcggcatccttctctgtaaacagaggccctcgtggttgttttcacactgcaatcttatttctctgctaatgaaaatgctcctagtctgtacaaagacagcctgtttcttcggcctctcggcgtaccatggtaacatccatgcgaactaattaattccctgaatggctggacacagacactaggcagtctcacagcatactctggaatgttctggcatctctgtgctgagaggctgaccagtatggactacagcagggcttatccaacatgagctcgtgtgacaaccacctggtaccttgttaaaacagtctttggggctgggggcagtggctcacgcctgtaatcccagcactttgggaggccgaggtgggcggatcacgaggtcaggcgttcgagaccattctgaccaacatggtgaaaccccctctactaaaaatacaaaaattagccaggcgtggtggcacgtgcctataatcccagctactcgggaggctgaggcaggagaattgcttgatgctgggaggtggaggttgcagtgagccaagatcacacttctgcactccaacctgggcaacagagcaagactctgtcttaaaaaaaaaaaaaagtatttggacccatcccccaagattctgactcaggcggagggaggcctggggcaggctgagaattgcgctctgagtgcccaagggatggcaggcttggcgcctgcgctctgggagcctgaaccgtctcaatgggcctctcaccatctcctatacactggtttggtccagacatgagaatgaagaatgcccctctcctggggctcatcacctcccttgactggtggtcctggctcctctcagggtggtcctctctctactcctccctccttccaggatctggtgatcaccttccattcctggatgcacacccttttgcaatgtgactgcagtttctcccacccagaggtgaagtctatttctccatcccctcaccctagccaacgggatggaagcagaggcttgagaagtacttacgcactgaactttggaaccttaaaaccgccaagcccagcctggcctactgaaagatgagaccatgtggggcagagatgggccagcccagcccaggcccccagtaaccaaccagtctgccaaccttcagaatgtgagaccgtcctagattgtccagccccaggccagccagatgagaggaaaaccacccaactgaccgacagaatagggagaaacaatccacgctgataagtcactgagtgctcgttaggtgccaaggctctgtcctaagagctttcatgtattaactccttaatcctcacaatggtcttagaatataaaatctatcctaaactgcagttgacatcctgaactgcaactgacagataaggaagcagaggcacagagcaatgacatattcagccaggacacacagctagtaagaagcagagctcagcttttaatcttgaggccacactcttacctactgtgtcctactatccaagtcccccacccccagccagcctgctgatcccccttccgtctgctctccacaggcagcgggaagcccgaggaatggacaaggtgcagggctcagtagcaatgagaccaaaagggcacagacacagagctatgaagtcagagctgaagaggccttgaaaaccaactagtccacatcccaggcctcacagatgagggaacagaggcccagagaagagaagtctgccgctgtcaccgggtcaattcctggccgattcaggatgaggccccacgtctcccagcctccaggactcccaccaccctctggccatctctttgactttagccttttaaaattttttcatattttatttatgtatttaaagacagggtcttgctatgttgcccaggctggccttgaactctgtggctcaagagatccttccgccctcagccttacaggcacgcaccactgaacctggatttggcttgacctttggctacagaaaactgagccccaagaagcccaatgccccagggacagaagatacccacctttggtgtgtgatgcgccatccccagggaatacataggcatcctccagtttggtgatatcaaacagacaaatgcagagtcccacgttgtacacgacctgcatgcatacaaacacagacacacaagtacacatggatacagagtgaccacaggcccagctcccaggcaatctgagcccctcacaaccacaagcaagccatgattatctccttttttaggtgaaaatgagtttgtggaggggaccagggtaaggaacctcagaaggatgcagagctctggaaggagcagggcctcgggtgacaaaacaggtccaggttccaaggccagctccaccacccactgtgtgactgccaagagggtcacttcacctctctgagcctcggtctccccatctgcaaatgtgaaataccaggctgcagagttataaaataaagagagataacagatgtgaagcccctagccttgtacctggctcgtgtattggaaaccatcatcatcaaggatctttgttattactagtgaggaccagtgacaggaatgctgcctaaacgtgcactctccgacatcccaaagccaatgccagagctcacagtttagtgagcgtcacagccacagctaaaccccactgtgctcacaacatctcagacacacagacgcagccacagggactgcagctggcagagggacacaagacctaggccccaagaatgtgcagaaatcccatgccctcaccccacaccactgtggttcaaacgactggctactcagtgtctccttatctgccaccgtgtcatcacccccacttagaacccttgcaagaactgtccaagaattagcaagaagctcagagaactcagagtcgtggccagacctcaccaagataatgccacaagatctgtgccctttgtcagaccatggatttctgtgtgggccccctctcaaacgattcatctaatcaaatactagaagacttgtcccaacaggacctctaccccaaagggactatattcccttcttggactttccacaccttctctctcaaggtccacacctccatcccccaacagaccctcatcttcaacctcacaaagtaaccagaagccatccacttaaagactccatgagttcccttcactatctagcaacccccaaatcaacctcacctcttctgaagggacctgtctcatgcgtaaagccagctaagcatcctctttctggctccttctcagcccatcaccttctgatgctccacggataaagaccaatacctcaactccgacatcctacagggctgtgtggcctggcccccacccacctgtccagcccaccctcccctctgcccaagccacaccagcctcttccacaccatggtgggtattttaccccttcttcttttttttttgaagcgaagtttcactcttgtcacccaggctggaaggcagtggtgcgatttcagctcactgcaacctctgcctcctgggttcaagtgattcttctccctctgcctcccaagtagccgggattacaggtgcctgccaccatgcccagctattttttgtatttttagtagacatggggcttcaccatgttggacaggctggtctcaaactcctgaccttgggtgatccacccgcctcagcctcccaaagtggtgggattacaggtgtgagcccccacgcccggcctgtattccttcttcacagggcctttgcacatgccagttcctgccttcagggctcttctctccacccagccccctcccaccctagaaaggaatcctctggttggctccacttcaccatcagatctcaccttcaaccccacctcctcaacaaagctgcaggtcccctcattccaggggacgttcctttgttccacaggtgtgtgaacacctgtccctcataacatttacctcaatttgtcaccaaacactcaactgtggacccttctcattcatatctgctttcccaccacactatagcacagagccttgtctctgtttcattcattcagtcaacaaatggccgggcacggtggctcacacctgtaattccagcactttgggaggctgaggcaggcagatcacctgaggtcagaagtttgagaccagcctggccaacatggtgaaaccccgtctctactaaaagtacaaaaattagccaggtgtggtggcaggtgcctgtaatcccagctactcaggaggctgaagcagaagaatagcttgaacctgggaggtggaggttgcagtgagccaagatcgcttcattgcaccccagcctgggcgacaagagcggcactcttgtctcacacacacaaacacaaaacagaaaattatgggacatctactcccgccaggctttggctaaatgaggggatacttcagggaagaaggcagataaggtccctgctctcaaggagagggaaaggaaaaagtctaagaaggaaagagagtgatgtgacacagaatacctggaaagggaggactgtcaaagaaacggcagtgggaagacctctcagctggtactaaggcacacaggcacagcacagagaaggcgcacaaatattggctgctggaatgaagggatgaacctcctgacagttaattctgtactactagaactctccatcttcacgaaaacaatgcattgacccgaggcgatggcgcacacctgtaatcccagcactttgggaggccgaggcggggggatcaagagatcgagaccatcctggccaaaatggtgaaaccccgtctctacgaaaaatacaaaaattagctgggggtggtggcgcgcgcctgtagttgtcccagctactcgggaggctggggcaggagaatcgcttgaacccgggaggcggaggttgcagtgagccgagatcgcgccactgcactccagcctggacgaaagagcaagactccgtctcaaaaacaaaaaacaaaacaaaacaaaacaaacaacaacaacaacaaaaacaatgcatggctggaaggacagcaatattttatccccattttaaaaatggaaactgaggcccagaaggggtcagtaacttgcctgcaattgcaaagtaatttagaattgtggaatctaacacttccagttacaactccctttttcaagcccattttcttttgatccccttcaatatctcctttcacggtgaagaaaagttctgagacattaagctgcttgcccacggccagggcttggcatgaccgaggccgggcctgcggccagtgggcggcgctcgctctcccacgccaaggcctgcccgcgagccgtgggcccagtaccttgttggccaacttcttgttcagctcctcggcaatggagtcgttgagcttcctctcaaactgccaaggggggatccggacggtgtccaccatttccaccaggacgaacatcccggcctgcgctgggggctctgggaacaggagggtcagtcacgcaccagggccgggggcagggagaatccccgagccccttggtcccgtcccagtcgctgaggcccccaggctggcggtgaggttgcacggggcggtctcgggggcccggtccgggccatgctccgctactacaacatgaggaaactgaggccagagggaggcactctccgtccacagctccgggtgcgcgcccgcgccgcgagaccccgccacgccacgccactccacgccacgccactccacgccccgcacccgcgccacgtgccgccgctcgtatcacgcaccacgcaccacgcaccgcgcacagccgctcgcgcttgcgcgactgcgggcgagattccgaatccatcgcttccggaaaactcaccaataagaaacctctgtccacccccaccccgccgaggcggggcggggctgcctgtgtgctcgcgcacagcgtggggcagagagagggcgaggagagggcggggcctcctcctttcacaggagcagagcggttcccccgcccccgacagcgctttctgttgctaggcgacctccgtgcaatcccgcctgattatccctcgcctggcctgtgctttacccgcattgctgtcctctgctcggcagccagagtgatcctttggaaacgcaaacatatattaccacatctaagttaaaaaaaaaacccaccgcaaacataacatgcttagcacccttaagtgttccggagacaaggagaattagcaggaaaggggcacttgggactgttctgagatgatggaaatgttctgtattttgttttcggtggtataacattgacgtatataattgtccaaactaattgaactcggcaagatttttgcatttatttgtagacattatgttccaattgaaaagttataataaattggaacaaaaaacacaacccattgaacttagaatactagatccaagccgggcgtggtggtgtacacccgtagtcccacctactcagaagcctgagacagcagcatcgcttgagtccaggagtttgaggttgcagtttgaggttgcatgctgcactctagcatgggtgacagtgacacctcatgtctaaacgaaaaagaagaagaaggagaggcggaagaagaagaaagaagaaggagaagaagaagaagaaagaagaaagaaaagggaaagaagaaagaaaagaagaaagaaagaagaaaagtccaaaacctttatggtagttcacacggccctgcaagatctgaccatacccccctcctatagccccatcatgctctgtctcgctgacattccagtctcattcatcttcacgtgatagcatgggctaagagaatttggtagcttacccaagatcacgtgttgacaagtggtagaggtgggcccagtctgaacctcactaccctgcctctaaaaattggtggggtgtagatgtatactgtagtgggtttttttgtttgtttgtttgtttgagacggaggatcgctctgtcgcccaggctggagtgcagtggcgcgatcttggctcactgcaagctccgcctctcgggttcacgccattctcctgccttagcctcccgagtagctgagactacaggcgcccaccatcacgcctggctaatttttcgtatttttttttagtagagacgaggtttcaccgtgttagccaggatggtctcgatctcctgacctcatgatctgcccacctcagccttccaaagtgctgggattacaggtgtgagccaccgcgcccgggcttgtagtgtgttttaaccaaaataacaatttaggtacttacaaaagatacttggggtaaatgacagaggcacttggaaatgtatggtgccttaatttgctgatttcacaagcatagtgatctctcagtgacactggctgcagtgatgctaaatgtggccttccctgcatctatgattttatttttaggtgtgcttctataggatttgaggtacttgaggacagcagcaaatgctacttcagctctttattcccacaatcccctaacacattcctaggaagcattgagtaaaggtgtttagggattaccattaattcttcctctcaaatgtttcctcttcattccactcccactgctattgcttcggttcaggccttttcttttttttttcttttgagacggaatcttgctctgtcgcccagactggagtgcagtggcatgatctcagctcaatgcaacctccacctcctgggttcaagcgattctcctgcctcagcttcccaaatagctggaattacaggtgcccaccaccacacttagctaacttttgtatttttagataagatttcaccatgttggccaggctggtttcgaactcctgacctcaggtgatcccccccatcttggcctctcaaagtgctgagattacaggcatgagccaccgtgactagccaatatctggtcatttaaaagtatgtggcggggccaggcgtggtggttcacacctgtaatcccatcactttgggaggccgaggtgggtggatcacaaggtcaggagattgagactatcctggctaacacggtgaaactctgtctctactaaaaatacaaaaaaaaaaaaaaaaaaaaaaaagtagccgggcgtggtggcacgcgcctgtggtcccagctacttgggaggctgaggcagaagaatcacttgaacccgggaggcagaggttgcagtgagctgagatcacaccactgcactccagcctggcgacaaagtgagactccgtctcaaaaaaaaaaagtgtgtggtggccaggcgtgtggctcatgcctgtaatcccagcactttgggaggccaaggtgggcggatcacctgaggtcaggagttcaagaccagcctgaccaacatggagaaaccccgtctctactaaatacaaaattagccgggcgtggtggcgcatgcctgtaatcccagctactcaggaaggctgaggcagaagaatcgcttgaacccaggaggtggcggttctggtgagcagagatcgcgctattgtactccagcttcggcaacaagagcaaaactccatcttgaaaaaaaaagtgtgtagcacctccctcctcccactctctctcttgctcctcttttccccatgtgatgtgcctgctcctcctttgatttccgatatcatgggaagcttcccgaggcatccctggaagctgagcctatgccagtaccatgcttgctgtaaagcctgtggaaccatgagccaactaaacctatttttttttttttgagacggattcttgctctgtcgcccaggctggagtgcagtggcatgatctcggctcactgcaagctccacctcccgggttcatgccattctcctgcctcagcctcccaagtagctaggactacaggcacccgctaccacgcccagctagttttttgtatttttagtagagatgaggtttcaccgtgttagtcaggatggcctcgatctcctcacctcatgatccacccgccttgggctcccaaagtgctgggattacaggcgtgagccaccgtgcccagccagcctattttctttataaattaaccagctctgttgcccaggctggagtgcagtggcgctatcagggctcactgcaacctccacctccccatttcaagcgattcttctgcctcagcctccagactagctgggattacaggcgtgcaccaccacacctggataattttggtactttcagtagagacaaggttttgccatgttggccaggctggtctgaaactcttgacctcaagtgatcctcagcttcggcctcccaaagtgctgggattacaggcatgagccaccactaccagccaggtatttcttttttctttctttttttttttgtgcgtgtcacccaggttggagtgcagaggtgccatctcagctcaccgcaacctccgcctcctgagttcaagcgattctcctgcctcagcctcctgagtggctgagagtataggcacatgccaccacgactggctaatttttgtagttttagtagacacggggtttcaccatgttgaccaggcttgtctcaaactcatgacctcaggtgatctgcctgcctcggcctcccaaagtgctgggattacaggcgtgagccaccacgccaggcccaggtatttctttatagcaacgtgagaacagcccaaaacagaacccacatgtgatgctgttctcattcattcgtgcattcattcaacatactgttgtgtacagttgttggagatgcaatgcagaagaaaaactgggcctcaaaccgagcttcaactcagagcacttggtgctggagttggggggcagcagggtgggcactgagagaaagagcgagtttgtactctcaggtaattaaaagtatttggagccaggcacagtggctcatgcctataatcccaacattttgggaggccgaggcaggaggattgcttaaggtcagtttgagaccagcctgggtaacaaagtgagatactgtctctactaaaaaaataaaataattagctgggtgtgatggcacttgcctgtagtcctagctacttgggaggcttagttgggaggattgcttgagcccaggaatttgaggcttcagtgagctttgatcatgccactgccctccagcctgggtgacagagcaagactctgtctctgaaataaataataatattcgggctgatttacatgggagatgacaactggtaagctaagaggttagagacctaagcagcatccagatcagagggccttatgattatattaacagctaacattcatgagcacttactatatgtcaagtactacttaggtgctttatatgcattaagtttgatcctcagaacagctgtatacagtaggagctatcatcgttatctcacttgcccaattgtctcattttgcctcatctaaggtaaacagaggttaagtaggtcattcaaggtcatacagccagtaagtggcaaacctgggacccaggccccagagtccctaatcacgaccaccaggctgtgccccgccatgacgttagaagtcaggagttcgagaccagcctggccaacctggtgaaaccctgtctctactaaaaatacaaaattaactgggcatggtggcgcatgcctgtaattccagctactcaggaggctgaggcagaatcacttgagcctgggaggcagaggttgcagtgagccgagattgcgccattgcactccagcctgggtgacaagagtgaagcactgtctcaaaagaaaaaaaaaaaaagaaggctaggataggaaaataacacagggacatatcacctgggtagacagcaaatgggaaggggctggagcggaggcaggaccaggaaggaggttattgtgtggccgcagcaagagatggtcagagcaaggctggtagccatggagagtaacgctggtcagagtccagaggtcttcagggggtggaatgggtgggatgacctggaggacagaagtgggaggagccgacgcctgtgggctttagccttcacctctgagatgggaaactgcagcggggccaggctgggggcaaggggccagagctgaagcggtcagtgtgtgggttaccaggctactgttacagtattccgctcacaggattgcaacagtgatgtgttggcacgtttggtttactcattggtaaaaccagagatcaggcctctttatgaattctccatgttgctccatggaaggtttgaggtacaaaaacacacaagcgggccgggcgcagtggctcacgcctctaatcccagcactttgggaggccaaaacaggtagatcacaaggtcaggagatcaagaccatcctggctaacatggtaaaaccccgtctctaccaaaaatacaaaaaattagccgggcgtggtggcgggagcctgtagtcccagctactcaggaggctgaggcaggagaatggcgtgaacccgggaggcagagcttgcagtgagccgagatcgtgccaccacactccagcctgggtgacagagcgagactccgtctcaaaaaaaaaaaaaaccaacaaaaaaaaaaccaccacaagtggcttcattttcaagctccgtgaccacagagcttctctacctattgcgtccctgcatgtgctctggcctaacccagtgtctgatacatgttatttgaatgaacaaaataataataaaagaattggccgggcgcagtggctcacgcctgtaatcccagcactttgggaggccaaggcaggtggatcacaaggccaggagttcaagaacatcctggccaagatggtgaaaccccatctctactaaaaatacaaaaaattagccaggcacggtggcatgtgcctataatcccagctactcaggaggctgaggcaggagaatcgcttgaacttggagggcagaggttgcagtgagctgagatcatgccactgcactccagcctgggcgacagagtgagactctgtctaaaaaaaaaaaaaaaaaagaattgctgaaagtataggagtgagggagaaatagggctggaaactcaccaaggcaggagaaagagagtcactcaggatgactaggagcatagcatttccatgccacagcacacgagttgagcacctgcagggatgcccatgaccctctcagtgccccacggcctctctgtaaaagggaagcagcaatagatcccatctcaaatgactgtccatcagcagagtgctggctgaaaaaaaaaacatggtacatcacataccagaaaaatgtgaagccatgaaaaagaatgagaatgtcctttgtgtattgatatagaatgatatttaggatatattgttaaatccaaaaatacaaggtgcagagcactgtgtaatggtgtaatgggatgttaccatttgtgtaaaaaagggagaaaaataattatctaatattattttatgcagaaaatagctctgaagggttcataagaagtggattacactggttgtctccggagtaaggaatgaacagctgaagcggggtgaggggcagtgggaggggagagaattttttcctaaatgcactttggaatctttttttttttttttttttttttttaaagagacaaggtcttggcccggcgcagtggctcacacttgtaatcccagcactttgggaggccgaggcggctagatcatctgatgtcaggatttcgagaccagcctggccaacataccccatctctactaaaattacaaaaaataagccgggcatggtggcgggcgcctctaattccagctactcgggaggctgaggcaggagaatcacttgaacccaggaggcaaaagttgcagcaagccgaggtcatgccattgcactccagcctgggcaacaagagcgaaactctgtctaaaaaacaaacaaacaaacaaacaaacaaacaaaaaacagagagagagagagacaaggtctcactctgtcatccaggttggagtgcagcagtgtgatcatagctcactgcagcctcaaactcctgggctcaagcaatcccccagtcttagcctcctgcatggctgtagctgtgtactataggcacgtaccactccacccggctaatttttttattttttatttttgtaggaatggtgtctcactatgttgcccacgctggtctcgaactcttgagctcaagtgatcttccctcctcggcctcccaaagtgctggcattagaggtgtgagccattgtgcccggccaggaatcttctttgtgtgggatcatgggactatattacctgttcaaaatgaataaaacaaaatttaaaaagcaattaagattttttttcacatcttaaaaaaaaagcttctcttggccccctttcctggctcccttttcctccagtgaatgctccatttttctgctccctttactagcaaaactgcttgaaacagtcgtctgtacttgttgggtcccttccaaacctctcttctctctccgacctgcttcagccagggttctgcccctgctgctccagggaatatgcttcctgagggtcattaatggactccgtgttgccaaatcagtggtaattctgtcctcagtggcagtttttgtcccagaaggtcagacaccctttcccctgtggcccagccatgttctccacccacctcacagactcctctctcggcctcacaacaatggtgggccttgggctcactttcggttctgggtacactcctgcgggtaccatgcctccacacagcacctgccgctcatgtctccaccccttcctgccagcctagcctctctccctgagcacggactccacagctccatctggtcactcgagtttaacatgcgtcacagtgggactcctggctggccctcccaacaaacctgctcttattcatcttccctgtctctttttttttttttttttttttgagacaaggtctgtcgccaaggctggaatgtagtattgagatcctcccacttcagcctctccagtagctgagactacaggcgtgcaccaccatactcagctaatttttgtattttttatatttatttattatttatttatttatttatttttgagacagagtttcactcttgttgcccaggctggagtataatagtgtgatctcggctcactgcaacttccgcctccagggttcaagtgattctcctggctcagcatcccaagtagctgggattacaggcatgtgccaccacgcctggctaattttttgaatttttttagtagagacagggtttcaccatgttggtcaggctggtctcgaatccctgacctcaggtgatccgcccgccttggccccccagagtgcagggattacaggcatcagccactgcgcccagcctcatttttgtattttttttagagatgaggtcttgccgtgttgcccaagctggtcttgaactgagctcaagcaatcctcctgcctcagcctcccaaagtgttgggattataggcgtgacccactgcgcccagcctcttccctgtcttagaaaatgacttctccactcttcgttgctcaggtccaggcttcagaatcattctccactctttgtctctcacactctttgtgaatccctgagcaaatcctgttgttgctgtctccaaaatgcatccagaatccaacctttctccatcaactccactgccatttttgggtcccagccaccctcaatcctccccgggccatcgcagcagccccagtacaggtttccaggttcctgtcctcacgccctgccgcctacaggcaacacagaagccagagaattgctgtttcagtgtcagtcaggaattttctctcttcccctgcagactgtgcaacaccccatctccctagaggaaaaagcccaggcctcaggatggccttcacagcccttggggtccccctgccccaggactcctccaacctcatgccccactcctccgcctggcggctgtttttcagacaccccagacccacccccactcaggtcctccacctgtgccgtttcctctcgctgaacacccttccagtcacctcctccagctccttgctgtaacgtcaccttggtgaggcttgccctggccccctgttttatttttgtttatttattttgtagagacaaagtctcattatgttgcccaggctggtcttaaactcctgagctcaagcgatcctcttgcctcagcctcccaaagtgccaggattacaggtgtgagctaccaagcccagccttgaccccctggtttaaccactcaccaccatccccacacttccctgtttaattttccctggcattgcctaggctagcattcctagtcttccaacatgacattatttatttattaagctatttttgtttgttaggcttttttttcttttttcgacaggtcttgctctgttgcccagcagagtgcagtgaattcctgggctcaaggaatcctcctgtctcaacctctccagtagatggaactataggcacttgtcaccatgtccaactttttttttttctttttgagatggagtctcgctctgtcaccaaggctggagtgcagtggcacgatctctgcttactgcaagctccgcctcctggattcacgccattctcctgcctcagcctcctgagtagctgggattacaggcacgtgccaccactcctggctaatttttgtatttttagtagagacagggttttgccatgttggccagcctggtcttgaactcctgacctcgggtgatccactcgcctcagcctcccaaagtgctgggattacaggcgtgagccaccgtgcctggccttatttatttgggtacttttaaaacctctctcacacacctataatcccagcgctttgggaggccgaggtgggcggatcacaaggtcaggagatcgagaccatcctggctaacacggtgaaaccccgtctctactaaaaaatacaaaaaattagccgggcatggtggtgggcgcctgtagtcccagctactcgggaggctgagacaggagaattgcctgaacccgggaggcggagcttgtagtgaaccgatattgcgccactgcactactccagcctgggtgacagagcgagactccatctcaaaacaacaacaacaacaacaacaaaagtccctgcatggaggccaaggcacaggcagaagtgccactgggaaaggagattctagaaaaggcctaaacactagacatcctgggccaggtgcggtggctcacacctgtaatcccagcactttgggaggccgaggctggcaaatgacttgaggtcaggagttcaaaccaggctggccaatatggtgaaactccatctctgctaaaaatacaaaaattagccaggtgtggtggcaggtgcctgtaatcccagctgctcaggaggctgaggcaggagaatcgcttgaacccaggaggtggaggttgcagtgagctgggattgcaccgttgtactccagcctgggtgtcgcagcaactccatctcaaaaaataaaaataaaaataaaaaccactagacatccagatgtcacccaggatgctgctccctggtttggcttgggccaaaagtgaaatgcagagagtcaccaggtatttggtgagggcctcagaggccctcctatagggtcctgtgctctggggggactgacaggaaatggaaatagacacgtagggagatcattccaggtggtgccattgagagggtcacatggataacatgatggggcataactagagaccactgccctcaccagggcaccagctatagctttgttcagaaggtgaggacgaaggggaaccctcagcattgagaaggctggaggcctatggcccagagggcccaggagtgtgtgtgaatggatggatggctgagggagacttggctcacagcccagaccagccctggagcagccaggagacagactgctaccccagcctcaggatctcagggcagggtgcgggggaggttgactgggccagagcccgctacccatcacagagagctactcccacggacctgggaggaattctggaaggagtctgagcagcagctgcagaaggagaccctgccccacggcccagggacctcagctgggggaaccccctcacctcccccattcttctgcctgcagtgattgctaccccagacaagggtgccaagctctgaagcctgctggccacaaagaggctctgccaggttgccagctatgctgtttgttggcagaagcatgtcattctttcccctctcatttcagatgaggagcagacctctcatcaggtcccccaaatcccatacgatgtggctgctgcccttctcctacatgctgttccagccacatctgcaatcgtaagttgcttgaatgtgcagagttgtttctcccctctgggtgttcccttccctgccttttttttttttttttttgagagggagtctcgctctgttgccaggcgggagtacagtggggtgatcttggctcaccgcaacctctgcctcccgggttcaagcgattctcctgcctcagcttcccgagtagctgatactacagggacacgccaccacacccagctaatttttgtacttttagtagagacgaggtttcaccatgttcgccaggatggtcttgatctcttgaccttgtgatccacccacctcggccttccaaagtgctgggattacaggtgtgagccaccacacccaacccccttattttttatttttgtaacttgtgattctacatttgtctgtggattttgtatcatctgtatcatttgtagatgagatgcgagctccatgaactcaaggagctcacctgcgttgtctgccgctgtcttcctggagccttggccatctggtacatggtaggcaggcaataaatatttgctgaacgtatgaatgaatgcacaaatgatgtggtcagatctgcaggaaaagatcttccctggcagaagggtggtagtcggattggaggagacaagtgggagacaaggagagaggaaggaaggaccactctgatttactgagtccaggttggaggtgagtcccactgtaggggagatgggcatctaggggattgaaaagacatttcaaagacagtgtggcatggcaggaagaataggaatgtaatgaagccagaatctctgtgtgtgtgtgtgtgtgtgtgtgtttgaaacggactcttgctctgtcgccaggctaagagtgcagtgccgcgatcttggctcgctgcaacctccgcctcccgggttcaagcgattctcctgcctcagcctcccgaatagctaagactataggcacttgtcacaatgcccagctagttttgtacttttaatagagacagggtttcaccatgttgtccaggatggtctcgatctcttgacctcgtgatcctcccgcctcagcctctcaaagtgctgagattacaggcatgagccaccgcgcctggctgaacctctgtgttttaataccagctccatcaccagctgtgatgctctgtgcctgagcatcctcatcatcttgtcacacggcaccaacaccttagaggtgtatgtatctcaaagggatgtggattcaatgccacataggtccaggcctggccaccacaggtgggctgaccgtcgataacaggagtgagaattgtaattcaggtggaatcagtagtcgcaggggctatttggctctaacccacctcttcggcttctccaaggcaagggaggctttcgaggagatgagcttttgaattctcagcttttcacaaaaatagcctccacgccggagcccctggtttcagccagacgatttctttgttactcagctgctggtgggtgggtgggtggggctgcggctgctctcttcctgaatgaacagctgtgccctctcatcgccggctcaggaataatcataacactgcttcaatgctcggcccagcctccttcattcactgctctctggctgcctggcagctctgggcacatgttcaacagcccatgggaggggccaagagaaaggagactggacgtgaagggaggggtcagggccttggccacagcagccgggcctcagcctcctgagagctggcctccgggaccctgcctcatgttccgcattcccttccctcaccctgtaccctgacactgcctcctttctgttcctccgccaccccaagctcatatacagccaagggccttccaacctcccgagcttcttccctctcgatgtcttctctctaggtcctgacaagctgggtcccgaggtcacctggagtgcagaaatgtccgcccagccactccccctctgctggccaatcagcttataacccatcactcaaacaacatgggggcccccgggagaagggccagcctgggcatccacattccagctcaagtcctgatgccagttctggttggccaagcctgggccagaggtcctacagaccaagagaggggagaggtgattccccaggtgacaacatcaaggcagttcccagcaaacaggatagtaagggtggccagggaaaactgccagcgtccaccatggctccagcgcttctgctgggagggcaggttggtgtgtttgaaaaacataggtccaccgggcgcagtggctcatgcctgtaatcccagcactgtgggaggccaaggtgggcggattgcctgaggtcaggagttcgagatcagtctggccaacatggtgaaaccctgtctctactaaaaatacaaaaaaagtagccggacgtggtggcgtcctcctgtaatcccagctattcaggaggctgaagcaggggaattgcttgaaccaggggtgtggaggttgcagtgagccaagatcgcaccactacactccagcctgggcaacagagcaagactccgtctaaaaaaaagaaaaaaaagaaaaaaaaaacataggtccagagatcaggcttggggcctctgctccagcactcctccacttcggagccgcgtgactcagtggagccccacgagctctctgagctgtttcctcatcccaccgaacccgatagagctgtcagaagaagcaggcaaggggctaggcgcagtggctcatgcctgtaattccagcactttgagaggccgaggcaggtggatcacctgaggtcaggagtacaagaccagcctggccaacatggcgaaacctcatctctactaaaaatacaaaaattagccagatgtggtggcacatgcctgtagtctcagctactcagcaggctgaggcacgggaattgcttgaacccgggaggcagaggttgcagtaagctgagatcacaccactgcactccagcctgggcgacagagtgagactccgtctcaaaaaaaaaaaaaaaaaagaaagatattattgctggaggagaggaaggcaaagaagaaagaagagacgatgttatcccagcactttgggaggccaaggtaggaggatcgcttgaggccaggagtttgagactagcctgggcaacataatgagaccccatctctataaaaattttttgaaaacttagccaggcatggtggtgcacccctgtagtcccagctactcatgaggctgaggcgggaggatcatttgagcccaggagttggaagctgcagtgaactgtgattgagccactgcattctagcctgggtaacaaagcaagaccctgtttcaaaaaagaaaaaataaataaataaaaaggaggaaagagagaggagaatttaaaggggaagggaggctcctctgggtagagttctcaggtggggctgtgaaataaaatcagaaccctggctgggcgcggtggctcacgtctgtaatcccagcactttgggaggctgaggtgggtggatcacgaggtcgggagatcgagaccattctggccaacatagtgaaaccctgtctctactaaaaatacaaaaaaattagctgggtgtggtggtgtgtgtctgtagtcccagctactcaggaggctgaggcaggagaatcgcttgaacccgggaagcggaggttgcagcgagctgagatcaagccactgccctccagcctgggcgacacagcaatactctctaaaaaaaataataataattcagaacatccccaggtaaccacaggcagcatttctcaacagtctctcctgcagaaccagaaacccagactgtctgagcctgattgtcctgagaggttctggaagggcaggggcgtgtggtgacactgggtgctcctgggttccagtacctctctagccagttggaagaatataggcaagtcaagtccttcctcctccctgagcctctaattcctcaccggtaaagtagggatgataaaaataaattattaaattgttgtgaggtttcaatgagggcaaagcttgctgcctgaaacagagcaagctcttgtggcgtcttagctatttgtattagttacttgtatttttctctgcggtttcccttttaactcccacctcctttttattttaaaaagactccttttgaaaatgccagcctgcctcagtagtaccaggagacaccagggaaaggcttccttctttccaagctgaggctgagaaggaatgggaagaacgaagagaagcacctttgtggaggcctcctccagctggccctgggtttaggaacatcacacactcaacctccaaaggccctcaggggctggcccaaggtcacacagcttgacagtgcggagcacttcgaagccagggcgtgtggtgtagaacaggctgggtgcctgaattcaatcagccctctctacttctctactcagagcctcccaggctgtggggtgttcacctgtgcaccttcaagccgatcacttccttggggtcctcgtcaattcaattccatggaaatattacaaggggtagacagagatgagccgtgtcagtgtctgtactagcagagggcaggaccactgagcgtccggaggggagacgagtgtctgggtgtccctgggaacctggcttgtctcgacccctctggggggctcctcttggctctgacctccattctcccagactcacccccactcatgcccacccaccccgaggggccccccagtgtgcactcacacactcacactcaaactcaccaccccacttgctctctaattaccaggaacgtggcgctgtctcaggttcttcctcccctgcattcctcagcagctgccctcactgggcagggcaaattataccgtggcccgaccataaaagccactttccaaaaaaagaagctttaaggcccccgagagacgcagctgagcctctcacctggcagtgcctgactcaggaggtgatgtctgggccatgagccacagagcctggtgtccctttaagcagggagtacaaattctcatccctccctcctgctctccctgacgtcagctggaaggcgtgtgagtgagtgaggggctgaggtaccgcccgcgcgagcacacacagacacacacacacacacacacacacacacacacacacatcttccagacccatcccctgcctgccagctccacgagccagagagagacagagcgcggacgccgcaggaggcagtgcctggaccccagctgcccaggagaggtgaggggctagatggggcctgaggacactgtgccagaaactgtggctttggccaggccgaaggagagaggtacaacgtgaaggtgggggctggatcagcccagttcagcttacagatagggtgttgggggtatagaggataccacctgtctctccctccctcattctctccccgtgacccccaaatgaggtgaggccgtgtggctcccgtgggagcatgtctgtgtctgtgtctgtgtctgtgtctgggacctgcgtctggctcaggaggcagagagcagcagatacctgccaagccgcagcctcctgtctgggaagcagcgggagagaggaccgccttgaccagagaaaccccagtgccgcttccagttgctgaaagcagagtgcagcagggacccttgctggtccttcccctggaatgttgggctgggaagggaccggctgggaaaggcagacagttggacctggagcagctgtcttatcctgactctattttttccccagtgtgatggggataattgtgttagttggcttagagaggtggctccgtgtggttattctcttcccccaggtaccaccccaccccgagcccttgcttcttgcctgcctgctggtctgctctgacctccacccttgccagggcacagcccccggagggcaggggtgataagtcagggcctgccaggcctctatcttgggatacagggacttagggtggccttggctggtgccaagcccatgaatggcctgaccctggcacaaggagtccaggcggagataaggggcgacgctggcacccacaaggagacagcaggttcctgccaagctgccagcctgcaggctcttcccagggagaggggagggggacacagagcgagaacaataataaatacagagtgtgaactgctgcatccactggggctggggtaggagaggaggaagtatgcccagtggggggtgggcaacctctctgagagcctggcatcatgctgcctggctgtgatccggcaggggcctctccctgggaaagggggtctccagctttctgggggatggggaactggtttcccaaacctgactccatcccagagtcctctggaccctccaaattctgaatgtttcctcctcccagctttgccctgccgccccccatttccttgaagtcttctccaaaaatgtctgttttctgtgtctgcccatcttgacccatcctcacccccttcaaaaaaaaaaaaaaagacaggtatagacaggaactgcctgcccgtgtcctgggaaaggccagaggacagttttggccacagatggctctcccaggcttggagagacatggctaggcaatgttcacggatgcgcagggggattccctgagggggacttggggacactctcagatggctggtggggaggggacacctgccagccagtctggagtagagccccctagggggtagagggcgctcaggacctggctgagccagtcatgtccacggtcacaactggggcagccaggacccagagccttgtaggaatggggcttggggcagttcagacctttgtccatgctgagcagcaaaatgggaggctaaaagtagtagggagctcttctgccccacagccagcaaaatgacactcctgggctggggggctggcatggcacaccagaaagctgtggggtcacactgattggagttcaagttcagatgctgtatgtatgtatgactttgaccagtctattcctctctctagggggtctcaaaatgggaaagtgacttgcctagggtcgcacagccaagcacaggtgggactggggtgcgtatttggctctcccagtgccgtcctggtttccccaccaccacccaagccacagaagcccaaaggccttaaccacagcagttccccagggcagcctgggcatgtgacaaggaggactacagggtctcttaaataattaagctcaagtattggctggggcagccctcatgcacagacgcaggcacacaggctgtgtgggggaaggaccaggtttaatcaggtcacctcaggctgcaagagcagttcaggcactgatgtattttaaggagcatctgggaggctgggggagggggaggagccagggaagtgtcagctcctgcccgggggtcctgagtttgttttctcactctcccagggcccaccatgcacaagctcccgatgttcgggaagcccttccagcgtcatccttgtgttccccgtcctttctatttttaaaccaccgtacccattcctcccatctgctcccctgtgatgggggcttggggctgggacatttttattgggggagttgtttttaagcttagcagagttggtgagtgactcacccaaggtcatggtctgagtcagcgcaaagccagaaataaaagccccagtcctggctccccaggaattccacacccgactcctccttctccaggggagggtgggcgccaccctcaaggaatggctggtcttcacctccccagacatctccagagggcagatatgctgcacctgacctgctgtgagagctcagcgaggctcttcacctctctgagcctcagtttactctttagaagatgggtgtactaagccaggcgcagtggctcatgcctgtaatcccagcactttgggaggctgaggcgggcggatcacgaggtcgggagatcgagaccatcctggctaacacggtgaaaccccgtctctactaaaaatacaaaaaaaaaaaaagaaaaattaaccgagcgtggtggcgggcacctgtattcccagctactcgggaggctgagccaggagaatggcgggaacctgggaggcggagcttgcagtgagccgagatcgcaccactgcactccagcctgggcgacacagcaagactccatctcaattaaaaaaaaaaaaaaagaagaagaagaatgggtgtactaatggtagttactgcatcgggttcttatgagtacgaaatgaaaaaatggggcaaagggcttggcacatggtcagtatcaatagatcactagatggtggctgaggctcacatcaggacttccaatgctgcttttttaaaattattattgtttttgagacagagtcttgctgtgtcgcccaggctggagtgcagtggcgcgatctcggctcactgcaagctccgtctcccgggttcacaccatcctccggcctcaggcctccccagtgactgggactacaggtgcctgccaccacgcccagttaattttttgtatttttagcagagacggggtttcaccgtgttagccaggatggtctcaatctcctgacctcgtgatccacccgccttagcctcccaaagtgctgggattacaggtgtgagccaccactcttggcccccaagctgcattttaataatctgtttctgttccccccgctggactggggtgctctaaggacagctgttgccccacgtttgttgttctggtgcctgcactcagtttgatttcatttacatatgtgtttttgaaaacctcggaggagaattggcctgaccttcattagtcatctggaccaacgatgattcacgtagctgaagagaaaggtgaggtccagagaggagcaggtgcctgccccaggtcacacagcactgggtggctgaccaggctgcctttccctaatccgctcagactgagaggactcatggtcaagagaagaccatacagtcaggcgcggtggctcacacttgtaatcccagcactttgggaggccaagggggttgatcacctgatgtcaggagttcaagatcagctggccaacatggtgaaactctgtctctactaaaaatacaaaaattagctgggcatggtggtgagcgtctgtgatcccagctactcgagaggctgaggcaggagaatcacttgaacccgggaggcagaggtttcagtgagccgagatcgcgccactggactccagcctgggagacagagtgagactcaaaaaaaaaaggatgggtgcggtggtttacgcctttaatcccagcactttgggaggctgaggcaggtggatcacaagatcaggagtttgagaccagcctggccaacatggtgaaaccccatctctactaaaaatacaaaaattaatcgggtgtggtggtgcacgcctgtaatcccagctactcaggaggctaaggaaggagaattgcttgaacccgggaggcagatgttgcagtgagccgagatcatgccactgcactccagcctgggtgacggagtaagattccatctcaaaaaaaaaaaaaaaaaaaaagaggactatggccaggcacagtgactcacactgtaatcctagcacttagggagactgaggctgcagtgagccatgatggtgccactgcactccggcctgggtgacaaagtgataccctgtctcaaaaaattaaaaagtaagagagaaccatgacaaccatttcctgccccattctgcatccttttaggggagctgagaattggagcagagtcccagcatgggctgtggttcatctgccaagaaggctgagtcaccacccagagtgccccatgacactgcatccagcctcaggttatctccaaacagcctgacatttgtgtttacttctaggcccctttgtctgcagagtcagtcatttggggtctggccttctgcagggaaatgaagatgagcttggctcagagcccaaactcagggccgggggacagggggatgcacactcgccggggggcctttgacacagctctgtggctcctcccgacaacctgggtaggatgttatgatcagccccatttgacagatgagggatctgaggcacaaaaagggagagttggccgggcgcagtggctcatgcctgaaatcccagcactttgggaggccaaggcgggtggatcacctgcagtcaggagttcgagaccagcctggccaacatggtgaaaccccatctctgattaaaaaaaaaaaaaaaaaaaaggctgggcgcagtggctcacgcctgtaatcccagcactttgggaagctgaggtgggcggatcatgaggttgggagatcaagaccatcctggccaacatggtgaaaccttgtctgtactaaaatacaaaaaattagctgggcgtggtggcacacgcctgtagtcccagctactcaggaggctgaggcagaagaatcgcttgaaccccacaggcagaggttgcagtgagccaagatcacgccactgcactccagcctgttcacagagcaagactccatctaaaaaaaaaaaaaaaaaaaaaaaaaacacacaaaaattagctgggtgtggtggtgggcgtctgtaatcccagccagctacttgggagtttgaggcaggagaatcacttgaacctgggaggcagaggttgcagtgagccgagatcacgccactgcactccagcctgagggacagagtgagactctgtctcaaaaaaaaaaaaaaaaagggggagggtcaccggactcagtggctcacgcctgtaatcccagcactttgggaggccgaggcgggcagatcacgaggtcaggagatcgagaccacagtgaaaccccgtctctactaaaaatacaaaaaattagccgtgcgtggtggcaggcgcctgtaatcccagctactcgggaggctgaggcaggagaaaggcgtgaacccgggaggcggagcttgcagtgagccaagatcgcgccactgcactccagcctgggcgacagagcgatcctccgtctcaaaaaaaaaaaaaaaaagggagggtcacaaaactgggttgcagtggaacaaggatttaaactcaggcagtctgggtctcaggctgccctcttacctgactgctcgttctgaagggcattattcctggcaggagatggccccagaaggacacacgaaatccccctggagctggcctttagctctgtttgagtggaaacaaatgttctagaaacatatcagtttctctgctcttgggggagtttattttcccatttagtttctcagggaaagttcttttggaaaaactccagcctcatcttggctgaaccaggaacagaatttatattcacaggaagagcactggtttactgttagacagatctgggactctgtcacctgctgtgtgactttgggcaagtcactcaacctctctgagcctcagctatctcatctgtaaaatggaaaaattgtaatttcaactctatagggtaactgggaggttgaaacgtagaaaataatttctgtaaagacttttgcacagtacatggcattcagccaatgaaagcctcattttcacttgccctctttggcagcaatgctctggagagggggtctgaactggagggttgctccgtatctccatcttcccttctctctggtctctcgggctgctgggcattgtacagctcctccaagcccacgccctggcagggcccagagagggaaaggccctgtaccagctcacacagtaggtcaatagcaatacagagcctggacacaggactcctgccttcccactggaaggctccagatcatactgattcaaattctggctcagactttgattggtgtgtgtccttgagtgagtgatttaacctctttgagcctcgattttcttgtctacaaagtggggtaataatagcaccagccttgaagagcagttgcaggaattaaataaaacaagagactgtggagctttgaggggtgggcgatccttagggaagaagagaggatgaaagtgaatggtgggcttggcccagctcggctgggtctctccttcccccactccctcttccctcaacacccaattccacagccttgtctaggaggggcaggctctcgcccagcgcccagcgcctactcagtgcttcccacatcccacctcaattctcatttcatcctcaaaagagcccttggaggagagagtcttttttttctctctccctctcttttttttgagactgggtcttgctctgtcgcccaggctgtagtgcagtagcttgattaccactcactgcagccttgacctcctgggctcaagcagtcctcccaccttagcctcctgagtagctgggaccacaggcatgcaccactacacccagctaattttttttttaagacagtgtctaactttgtcccccaggctgcactgcagaggcgtgatctccgctctctgcagcctcgacctcctgggttcaagcaatcctgctgcctcagatgcccaagtagctgggactacaggtgcgccaccgccatgcctagctaatttttgtaatttttatagacacaagctttcaccatgttgccccagctggtcttaagctcctgggcttaaaagatcctcctgccttggcctcccaaagtgctgggattattggcgtaagccaccttgcctgttggaggaggaattcttgtgatccccatttcccagataaggaaactgaggctgcaatgggcgacaggggagaagcctgttctctctgccttgtggaagctgcctgtctcctgcccctgcctgagctcttgccctgaggccaactcgctgcctcctcttggtcctgtctttgctgtggctcttctgcctcccttctgctctctggtctctctgccacctgagagcccatcatggggaggctgccctcgaccctggcccagaatcatcagtgggttcttcccccatccctgcagcccacttagcatgaggccctggagccgcacagtcacttgggtgggaggtctggctggccagagcaggccccagctcagccgcgtctccctggtaaccaccatgagctgcatctccccctctccatcctcctcttccctccctgcccctccatggcctcccagcactggctgtcactgccaagcacaataccaggcctgggaaaactgcagttgggaggccaggaccgccctgctccctagagagagggaggcagggcctctgtccccatcgcctgccaaccctcacccaggtggggcaggtggcactcaaggagtgcactgcagggcgtctggtgcttcccagctgtgtcatcttgggtaactttgctgccccgtcttcttttttttggtaacagctttactgagatatcgttcatataccataaaattcacctgcttaaggcgttcagttcagttgtttttagtacattcacagagttgtgcagccatcaccacagccaattatagaacatttccattccccaaaaagaaacgttctacccattggcagccctcccctctccctccttcccagccctggtgaccaccagtggactccctgtctatgaggatttgcctcagagatggaatcatgccctctgtgtttttgggactggcttcttctctgtgaccttgcaaacattctcgcataatgtgtgcagggttcatctagggtgtagcgtgaatcagtatttcttcttttctttttcttatggccaagtgctagtctaccatgtgtctagaccataatttattatccattcatctgctgatgggctcgggctgttttcacttgttggctgttgtgaatgagcactggtgtgaccattcatgaacatttatgtccaagtgtttgtgtggttgcttttttttttttttttttttttgacagagtctgggtcagtcacccaccctggagtgcaatggtgtggtcttggcttgctgcaacctctgcctcccgggttgaagcaattctcatgcctcagccttctgagtagctgggactacaggtggatgccatcacacccgattaatttttgtatttttaggagagacggggtttcaccatgttggccaggctggtctcgaactcctgacctcaagtgatctgcccaccttggcctcccaaagtgctgggattacaggtgtgcgccaccacacccggctgtttgtgtggttgcttcttgaagcttccgtattcttctctctaaaatgaggataatgggtgaaaaggccaaacctggtttgccctgccaacctggcatgagcttagacagaaaagccccacttcagccttttcagggttgagggtctgtgaggcaagccaagcccggggctccagtgccttcctgactggagggggagggttttgggaggagggaggatactactccacctccacccctaggcatggcagagccagctcagcatcttggttcagaaaaattccacttgctgtataatcttgagcagaggccagaagacctgctccagtggactcctggactcatccaggcctgccatgcagggtgttcaggctttctgagctctggactccaaggaaccttacaggaaccttgtgggtgattttccttccctgggaactccaagctctggggtgggaggggtataaattggaggccacaggcctgtaaccccagcattttgggaggccgagggaggtagatcaactgaggtcaggagttcgagatcagcctggccaacatggtgaaaccccgtctctactaaaaatacacaaattagccaggcgtgttggcaggcacctgtaatcccagctacttgggaggttgaggcaggagaatcacttgaacctgggaggcagaagtgacagtgagccgagatcacaccactgtactccagcctgggtgacagagtgagactctgtctcaaaaaaaaaaaaaaaaaaattagctaggtgtgagggtgcatgcttgtaaattccagctacttgggaggctgaggtgggaggatcgcttcagcccaggagatggaggctgcagtgagtcatgaccatgtcagtgttccccagactggatgagaccatgtctcaaaaaaaaaaaaaaaaacaaaggatcaatgctagccccattttacagaacagaaaataagagtcagaagttaagagtgttgaccaatgcgctggacgagttggctcatgcctgtaatcccagcactttgggagaccaaggtgggcagatcacctgaggtcaggagttcgagacctgcctggccaacatggtgagaccccatctctactaaaaatacaaaaaatcagccaggtgtggtggcaggcgcctgtaatcccagctactcaggaggctgaggcaggagaatcgcttatacccaggaggcagaggctgcagtgagccgagatcgtgccaatgcactccagcctgggcaacaagagtgagactctgtctaaaacaaaaaaaaaaatttgaccagagccaaggctatgtagccagaaggtggaggatgcaggattcaaacccaggcctgggtgtggagcccatactcccacctgccatgttggagctgatgtgtggcttggcttgcttgaagtcaccaagttgtaggtgacaaagctaaggtttgaacccagatgtgtgactctgaaacaactgacccttatgccgaaccatattctagaccatcactggccaccctacccagccacctcccaggcccctggagttaagagaattgaactgcaaactggaaggacttaggggtcatatgacccctagttttgtttttctggtgaggactctgaaccacgatggggtcgtttgtgacttgaagtaacgccgtgaggatagcaggtgggaccagccctcctcactgtggccagggttcaggagctgctcccctggactaggctcacctgtgcctctttcttcctcttccagacggagcccgtggctggtgaggccgcagagcagggccaggccgggccctgcccgcaaggacgaccaaacccctcaccggcccctgggccccagagcccaccaggctctcctgctggccccaccatgacttcagagtcgacgtcacccccagttgtgcccccgctccactcccccaagtccccagtctggcccaccttccccttccacagggagggcagcagggtctgggagcggggtggtgtcccacctcgggacctacccagccctctgcccaccaagcggaccaggacctattcagcgtgagtacctgccccttgcccaggcccctgacccttgtcaggacaggggctgaccatccccatcctcttccaactcctgttgtcctcacctgggacaggggaactgtgtgtggaccttccagtaactggatggatgtttgtcccatacatccattcatccatccacccatccacccatccatccatccatccatccatccatccacccacccacccacccacccacccacccacccacccacccacccatccatccatccatccatccatccatccatccatccatccatccacctgtccatccatccatccatccatccacccacccatccatccgtccatccatccgtctgtccgtccatccatccacccatccattcatctatccatctattccacaaagatttactgagctcctgaccagtcaggatccataagctgcttacaatccagagggaacttgttcaccatccattaccacccagcagggttaggggtgaggacccagcatggggtaccagaggagatgatgtcctggttgggtatggaagatgtatcaggtgaagaagggagctggcgattctgggcagatggtacagcatgggcaaaacccaagacacggagttaacctttggagaggggtaggtaggcgggacaaagactaggcctgggcctttggggtaggtcaccgatgaggtggcataggggagtaatacgggtccatatgggggaggccaaaggatcttttgggaaggacagggtctcgctctgttgcccaggctggagtgcagtggtgcagtcatggctcactgcagcctcaacttcctggccttcagcaatcctcccacctcagcctcctgagtagctgggacgactggtgcgtgccaccacccccagctcatttttttaaaaaaatgtttgggccagacatggtggctcatgcctgtaatcccagcactttgggaggtcgagacaggtggatcagttaaggacaggagatcaagccagtatggccaacatggccaaacactgcaaaaccctgtctctactaaaaatgccaaaacttagctgggtgtggtggtgggtgcctgtaatcccagctactcaggaggctgaggcatgagaattgcttgaacctgggaggtgtaggttgcagtgagctgagattgcacccctgcactccagcctgggtgacagagaaagaccctgtctcaaaaaaaaaaaaaaattttttttgtagagatgggagtctccctatgttgtccaggctggtctcaaactcctaggcccaagtgatcctcccgcctcagcctcccaaagtgctgggattacaggtgtgagccaccgtgcctggcctcattatcgttgaatggggatgactgtgtggtttctggcctgggtcaagtttctcacagccctgaggcacgctcagcagaaagggcccagaatggctgtagggatccggggaggagttcctcaggccacagctccaatcccactaccctatctccaggacagcccgggcctcagctggccccgtgttcaagggcgtctgtaagcagttctcacgctcacagggccatggcttcatcacccccgagaacgggtccgaggacatcttcgtacatgtgtctgagtgagtcccctccacctccctgttcttggcccctgccctagtgtccccaatggtgcctcaaaaatggctctccaggcctctcctctcactggctgaggtctgtattcatggtgccttttctgtcctctgagtaggggctgagggtgcgttgggagaggagggctggggccagggagggagtgggcacgcagaccccagaagcctgggagccagcggctgggccctggatcctgttacagcatcgtggaggtctgagagggctaggccagctcctgagcctcttggcctggaaagggccttcatgtgggtgggccaggaggtagagatggatatcatgtctaggcctggccccacccacttctaatgccaggaggaccgggggaggtcactgagcttctcagagctttagggacctccccgtgccctttcacatctcggtccccaagggacccagaaagggaggtacagtttatgagtcccattttattttattttatttattttttgagagggagtctcgctctgtcgcccaggctggagtgcaatggcacaatctcggctcagtgcaacctccacttcctgggttcaagcgattctcccacctcagcctcccaagtagctggaattacaggtgccagccaccatgcctggctaatttttgtacttttagtagagccagggtttcgccatgttggccaggttggtgtcaaactcctgactcaggtgatccgcccgcctcagactcccaaagtgctgggattacaggcgtgagccactgtgcccagccatgagtcccgttttacagattcgagagctgaggttctgagaagcaaggcctggccaggcctcctggcctagggaggcttagggccaggctgcccccaaggccaggccacaggattgtctggggcacagggcctgctgctgggctaatacccctcttcctgcccgccagcatcgagggggagtacgtgccagtggagggcgacgaggtgacctacaagatgtgccctatccctcccaagaaccagaagttccaggccgtggaggtggtgctcactcagctggccccccacactccccacgagacgtggtctggccaggtcgtgggctcctaggctgagtggttcacaggccagctggccgggggttggggagccacacagggtgaacgggcagcagccggctccatgccccactgccctggctgatgagtccttcggtggcctcagtgtgcacgtctgtctgtccgtctgtgcttgtggctatgagcgtgtgcctccacccaccccacgacccgtgactactgtgcaagctggccaggaggtaggtggagggcaaggccaccagacctggcttcgcgctgtccactgcctctctcctcccctccctgccgcagacacgcaggacccgctcgccctcctgcttacccgtccccacggtgactgagctgcgagagcctgcgctgggctcactccctggcctccgcccctgagctgtcccgtgtcagtccctggcccggagccaggctctctgtgctacttacatctccctcccttggctgaggtcccccgccacagcctggacccttccctcagaagcctggccgatgggtgcttttggggagcctctcagcctctggcaggggaggaggactgacacggaggcacacaggcctctcactgccgaggccagccttccttcctgctctgctctctcagtgccatggagaggcaggaagaggggcctgagggcggaaggggtttggggctcccatttcgccggccagtccctcctcctcagcctggcagtggcgctgggctccttcccctgggctgtactgagccgagcccaggggtttgcagagggtgggggtccatctctccagcttggctgcagacctcctttaccctgactcacaagccccactgatgctctgggccatgcccggctgacggctgctgtggatgccgcgagggacggacacacgtccggggcacccacgaggagggcccccagcctgggagactggccctgcggcctccacgtcaaactctcttcccaaagccctaacagaccagtggccgaggtgtggctcttattgcatccatccctgaagatgtgtggctgttgctgtcacctggagtccttttggggccaagatgtgtgtgcacccggggtcgtggccattcactcccaggcaggggtgagggtggcctggcccaggaggccaggaaggagggccctgtctgcctccacctctgggtgcaccccctgcctaccaccctcccttctagagagcacatcgcctgaccggggagaagtggggccgtggttcgagggagggctggccaggggtgggacccttatgagactcagtctgtgagtaaaactgggggctcaaatgcccaggatgaggggatcagtgactgtctaggaggatcccttgccttgtaggtgccccaagaccgcagggtagaaatcagccgggatgcctgcatcccacccccggccccagggcccaccacataaaatctgggagcccagagctgctgaggtgtggtcagctcccctaaaatgggcacggcccagcctgtcccatgaggaataaaggcccctggcccccttgagagagggcttgttggtgagggctgactcctgggggcccccaaggctcccctcctgtggggaggccaccttttaaggcaccactagcagtcaggatatggtggcatcagccctggggcctcctgggtggcaggggggcagcagctctcctccccactcacaggccctgcagtgctcatttggaattcctcccaagacccctggccacccagaccccccattcttcctaacactggcaataaaccctcaactgtgacccaccgtggcctgaagctgcctctgtgtcagggtccccaaggaaggggctgggggtggacacaccaaaccagctcttctttggggattcaatggccccctcctcgactgcatcagacttcctttgtgacctgggccaagtccctgccctctctgtgctgtttacttgtcggcaaagtagtggctgtgacagcatgtgactggggccaattatccattcagacacagctgacacctactttgttctgggaactttaatactgtgacaaagttctctaaaataggcaccttccccaccgtacctcatcgcccagggcaggcaggcagggcaggctagatctcgtaccgatacttgagcacgcctcctcctggtgcagaaagaaacctcttctgtaccgaaatacaagcagcagctgtggcctgggccaccaggtggagcatggggaacactctgggccctgggaggacgaagccagtgccactaggagcagactggctggggacggttgtccacacagactctggccccatctgggtgggcttgcagcaggcgtcctgggccagaggaggggccctggcatctatccaacaccaggacctctctttaggcctaggccaaactcttcagaagtcacgacacacagatgtgggccccggtcacgcctcatgagctgtctctgggaagaaaatctcccggcatcgctgcaccgtgtcctgaggagacaccacgctgtggccaacagtccgggggtcggcaaagatctcaaagtcgttcccaccctgcacacagaggatgggcagaggagggatatttaggtggagtccctctccacattggctgctacggacctgcccctcctcctgccccttcagaactgccaggaagcccaccttctcaccccttgtcagacctgccctgacttgctgtgtgatgtagaacaagtgcccagcctcttggggccccagctttccaatctgtgtaacggggctggtgagcaatggttgttctccaggagccccctaagaggtctgttgtgttgccccatgttctggccttgacctcaggtgatttgcattggagaagccaccagacctggcttctcactgtccactgcgttaggggaaacctggggtgggccacactcacagggctagtctcgttcccaaagaagtggatggtgtcgaagctgtcctggtccaggctatccaggcagtagcgcttgtcccagccctcggggaagacgtcaaagctgatcatgcctcctgtgggcaggggtggggactgttattccctgctgggaggggcagacaaggctaacagaagggctgcttgttcattcattcattcaacaacccttgagtgcctactgagggccaggaataggacacgggacagacttaggatcctcaaggaagttatgacacaggttggagaatgaacaagaccatgaagtgggtgggggaagctcatccggccagggtgatcaggggagacctctgaggaggtggcaatgagctgggacctgagccgagtggggatggctggggcgcaaagtacagcccctgaggcagggaagagaacgagcgtcccagcaacggaggaggccaggagaggtagggggaggacgggcaggccctgggcatcacatggagagggaggaagagcctggggaggactttgggcagaggcatgtcatgactccagctccagcctctctggccactggggaatgggtgaggtggcagaggggagcccagttgtgtcagggtcagttggagcctgggacaagctgctggcagtggaagtgtgagcaccggtgggattccagacagggggccctgtgacttctaggagtttggacttgggaaggggtgggggagagcattcaagggagaggctctggttttttggcctgagaaactgggtgggcatggggcgtttacctctgaggtatgaggtggggaaaactgggggccagtaaacactgaacccctttctgcatcgggctcagccaccctctctgccctgagccggggaggggctactaaagctactgggggccacagcctggtcttggcaggggcagatggtttgaggaggggccctgtaccaggcctcccaggtcctctgcagggtggacgtacctcgagagaacctcagccctttgccagcaaactctgttttcagggcttccacgaacttctcccggatcttctctttctgcagaggggagggagcggatggcagctcagaggacctgctgtgtgccaggccctggctgggcacacagtccacgtgagcgtgctgagcattctaggtgggtaggacccccaactgtgcagatgcgcaaactaaggctcagagaggggttgtgccttggtcaaggtcacctagccctggtggcagagccagggcctaaatccagaagggatggggctgccaatggggctgtgggctgggtcttctgaaggagacctcagaggagagacacgagtaggaagggcgaggagacggggcaatctggatcccacatcagtgtccacctcagggcagggctcgatcctgttatctgtaaccagctcaacaaagcaaatactcagaaatgagcatgggagctgagagcaggagacctgtgaggaggtggctgtgggcattcacagagggcagcaggatgccaggcatggcagagcacccagcatgcaaagacgcggctgggctggggactaggtagtgcccaggccggccccgtccctctgagcaggaactctggcagcagctccgcaaacgtggagcagggggacattctccccgccagcccccaggatctccatctcagagggccacaaaggggagcagctatgcccagtctgaggggcagctgaacatctaaaccagccagcaggacattcagctgacagagggtctcgagaccgggctgggaagtctgaccacatcctgggggaccaagaggcgctgaggattctggagcacaggagtggggtggctggtatggccctgtagtcagagccgggtaggactggggcggaaaggcaggcagtggtcagcagcatggagcttggagaagtggcccagggaggatgtcagatataaagccagacaccccctagcctgagatctgaaggcaagtagccaggcttggctactgtactcctcagctgtgtgaccatgggcaagtcacctgacctctctgagcctatttcctggactaaaggacagagaagtaatgaatgacccctgcctgcttccctctcatggaaaagcagagataagggatgtgagccccctgccagccttggcctggctttccagcaggtaggtgggccccagggaggccagagtccccaccagcgtccactctgtaggcccagggctaccactaagactgccccaggctctgaagaaatgtttccacatggccgaggctggctgcacctcccttctctggagaacaaggctggtcactagacagccccatgtgcaaggccagcctcagcagggaccggcactgtgacctcgagctggttactcaccctctggcctcagtgtcctcatctaaggaagggaagtactcatccccgcttcagggttctggaagatgagctgggttaagcatttatttacaccagctctgggccagggcagggcgcacagatcaggaaggttcatgctcctccagtgccagaagggagcaaacaagagccaaaaatacttgagaaggctctggggtggggagttagggtgtggctccccaactccatgccaggtggggtccatgtgccacctcatccagtgctcatgacagcccatgggatgggaactgctattacccccaccttacacataaggaaactgaggcacacgttagtaaagatgcttttccaggttacaaggcaaatgggtagagctgggactgggaacctgggtggacaggaagggataacttctcagcacgtgtattgggggggaagggttctttgcctaagaccaatcctaactaccccaaaaaaaaagtccccgtggccgggcgcagcggctcacatctataatcccagcactttgggaggccaaggtaggcagatcacttgaggccagggatttgagaccagcctgaacatggcaaaaccctgtccctactaaaaaatacaaaaattaggccgggcgcggtggctcatgcccgtaattccagcactttggaaggccaaggagggcggatcacaaggttaagagatcgagaccatcctagccagcatggtgaaaccctgtctctactaaaaatacaaaaattagctgggcgtggtggcaggcgcttgtagtcccagctacttgggaggctgaggcaggagaatcgcttgaacccgggaggcagaggttgcagcgagccgagatctcaccactgcactccagcctggcgacagagtgagactccatctcaaaaaaaaaaaaaaaaaaaaaacccaaaaccaaaccaaaaccaaaaccaaaaccaaaaaacttagccaggtgtggtggcatgggcctgtaatcccagctactcaggaggctgaggcaggagaattgcttgagcctaggcagcgaaggttgcagtgagctgagatcatgctactgcactccagcctgggtgacagagcaagactctatcaagaaaaaaaaaaaaagtccctgtgcctgtgggcactgcctcccctaagcctcagggcacaggtgggagtcaggagtcactgagctgagaggcaccctcagggaggcaccagagggccaatggcaaaatcaaatctttgctctgcaaccaactccctatgtgacctcgggcagggcccttctcatcgagccttggtttcctcatttataaaatggagataatacagctcatttcattcattcaaaaggtatttcttgccgggcacagtggctcacacctataatcactttggaaggttgaggtgggtggattgcctgagctcaggagttcgagaccagcctgggcaacacggtgaaaccccatctctactaaaatacaaaatattaggccgggcacagtggccacgcctgtaatcccagcactttggcaggctgaggcaggcggatcacgaggtcagaagatcgagaccatcctggccaacatggtgaaaccccgtctctactaaaaatacaaaaaattagctgggcattgtggtgcatgtaatcctagctacttgggaaggctgaggcagaagaatcgcttgaaccagcgagtcaggggttccagtgagccgagattgtgccactgcactccagcctggtgacagagcaagactctgtctaaaaaaaaaaaattagccgggcgtggcagcatgtgcctgtagtcccagatacttgggaggctggggcagaagaattgtttgaacccgggaggtggaggttgcagtgagccgagattgtgccacagcactccagcctgggtgacagagcgaaactcagtctccaaaaaaaaaaaaacaaaaaagggtatttctttgcgaggtgcggtggctcacacctacagtcttggtgctttggaaggctgagggagaaggcccacttgaggctaggagttcaaaactggcctgggagacacagcgagatcccatccctacaaaatattttaaaaattagcctggcatggtgtaatgcacctgtggtcccagctactcaggaggctgaggtgtgaggatcgcttgagcctgagagatcgaggctgcagtgagctaggactggatcactgcactgcactccagactgggcaacagagtaagaccctgtctcaaaaacaataacaacaaaaagtatgccatgtgccaagcataggggatccagaggagaacaagccgcagaaatccctcatggagcttacagttctgggacagggacagacagtaattcggattttaaaagtagatgatgaaagttgtccaaggctacttggggagaaataaggtaggggaaggaggaggaagtgcagcagtggatgtgatactctcggaggccccactgggaaaaggatgtgaaaggggatttgaaggaggggagggacgagctgggcctgagagaagtgtgctctgacagaggcacagcactctagaatcagccaagaggccatagctggagcagagggcagggagagccaccgaacaggtcagagagaatgacagaatgacgtggacgggccagcatggaaaggtctggatgggtggattgctccatccccgagagggagggaggcggtggggagaaaagcaaaatgcacatcaaggtttagcaggggcagtacaggcagaggtgagaggaccttggtgtgttccaggaatagaaagtggaacttgaagggcatagaaaggagacgcggagagccagagccagacagagccctcagggcttgaggcaccatagcagggggctaggacatcatccagttggcaacgaggagccaaggaaggattgtaaatgagggggtggggctggggggtgaatgcggctgacatgtttgaaaggttctaatgggctgtgcgtggtggctcatgcctgtaatcccagaactttgggaggccgaggcaggcggatcaccttaggtcaggagttcgagaccagcctggccaacgtggtgaaaccctgcctctactaaaaaagtacaaaaattagctgggtgtagtggtgcatgcctgtaattccagctacttgggaggctgaggcaggagaatcgcttgaacacgggaggtgggagttgcagtgagctgagatcatgccactgcactccagcctctatctatttttttggagactctatctccaaaaagaaaaagaaagaaaggttctcagtggcattgctggagagcaggctggcagggctatgcaaggggtagggagggacagagaggaaagttggggcagcctggggctggggaaaggggtaatctagaaagtggggtctcagttctgcagatgaacagatttgacactgactcgagaggctgatgaaataagagttgttggacccgatgtgggtgggaaggtggaaatgatgttcccaggtgttggcctggctcagtgggtggctgcctggtgctggtcacctaggaagggggaccagcagcattgtaaagatgcaggtgtgtctacaacaatggcacacagtaggtgcctgaaagggtatcagttaaatcagaacacatcccccagtctattttactgatgagaaaataaaggctcagataaattgagtgactgaggccctacaggcctaagattgcatagctagtggtaccttgtccagttcggagaactcgatcctctcctccagggtgcagctccggccgatgggcgagatgttcagcatgccattccggaactcgatgaaggttccactggtggtgagggagggcggggagccagagagaaccagaacggccctgaattcaaatctagccccacctgggaccccagcctcctaggacttcaaaggggacaagaatccacactccccgcaaaaaatcaggggcccaagtatctccaggttccctctgagggacccacacttctcctccctcaggccccaggttctggagaccaggctggtgggacctcaccgcttcttgggcagcctgagcagggccatgtagctgaggcagaagttgatcaagtcctgcagcagctcctcccccaggtggttctggatggtctggccaaggaacacagggctctgagcgtggcctgggctgcctgaccctgagacaggcacagtgtctctcccccagcccaggacagaagcccccttggactgctccactatggaaaagtggggtgcccctggctaagccctgggggacactgacctgcttggagagcagtcgtccgtgcttatactgcaccgtcccgttctcggcaaacacataatcaaacttctcaatgacttcagggtcacatagaggacaggaggaagaaagagtgtgagaccacgtctgtgaccccacgggcaagtaaagagagaggaagcctacacccttggtttcacattcaaaggccatttgttcaatctgaggctacaacctgcttctccagcctttctccctctgcagcttggttttcttccaaagaagccagtcacacctgtgggcctctgcacacaggcatccatgcaggtccttctgtcagaaccgcccttccctggcttccctgccactcatcctccaagattcagcttcctcctctggaagccatgcagtactctctggcagagggcatcatttccactctgtgctcccctgcccctctgcagaggggctcccttctgttctatagctgccatcgcactgggttctcaaggctggctgcatgggagagttggaggtttcagctctgactgccaggcgaagcagttggagtgggtgggaggtgggaggcacagggagcacagcccagaggctgttgcggggtctgggtggcacgtggttacctggactatgatggcgtggtggagatggagacagaggggacagggagatattttggggtttgctgatggaataaacaagaggtggggaaaaggaaaaattcgaggaagtttctggcttgaggaactggaggcacccactttgactgggctgagaccaggggctctgaggtccagtcgatttcctggataacttcagagtcatcctctgttaaatgggatgatcacgcctactctaaagtgtgattgtggggatcaaatgcaatgacattaacacatgatcatttttgacaaatggtagccatcattggttctgcccttttatttatttatttatttatttatttatttatttatttatttttggatgtgtagcctcactctgtcacccaggctggagtgcagtggcctgatcttggctcactgcaacctccgcctcccaggttcaagcgattctcctgcctcagcctcccgagtagctgggattacaggcacgtgccaacacacccagctaattttttgtatttttagtagagacggggtttcaccatgttggccaggctggtcttgaactcctcatctcaagtgatccatccgcctcggcctcccaaagtgctgggattacaggcgtaagccactgtgccggctggttcagcccattttaaagatgaggaaatcaagactcagcagggagaaatgagaatgtaaactgcataagggcaggttctgcttgctttgccccagccatttctcaagggcctggcacaatccctggcaataggaagtgctcaagtgtttgtcaaatcagtaagggaactatctggctgctctctctctatttcttctgccccacagaagcaggactggatctttgaagacgcccaggaatctctcaaactccccactccctctacccccagcctcactcctaccttcatccccgtcacccagctgctcagcgatcttacagtagtcagagccgcccaccacaccgatctgcactctacttcgtagcttctgcaggaaggcggccacctcagggtcaattttctatggggggagaggggaagcatagcattctggctttcaacatgtctgggacctccacttagccctcacttagtgctgagaacccaggaagcccacattctacctggatctgaagcagtacaaagcttagacaccaaaaattagacaggttggacactgtggctcaggtctgcaatcccaacactttgcgaggccaaggcagaaagatcacttgacccaggagtttgagactagagcctaggcagcaaaggaagacattgtctctatgaaaaaaaaaatttttttggagacgaatttttgttctgtcatccaggctggagtgcagtggtgtgattctggctcactgaaacctctgcccaggctggagtgcgatggcgtgatctcagctcactgcaacctctgcctcccgggttcaagcgattcttctgcctcagcctcccgagtagctgggattacaggcgcctgccaccatgcccagctaagttttaaatttttagtagagacagggtttcgccatgttggccaggctggtcttgaactcctgacttcaggtgatccatccgcctgggcctcccaaagtgctgggattataggcgtgagccaccgtgcctggccgaaagtttttaaaaattagccaggcatgggctggcacggtggttcacaccagtaatcccagcactttgggaggcccaggagggtggatcacttgatgtcaggagttcgagaccagcctggccaacatggtgaaactctgtttctactaaaaattaaaaaaaaaaaaaaaaaaaaattggccatgcgcggtggctcacgcctgtaatcccagcactttgggaggccgaggcgggtggatcacaaggtcaggggattgagaccatcctggctaacatggtgaaaccctgtctctactaaaaaatacaaaaaaattagccgggcgtggtggcacgcgcctgtagtcccagctactcgggagggctgaggcaggagaatggcgtgaacttgggaggcagagcttgcagtgagctgagatcacgccactgcactccagcctgggcgacagagcgagactccgtctcaaaaaaaaaaagaaaaaaaaattagctgggcgtggtggcaagcgcctgtaatcccagctactcaggaggctgaggcaggagaatcccttgaacccgggaggtggaggttgcagtgaaccgagatcatgccatcgcactccagcctgggggacaagaatgagatttcatctccaaaaaaaaaaaaaaaaaaagatattagccaggcatggtggcacttgcctgtagtcccagctaccagctactccagaagctgaggcgggagaatcacttgagcccaggagttcgaggctgcagtgagctgtgatagcgccactgcactccagactgagtgacagagtgagaccccggtctctaacaacaacaacaacaaaatagcatcacaatcaagttgatttttgaggagccaaagaacaataacaaaaatagcctcacaaaacaatgcttaatatttgctctgggcaaatagatccagataaaggcctggagggaagccacaccacagtaaccacattaacttggggagccggccaggctccccaagtgcggtcagagtggactttacccttctctgttacgtttccattctttaccaggagagtgtgctcatgaaaatgtgtacgattaaaaattaactttcaaaaggcactaagcacagaggctggcccgtagacaaattaataggctgttcttattcaggttgttatgtttgtggagcaaaaggtcagagggaggagtcagaactgtgcctaggccctgccacttttgttccagttcttttattcatttatttgtttgtttgagacggagtttcactcttgttgcccaggctggagtgcaatggcacaatcttggctcactgcaacctccacctcctgggttcaatcgattctcctgcctctgcctccccagtagctgggactacaggcacacgacaccacgcccatctactttttgttttgttttgttttgagactgagtctccctctgtcgcccaggctgtagtgcaatggcacattctcggctcactgcaacttctgcctctcgggttcaagtgattctcctgcctcagcctcccgactagctaggattacaggtgcatgccaccacgcctggctaattttttgtatttttagtagagatagggtttcacgatattggtcaggctggtcttaaactcctgacatcgtgatctgtctgcctcggcctcccaaagtgctgggattacaggcgtgagccactgaacccagcctaatttttgtatttttagtagagacggggtttcaccatgttagccagactgatctagaattcctgacctcaggtgatctgcccaccatggcctcccaaagtgctgggattacaggcgtgacccacaagcaaatggcccacctttgctccagtcctgctatcttcctcccggatccacccctgcaggaaggcctccagggcccaagccagggcaacttcaccacagaggatggggaggggcattcgggacctaagagaggaggctaagacctgggttcccatcagccactgcccagtaaagcccatcccactgctcccccacgctaggagacctgttggttggtccttgaggcccccagactggagggtgggttacccacagggagggcatgaatgaagcctgcttactgatgcatccagctggtgagggggcttttggggactcactcacaatgagacccaatgggagaaaaactgagcagaggtaagaggctcaacacagcgagagtctttagaagaaaactagactcttaccctcactattcctcagtgtcctcacagcaaacaggctaggcctggagcctctcacatccgggtgggtagagctcataaacgaatttgaaaagaagccctctctgctccacgactgggggtggaaggtaaagtgggtgatggggcttcagaaactagacccagtcccaggcctgcaaaggtcggggtgggaagactccgagacgccctgtctcccaccagatgatgactgacgcttggctgtgtgcttgtgtgcctggaaccccggttcagaagccagctccgaccactggtgtgtcgtagggctgaactagggtgggatccccagctcaggaggcgggggcgtccgtgagccaggccttagcggcgtgtgacagcctccggggagggagggagctaggcccctgctcgcagcctcctccctccagtactggatccaaccccagcactcccaagcagaacacaggcaaggacgctgcggggtcagtgacgtgagggccaggtcccctcactcccggtgggtggccccggatgtcagggggccgggtaccgccgcatcccacactcggtccccgggtgtccacggtcacgctgctgcagaccccacactcagcctcgggggtgtccgcgtgacactcccggtgggagcttccaatcttcagggtcctacctggcgagccggcgtgagggtcccgtccacgtcaaacaggcagaggacgcgctccttcctgcgggctgcctgggcggtgactgccatggctgcaggtccgcgcgcggggcggagtcccgaagatgcaacggcagaagcagctcagggataagtgctgacggggcggggcattagggggcgggggccagtggactgcctgattggccaaatgaagaaggggcgtggtctaggcgggcggaagcgggggcggggcctgagctgattctcattgctcgtggaggaggggagcgcggcatagggcggagcggagccctccaggttcgattggattgctccaaacaaggcggaggaggggcctgagcctgggacccgcaggaagtgagagggtgaacccggaagacaggggcggggccagaattggcgggttgatagtgattgcaacaggccgattaaggggctgggtcgggcggggcctctaccatgcagtgcagtgtctagtgggccaggcgggactaagagagccgaggtcgctccccatccactctcacaaggtttgtggccatctctttgccttacaaattttgagctgtaggaaatctcctgagccgggcgcggtggttcacgcctgtaatcccagcagtttgggacgtcgaggcgggcggatcaccggaggtcgggagttcgagaccagcctgaccaacatggagaaacctcgtctctactaaaaatacaaaattagccaggcgtggtggcgcatgcctgtaatctcagctactcaggaggctgaggcaggagaatcgcttgattccaaagaagccagaggttgcggtgagccgagatcgcgccattgcactccagcctcaaaaaaaaaaaaaaaaaaaaaaaaggaaagaaaagaaaagaaaagaaatctcctgaaggcctttgttcaggctttggactttgccaaattgccttccccgagccttattccttctgtccacacataagacccagctctcagggtctacctgcttggagaccttcccagattcttcttttttttttttttttttgagacagagtctcgctctgcccaggctggagtgcagtggcgggatctctgcccactgcaacctccacctcccaggttcaagcggttctcatgcctcagcctcccgagtagctgggactgcaggcgtgtgccaccatgcccacctaatttttgtatttttacttacttttatgtttattttattttatttttgacacagggcgatttatttatttatttatttattttttgagactgggtctcactctgttactcaggttggagtgcagtggcatgatctcggctcactgcaacctccgcctcccaggttcaagtgattctcctgcctcagcctcctgagtagctgaaactacagaagtgcgccaccacgcccggctaattttttttttttttctgagacggagttttgctcttgttgcccaggctggagtgcaatggcacgatttcagctcaccacaacctctgcctcccgagtagctgggattattttgtacccactaccacgctcggctaattttttatttttagtagtaattttgtattttcaccatgttagccaggctggtctcgaactcctggcctcaggtgatctgcctgcctcagcctcccaaagtgctgggaatacaggcatgagccaccgttcccagccaaccttcccagattcgattcacaattatttaaccattgcgttgactttggagtcacttccaggttgccttcccagctccaccactatgggaccttggacaagtcacttcacctctcagaggctttgtgtcttcacctataaaatgggatgaacaccacctgctttgtaggtttgcttttcacatgaaaataagtaacaggctgggcgcggtggctcacgcctgtaatcccaacactttgggaggccgggatgggcagatcacctgaggtcatgagtttgagaccagcctggccaacatggggaaagtccgtctctactaaaaatacaaaaattagccgggcatggtggcaggagcctgtaatcccagctacttgggaggctgaggcggaagaatcacttgaacctggaaggcggtggttgcagtgagccgagatgatgccactgcgctccagcctgggagacagagggagactacacctcaaacaaacaaactaactaactaactaactaactaaataaataaaaaataactgtagagctggatgcggtggctcacacctgtaattccagcactttgggaggccaaggtgggtggattgtttgaacccaggaggtcaagatcagcctgggcaacatggcaaaatgctgtctctactaaaaatacaaaacattagccaggcatggtggcaggcacctgtggtccctcaggaggctaaagtaggagaatcaccaggaagctgaggctgcaacgaaccagatcttcccactgcactccagcctgggtaacagtagtgagaccctgtctcaagaaaaaaaaaaaaaaagatgcagatcatatgagtcagcaattcaacttttagaaaataggaaaagaggctgagcgcggtagctcacgcctgtaatcccagcactttgggaggccaaggcaggcagaccacaaggtcaagagatcgagaccatcctggctaacgtggtgaaaccccgtctctactaaaaatacaaaaaattagccaggcgtagtggcggcacctgtagtcccagcgactcgggaggctgaggcaggagaatcccttgaacccgggaggcggagcttgcagtgagccgagatcacaccactgcactccagcctgggccatagagcgagactctgactcggaaaaaaaaaaaaaaaggccaagcgcggtggttcacacctgtaatccagcactttggaaggctgaggtgggcggatcacgaggtcaggagatcgagaccatcctggctaacacggtgaaaccccatctctactaacaatacaaaaaattagctgggcgtggtggcgggtgcctatagtcccagttacttgggaggctgaggcaggagaatggtgtgaacctgggaggcggagcttgcagtgaaccgagatcacgccactgcactccagcctgggcaacagagcagactccgtctcaaaaaaaaaagaaagaaaagaaaatagaaaaaaggaaaaatatatggaattgtttaaatgaactaagacccatctagaagatgaaatgctacgaagccatgaaaatctgcattgtgtttgtgtgcgtgtgcacgtgtgtgtgtagaatagggtctcactattttgtccaggctgctctcaaactcctggcctcaaatggtcctcacgactcagcctcccaaagtgttgggattacaggcgtgaccaaagtgttgggattacaggcctgacccaccgcaccagatccccaggcacaattcttgtttttctgactgggtagctatggtgtcctggagagatcattggagttggaatcaggagaactgcagtttggtcctggctaactacaaggacttggacaggtcacctttcctcactgggcttcagtttctttatatgtaatgaggatataaaccccacgtgatagaactatgaaaggtgatgagaggtgacagcgtactagcagccctcgctcactcttggtgcctcctcagcctcggcgcccattctggccgtgcttgaggagcccttcagcccgccgctgcactgtgggagcccttctctgggctggccgaggccggcgccggctccctcggcttgcggggaggtgtggagggagaggcacgggcgggaaccggggctgcaagcggcgcttgcgggccagctagagttctgggtgggcgtgggcttggcgggccccgcactcagagcggcaggccgtgccggcccgagcagtgaagggcttagcagccgggccagcagctgtggagggtgcgccgggtcccccagcagtgccggcccaccggcgctgcgctcgatttctcacagggccttagctgcctccccgctgggcagggatcggacctgcagcccgccatgcctgagcctaccctctgccgtgggctcctgcgcagacagagcctccccggcgagcgccgccccctgctccatggcgcccagtcccatcgaccacccaagggttgaggagtacgggcgcacggcgcgggactggcaggcagctccacctgcagccccggtacaagatgcactgggtgaagccaactgggctcctgagtctggtggggacttggagaacctttatgtctagctaagggattgtaaatacaccaatcggcactctgtatctagctcaaggtttgtaaacacaccaatcagcagcctgtctagctcagggtttgtgaatgcaccagtcgacactctgtgtctagctactctggtggggacttggagaacctttgtgtccacactctgtaactagctaatctagttgggacgtggagaacttttgtgtctagctcagggattgtaaaggcaccaatcagcaccctgtcaaaacggaccaatcagctctctgtaaaacagaccaattggctctctgtaaaatggaccaatcagcaggatgtgggtgggggggtgggggaggcggtgggggggtgggggaggcggtgggggggtgggggtgggggcgggggtggtggcgggggtggggggggcggtgggggcggggggtggcgggggtgggggtggggggccagataagagaataaaagcaggctgccaggagccagcagtagcaaccctctggggtccccttccgcaacgtggaaggtttgttctttcgctctttgggtccacactgcctttataaactgcaacactcactgcgaaggtctgcagcttcactcctgaagccagtgagaccacgaacacaaccagcaggaacgaataactccagactggcagccttaagagctgtaacactcaccgcgaaggtctgcagcttcactcctgatccagcgcgaccacgaagccaccggaaggaagaaactccgaacacatccgaacgtcagaaggaacaaactccggacacgccacctttaagaactgtaacacggccggcgcggtggctcacgcctgtaatcccagcactttgggaggccgaggcggtggatcacgaggtcaggagtccgagaccagcctgaccaacatggtgaaaccccgtctctactaaaaatacaaaaatgagccgggcgtggtggcgcatgcctgtaatcccagctactcaggtgactgaggcaggagcatcgcatgaacccagaggttgcagtgagccgagatcgcaccacggccctccagcctgggtgacagagcaagactctgtgtcaaaaaaaccaaaacaaaacaaaacaaaaaaaccaaactgatgcttattcctttactatgggctgaccaccacacaggacacttgaccaggtatcttcattttcctgttccatgactgaagaattcctagaaacagatgaagtgcgaattcctgggccctacccgtagtgcttggatccagaacatctggaaggtggcccactgggcaggaggggtgtttgtctcctatggactggatctacttttctaaaaacagagataagcacccttggccccttaacaaatgggctgctgcttacaaatccctttcctacagtctctccgtaaattctcataacaaccctttccacatgatgaggccactgaaacccagagagggagagttatttgccctgaggtacagacccaggaggaccaggtaggggggtacctgtggccaccactatgtccctttaagaggaaggcttatccctggcaactggtgctcagatggggaccaggggaggccttggggagaaaagagcaggactggtgggagggaagactggacagaaaggtgacttggagggacccagaagcagccaggactgaaagtggagggcctgagacctgagcctggcaggattctgctatgaagggaagaccctgggctcttctgggccagacgtgtggctgaagtttggaggttagtgactttcatgtttggtttgggtatttcacttatttttcccaacacatctctgaagacagtacccttatactcaatttccagaggtaggtactgacatcagagaggtgcaacggcttttctagggtcacacagcacagtcgagattcctcctcaatcccgtcttgccaaaacccaggcttttccctggctcctggctattggtaggaggccatgtgtcagtatgtccaagtaccggggctgtcaatgctctttttctgtacctggtatgtgacttgttgtgtatgggtggtccaggcccaaggggcagcctctgtgtagtggggtttacaggcaggctgacaattcctggagttctcctcccactagtgactttgggcaggtccgtcccactctgtgaggtggttgttcatttacaaatggggtcaacaccctcctaaaggctgtgtagttaagaagcctaggggcagcaggggaggtgtgctgtgtgctgaggttgggggagactgtggaattcttttttttttttagatctgttgcccaggctggagtgcagtggcaccatctcacaatcttggctcaatccaacctctccctcgtgggttcaagtgattctcctgcctcagcctcccaagtagctgggattacaggtgcataccaccacacccagctgatttttgtatttttagtagagatggggtttcaccatgttggccaggctggtcttgaactcttgacctcaggtgatccacctgcctcggcctcccaaagtgctgggattacaggcgtgagccacctcgcctagcctgtttttttgttttgttttgttttttaactttcaaaatgcctgcagtgcttacattgtcttcataataagcaagtccattgagtctaaacaagctgaagggcgcatgctcagatggctttgtaggaggaggtggaggctgttgctgggttggctcaggccctctggggctctctcagacaaagtcaatggggacagatatggcccaggccacaacactgttccacctgggggtgtggaacaggagcggcagcctgacagctggggtcagtgggctctgcaggtcagagctcagcccagaactgtgagaacttgggcaagtctctctcctgaccttgcaagccaggggtggggagcggagtcatactgggagggacttagaaaggatccaaggcctttgtcatggtctcaaaatctggggctcaccccatgccaggccctgccctgggaggtactgggctggtgggagtgcgttggaggtacccatgggagcaaggagtcagctctgggtggggacaggtcagaggacatgctggaactgggcctttagcactgaacaaggggtggggaaaggggatccaggtggagcaaagggtgaaccgaggcttggaggtgtgaaaccaccagaagtgacagggactgggactggcacttgcagcagggtgtggggtaggaccagggtgttcaaggacaagattgagaagggccttgataaatgaggacaaggagcttggctcttaccccatgggctcgtaggggtccacacgggctgaggggagctggacgagcttactgaggtggccgagggtacccaagaccagcctggcattgggcgttgggcagtaggctgggcctggtgtgcagggccacagcgccacctggtatccatcactctcactgcagcgtgaccttgcctggctctccaacctgggaaccagtggggcagcctcgtcccagacacactctgcctctttcaagagcagccaggccctgtggtcaagggctcacattcagtggtgtgccaggcccccactaggctccttgcgtgccctctcagctacagcctgtctgggcatgaggagccacagacccaacctagagccaagttgaaagtagaagtgaacacttttcggtgaggtcagacactcctgatagctagaggaagactacagaatttgaagttatcagacaaagcattgagctgcaccagctgcaaggtgaccttgggcaagtgatattctctctgggcccttcccccatctttataaaccagtggtgaaattcagacatccttccctgttatcacttcttatctacaggctcacaaccatgcaaaggagacacatccagaccccgcagacttaagctccaagggacccaaaagcctggggtcgcttcatgttggatgcatgtgcagacctgattcaggcaccacacaaatgcagcaggcagaaatgagtttattgcattagaaaaaacaccgaagaaaaccagcaaacacctccgtactagacagttattagtttgactcaagggcataaagaaaactcagatactagatttacttctgggagggggtgatggtctcatccctcctccttccctatccctttaaaccaatggacctctagaggtgtccactgtgcaatacaagtacgtgactcatttcccactgtgcttgcctgttcacatccaggcggagttagccggccacaatgagaatgctcctgaggtgctgggacagacagaaatatgaggatgggggtaggcagttgaagcctgagcctgagtttacagttagtgacagtggggctcccattgggagggagagcttgccttcccatggagctctgggaaaactgctcagtgatccctcgctctctccccacaaaccctagggcacaggcaatggcacaaatggctgctagtataaaggggggtgctgttcaaggaaatgagtgactcaaattctctcaggggagctgctcctgggcctaagggtgcctgctggaagaagcaaaccagctgaaactggatctggaagaggagaaaggaaaaaaaggagctgtgatgcccagaaccccctggatggaggggagaccccaggcagaggggtctggagcaggaccctggaagacagagccgggccaggcaagctggaaaagggaacaacactggtgggaaacagagcactggccaagaccaacctgctaccctcccagaagaaacccagcctcagaaattctttctctttgggcccaattctgctagtagatcacgattatacaacagtccctgctagtttctgctctaatctattatttccatgcacactggcaaatcagtgggtcttgagaagaattaaactttcaatcatttggcctctacaagttcccaaatgtcacttgacatcccagctggtcatgcccctggtcaggtcgcaggcaaacactaaggcagaatgacagctcctctagctgagactctgatgatgccaaactgtgatggctgatggcaaagatagcagcagatggggccagaaatgagcagcagctttttggagaggaaaggcctgtgctgcaggccagacttccaggaagaagcaacataattcaagtccgtgaagtggcaagaagttgtggcagaaggggcccaaggatgtcagacctgagactcgggtacctggcagctctgagtcccttcttcatgctcactttcccaaaatctaactaatggaggttcatgtgaagagagggaatctgaaagtctctctggattagagactcccagactgttccctgcctataacctcctagcagcaagtcattaggaaaatgcccactcatgccccaacctgcctttctaaagtcagaatgacaatggaaataacgtgtgccttcaaaacacatttaggataagggaaagtccctatcagaaacgattctaaattttcttgccaccttgattccacttcattattttatttttttgagatggagtctcactctgtcgcccaggctggagtgcactggtacgatctcggctcatcgcaatctctgcctcccgggttcaagcgattctcctgcctcagcctcccgagtagctgggactacaggcatgcgccaccatgcccggctaattttgtatttttagtagagacggggtttcaccatgttggccaggctggtcttgaagtcctgacctcaagtgatctgcctgcctcggcctcccaaagtgctgggattacaggcgtgagccaccgcgccaggcctcacatcattattctttccatatcaaaggtattttctggccaggcagcggtggctcacgcctgtaatcccagcactttgggaggccaagggaggtggatcacccgaggtcaggagttccagaccagcctggccaacatggcaaaaccctgtctctactgaaaatacaaaaattagcctggcgtggtggcacgcacctgtaatcccagctactcaggaggctgaggcaggagaaccacttgaacccgggaggcagaggtttctgtgagccgagatcatgccactgcacaccagcctgggtggtagagcgagactccgtctcaaaaaataataataattaaaataaaggtattttcttagagtagggaatcccatcccttggtccccagctaaatactgcgtactggcctggatggtggttacagagactgcaaataacctacatcattacttgcagaaaaaaggccttggcttctactgctccagcccagacctcatccactatgacaagtcagctatgctgaggaaaggcagcctcgggggcacaaaggcaggtttcccaaaattgatggcagaagattggcaatccatctaggtggaaggaggaatggctttatcctgttttctccggcctgatacttgctgttactcccttgatccggaggcaatggctgctccagcactggccttgcttggggtaagggagggaaggtgagggttgtggctaaagagttcacttaggttaggttagtcatcctgaaaccggtcccatgtacaccaaatcctttggttcatttaacactagagtaactttctgggtcacggatgttctaaaactcacctgggtcccacaggtgttggcaacttgggtagagcgtgagtgttctctggaaagagttctaggttggggctcccgcaaactgtgactcgctttctgtttaaacaaagcccctccctttctccagtgtggaggacgcttaggaaacagcatccgaagtgaacgcacagacaagacagccttaataaattagtataatactattagaaggggtggcattttgttctgtttcttagcagcattgtctacatgcggcctgacggtctccttccctgggaaatttaaaaagccgtcccctggttgttagctctgatgtaaaattataaaatagaaatctggtagggtttgttttgtttaaaaaggaaaagccctggtgaggcagggcggtcccaggcagtcctgttagctctggccgttgggtctgcccacggagctccctcctggaggctggatctgaatggagtccaggaggggccgaagtgcctgtccaaagggcctgcaaggaaacagagacatgagaggggtgggctctgggatgtggcctgctgtcacacaccctgtgctgcccacgggtagggaggaggagtgggcgctggtggcagcagaagcagcaccagcagagaaggaaagagcacctcgtcacctcacttaagtttcccacctgcctcacacaccagtgttcttggagggcatctccgaaatagcctgctctgtatttttattcctggggttccctcctcagggcctcacctctagagcctggttagctgtggtttgagtaggctggctacatacagagaactagaactttctgtgctctatgtgcttgtgtctttaattcttgtgagcaaggaaaagatcaaatttaagcagggaaactgaggcaaaagaagaaggatatgaaaaagaagacaaaggggtgaggacagcaaacaactggatttttttcaggaacagaggtctgccagggagtacagggaatattctggaaaaatgacaaatgctaggttgccagtattaatgcctttctttcaccccgagacagagtctacctctagcctagagctggagtgcaatggcgtgatctcggctcactgcaacctctgcctcccaggttcaagcaattctcctgcctcagcctcccaagtagctgggattacaggcgtgtgccactgcgcctggctaattttttattttcagtagagatggggtttcaccatgttggccaggctggtctcgaactcctgacctagtgatttgcctgcctcggcctcccaaagtgctgggattacaggcataagccactgggcccagccattaatgcctttctaaagggctcctaggttgctctcatagtaacagatggaaaaaaaactacaaatactggagtttggagattttccatgattacgacaaccataaataaggttgcccagaaacttttaagtcactggctttttcttcagtggatactgccatttttgaaaatcatctccaagtttaaaggaatgaagtactccaaagtatagacagatgaaacctagaaatatcaaacctaattcagacaattccaggaactccacccataaaataataacagcaagcacattctggctactccagtttgctcagggccccataacctagatccctcagcagagctgtgggactgcaaactcatcgaatgataggaaggcagggccagggctggcggactggggctcctcaccagcctgccacagcagtcttgtttcctatgcaagctcttcctgggaggtgcttcctcagatataaaaaacagcccaccttgctgggcctgcgaaacctgccaggatctctgttaaataaggccatagttcaggagaaactgagtcagaataagcaaatagacatggtcctggaatgagcagaggctgcctgaatgcaatcaacgacaagagggaggaggtgagatggctttgggggtgttgtgtcctttcggggtatactgcctggatgatggcctgcaaggctcttccaaggcttggggcacatcaggacagaacgggctgcagagtgttcttgaagaaaacatctgttgaagactgtgctgtaatacatgagggcaagtagaacatagtcgttgagggagtcaaagagttcatggtgcagtgtaaggcaaatcaacagctacaaaacaagcccccctactatttttctgaccccctctctgcttgtctaacattgcattttaactgggtgcttctggggagtggcatgcttcgagcacaatgggaaggacagacgccagaattttgtgcaagtactgaagccagtgtcaagtgggtgtctaagttctgtggttgcacttaggccagggcagagctggtctagctctgaaggtggaggtaatacaggtgcgcatcagtgggtggagagaatggcagccttctgtgttagaatctgccatcagggcaggcccacctggttgggagggcagatgggggccagaggaagggcactgaccgtgaatggggattttcaaggttggccaaggcaggggcagggacagaggccacacttacgtggagagaacttcagagggcaggtctgtgatgtaagaaggaatcttccgcccagtcaggaactgtgccacttcgttgctgaaggtgttacaattgtgttcaaagaggttgtaggcctcacctctgtacattgaaaggtttgcagaacatatatgagaaaccagcaagcgtctatgtggaataactcaagctaggcagtggaatggtgagcacagctcaatgtgaggattgcgatttcccccgtctcatacaccagaaaagcactgcttatttgccattctggccattaataactgatttttaagggaaggagaaactgaaaagacatggctcctacacagtttcaggagaatctttacattcacgaaagcagaactctggtgttaagagtttacccccttactgaaactgactaaagcctctttcagagaaaatctgctcaagtttgtatttcatcccaacccttctgcaaaccctgtttttccttctgcaaacttggccaatacaggttgagaacagcgcttgccgattttaatcagaaggaatgaaaagcccttatttcctctaaaaatttcggcagaaagtatgtttgaaggaatatctgtatcagaacaacttagagctttttgtagcataactgccttttcttcatgttaattcagcaaaaaactgggccaggccttgtgctgagtgataaggatgatatgacatggcagctgcctccaggggttattatctagctggggagacagacacaactaaccaccacccctgtctccactcaccggaacagggactcccccagggaggagaggtactccagaaagatttcttctgtgacttctgtactccccacatcaaccacagagtctggaggcccaagcaatgtccctccctaaaagagccaacccaaaggaagtcattaagttaccatctccactgaatgttttaatgtggcttcccacagtagaccacaaacactctgttctgactttttttttttttttttttttttagacggagtttctctcgtcacccaggctggagtataatctcagctcactgcaacctccgcctcccgggttcaagtgatcctcctgcctcagcctcccaagtagctgagattacaggtgcctgccaccatgcccagataattttttgtatttttagtagatacagggtttcaccatgttggtcaggctggtctcaaactcctgaccttaagtgatccacccacctcggccttccaaagtgctgagatcacaggtgtgagctactgcgcccagcctgacttcatatttaacttgcagagcaccctgggcagatgaagaatgtgatttgctatttgtatcaaggatgcaatagcttagaagcattggtgtccaatcctttggcttccctgaggcacactggaagaagaatggtcttgggccacacacaaaatacactaacactaatgatagctgatgagcttaaaaaaaaaaaaagataaaaaaatctcatgttttaagaaactttacaaattcacgtcaggccactttcaaagctgtcctgggctgcgtatggcccacaggctgcaggttggacaagcttggcttagagctttctggtcacttttcattgccatgtgctggcagcatagttaggagtcggacttcttgtttctagaggggagggggggccctgggaag

SEQ ID NO: 22 - DNA, 1342 bp, Homo sapiens
  Genbank Accession No. AF030455
actcaaactcctctctctgggaaaacgcggtgcttgctcctcccggagtggccttggcagggtgttggagccctcggtctgccccgtccggtctctggggccaaggctgggtttccctcatgtatggcaagagctctactcgtgcggtgcttcttctccttggcatacagctcacagctctttggcctatagcagctgtggaaatttatacctcccgggtgctggaggctgttaatgggacagatgctcggttaaaatgcactttctccagctttgcccctgtgggtgatgctctaacagtgacctggaattttcgtcctctagacgggggacctgagcagtttgtattctactaccacatagatcccttccaacccatgagtgggcggtttaaggaccgggtgtcttgggatgggaatcctgagcggtacgatgcctccatccttctctggaaactgcagttcgacgacaatgggacatacacctgccaggtgaagaacccacctgatgttgatggggtgataggggagatccggctcagcgtcgtgcacactgtacgcttctctgagatccacttcctggctctggccattggctctgcctgtgcactgatgatcataatagtaattgtagtggtcctcttccagcattaccggaaaaagcgatgggccgaaagagctcataaagtggtggagataaaatcaaaagaagaggaaaggctcaaccaagagaaaaaggtctctgtttatttagaagacacagactaacaattttagatggaagctgagatgatttccaagaacaagaaccctagtatttcttgaagttaatggaaacttttctttggcttttccagttgtgacccgttttccaaccagttctgcagcatattagattctagacaagcaacacccctctggagccagcacagtgctcctccatatcaccagtcatacacagcctcattattaaggtcttatttaatttcagagtgtaaattttttcaagttgctcattaggttttataaacaagaagcctacatttttggccccttaaracacctaccttacagtgttatgaccttgtatacacatatattggtatcaaaggggataaaagcccaatttgtctgttacatttcctttcacgtatttcttttagcagcacttctgctactaaagttaatgtgtttactctctttccttcccacattctcaattaaaaggtgagctaagcctcctcggtgtttctgattaacagtaaatcctaaattcaaactgttaaatgacatttttatttttatgtctctccttaactatgagacacatcttgttttactga

SEQ ID NO: 23 - DNA, 400 bp, Homo sapiens
  Genbank Accession No. M21056
ggctcagtcctctccttcatccctctccaggcaaaaacaaagagtgtgaggccttcatttgcaactgcgaccgcaacgctgccatctgcttttcaaaagctccatataacaaggcacacaagaacctggacaccaagaagtattgtcagagttgaatatcacctctcaaaagcatcacctctatctgcctcatctcacactgtactctccaataaagcaccttgttgaaagacctcatgtttggatattgttttattctctgtctataaactaggtctctgcctactcttttatttttatgtatttattttttctaggtggagtcttgctctgtggcccaggtggatgagtgatgccaccttgcctcactgcaacctccgctcccgggctcaagcaatcc

SEQ ID NO: 24 - DNA, 606 bp, Homo sapiens
  Genbank Accession No. AL079368
ttttttttttttttttttgttagttgtttttgtttttaatttgtctccaataagcaatgtcttctgcccatccagcatgggaagtgggtcccacgtgaagggggcccaccacccactttgggagctgaaggcagagactcgggagccctcccggctcagactccccctacgctgggtgtgggttatattcagaggcccacacccacactggggtaggttaggtatggcttacttagccacaggcccccgggggcaggagacagagtgggggcccgaagctccctgtcctggaagagttaaaagttctggggtagccctggccccgtgggggcagttagagatgagggaggttactccttagcccaggagtggttagagacagtggggctggtagagatgcctgtttaagccttggcctctaggaggtgttagagatttgctggggttgggggccagggttgctgggaccccagttttggggagggagctgtaagagatgggggatgttctgagctccccagacaagactgctggttaagagggggccaggcccgttggggctcagctgtccctgaggagctcggcatcccgctggagatgcagctg

SEQ ID NO: 25 - DNA, 594 bp, Homo sapiens
  Genbank Accession No. X57655
ggggagcgctccctgcgagtggcgcaggtaacagaccgcaggggcaagacagccggcgcgggaggaggatggcgctgtcggtgctgcgcttggcgctgctgctcctggcagtcaccttcgcagcctctctgatccctcaatttggtctgttttcaaaatatagaacgccaaactgctctcagtatagattaccaggatgtcccagacactttaaccctgtgtgtggcagtgacatgtccacttatgccaatgaatgtactctgtgcatgaaaatcagggaaggtggtcataatattaaaatcattcgaaatggaccctgctgatggagcagtttacagaaaagaagatggagagaccaccttcactggcagactagataaattgcatttcccctttttctcttttcctgtgtttctttacatgagatttgttaacacacattttctgagagcaggtatggaagacagccatgtgtagtgatggataatttaaagaaaaaaagaatccttgtttcttggcttttgctcctggagttaagcttactgcccaggtgacttgtgcattgctttatttagttgaaataaaatcagcatt

SEQ ID NO: 26 - DNA, 2326 bp, Homo sapiens
  Genbank Accession No. Z47553
ccagatcgcagctgaaggatctgttgagcgcttcaggaaaggcggacaggcgacactaacaggtgaagatctcgggagaccatgactaagaaaagaattgctgtgattgggggaggagtgagcgggctctcttccatcaagtgctgcgtagaagaaggcttggaacctgtctgctttgaaaggactgatgacatcggagggctctggaggttccaggaaaatcctgaagaaggaagggccagtatttacaaatcagtgatcatcaatacttctaaagagatgatgtgcttcagtgactatccaatcccagatcattatcccaacttcatgcataatgcccaggtcctggagtatttcaggatgtatgccaaagaatttgaccttctaaagtatattcgatttaagaccactgtgtgcagtgtgaagaagcagcctgattttgccacttcaggccaatgggaagtggtcactgaatctgaagggaaaaaggagatgaatgtctttgatggagtcatggtttgcactggccatcacaccaatgctcatctacctctggaaagcttccctggaattgagaagttcaaagggcagtacttccacagtcgagactataagaacccagagggattcactggaaagagagtcattataattggcattgggaattctggaggggatctggctgtagagattagccaaacagccaagcaggttttcctcagcaccaggagaggggcttggatcctgaatcgtgtaggggactacggatatcctgctgatgtgttgttctcttctcgacttacacattttatatggaagatctgtggccaatcattagcaaacaaatatttggaaaaaaagataaaccaaaggtttgaccatgaaatgtttggcctgaagcctaaacacagagctctgagtcagcatccaaccttaaatgatgacctgccaaatcgtatcatttctggcttggtgaaagtgaaaggaaatgtgaaggaattcacggagacagctgccatatttgaggatggctccagggaggatgacattgatgctgttatctttgccacaggctatagctttgactttccatttctggaagattccgtcaaagtggtcaaaaacaagatacccctgtataaaaaggtcttccctcctaacctggaaaggccaactcttgcaatcataggcttgattcagcccttaggagccattatgcccatttcagagctccaaggacgctgggccactcaggtatttaaaggtctaaagacattgccctcacagagtgaaatgatggcagaaatatctaaagctcaagaggaaattgacaaaaggtatgtggagagccaacgccataccattcagggagactacatagataccatggaagagcttgctgatttggtgggggtcaggcccaatctgctgtctctggccttcactgaccccaagctggcattacacttattactgggaccctgcactccaatccactatcgtgtacagggccctggaaagtgggatggggctcgaaaagctatcctcaccacagatgatcgcatcaggaagcctctgatgacaagagtagttgaaaggagtagttctatgacttcaacaatgacaataggcaagtttatgctagctcttgccttctttgctataattatagcttacttctagttgtcctattgtcactgccctgtttttcattgggaagcttatctacagatgccttcagaatctgacgagattgactctcagtttcatattgcccagaaatctactttaatgtctctttcgaaagcattaattcactttcctttttcctacaatgaaacctgttttccatttgtattaactcatctcccttccactcatgatccgtcactcttccttgtggtaatccctagactgggagctcaggtactcttttagtcatctttgtatgtctttagcagagttcttgacatgtggtaggtgcttaataaatgtttgttgtttatcaaattttatggtagggagagtaagtcagcatcggtataaaatcgcttactccacgtaactcttcttctgatagggtttgattttctattagaagctcaattttagttttttttcatattataactaaatatgtttcctgagagataagagaaataatgttcctacaatagttgtatgtatctaagataagacatatagatgcttaagacattttgtttcacttgctattcactagtgtacttgaaacatggtcatttttagcccttttccttaggaaccatgtctttattttctcaataaagaaattactttcaactca

SEQ ID NO: 27 - DNA, 1154 bp, Homo sapiens
  Genbank Accession No. M13928
gcagccaaagccccaggagccctaggttccaaccaactgatgcccctgtgcccactggcccacgccatgcagccccagtccgttctgcacagcggctacttccacccactacttcgggcctggcagacagccaccaccaccctcaatgcctccaacctcatctaccccatctttgtcacggatgttcctgatgacatacagcctatcaccagcctcccaggagtggccaggtatggtgtgaagcggctggaagagatgctgaggcccttggtggaagagggcctacgctgtgtcttgatctttggcgtccccagcagagttcccaaggacgagcggggttccgcagctgactccgaggagtccccagctattgaggcaatccatctgttgaggaagaccttccccaacctcctggtggcctgtgatgtctgcctgtgtccctacacctcccatggtcactgcgggctcctgagtgaaaacggagcattccgggctgaggagagccgccagcggctggctgaggtggcattggcgtatgccaaggcaggatgtcaggtggtagccccgtcggacatgatggatggacgcgtggaagccatcaaagaggccctgatggcacatggacttggcaacagggtatcggtgatgagctacagtgccaaatttgcttcctgtttctatggccctttccgggatgcagctaagtcaagcccagcttttggggaccgccgctgctaccagctgccccctggagcacgaggcctggctctccgagctgtggaccgggatgtacgggaaggagctgacatgctcatggtgaagccgggaatgccctacctggacatcgtgcgggaggtaaaggacaagcaccctgacctccctctcgccgtgtaccacgtctctggagagtttgccatgctgtggcatggagcccaggccggggcatttgatctcaaggctgccgtactggaggccatgactgccttccgcagagcaggtgctgacatcatcatcacctactacacaccgcagctgctgcagtggctgaaggaggaatgatggaggacagtgccaggcccaagaactagaactttcaaacgttcccggggcctcagacaagtgacaaccaaagtaaatgctgcttttagaactgt

SEQ ID NO: 28 - DNA, 479 bp, Homo sapiens
  Genbank Accession No. AI123839
ttgataatgtagaagtttattagagcatgaagagaggaattacctacaagtaattctggagagctggctacctggggccagatgcctcataggcagaaattcattgaatagcatgcatttattatatgttgccagcagtttaagcatttgagctcaaacctcctattagaccctccccagcatgcgggccacccaccagttacaaggcatgatgacacgacagatgactcgtcctccttggtagcagtatggatatgggtagtagcctagtaaaggttcacagacgcggcggcaatagcggttgcctcgacggcagtaaatacaacaataacctctctcatccagcatgggatcaaattctattccattttcagtatagtcatttattggaagttcttcctcacttgcttgtctggcgtgacgggtcttctctactttcacttgagggaccacccactgttcattttgttcaatccctt

SEQ ID NO: 29 - DNA, 93273 bp, Homo sapiens
  Genbank Accession No. AC004084
aagcttctggaccctgagagagattgtttcttttctttactatttttttttttttttttgagacggagtctcactctgtcgccaggctggcgcgatcttggctcactgcaacctctgcctcacgggttcaagcgattttcctgcttcagcctcctgagtagctgggactacaggcacacgccaccatgcccagctcatttttgtatttttagtagagatggggtttcaccatgttggccaggatggcctccatctcttgaccttgtgatccgcccgactcggcctcccaaaatgctgggattacaggcgtgagccatcaagtctggcgagagagattgtttctagatgagggtgggggcgggtgtccttagcccaaagcttgtgccagtctctatcagaaataaatgcccccaaaacctccctgcctgtccgtgacatcatacacctgggtagtctttctgcaccactgcccctggctcctcctcctgaggaggtccccaagggcgtgtaggaccagggcaaggctcaggatcctagagaaattgtcacctggggcaggacaggcctctctgccagccttactgtccctatctgtaaaatgaggatggtaaccctgaacccacaaggccacagtaaggctaaaccaaggtgaacatgccttgggaactgtgaagactgtggaccccaagccggttgcggtggctcaggcctgtaatcccagcactttgggaggctggggcaggaggatcacttgagcccaggagttcgagaccagcctgggcaacatggtgaaaccccatctctacaaaaatgacaaaaaattagctgggcttgttggcggactgtggacctgagataggtgctcctcttgtcctgcccagacagcctcctggtaaccaaaggccacggggcagcatggctggcacttgctatctttccacccaagcatcctccctgtggcccctctccccagcctgccccctgttggctggaggacttcaagccctctgcctccacttcctccagtcctttcctagagtacacaaaaggcaaaaggcgcagtttcttttttttttttttttgaaatggagtttcaccttatagcccaggctggagtgcagtggcaccatcatagctcactgcagtctctaattcctgggctccggtgatcctcccacctcagcctcctgagagtagctgggactacaggcacatgacaccacgtccggctaatttttaatttttttgtagacatggaatctcactctgttgcccaggctggaaagaacagtggtgtgatcatagctcattgcagcctcaaactcctgggtgcaagcgatccttccgcctcagcctcctaagtagcttggactataggtacatggcactatgtctggctaattttttaaatttctttagagacggggtctcactatgttgctcaggctggtctcgaactcctggtctcaagtgatcctcctgctgggattacaggcgtgagccactgtgcccagctcatgcaacacttatcatcaggattcccgctcagtgctcggaccccaggggccagagatgcccgcggcaaggttctgccctcaaggtgcccactgctttagggggaagacagtgatactaccacatacgagagaaggagaggacacaactgtcagggagatggactgttggagcttcagaggcagcatcttggggaggggtggctttgaagaaggagcaggacacttgggccaggtggaccttggaagggtgccaggcaggaggagcaatgtgttgctgatgggcaggtaggggagggtgagggatggagaggagaccggaagacaccctgtgccatgtggaggggccctgccctctggggcaagaggtcagggaggagggcgctggctgggttcccaggcgcctgcccctgagaggcgcaaatggccctaatggggttattaggtggagcagatgcgtccggctcctgctgtggaggaaatgctcaaatgatcttgaagctaccaggctgggagaggcaggcaggggatggggcagacaggctgccaacctctgtgagataagccctggcctctccagaaccttttcagaacagctatgagccccaaccctggggctgagtgacccaagacagatcactcacactctctgggcctcagtttccccttacataaaaagcaatggttaggccaggtgcagatggctcacacctctaatcctagtgctttgggaggccgaggtgggaggattgcttgagaccaagagttcaagtccagcctgggcaacatagcaacaccccatctctacaaaacataaatacaaaaattaaggccgggcatggtggttaaggccaggcgtggtggctcacacttggaatcccagcattttgggaggcagaagcaggaggatcacctgaggtcaggagtttgagaccaacctggccaacatggtgaaaacccgtctctatacaaaatacaaaaattagccagccatggtggtgcatgcctgtaatccctgctactgtggaggctgaggcaggagaatcacttgaacccaggtgaaggaggtttcagtgagcccagatcatgccactgcactccagcctgggtgacagagaacaagactgtctcaaaacaaaacaaaagaaaaattagccagacatggtagtgtgaacctgcagtcccagctactatggaggctgaggtgggaggatcgcttgagcccaggagttggaggctgcagtgagctatgatggcacccctggacaccagcctctgtgacagagcaagagcctgtctctaaagtgaaacatttttaaaagggaaggtgaactgggtgaactctaagaccctgcatttcacaaacccttgcccacaccctgtatgcgtcgggccctggcagggaacagatggtacgctgtctcacagggcaattgagagcgtttacaaaggtgtgggcagcattaggggaatggaccagagagggtgacctgggacttggctgtaatggggccctaagcactcctggatctgattgctgatgggagggagggagtgatgtttcccgatcagcaggcagtggccatgggtgaggggccccgcaggagcaggggctgtaagtgaaaggatgaagccaccagagggaactgggctctgtcttcccgccccctgatctcatgccagtgcctccactggccacacctcacgagaagcaagagagtgtggggtctcagtggtgcagtccctggaggccaatctgctgggtcagagcaggacagagaaagcgaaatggacatacataggcacacgccgctttcatgctgatccagtggaattagatccttcctgttctttcttttgtttctttctttttttttttttccttttttagagatggggtctcactctgttgcccaggctggggtatggtggcgtcttcatagctcactgtagcactgaactcctgggctcagacgatcctcccaccttggcctcccaaagtgctgggattacaggcactacaggcacctagctaactttctttttttttgtagaggcagggtctctgtcacccaggcttgagtgcagtggcacgatcatagctcactgcagccttgaacccctgggttcaagcaatcctcccacctcagcctcccaagtagctgggattataggtatgtgccaccacccagctaatttttttattttttgtagagatggggtctcgccatgttgcccaggctagtttggaactcctgagctcaagagatgctcccaccttggcctcccaaagtgctgaaattacaagcataagccaccatgcctggcctcccttcctggccttctggtctccagactggagagagggccacaaagtcctgcccagacagagggctgctaaggctggggtggggctgggggtgtgcagaagggactctgcaggggctgacctctgagtctcaggggacagggcaaatttttttttttttttttttttgagatggagtcttgctctgtcgcccaggctggggtacaatggcacgatctcggctcactgcaatctccgcctcctgggttcaagtgattctcatctctcagcctcccgagtagctgggattacaggagtgcaccaccatgcccagctgatttttgtatttttagaagagattgggcggggtggggggtggggtgcgggtttcaccgtgtggcccaagctggtcttaaactcctgacctcaagtgatcctcccgcttcagcctccccaggtgctgggattacaggcatgagccaccgcacccagcggggacaggacaattttgccagctggagaaggggtggcccccaaatgtccccttcaccaccctcctgcctcttccttcagaacactttttccctgaagccctcctgggtagccccctacccacatgcctcagcattgggaggtggggagtatgggggtcccccttctcctcagccccatttggagtaatgtcctgtgcagctgagctcacaactcctcctccacctgtcccttcacccggtgtcactggcaattgctcacttcctgggcctgcacccactcagctcccctatccctgggcatcagcctcagccagctcccatgtggactaatgacccttgtccctcccctcagctgcctctcttttttctttttttttttgtttttgagacagagtctggctctgtcacccaggctggagtgcagtggcgcgatcttgactcattgcaacctccacctcctgggttcaagcgattctcgtgcctcaccctcctgaatagctgggattacaggcacccgccaccatgcccggctgatttttgtatttcggtagagacggggtttcaccatgttggccaggctggtctcgaactcccgacctcaggtgatccgcccgcctcagcctcccaaattgctgggattacaggcgtgaggcagctcgcccggcaaccctcagctgcctctcatatgtccttggccccctggccatctctgaccagacatctaggaggcccctcctgccaatatgcttccccgacagccgagcccaaagctgtctccagccctgcaccttgtcttggttccccgggtgctccctttcccaggctgaagcctggacagccccaggcaacctctctccctgaccacagtaccacctggccacatgccacactgctttctacagcagacaccgcctggccacaaagacttaagccacttccttgggtcctgctcagcgctgagcccagagcccaatctcagggactgggagagagcccagtacatggccagtgggattctcccgatgaaaatcccagccatgtgaggctggcttctccaggttctctgaggcctgtgagtccagggctggctggagggaaagcccctggccccttaaagccactgatcatgttcccatagagggatcctgagaatcccctgggagagaaaatgtcggagccccagtcctggcacccccctgaccagcttgggcctcagcctcctcatccatgaaaagaatggacaagaggggtctgggtgcggtggctcacgcctgtaatcccagcactttgggaggccgaggcaggtggatcacgaggtcaggagttcaagaccagcctggtcaacatggcaaaaccctgtctctactaaaaatacaaaaattagccggttgtggtggtgcacgcctgtagttgcagctatttaggaggctgaggcaggaggatcactggaacccgagaggcagaagttgcagtgagctgagattgtgccacggcactccagcttgggcaacagagccagactccatcttaaaaaaaaaaaaaaaaaggagggacaagaggcaccagaagctcctgtccaggtctgggcctccctcgggtcactcagttctcccccattcattcattcattcactcactcactcactcactcattcccacactctgcagccactgacttcagctagccatgctggatgctaagacatagagtcccggccatcaaggccagtccatctcagtggcatctagacatgcacagagatgaccagggagcccctaagggacagagaggcacaccctgcctcctgatcccatctgggggctgcatgaaggaggggcacaggtgtggccttgaaggctgggtaaggggctgggagggctttaggggcattccagggggcaggcacagaactggaggctctgccgggcacacccacaggagagcaatggctggagggtgacatggactgcagggtatggccgcggcagcccagctcttaaggggacagcctgggaaaacagacttagggacaatgatcttgagccatgaaatgatgtccaatgccccttgtgccacctgacatcagcctgattaatgaacaaacagaagaggaagcaggggacctgtgtgtccctctctctgcttttctgtctttctctgtgccccctctcttatgtctctcacgctgtctcttgtccttgtttttaactctgacacgccatgggaacttggctcacttcctagggggaagctgtctggactggcccagagatgggcaccaccatttcccaataaacacaagataaaatcagagagctggcgtcagttccgcggctggactcagtggggaaaagggcaaaaagcagcagaaataagagcagggaaagaacaggggctgggtgcgatgggtcccatctgtaatcccagcgctttgggaggcctaggtgggaggatcgcttgagcccagaagttcaagaccagcctgggcaacatacggagacccccatttctaaaagttttaaaaattagctgggcatggtagtgtgcatctgtggtcccagctactcaggaggccgaggtgtgaggatcgcttgagcccaggaggtcgaggctgcagtgagctatgattgcatcactgcactccagcccgggcacagagcaagaccctgagaccttggcagaaagaaagaaagagagaaagagagaggagagggagagggagaaagaaaggggggggggagagagagagagagagagagagagagagagatgaaagagaaggaaggaaagaaagaaaggaaagaggaaggaaggaaggagaggggaaggaaggaaggaggggaaggaaggagagagagaggaaggaaggaaggagggaagggaaggaaaggggaggggaggggaggagagggagaaaagaaaggaaaaagaacaagtggatatcaggggacagaaaccattcgcagcccatccctcggcagatgcagagggagtcagcctggaacccccacccctgctggctgtgccccaccgctgtctccacaccccagcccttcctccagacacccagccgtccactgcagcaggagcacaggtgccccaaggggaccactgttggaaaccaggcagccctccactcacccacacagtgtgtctctgcaccccctctttgacatgggagccggggcgctcttggaaataagggtttacctgaggggtctgaagacttgggcagtggggggctgtgaagccttggggagggaatcagcttgtccaagagtctggggcagcgagtggtcgggggctacccggtctgaccccatcaaagtaaccttcctgggggatatttccattgcagggtcacaaaaaggcaagggctaaggttcgagagaggggtctctctggggtcccagccaccctgctgtgggggcccttcccaagcaggacactccaggcaaaggggatggctttcccttgctcccaggacccataggtgaggtcctggctgaaatgcagactgtccccagatgcccctggggacttgtagggatttgggggattcagtgttgcaggagctggaacccttgcttgctggacgggtttgagctgtgctgtcaggcccaaagctcctgggtctccaggatggtgcatgagggctcacaccagctgctccctgccacccttcccccacctcccaccccttctcctggagccccagtcctctggtcctccaagctcagcttcaaatgcttccaccacttctgtctcttctacctcgagtggtttgtcaacctccaaatcacacggagttcaagaatatctcaaatatcgaccaggtgcggtgtctcacacctgtaatgccagcactttgggaggccgaggcgggtagatcacctgaggtcaggagttcgagaccaggctggccaacttggtgaaactctgtctctactaaaaatacaaaaattagccgggcgtgatggtgggtgcctgtaatcccatctacttgggaggctgaggcaggagaatctcttgaaccagagaggtggagattgcaatgagccaagattgtgccactgcactccagcctgggcaacagagtgagactctgtctcaacaacaacaacaacaaaaagaatatctcgaatacaaaaattagctggccgtggtagtttacacctgtaatccgagctactcaggaggctgaggcagtagaatctcttgaacctgggaggcagaggttgcactgagctgagatagcgccactgcactccagcctgggtgacagagcaagacgtcatctcgaaaaaaaaaaaaaaaaagagtatatcgaatccaaaggaatatcttgaatacaaaaattagctgggcatggtggcacacgcctgtaatcctagctactcaagaggttgagacaggagaatcgcttgaacccaggtgttggaggttgcactgagctgagattgcgccactgcactccagcctgggtgacaaagcgagactgtgtctcaaaaaataaataaataaataaaatacaaaattaaaagaaagattatctcctggccacccatggtgcaggctgtaatctgtctctgctggggtgaagggacggagggagacagatgagaaatgagaacacagacctggagacccagaccacccatgccagctacccaggtcacttcagcccagcccagggagcccctgcccagcacaagcctcatgcacagtagcagtgttaagatgggcccttgggctgggtgcagtggctcacacctgtaatcccagcactttgggaagctgaggtgggtggatcacttgaggtcaggagttcgagaccagcctggccaacatggcgaaaccctcgtctctactaaatatacaaaaattaactgggcgtggtggtgagtgcctgtaatcccagctattcgggagcctgaggcagggcaattgcttgaactcgggaggcggaggttgcagtgagccgagatcgcaccactgtacgacagagcaagactccgtctcaaaaaaacaataaaaagaattgctggaggagggaagtgaggggcggctggcgggggactgctgggctggtgcagagggagtcaaggatccctaagagagaggcaggggtgttcagctctcccacctctgctggatgaacttgcagagcctccttggtgacccagcccgtgcagagactctgctcaggacaacacccttgcctgggacccccacatctctggccccagcagcagcccttgggggggtccctttatcttcctccactcctacccaggcttcctcagcaggagatgaggcaggacatttccactccctttctggaaggttcagaaggttaatgagagctaagcacgtaagtccatttagggggatgaacttctgggaagagaggaacctgggtctgggctgacgtccaagggcgggctgggtgacggtccctctgatcacggaccctgtccacccactgcccagggccctgcctcgacccctctgaccagccaccgagccccagagggatctccatgaatgtcagagacattgactggaggccttatctccagtgggagaccccttctcttcccactgtgggccggttccagcctgggctgtccaggaagtgacctctcagggcctgggaagggtgtggccagtggttcttggttgtactcaactcatctgccttgggtctaaggctggggtgaatggaagggcccacctggaccctggagggacaccaggctcatactaaaatcccaaaaagtgaaaagctttccccaggcccaagcagagaaactggaccttgaagctacatctctggacttagtcctcaaagtaggagacatttgcctctaagctgttctctcccaccccacctttctgtgagccgccggttccctgttgtccacatcaagctgtgtgctgggcactgggtgcaggaatagcttgaccacagtctctatcctgggggtaaaggggtgaccagcccacagagggatggactgcaaacagacagtcccaaagtgccatgagagaagctctcagggcctgggcgtgatggttcatgcctggaatcccagcactttgggaggccgaggtgggtggatcagttgaggtcaggagttcgagaccagcctggccaataaggtgaaactccatctctactaaaaataaataaataaataaataaataaataaataatacaaaaattagccgggcatggtggcatgcacctgtaaacccagctacttgggaggctgaggcaggagaatcacttgaaccccagaggcagaggctgaagtgagccaagattacgtcactgcattccagcctgggtgacagagcgagactccatctcaaaaaaaaaaaagaaaaaaaaagagagagagagagacagagagaggctctcagcctgcaggaaagggctctctccttgttggccaaaccagtgggccctttagctgtgacgggtgtgccctgggcccaccaggacaggagcaaggtcaggagggctgctctgctctgcaaaacagaggctgatggatctgaagtttctgttactgggagaataaagggaggtgaaggagacacgtggtaggtcccctgggaaggtggtgggaatgcgtgtgaaatgctcggagaggcacagtaagaaccggcctgtttagaatccagtgttctacgcctgccccaggccccagcaattctgcttctagacatctccccatgagaaatgagcactgagcctcccaaacatgtcttttgggaggccaaggggagcggatcacttgaggtcaggagttcgagaccagcctggccaacatattgaaaccccgtctctactaaaaatacaaaaattagccaggcctggtggtgggtgcctatagtcccagctactcgggaggctgaggcaggagaatcgcttgaacccgggaggtgaaggctgcattgagccaagactgcgccattgcactccagcctgggccacagagtgagactccgtctcaaaaaaaaaaaaaaaaaaaaaaaaaaaaaaaaaaaaaagacatgtgcaagaacattcagatcagccttcttcataacagacccaaacaggaaacaacagacgttcatccacaggctagtgaagaggcaggttgtgaggcctccgcccacaccgtagaatgctattcagcaacatgggtgactcccctgcatgtaacattggcaaagaagccaggcacaagcaaatatacactgtaggattccatttatataaagagcaaaagcaggcaggagggccgggtgggcacagtggctcacacctgtaatcccagcactttgggaggccagttgtggggggtgaatcgcttgagcccaggagtttgagaccagcttgggcaacatagcaagaccccatcttaaaaaaaataaaaaattagccgggcgtggtggtgcaggcctatcatcccagcttctcaggaggctgaggcaggaggatcacttgagcccagaagttggaggctgcagtgagctgtgattgtgccactgcactccagtctgggcaacagagtgagaccctatctcaaaaaagcaggtgggagtaatccatggcaataaattgggatagaagttattcctggaggtggtgatgactggtatgggacacaaggaagccacctggggctgggaatgttctagttcttgatctgggagatggtcatgcagtcgtaggcctgtgtaagacttcctcgagctgtccacataagatttgtgcactttagctgaatgtggtggcttacacctggaatcctagcactttgagaggctgaggtgggcagattgctagaagccaggagttcgagaccagcctgagcaacatagtgagccccctgactctaaaaaaaaaaattttttttttaattatctgagcatgctggcatgcacctgtagtcccagctacttaggaggccgaggtggaaagattgcttgggcccaggagattgaggctgcagtgagctatgatcacaccacttcactccagcctgggcaacggagcgagaccctatctcaaaaaaataaataaaatatttgtgcagttctctgtatggaaaatgtccgcgtctatccagcaagcatccatggagtagtccggagaattgtgggttagcaagacactgggtggcgtcaggcactggagggatgggggaacagccatgcagaagatcccaggcagagccaaggcagagatgcagggggatggtggggggcctactggggacggctaagtggtggggggacaccagtgatccaagggctgggggcatctagaggccagggttccagttacccacagtgcccaggatctgtgggagtagtggatgggggagtagtggatgggcaagggtcaagctgaaaaacaagagccgtcaccctcaccctcccatgcccccactgtggctccatcctggctcctggttctcatcagccgagccggggccctttccccggctgggggtggccatggcgccagaagccacgacaggtttacgttccagccgcagctgcactggccccatcccaagtcccctcgcttgcacacacatttttctccttgtggagtcagaagaggcctggccaggtcctgcacagggttctggaggcgagagtccccttggaatcacgttgtctcccccagccctgggccgtctctgctcggggagtgccctcagagctgcacctggactctttcccccacttcccagcccttcctcctgttctttctcccagccccagctcttctccttcaagcccagctcaggtgccgcctcttccaggaagccccctgggaccatctccagtctcagtgggagcagtgtgtccttggttgctcccagagtctatagcagagatctggacccaagactctggactcccagctttgggctggcaggtaacacccactcctggggcttcacttgccttgctacgtgtcagtttctcactttgaggggggcagcagtattcacccacaggggctggggctgaggggattaagggactgcagctccaggaggcctcagaggaaagggtacaggtgggatgcagtggctcacagctctaatcccagcactttgggaggccaaggtgggaggatggctcaagtccaggagttggagaccaacctgggcaacacagtgagacatcatctctacaaaaaatttagaagtttttttctttttcttttcttttttttaattattatttttttgagacagagtcttactctgtcactcaggcactctgtcatctcagctcagtcaggcactctgtcatctcagctcactgcaacctccacctcccagggtcaagtgattctcgtgcctcagcctccggagtagctggggttacaggcatgagccatcatgcccagccctccaaaaagaaaaacaataacaacaacaacaattttggccgggcatggtggctcacacctgtaatcccagcaccttgggaggccgaggatcacttgaggtcagtagttggagaccagcctggccaacatggtgaaaccccgtctctgcgaaaaacacacaaaaaattagctgggtatggtggcaggtgcctgtgatcccagctacttgggaggctgaggcaggagaattgcttgaacctgggaggcagaggatgcaatgagccaagaccatgccgctgcactccagcctggggaacagagcgagactctgtgtcaaaaaacaaacaaacaaacaaacattttaaaagaaagaaagaaaaaagggaaaggagagcatcagagtctaagaccacacagaaaccctctctttactgcagtgctgaagccctcgtggcagccatgccctgctcacttttgggatacccatggactcagggcacgtcctaaggtgacacctgtcaccagccctatgcctgcctctgtccaccttcgcctctgctggagaccattctcagacacccctgagatgcccagaggggacctgcaacctcttcccaccccacctcccacaaggggtccctgggaaaactgggggtaacattccccccaccctcacccctacttcagtctgggtgttcagagcagccctctctgtctctgaattttctctttttttccttttggagacggagtctcactctgtcaccaaggctggaatgcagtggcgcaatctcagctcactgcaacctccatgtcccgggttcaagcgattctcacacttcagcctcccaagtagctgggattataggttcacgccaccactcctggcctctgaattttctctcttggtgttttgtttttgtttttgtttttttgacagagttttgctcttgttgcccaggctggagtgtaatggctctatctccgctcactgcaacctccgcctcccaggttcaagcgattctcctgcctcagcctcccgagtaactgggatcacaggcacctgccaccacacctggctaattttttgtatttttagtagagacggggtttcaatatgtttgccaggctggtctcgaactcctagcctcaggtgatccacttgccttggcctcccaaagtgctgggattacaggcatgagcttgaattttctctttctgatttcctgtctctctgcctctgactatgtttctccatctgtttctctctgtctgtctgtctgtctttcagagcagggtctttctttctgacatctgcagggctgggggctgtgctaggaggggggtgaatttgtcattgtgactttgcagctccttatctatgaagtggacaggacaccagcatctagtcctggagtccttggtgaggacctggtgtgttgaggctggccggtgcagagttaagggttcagtccatgatttctgcatccatgaaagggggatggtgaggctgggcatggttgctcatgcctgtagtcctagcgttttgggaggccgaggcgggtggatgacttgaggtcaggagttcaagaccagcctggccaacatggtgaaaccctgtctctactaaaaatacaaaaattagcagggcacagtggctcatgcctgtaatcccagcactttgggaggctgaggcaggcgcatcacttgaggtcagaagttcaagaccagcctggccaacatggcaaaaccctgtctctactaaaaatacaaaaattagcagggtgtggttgcacacacctgtaattccagctactcgggaggctgaggcaggagaatcacttgaacccgggaggtggaggttgcagtgagccgagattgcaccattgcactccagcctgggccacagagcaaaactccgtctcaaaaaaaaaaaagggggggggggtggggggtggatggtaagaacagccctgctcctctgcttctggggattaacgtgaggatgtcctgagtgcttgccccagggaggtgccctctgcggtctggtgaccaagtggctcaccagcacactttgttcccagccactctgcggggtgcagccaccattgccccactctacagattaggcagatgaagcccagagaggctgaataactggcccagtcacacagcagctcctatcatagcagtggaggtaggatttgaactcaattgtctggctccagagcctgcgatcttcactgtggacctcgtttttttgtggtttttgctttttgtttttggagacagggtcttgctctatcacccaggctggagtgcagtggcacaatcacagctcaccgcagccgtgacctctgagctcaagcgatcctcccacctctgcctctcgagtagctgggactacaggtgtgcaccaccatgcctggctaacttttttatttttcgtaaaggcagggtctcgctatgtagcctaggctggtcttgaactccaggcttcaagcgatcctttgccttggcctcccaaagtgctgggatgacaggcattgagccaccaagcccagcctgtggaccttggctttaaaactgttattcaatggtatctgtctaaatggtgcagtaggagtgtcaagattggtggggtagggggaagctggggatagagccccatctggcctctgatgactcccagcctgcccctctggccagccaggattttttttgtttttgagacagagtcttgctctgttgcccaggctagagtgcagtggcatgatctcgactcactgcaacctctgcctcccgggttcaagtgattctctgcctcagtctcccgagtagctgggattacaggcaagcaataccatttcgggttaatttttgtatttttagtagagacggggtttcatcatcttggccgggctggtcttgaactcctgacctcgtgatccacctgccttggcctcccaaagtgctgggattacaggcatgagccaccgctcccggccaggacttcttaacatacagctgtcacttggcccagagcctgccagggatccttgtgtccctggctcctgtgcccagcttttgcgggttcacagctggactcctctggccttcggagagggctgcccttgccaccctctgcacctgccacccaacttcctcctcatcccctgctcctgcctcctatccagttttctgggcttctccttcttcctccagcaatgccttcggtctcattcttccaggacctgcctcctcccaaagtcctctctgggggctctgggccatggcacagcaggacagccaccagaagccactgtcagagtctgatgggtgaggacggaaggtggctcaggagtccttccttagggcagggacaattttttccaggagaggtgtgaggcctccagctcctaaaactcctcgaggtttaagagcgggaaatgctttggctccaaaatattaaaactgcaacattaaaaaatagtaaatgtttactgaaatgtcttctaaatataactttatgtcagtttcatccattgttaaatttagtatttcaaaatgcatcactgcgcatgaaaacaattcgtaggtcacgttttctcactttggaagaatcctaagattctcaagaatgcatgatgttggtagagaagtcaaggccaggagcaaatcaaatgagttacttgcccccaaaccgctttgaaagttgttataaaattcttttcaattgttttatattgccaaaatatattacagagttggggtatgggtgcattttttttattctggtaaaaaaacacataaggtttaccatcttaactattttatttatttattgttattattttgagacggagtctcactgtgtctccagcctggagtgctgtggtgccatctggtctcactgcaagctccgcctcctgggttcaagtgattctcctgcctcagcctcccgagtagtgggactacaggtgtgcaccaccacgcccaggtaatctttgtatttttagtagagacggggtttcaccatgttggccaggatggtctccaactactgacctcaagtgatcctcccgcctcggcctcccaaagtgctgggattacaggtgtgagccaccacgcccggccttatttctttattttttaaaattttatcatgttgcccaggctggtcttgaactcctgagctcaagtgatctgcccgcctcagcctcccaaagtgctgggattacaggcatgagtcaccacacccagcccatcttaagctttttttttttttgacgaggtctctctctgtcgctcaggctggagtacagtggtgccatctcagctcaccgcaacctccacctcccaggttcaagcgattctccctgcctcagcctcctgaatagctaggactacaggcgtgcaccaccacgcccagctaatttttatatttatagtagagacagagtttcaccatgttggccaggctggtctcaaactcccgacctgaggcgatccacgcacctcggcctcccaaagtgctgggattccaggcgtgagccaccgcccctggcccatcttcactatttttaagtggacagcacagtagtattaactatatgcacatctctggaactttttcaccttgcaatactgaaaccctacactcaacaaacaactcgctttccctgccccgctccagcccctgaaaacttctactctcctttctgcttctgtgaattgtactattccagatacctcccataggtggaagcaaccactatttgtctttctttgtaactggctcatttcacttagcattatgtcttcaagtttcatccatcttttagcatgtgtaagaatttccttccttgctttttttgtttgtttgtttgtttgttgagactgagtttcactcttgtcacccaggctagagtgcgatggtgcgaacttggctcactgcgacctctgcctcccgggttctcaagcaattctcttgcctcagcctccagagtagctgggattacaggcgcctgccaccacgcccggctaatttttgtatttttagtagatatggggtttcgccatgttggccaggctggtctcgaactcctgaccgcaggtgatccacccacctcggcctcccaaagtgctgggattacaggtgtgagccaccacacctggcctcgtttttgtttttgtttttggagacagagttttgctctgtcgcccacgctggagtgcagtggtgcgatcatacctcactgcagcctcccctcctaggctcaagcaatcttcctgcctcggctttctgagtagctcgaactacagacacacactaccatgcctggctaatatttttattttttgtagcaaagcggggggggcggtctcattatgttgccctggctgggcttgaactcctggcctcaagtgatcctcccatctcaagcctcccaaagtgctgggattataggcgtgagccgctgtgcccagcctcaaacactttttctttcaacgacattgacctatagtgaatatcaaaagcatgcactgaaactcaagagctgcctcctctcctgtgccaggatgcactgtaacttttgtgagccctaggcatgtttgacttatatgggttaagatattcaaatacggctaggcacggtggctcacgcctgtaatcctaacactctgggagaccgaagtgggcagctcacctgaggtcaggtgtttgaggccagcctggccaacctggtgaaactccatctctactaaaaatatagaaattacccaggcgtggtggcaggtccctgaagctctggctactcgggaggctgaggcaggagaatgccttgagtcagggaggcagaggttgcagtgagctgagattgcgctactgcactccagcctgggtgatagagcgagactccaccccaaaaaaaagaaaaaaaaaaagatattcaaatgtgtattttacaactgctttggcataaagactgactgataccagtcaggtcagattcattattacatattcattactattatactcctgtttgactaggcatggtgggctcacatctgtggtcccagccctttgggaggccaaggcaggaggataacttgaggccaggagttctagaccagcctgggcaacataatgagacccccccatttctactaaaaataaaaaaaattagccagtcgtggtggtatgcatctgtagtcccagctacatgggaggctgaggtggtaggattgcttgaacccaggagattgaggctgcagtgagctatgattgcaccactgcacttcagccaacagagcaagaccctgtttcaaatgcatacatacatacatacatacatacatacatacatacatacatacatacacacacaaaccaaccaatacaaaaaatacatattttttttcttctgattctaaaaaacataaaaacagccaggcacgatggctcatgcctgtattcccagcactttgggaggccgaggtgggcagattacctgaggtcaggagttcaagaccagcctggccaacatggtgaaaccccgtatctaccgaaaatacaaaaattagctgggcgtggtggcgcgtgtctgcaatcccagctacttgggaggctgagacagaagaattgcttgaaaccgggaggtggaggttgcagtgtcccccacccgctcctccaggggcgcccccctcgagccaccgcgccggctgcccggcgagtgtcagcgcctcctcacaggccccgcccctcacgccccagacggccaatgagagctgcggccccggccggcccctccccgccctggggaaccccggtcgcggattggccggcccgggcgcagtgtcccccgcacgtggggcgggggcggggtgagtggagtggcagcgaagccccgcccccgccgccccgcccccgcccccgcctcctccccggcccgccccccctgcggcgcccagtccagcgcccgccgcccgccaccccggaccccggtgtctggcttcccccgagccgggaccccgcgatggccaagcgcagctcgctgtacatccgcatcgtggaggggaagaaccttcccgccaaggacatgtgagcgcggccgggggtgggagccccaactttccggggaggggcactgccccccaggacgcctgacctttccggggtccacccacaagagcagcggagagggtgcgggagtgcggccgtggtgggggatcggcggggaaaatggggacagtgacattttctgcgagcaccagacggggtgacaaccggggggcggggaagtgtcaccgagggctgggggctgggaactagggagggggcgagaatttgtcagcgatccgcggctcacgaggggaaggggctttgatacgacaggagctggagcctggtgacatgccaggggagacgtcttggtgatctgcccccggggtggggttggagggggtggggtgtcccccgcaaaggaagggtcttcctcagtagttccagagcccttaatgccccggagtgtgcacctgaagcacatgctggggcccaggggctgtccccgggcttcctgctgtgcactcgggtaggagggactggcatgaaaagtgagaacaggtcaggaggccaggacaggtgcaggactgtgggggagggaaggagggtagtaactgtcgcttgtgccttgggctttacacctgagccatcccgttaatcctctcaaccaccccgaaaggcagttctcaccgttccattttacagatgagaaaactgaggttcagagaagtgaaatgctggctccaagtggcaggcagagcagggtcttgagtctagggcttctcacaggcactgacatctggggattcccctggtccctggctcgatttggggaggtttcaggggccccttgttccactcttctcagtaggtcccagggctgagctgcctgacccctggcccaccatcttggtcttgccaccaccttcctcatgtacagggtttaggcagctccctgctcctagagcctcagtttcccctctttaaactaggggttgaaatccctctagaggaagagggggaaagctcaaaggtcaaggggcactggctgcctggaggaggagggtgggagggggaaaagtggagcctggaggaaacagagtccaaagtctgcggagctgcagttccccaccccttgggcgccccgggctcctggatgcagagaagtggctagcatgggcgagggggtggagcagggtggggggcaggcactctgctcacacatatgtgtggtggcccctgtgagcttcccagcaagctcagcgcgccaccaccgagagcagtgccagttccaggccccctgggtctgatgaggcccagctcagcagttaccatgacaatggggaagggagggcggtgggcatccctaggtgggtttctgtgcttggaggcttggagggaggtggagtgggagctggcagtgaggtctgtgggctgggaaggcagaggagctctgagaagggtctcagcctccaggcccagcctttaagcatcccccaccccctgccaccactggcactaggaactaggtcatcttggcctcttctggctggtggcagctgggaggagcagcccctccgtctgtcgcctccctcctcatccccacagcagctggccctagtgcctggctccaggctctctccagcccagggcagccaagctcttggagtatgcccacctgggagcccagtgcctggttccctgctcagtgctttgcctctgggcaaaggggttccatctacagtggaggggtgccttgggatctcgatcctcaccctgcccccctgcctgccctactcacgtctcctttgcaaccccccatcttcccctgtggcttgcatacacagctctgacctgtcagccgcctcttaaaccatcccataaacagtccctcacatttgggctactacccttcacatctctgctttcgcccatgtgtggggacttctgccttcaagcactccacttcagtcctccagaaaactcctattcaatgttcaggactcctctcaaacttctcctctccagggcattccctggtgcacttgggcagagttcctggctcctgggctcgccccccagcactctgggtggcaggaggaagcccctggtgtggcagttcctccgtcaagtggtgcctgtcccagctgactgtgagggcagggactgtgactcattcatcctggcaccgggtccatagtagggagaataagaggtgttgaggacttcccccacttaatcctcttcttagctttataagatagacatcctcggtttgcacattgggaaactgaggctcaggaagtgaaagccatttgccaagagggtaggtagagaagtcaggattcagattgcctttgctgccgacaggctgagaattctagcctgcccgcggctgtaagcttgcggctgggctcagtgagactgagcctcggaaatcctatgcaaagcatttggctgcagagtcgggcaggggtttggagatcatgggaggccctcactgagtactggagtttcactgtgcctcaccagcaccccgcctatcttctgagtagcatctcctccagccagggcttgcatacttctgaggacggggagcttaccacctctcatagtggctcatcttgactgagacagttccaaggaggaagctgtcctcacacagctgccagctgccttcctgccacttctactcatgggtttttattcctctaaagggcacagagcgcagctgcattccccatcactgctagctttcagagcctgcctgttcctgccctgcctcttcccactgtctctcgtccccacgtacctaggcatgagccaagtctcacgatgaccagtgacccactgcctcccacaccaagtggccttggggactggcccaaggctattttggggttttccctgatcctcctccttccacagcctagaaactgccaaggccctaaatatagaggccccgaaagtagccagacaggccagtggcaactggctcgggaaaggagaaatgagagaggtgggcagcctaggggcctcaacactagcgaagaaggaactgggcagggcttgggtcccagcagcccactggggaccatggttgggcccaggtccttaaatctggcctcggcctgctctcaataccgtcctcttgccccacctctgatccagccacccggctcttcatagtccttgcacaccaggactcgggtcctgcttccaggcctttggaagcagtagcagcccagtagtgggggacagcccacggaggtcccctgttgcctcagtcagtactgacctctccctgcctcctctgagctccttgtatggctcctggatcctgcagcccagcccagcacctggggtgttcagaggcctgatttcttagcacctggagtcccctggctttgggttgatgggatgcaagctggggtattcacagggggctccctggaggtgggggggctctttattttattttatttatttctgagacagagtcttgctctgttgcccaggctggagtgcagtggcgcgatcttggttcactgcaacctcctcctccggggttcaagggattctcctacctcagcctccccagtagctgggattacagttgtacaccaccactacattcggctaatttttgtatttttcgtagagacggggtttcaccatgttggccaggctggtctggaactcctggcctcacgtgatctgcccacctcggcctcccaaagtgctgggattacaggtgtgagccaccacacctggccaggaggcgggggctctcaaatgatggcagggggaggttacagtcagagatgcaggcagtgctaacatttactgagcacctccaagaccttctctatcgtctcaggacccagggaagctcccttgtgagccggggttacacttcccactttataggtaggaaaactgagtcttggcagacatttcctgtctgtgtccaggagcttctggagctgttggccccaaatgccttcctctgccggaagagccccttttgagaagggaagcagagcttggaattccccaccctccaccatagaaacacacttcctttttatggacactcactggaggaacagctctagagacagccctcacttccccacttttacccagtaccagccctaggcctggctgcaggcctctcccagccccaggagccccccaacacctgagacccttggaactgagcgcgtgtttgccttcccccatccctctttccccagatagctgttacatttattcattcattcactcatcagttcattcattcgtctgttcattcggcaaacactcatgggctgcctgtcttgtgccaagccctgtgggggccctggggacacaggcatgctccccgcccccaggtagctcagagcaggtgggaggcagctgatcactaccaggctggatgaagtgggcaggcagtgtgagtccagggaggccttcctccccgtcccgtcctggggcgagggagggctctggcagggctgaaggccgactgggcaggagggacagcggtgtcggtgttggggagagcaggagcaaaggctccagggagagtaaggggctcgactttggtgtggagggtaggggtcccaggagcttgcctggggctgttgcttggagctgttctggggggaggcagggatggggtaggagggcatagaaaggaggaacattctatcccctgcgggaggaacaggaggtttccagtaccccctccctctgtccctctccctcccctccctgtctttcccctctccctctctacccctccctctcaccccctccctcagtctccccctccctctttactccctccctcttactctcctccctctctccccctccctctcaccccctcactcagtctccccctccctcttactctcctccctcttactctcctccctctctccccttctctctctcccccctctctcaccccctcagtctccccctctgtcttactctccttcctcttactctcctccctctctccccctcctactctcctccctctctccccctccctcttactcttctccctctctccccctccctctcaccccttccctctcaccccctcactcagtctccccctccctcttaactccctccctcttactctcctccctctctctccccctccctatcatcccccttccctcttaacccccacctctctcctcctccctctcagccccctccctctgtctcctcttccctctctctcccctccctgtccgtccccttccctttctgcccctccctctgtccctctcccagtctctccccttcctccttccccctcctttcttctcctttctctctctacccatccctgtctcctccccctccttctccctcccccacaactctccctccatctactcccacccctgcccgcgaaaggggagccctggtgcgggttcctgcccttccctcaccatccgctcccgggctgagggcggggacccgtgtttggccggaggagcctgcggtctccaaaccaggacgcggagactcagcctccgctctcttcccccgcactcctcccgaggggccaaaagagagaggagcctgccgcgagtccccgccccgatcccgccctccagccccgcccctgcctatttgtcttgcagcactggcagcagcgacccctactgcatcgtgaaggtggacaatgagcccatcatcaggtaccgcccccacccccaggaccgaggggcgctcagcctctcatcggcccgcgctctccccgcaaaggggtgtgaattttgttgtgaattttgtacggagatgggagggggcttggagcacctggtcccctgcctgggattccccacctcgccctgacaccccagctaggagacgagaaagaccttcagggaactggcctggccaagggcctctcaccggactctgttcatctttgcaatcatcacaacctccgggagagacagatgctgacgatttgtaacaacaactctcatttgtggagccagcccttttccagcccgtttagttcttgcaattaacgagatacgatatcattagaccccatttacagacaaggaaactgaggctcagggagactaactaccctggccaaggtcacatgcctagtaaaagtggcagagccacactggagcccaagtctggccaccccagagcctgagctcccttcccctccctggtcctggctctaacttgggtcctgtcccctgagagtcccatagtaatatccctcgaggctgggcaaccccagcctgcaccctgaagagtcacttcctccggatgtcccttcctccatggtccccatgatctctgggcagggtctttgctgtcctctcccttccttcacccagactcctgcttccaagttcatggccagagctgccctactgttctgcccacctgagtgcccgtggcctactcggagcccagtccttccctacctgcaagcaggctcccagcctttctcgagggtctgcaaaccacagacagcaagagggagaggggtgtgatggctctgatgctcttgatacagaagggaaacagatgacctgtctgagaagtggttggcatgaggtggtcccctgaactttcatccgggtccttgggggcttcagcgtctccccacccgctagcccactgagtcccatgagttgaaggctctgatccaaagggggttgaccagagctgggtgaggatgtgaggccaggctcaccagtcgccctgaggcagctaccctgtgacctgaggccaatgcctgccaactgtgatctcagtttccccaaatgtaaaacagctggagaggctgggcacggtggctcacgcctgtcatcccagcactttggaagtccgaggtgggcagatcacctgaggtcaggagttcgagagcagcctggccaacagggtgaaaccctgtctctacgaaaatgacaaaattagctgggcgtggtggcaggcgtctgtaatcccagctactcaggaagctgaggcagggagaattgcttgaacttgggaggcagaggttgcagtgagccgagatgacgccactgcactctagcctgggcgacagagcgagactccgtctcaaaaaaataaaataaaataaataacagccagagagaggcagccctgtgccccatccagagcagagaggaatgtcagggggtagtgattggtggtgaacagtagggtctggcacagggaaaacacatcagtggcagagccagttgttataggggtaggggaagatacaaggaaggtcccagcccagggcctgggagtggcctgggcacatgctgagggttctctcctgcaggacagccacagtgtggaagaccctgtgccccttctggggtgaggagtaccaagtgcacctgccgcccaccttccacgctgtggctttctatgtcatggatgaggatgccctcaggtgagtgcccccctctccagctgggacccagacctggccatctgattgctccctggcccatttcaccaccaggactcctgggtcctttttggcatcctctttgcagcctggagggaggcagagcctgggggcctgggagggcgaaaagcttgagcgtgtgggtgtgcacatgcgtggctccatggtgcatgcgccacatacacacgtgtgtgtgcaggcatgcaggcacaagtgtgcatggactacacgtgtgcgtgcaggtgtgagctgtgagatgggcacccagagagtgtgagcttggcatgtgtgggcatgtgagaaacctatcacatccccctagagggtccagaacccacagcctacagaagggccacaggtccagctctgttgggttactctggaaatgacagcggtgtccaccaccctgctccccccgggggggccctgaacttggtgggaggtcccagagggcagatactgaagccctgcccagctctgcctccgtctccctcctctagccgggacgacgttatcggaaaggtctgccttacaagggacaccatagcctctcaccctaagggtaagttctcccttccctcccacactggtctgcccagtccctggcctccctcccactcggagacctcccctctaggctccgtctggtctcctgctcagggaaagccatttctactctccccagaagccggggccacgttctgtactcctggcctctgttctgcagcatgttcccaagcctggttgttactgcctcttccctagggtgaagaggggctgctatgggtggaatctgaggcctctgctggcagaagaaggggcctccttacactctattgctgaagcatagggaccccttctccaaatcaggccagctccttctgtaacccatggggtcgcctccatctgggccctagtactactgtgtcctaagtctgaagggttggcctagagccagtccaggctggagatccctttcaattatttctgggatgcagacattgttttgtgttattgtttttacaatttttatatagtttttttttaaaaaaaaggccgggcacggtggctcacacctgtaatcccagcactttgggaggccgagacgggtggatcacgaggtcaggagatcgagaccatcctggctaacacggtgaaaccccgtctctactaaaagtacaaaaaaattagctgggcgtggtggcaggcacctgtagtcctagctacttggcaggctgaagcaggagaatggcgtgaacctgggaggcagagcttgcagtaagccgagatcgcgccactgcactccagcctcagcaacggagcaagactccacctcaaaaataaataaataaataaatgaataaaaaataaaaataaaaaataaaatagaggccgggtgcagtggctcacacctataatcccagcactttgggaggccgaggcaggtggatcacctgaggtcaggagttcaagaccagcctggccaacatggcaaaactctgtctctactaaaaatacaaaaaattagctagacgtcgtggtgggcacctgtaatcccagctactcaggaggctgaggcaggagaatcgcttgaatccaggaggcggaggttgcagtgagtcaagatcacgccattgcactccaacctggatgacagagcaagactccatctccaaaagaaaaacaaagatggaggccgggcatggtggctcacacctgtaatcccagtggcaattcacttgagattaggagttcaagaccagcctcgtcaacatggtgaaaccctgtctctactaaaaatataaaaattacctgggcatggtggcactcacctgtaatcccagctactggggaggctgagacaggaaaatctcttgaacctgggaggcagaggttgcagtgagccaagattgcgccattgcactccagcctaggtgacagagcaagactctgtctcaaaaaaaaaaaaaaaatggagatgggggtctcactctgttgcccaggctggtcttgaactcctggctttgacagatcctcccgccttggcctccccaaggtgctaggattacaggtgtgaaccaccatgctctgcccagacatgattttgagggttcagccctgtgccagcctcactgtgtgtgaccttgggcagctcctgccttctctgggcttctctgagggttcaaaccagttcttggatgctatgggctctgccatcagagcctttgtgtcccgcttactggcttccccccacacacacctgctcctgtctgtgacgctgggccagggatgggggcagggcgaaggcaggttctgctgactcatggggcttcttggcccctgccaggccaaaggcctttcccacctgcagccttagccagcgtctctggggtctagtggccccagtgaggctattcctccgggtgccttgtagcttccccatccaggcctcagaccctcctcccagcagccctaggggagggccactctcttcctgttttaagatggggaatctggctgggtacagcggcccacactcataatcgcagcaccttaggaggctgaggtgggaggatggcttgaggtcaggagtttgagaccagcctgagcaacatagcaagactccatcttttttttttttttttgagatggggtcttgctctatcacccagtctggagtgcaatggcgcaatcttgtagcttctccatctgggcctcagaccctcctcccagcagccctaggggagggccactctcttcccgttttaagatggggaatctggctgggtacagcggcccacgcttataatcccagcaccttgggaggctgaggtgggaggattgcttgaggtcaggagtttgagaccagcctgagcaacatagcaagactccatcttttttttttttttttttttttttttttgagatggggtcttgctctgtcacccagtctggagtgcaatggcgcgatctgagttcaagcaattctcctgcctcagcctcctgaggagctgggattacaggcatgcgccaccacacctggctcacagggtgagaccctgtctctaaaaacaacaatgacaaaaatgacacaatagtgagggcacaggccccagtcactgacttagattctaatcccagctcttccaactgctgtttcttcacctgcggagcctcagtttccccatctgtcattggggataccagcccctgcttcacgggtgcctaggggattcccagattatggaggtgcctcaaggtgcccggtggggctgagggtggagggtatgggggttcagggccgggtccctggctgagctgaccccacaggtttcagcgggtgggcccacctgacggaggtcgaccccgatgaggaggtgcagggcgagatccacctgcggctggaagtgtggccaggggcccgggcctgccggctacgctgctctgtgctggaggccaggtgagactcaggggcctgggggcgggcagtgggtcccctgcaactagagaaacccaatgaggaagctgagccccccctcgccccacctctacctcctggtcccagagctggccacctcccatcaaagcctgctctcaagagagggtctcgccaggcacggcgtctcacacctgtaatcccagcactttgggaggccgaggcaggtggttcacctgaggccaggagttcaagaccagcctgaccaccatggtgagaccctgtctctactaaaaatacaacaattagccgggcatggtggcaggcgcctgtaatctcagctactcaggaggctgaggcaggagtcttgaacccaggaggcagaggttgcagtgagccgagatggcgccactgcactccagcctgggtgacaagtgagactgtatctcaaaaaaaataaaaaagaaagaaaaaagagagggtctggggaggttttctggacttgaagatgtttcttgggtgatttccactcaaggggatatgtcccttaagggacagtctaatgttctcatggaggaactggagccatcacagaggagtggagtagggggtacgggtgaggagaccccgaactctgatcacacagcctcagtcccccagtgctaaggccggcttcccaatgtttaacgccagtgtgaacttggcctggtgggagtgtgtgtaagtgggtccctcgtggggctgggggggtgaaaagagttgctgaaaatctcccgatgggcaaattgagggttcccccaaggagggacagtggtttggatggttccatgggcctgagtcacctgtgacagggccaccccccaacccccagggtatttttagctgcaggctgcactcccgtctgggcctgggtctgagtcacactctgtccgtgagaagcgtcttcccttccggctccaaagccacctccccaagtcgctccctgcctgtgaggcaggagggtggcccccagccaggcagccctatggagccagtgttccccccacccttgaggggtcagcctgtctaagaggaagacttcccttgggcagaggggtgtgggtcggatgatgcccatgttgctcactctggcttcccgggagttattttctgtcctgggaaaatagaaatggatggaaaatgtctgggcttgggccgagcctcagccacacccccacactcgctcactctctggcctctgccattcattcccccccgccccaccccaactcacccgcttcctccatccttatcctttccaggcaccaggctgtctgggggacaggcatgcacacgtgtgcacccgctcacacacgtggccgggctctgggacctcggggccacttctcccaggcacccatcgtcacccacggagatgggaggcatggaagcatgtctccctggccctcccctctctccaggaatctcccctgtcccatcctctggggccacagtggttggccttctagtgagtcctagcaggggagaggaatgtccaggcctctctcagagtgaggggacttgtcccccgttgtcctcggcaatgagactcctgctgcaattccaagtcagcctaagaaggtccatttgctgcggagaaagaaagaacatttcctcattttttctggggagattctcaatatttcagtaaaaccttggggtttttttgttttttgtttttgagacaatctcgctccatcatccaggctggagtgcagtggcccgatctcagctcactgcaacctccaccttctgggttcaagcaattctcctgcctcagccttcctagtagctgggattacaggtgtgcaccaccacgcccggctaattttttgtatttttagtagagacgggacttcactatgttggctaggctggtctcaaactcctgacctcaggtgatctgcctgcctcggcctcccaaagtactgggattacaggcgtgagccactgcacccgggctgtttttttttttttttggtgggtttttttttttttttttgaggcagtctggctctgtcgcccaggctggagtgcagtggcgttttcttggctcactgcaacctctgcctccccaggttcaagcaattctcctgcctcagcctccttcccaaagtgctgggattataggaatgagccgctgcacccagcctcaatgttggggttttattagacagtcttgagggggaagaaaggcaggtatgagaggcttaaaatatcaaagatgagtgggctttggtgttcttccacaaagagttctaagtaggggatactggcagggtgcagtggctcactccagtaatcccaatgctttgggaagcagaggtgggaggatctcttgaggcccagatttcgagaccagcctgggcaacagcaagatcctgtctctacaaaaaaatttaaaaattagccaggtgtggtggggcacacctatagtgccaactacttgggaggctgacgtgggaggattgcttgagcccaggaggtggaggctgcagtgggctatgattgcatcactgtactccagcctgagcaacagagcaagaccctgtttaaaaaaaaaaaagaaaagaataaaagggccaggcgcggtggctcacgcctgtaatcccagcactttgggaggccaaggcaggcggatcacctgaggtcaggagttcgacggcagcctggccaacctggtgaaaccccatctctactgaaaatacgaaaattagctgggcgtggtggcaggcacctgtaatcccagctactcaggaggctgaggcaggagaattacttgaacccaggaggcggaggttgcagtgagctaagattgcaccattacactccagcctgggcgacagagcgagactccgtctcaaaaaaaaaaaaaagctggaggtaccgtgactagactagggctgtgtaaagacccatctatggagaaagggcagggccgcccatcagggctgcacctcactcctccaccaagtagagcgacattactggcagaccttccagaagcacactctctccctaaacctgagcccagtagcgattcacttttccattttcctgatgaggaaaccgaggcttagggcagccctgggaatggagggccaggaacattgccccggcggttccctggcagcactcacaccacccccctgccacccccagagcaaggtgcagccactggcctgcagctgcctggtgggaaggacccatggcctctaccttggggggttcttggccacatgggcgatcctggggagatggctatgggagcagggcccagaaagggcagaggcgcactgtccacagtcagcttgccagggacggaatgtgcccaggccttttggcccgctgtggttacataatggtggtgacaagtgggggagggggggctcagggacaagtcgatgatccaatcttcctccacccccaccagggatctggccccaaaggaccgcaatggcacatctgaccccttcgtccgagtgcgctacaagggccggacacgggagacctcggtgaggaggccagagggcagggaaggggcagggggctcgggggacgtgccagccacgcccccattgctcacctcaccacctgctcaccatccgctggtgggacagattgggggccacagtcttacagaggaggcccagggaaaggggcgacctctctgaggggacataataggtcccaggacctctggggccaacttcgctcactcccggccatgcttgggggtaaggaggggattgagacagccggagtggggtttatttgggaacacccatgctgccttccccacccccaggaaatgtccagagtcgagggcccagtcctgggcacgaagacaccaaggtctgctttctcagtatagtcgctactgtttttgttttgtgtttcgtggtgttttttttttttgaggccaagtcccaccctgtcacccaggctggagtgcagtggctggatctcagctcactgcaggctctgccttcccagttcgagagattctcctgcctcagccacacgagtagctaggattacaggcacacaccaccacaccaggctaatttcttgtatttttagtagagatggggtgtcaccatgttggccaggctggtctcgaactcctggcctcaagtgatcctcccgcctcggcttcccaaagtgctgggattacaggcatgagccaccacgcccagccctgtgttttttgtttgttttgctctgtttttaaatattttatttccacaggttattggggaacaggtggtgtttggttacataagttcttagtggtgatttatgagattttggttcacccatcacccgagcagtatacactgcacccaatttgtagtcttttatccctcacccccttcccacccttcccccccgagtccccaaagtccactgtgtcattgttatgcctttgcgtcctcatagcttagctcccacatatgagtgagaacataacgatgtttggttttccattcctgagttactacacttagaataatagtctccaatccaatccaggttgctgtgaatgccattaattcattccttttttatggctgagtagcattccatcgtatatatataccacagtttctttatccacttgttgattgatggacatttgggctggtttcacatttttgcaattgcgaattgtgctgctataaacatgcgtctgcaagtatcttggccaggcacagtggctcacacctgtaatcccagcactgtgggaggccaaggctggcagattgcttgaggccaggagtttgagaccagcctggccaacatggtgaaaccccgtctctactaaaaatacaaaaattagccgggcgtagcggcaggcccctgaaatcccagctacttgggaggctgaggcaggagaatcacttgaacccagaaggcggaggttgcagtgagctgagatcgtgccactgcactccagcctgggcgacagagcaagactgtttcaaaaacaaaacaaaacaaacaaaacaaaaacatatgtgtgcaagtatcttttttgtataatgacttctcctctgggtagatacccagtagtgggattgctgaatcaaatggtagttctacttttagttagttaaggaatctctgctgttttccatagtggttgtactagttcacattcccaccagcagtgtagaggtattccctgttcaccacatcctcatagtcagtactgttttgctgtgtggccttggatacatcttttccccaccctgggcctcagttctccctatctcagaaatgggtgggagcttggacaggacattttaacctctgctgtggcagaacctccaaggcgaggaggctgtggggaacccaggggaggtgtctagggcagctctgccttttgcccagcctggggagtccagtggcatcagcctaaccccctcccaccctcctctgggcagatcgtgaagaagtcatgctacccacgctggaatgagacgtttgaatttgagctgcaggagggggccatggaggcgctgtgcgtggaggcctgggactgggacctcgtcagccgaaacgacttcctgggcaaagtgagcaccaccgccccgccccccctcctcccccggcaggctgcacctgctgtcccccagcacctggttccattgcagccatcccatcctcagggacctccctccggcttccccaaagagggacactcaggaagcctgggactacccccccacctccaccgtctgccagaaccagcaacttccccccaccaccactggcactaagggacccttggaaggggcccctccccactaagaacagtagttggcaaggaggagacgtaagtctcccagctccaggaaaactgccactatgtccccaggtggtgattgatgtccagagactgcgggtggtgcagcaggaggagggctggttccggctgcagcccgaccagtccaagagccggcggcatgacgagtaagtgcatggagctgggcagccggcctcaaggggtggggtgggggctgacatttgggaattggctacaggcaggcagggcaggggggctgctcagggtgagaggtcaggagacacagctccagggtcccctcagcccctgcccacctagccagcatccttctcatcctggctgggctccacctcccctttatttattatttatttatttatttatttatttattttgagatggagtctcactctgtccccaggctggagtgcaatggcgtgatctcgactcactgcaggctccgcctcccgggttcacgccattctcctgcctcagcctcccaagtagctgggactacaggcacccgccaccgcgcctggctaattttttgcatttttagtagagacagggtttcaccatgttagccaggatggtctcaatctcctgaccttgtgatccacccgtctcggcctccccaagtactgggattacaggcatgagccaccgcgcccggcccctcccctttatttttccccacagttctgcattcagctcagctctgtcttctgtcttttttttgagatggagtcttgctctgtcacccaggctggagtgcagcggcacaatcttggctcactgcaacctccacctcctgggttccagtgattctcctgcctcaacctcctgattagctgggattacaactgtgtaccaccatgctcggctaatttttgtatttttagtagagacagggtttcaccatgttggccaagttggtctcgaacacctgacctcaagtgatccacccgcctcagcctgccaaagtgctaggattacaggcatgggccaccgcacccggccaggtctgtcttcttagctcacaggttgtacaggtctcctgctttctgcctctggaagtttctgaaacaagtactcagacattttccctttgaaaatttccctgcagacctcagaccagggttcaagtctccaaggtcagtgtccctgagccatagctccaactccctatgccagattctccaggaatagggttgtgagataagataggacaccattggccaggagcggtggctcatgcctgtaatcccagctctttgggaggccaaggtgggaggatcacttggggcaaggagtttgagaccagcctgggcaagaaagtgagactccgtctctacaaagaaataaaaattaggccaggcgcggtggctcacgcctgtaatcccagcactttgggaggtcgaggtgggcggatcacctgaggtcaggagtttgagaccagcctggccagatggcgggcacctgtaatcccagctactcgggaggctgaggcaggcagaattgcttgaaccccggaggcagaggttgtagtgagccaagattgcgccattgcactctagcctgggtgacaaagctagactctgtctcaaaaaaaaaaagaacacccagttaaaacttaatttcagataaaagtgaatatatatatatatattttttttttttgagcagaatctcactctgtcacctaggctggatggagtgcagtggcacggtcttggctcactgcaacctccgcctcccaggttcaagcaattctcatgcctcagcctcccgagtagctgggattacaggcatgcacctgtaccaccagtacagtggtgatcatagctcactgtagcctccacctcctgggtgcaagcaatcctcccatctcaacgttccaggtagctaggaccacctcacctggctaaatttttgtagttttagtagagatggggtttcaccgtgatggccgggcttgatgttgaactcctgacctgtgcccggctgtgaatacatttttagtataaaaataaccgctgggcgtggtgactcacacctgtaatcccaacactttgggagtttgaggtgggtggatcacttgaggtcacgagttcgagaccagcctggccaacatggtgaaacaccgtctctactaaaagtacaaaaaatggtggtgggcacctgtaatccaagctactgaagcaggataattgctggaacccaggagacggaggttgcagtgagccgagatcccaccactgcactccagcctcggtgacaagagcgaaactccgtctcaagaaaacagccgggtgcggtggctcatgcctgtaatctcagcacttgggtggccgaggcgggtggatcatgagatcaggagttcaagaccagcctggccaacatggtgaaatcccgtctgtactaaaaatacaaaaattacccggatgtggtggcgggcacctctaatctcagctacttgggaggctgaggatggagaattgcttgaacctgggaggtggaggttgcagtgagccgagatcgcgccactgcactccagcctgggtgacagagcaggactccatctcagcaaaaaaaataaataaataaagaagaagaatgaccatgcactatttgagacatacctatactaaaaatattcaaatttaggccgggtatggtggcgcatgcctgtaatctcagcactttgggaggccaaggcaggaggatcacttgagcccaggagttcaagaccagcctgggcaacatagcaagaccccatctctaaaaaaaatagttaataagtaaataaaatattcacatttaactgggtgtcttgggtcgttttgctaaatctggcagcctgtctaggaagccctctggcttgtttgctgtggcccacagccctcagtggttcagctttgtgtctctgtggaaggccccagacccccattcccccagttcccatccttaacagggtaggagcaggtggacggctgtgtgtccagcacaaggtgtttccatagaaccagccaaggcccatcttgccatcttattttttatttatttattttttttgagacagagtcaggctggagtgcagtggtgcaatcctggctcactgcaacctctgcttcctgggttgaagcgcttctcctgcctcagtctcccgagtagctgcgattacaggcacctgccacaatgcctggctaatttttatatatatattttaatagagatggggttttgccgtgtcagccaggctggtcttgaactcatgacctcaagtgatctgcctgccttggcctcccaaagtgctgagattacaggcatgagccactgcgccaggcccaggggcccatcttaacactgctctgtgcatgaggcttgtgctgagcaggggcagtgggagccttagaggttcccacttggtccacaccctcctctgttctgaacttccctatcagaacagggaagctgggaagctcaatctaatgtggcaagttgctgaactggctcgggtctcagtttccctattaggttgttatgagggttggggatttgcttctgctgtaaagtgtacccatgatggtgccattatctgggagttgatcgattcaaggctggcagctccacatacccaagcttggaggtggcagggagccagacctcgaagacagatagaggtgccttgagtgcagagacaggaagggctttccaaaaacagaaaataaatagcccgggcagagcctgggaggcagagctatgcagggcacccgaatggagggcatgagcagagagatgggaagccggccccaagggaaaaaactggaggccatgggagtggtgtggtccagggtggcctgagtccctggtggagaggtcacaggctgcccattccaggggcaacctgggctccttgcagctggaggtgcggctgcgggacgagacggtgctgccctccagctactaccagccactggtgcacctgctgtgccacgaggtcaagctgggcatgcaggtgagggggcctgggcagggtgggagggtccagtggcagtaggcctgtgcgcctgtccttctaaacccattctgcagagcagaaacctgaggtctggggatgtgaggagctggccagtgtccagggccccgtccccagccaccccttgggaaaatgtcctctttccctgggatgctctgaggtcctcagagggaaaggttcgggatcccccgtgcctctccctgcagggcccagggcagctgatcccactcatcgaggagacaaccagcaccgagtgtcgccaggacgtggccacgaacctgctcaagctcttcctggggcaggggctggccaaggacttcctggacctgctcttccagctggagctgagtcgcaccagtgaggcctgggaaggagctgggcccagaacactgggaggtgtttgggggtgggtgggctcctcctgtaggaggcagatggggtcagagaggagggaggcaaggaacagagccaagggcagaggtgggacagtgggctgggaggtggcgaggaggccctttattgagggggcagcttagatcccccagacccaaggtcctctgtcctccccctcccccaaaagctttgtgccagccccctacccactgggacctctgtttgtaggtgagaccaacaccctgttccggagcaactctctggcctcaaagtccatggagtcttttctgaaggtgaggttgcatgctatattgtcctcatcttgcaaatgacaacactgagcttcagagagagcgacagagggggctgggacttcgactccagtgttctatctccatattcagagccctttaaggttaagaatgtggctgggcatagtggctcatgcctgtaattgcagcactttgggaggctgaggcgggcggatcacttaaggtcaggagttcgagaccagcctggccaacatggcgaaaccccatctctactaaaaatacaaaaattagcctggcatggtggtgggcacctgtaatcccagctactggggaggctgaggcaggagactcacttgaaccaggagccagaggttgtagtgagctcacgccactgcactctagcctgggccccgtgctagactccgtgccccgcacccccccgccaaagagaatgtgtgtgtgattaaaatttaagttagaaacagtctcactatgttgcccaggctggtctcaaactcctggcctcaagtgatcctcctgccttggcctcccaaagtgctaggattacaagggctaagaattctagtcattcattcactcactcattcattcattcatgaataaacagtttgccctgcatgtgtacctggccccctcctgggcggatacggcaggcaaacggtgcaagggcggctgccgggagaaggtgggcagctggagtgggactgggggagacaggatcaatgtgacctgcggtggtccccaggtggccgggatgcagtacctgcacggcgtcctgggccccatcatcaacaaggtgtttgaggagaagaagtacgtggagctggaccccagcaaagtggaagttaaggatgtagggtgaggccgggggtaactccgggggttgcggggtgcagcggcagcgggttgggatcaggccctgtcagcatgtgtgtttgtgcttctgcccacccgtgtattgtcccctgtgtccgtgtcctggctgctgtgagagccactgttcctgtcgtggccctggcgctgaccgcgacctcctctgccaacccgccccgttccacgcaggtgctccgggctgcaccgcccgcagaccgaggccgaggtgctggagcagagcgcgcagacgctgcgcgcccacctgggggccctgctgagcgcgctcagccgctcggttcgcgcgtgccccgccgtggtgcgcgccaccttccgccagctcttccggcgcgtgcgcgagcgcttccccggcgcccagcacgaggtgcgccctccacccgctgggctaggctgggccgggttggactgggctggggcgaggggtgaagagcggtggggaaccagggagcgctggggaggaccagggaggctgtgtcccagcgagggtcagggacggagcctaaggggagggggctgccaggagccctggtctgggggactccagcccgcgctcttccgcacccctccagaatgtaccgttcatcgccgtcaccagcttcctgtgcctgcgcttcttctctcccgccatcatgtcgcccaagctcttccacctgcgggagcgccacgcggacgcccgcaccagccgcaccctgctcctgttggccaaggtgcgggccttgcggccacgggcgggatgcacagctgggtgtcctaccggggccctggcctggactgcagagtgttccctaccggctccggcgaggccactggcacgcccacgtaggcactgcacttcacagtttgcaaagccttcctgtggccaccttgtccgagctccatctcatggataagggaacaggcctgcgaggggtcgggggttgccaaggccgcccatcctgtcggtggtgaatcgcgggaccttccaacccgggtctgcttactctcaggctgcacctatgtgcccggcaccggggtggggtatccagtgcagcagcgtcagttaaacctacagaggaaggcccaagtggtctcagttatcataaggcatcaaacttcatgatcctctcctccctgatccctagtggggctggagggggcccgtgggcatggcctctgtcgcctctgcctccatcgctacagctgagtagcaaagacttggggagggggggtgggagggggtgcagtggctaggcccctaacctagcagaacctggtccttcctgctgtcacctccaggcagtccagaacgtgggcaacatggacacgccggcttccagggccaaggaggcttggatggagccgctgcagcccaccgtgcgccagggcgtggcgcagctgaaggacttcatcaccaagctcgtggacatcgaggagaaggacggtgagcgccgccctgcacagacccgcccacagcctactgctgacgtttgctccgcccttgagccccacggccccacccatcgctgtttgttcctctcgtggccccacccaccgcctgctgttggcatttgccaggcccccaggccatagacccacgcaccactgtttgtgcttcctgtgaccctgcccactgcctgccgttgacgtttgccccacccctagacccatggcctcacccccccatcatctgtgcctgttgacatttgctccatccttgagctccatagccccgccgccacctgctactgacgcgtgtcccgcctccaagcccatggccctgccttcctcagtccacctgtcaccccgctcaccaccagctgttgatgtttgctccgcccttgagccccatgactacgtccactgcctgctgttcagatttgctcagcccttgagccccatggcccctcccactgctccgctccccccagtcccgccctcattcactgggctctctattcccctccctccctggcctggcagagctggacctgcagcggacgctgagtttgcaggcgccacctgtgaaggaggggccactcttcatccacaggaccaagggcaagggccccctcatgtcctcctccttcaagaagctctacttctccctcactaccgaggccctcagcttcgcgaagacgcccagctccaaggtgggtaaggagggaggccggcaagtggggctctgagaggccggcaagtggggcacctgtccccttccttggagcccacttgaggtacccccggggtcagagaagaaagccaggtgtgtggcatctgtactgcttgtcagaacctaggagggccgggcacggtggctcacgcctgtaatcccaggactttgggaggccaaggcgggtggatcacttgaggttaggagtttgagaccagcctggccaacatggagaaaccccgtctctactaaaaatacaaaaaatcagccgggtgtggtggtgggtgcctgtagtcccagctactcgggaggctgaggcacgggaatcgcttgaacctgggaggcagaagttgcagtgagccgagatcgtgccactgcactccaacctgggcaacagagcaagactccgtctcaaaaaaaaaaaaaaatgaatatggaggaagttgatccgaccttaccaggttgttgaggattaaatgagatgatctccctaagggtcctgacccagggcctggcaggtagtactatttaggcaaatgatattattaaagatagtataacacctgggctgctagatgagtaggaggctagggcaaacaggggaggcttcctggaagaggcaggaatatagaagcacggaatcttggcgtgggaagggatctcaaagatcaccttgtacagccctctctgagtcatgggtcacctctcctgcaagctctcttctcccaggaagggctgaggacatacttcctccatggacctggtgactgcttgctgtacacctgcccccagtcatgcaggctatggggatgatcttccaggagtacccagcctagccctgggcaggtgagagcgggctaggagtaggtgaaggagctcatcacccaccctgttctcaaccagctcaggggtcccaccagatgaggggcccgccccttccgaccaaagacccctcagatgatgataattattttttgagacagggtcttgctctgtcacccaggctgtagtgcagtggtgcgatcatagctgaccgcagcctcaaactcctgggctcaagcaatcttcctacctcagcctcccgagtagctgggactacaggcacgtgccaccaagcccagctaattttctttaacttgtttttttgcggagatgggggtcttgctatgctgcccaggctgttctcaaactcctggcctcaggcaatcctcctgccccagcctcccaaagcactcagatgattattatacatcttccttgcccgggttgaaagatttgactacatgggcattatttaattctggtcccattttttttttttttttttgagatggagtttcgcttttgttgcccaggccagagtgcaatggtgcgatcttggctcaccgcaacctccgcctcccgggttcaagcgattctcctgcctcagcctcccaagtagctgggattacaggcatgcgccaccatgcccggctaattttgtatttttagtagacacggagtttctccatgtttgtcaggctggtctcgaactcccaacctcaggtgatccgccttcctcggcctcccaaaatgctgggattacatgtgtgagccactgcgcccggtcaggatatgactattagagcctgggactacaaaagcaagttggaaaattaataaatgcaaagtcacaaagccttggccacggccggattgactttgtagcccagggaatgctggctgtcccgacactaccccaaagctcacacctccgaggccttctgggagtgccccccagagaggccaggtcctcctctcccagttgctgggcacttggtcaccatcagtctgtgctctgccctggctgaggtcactctaggtctcccatgcctacccctggtgctgcagaaggggagactgaggcccagagatggggaatggaatgcccctggctaggtccctgagcctcctcctccctgcctgccccagaaaagcgccctcatcaagttagccaacatccgggcagcggaaaaggttgaggaaaagagctttggcggctcgcacgtcatgcaggtcatctacacggacgacgccggcaggccccagactgcctacctgcagtgcaaggtgcggggaggggagcgggtgctgtgggagtggcctgaacactgtgggagtggcttgggtgctgtgggagaggcctgggtactgtgggagtggcatgagtcctgtgggagtggcctgggtattgtgggagaggcctgagctctatgggagtggcctgggtgctgtgggagtgccctggatgctatgggaatgccctgggtgctgtgggaatggcctgggtgctatgggagtggcctgagctctgtgggagtggcctggctgctgtgggaggggcctgagtggactggatgagaaaagagttgattgagcctctcgccccgctaccagctacatcaggcaccgcagggtgtgtctaatctgggaggtgtcagggacaggtgtgtgtgtgttgtccaggggcctgagccccaggaggccatggcccatgtctttccctgggcatccatgtgtaggtctgtgtgtgggccagtgcacagacaggtgcatccccgtgatgtgaaatttgcacgttgcacccgtttgttggaaactatttccagataactgagatgggggccaggcacgctggttcacacggataatcccagcattctagccagggcaacagagcaagaccctttctctaaaacagaaagtctgagctggcacctgtgtggttctgtgtctgtatcaatgcctaataatcaaacattatgctgtgtgtggctctgggtgcatgttctcaagtaaccttctcatctgttccttcattaactcattcattcatttatttaaaaaatacttactgagcacctcctaggttccaggcatgattctaggtgaggggatacagtggcagatgaggcagagtcctgccctcatggagctgatatccctgggagaagatgggccaataaatacatgtttcaaatgtcggctcatgcctgtagtcccagtactttgggaggctgaggtgggaggatcactggaggccaggagtttgagaacagcctggccaatgtagcaagaccccatctctacaaaaagaaaattagccagccatgatggcatgcacctgtgattccagctacttgggaggctgaggcaggaagatagcctgaacccaggagttggaggctgcagtgaactatgatcacaccactgcactccagcctgggctacagagtgagaccctgtctcttaaaaaaaaaaaaaaaaaaaaaaagcaaattgtgatagtgacttgaagggaaccagaagggactaagggagcaagggagggaagagagtggggtatagcagggagcggtctgtcctattaagagtggtcagggagagtacaaaaagtagccgggcgtggtggcgcgtgcctgtagtcccagctacttgggggactgaggcaggagaatcgcttgaacctgggaggtggaggttgcagtgagctgagatcgagccactgcactccagcctggtgacagagcaagactccatctcaaaataaataaataaataaattagtaaattaattaaagagtggtcagggaggacctctgatgggtggcattcgagcagggaccttgtaggagggtgacccagatgtgtatctagggcagggacatgtgggacaagtaagaagcctgcagctggaggggggttggtttcttccgggcttgaccgtcctggcctgtggggaggaagtcacagacctgtccctctgtgcccttccctctaggagttcccttttgtgtcagggtccagagccactgggaaaagtagtgatatcagggccaccaaatatatttggcgggttctggaatacctacgagcgtggccaaggggccagcggcggctgaaatccagcccacaccagcctcctgggctcctaccatcccggtgccttccgtggggacaagtggagctgctgccaccaaaaagacgagacaggtgggagaggagaggatgaagtcttgctgtgttgcccaggctggtcttgaattcctgggctcaagcgatcctcccacctcggccccctataatcccagctgtagtcccagcactttgggaggctgaggtgggaggatcactggaggccaggagtttgagaacagcctggccaatgtagcaagaccccatctctaaaaaaaagaaattagccagccatgatggcattataggagtgagccactgggattataggagtgagccactgcacctgctctccaggccatgcgcccgggcatgggtcatgttccactggaggaagggcagcttctcctaattcactcaaagctgctgggttggtatggtggggaacatggcgtctggcgtggtacaggagctccgggttaggagggcttcctggaggaggtgacgtctgaacaggcctccccctccgtgtcccctgcagtgtgtgaatgagcttaaccagtggctgtctgcgctgcggaaggtgagcatcaacaacaccggactgctgggctcctaccaccctggcgtcttccgtggggacaagtggagctgctgccaccaaaaagagaagacaggtgggagaggaggcctgggtcccgggctctgcatccgtccactgcgagatcgggcctctggctgaacgatgggaccctgagccctgctgtgcacctgcagggacctaacccaaggaatgctggcccctccaggagctctgggcagaaagggtcccgtggaccaggtggtttgggaaatgccacgtgatgtgtgtgatgcatatgagtgaattcgctgaaggctctaaggaggcccgaaaacacgttagaatcccatttctttttatttcttcttcttcttttttttttttttttcagacacagactttcactctgtcccccagactggagtgcaatggcacgatctcagctcgctgcaatctccacctcccagggtcaagcaattctcctgcctcaacctccccagtagctgggatgaccggcactcgccaccaagcctagctaatttttttgtattttgactagagatggggtttcaccatgttagtcaagctgctcttgttttgttgttgttgttgttgttgttgttgtttgatactgagtctcgctctatcatccaggctggagtgcagtggtgcaatctcggctcactgcaatctccgtttcccaggctcaagtgattctcctgcctcagcctcccaagtactacaggcgcaagccactgcacccagctaagttttgtattttcagtaaagacggggttttaccatgttggccaggctggtcttgaactcccgacctcgtgatctgccctcctcggcctcccaaagtgctgggattacaggtgtgagccaccacgcccggcctagaattccatttctaacgtggtttaacccagcacttcccaaactcttttgacagggaacccttctcttccccaaggagcttcttacagaacaggtgttctcaggaacacattcgaggagccccctgctaatgagagccagagagcccctgggttcgcacactccaatgctggccatctgaatccggagctgtgggtgggaggggaccctgagccttgggcactagagggatggcatagaggagcttctagaaggactccatactgtgtagtactctaggttctagcacctgtggttcttgagcacttgaaatgtggcttgaggagctgaggggttttttttgtttaattttttaaaacagggtcttgctctgttgcccaggctggagtgcagtggcacgatcatagctcactgcagcctccaactcctgggctcaagcaatcctcctgcctcagcctctgagtaggctggcactaccggcatgcatcaccacacctggctcatttaaaaaaaattgttttagaggtggggtcttgctatgttgttcaggctggtcttgaattcccaggctcaagcaatcctcctgcctcggcctcccaaagtgctgagattgtaggagtgagccaccacatctggcctgagtttcacatttttaaaaatgttgttttggccgggtgtggtggctcacgcctgtaatcctagcactttgggaggccaaggtaggcggattgcctgagctcaggagttagagacgagcctaggcaacatggtgaaaccccatctctgctaaaatacaaaaaaaaaattagccaggcatggcggcgtgggcctgtagtcccagctactcaggaggctgaggcaggagaactgcttgaacctgggaggcagaggttgcagtaagccaagatggtgccactgcactccagcctggcgacagagcgagactccatctcaaaaaaaaaaaaacaaaaaaacaaaaaaaaaaacagaaaagaaacaaaaaacgttgttttaattttaattaactcaaatagcttcatgtggctagctgccgccctgtagaacagcacagttctagaactttcgagaccttctccctgttatccacacttactttacagagtagactcagcacttcgagtcccctgtccttcaggccaggccaaaccttggtccccagagcccagtgtggcagaggccatcgaaaactgacccacgcactctagcccagccctggatttacagccaagcactgtatagggatgggtgactcttttgtttttgtttttgttttgagttgggtctctcactctctcacccaggctggagtgcagtggcataatcatagctcactgtagccttgacctcctgggctcaagccatcctcctgcctcagcctcctgcagaactgggactacaggcacatgccaccacacccagctattttttattttatttttttgtagagtcagggtctcactatgttgcccagactggtcttgaactcctggcctcaagctatcttcctgcctcagcctcccaaagtgctgggattacaggtgtgagccactgtgcctggcctcttggtgactctttgcaagggcattgctggctggctgatatggcctgcagcctctgcctgtaaccatcagagcgatactctcattatcggcaaggtgggacccaccctggcccaagagacagggcctgttattccactgtatggaggagaagctgaggcttagggaaggcagatgacttggcaaggtcataaagacagcaagctgcaggaccagctcattctaaggcatgaaccccctgtggcccacctcaccatgatgttaacatttcagcctgctcccttccaggcagacagtcttccagaaagttacccggctccctggctgggcgcggtggctcacgcctgtaatctcagcactttgggaggccgagacgggcaaatcacgaggtcaggagatcgagaccatcccggctaacacaatgaaaccccgtctctactaaaaatataaaaaattagctgggcgtggtggcgggcatctgcagtcccagctactcaggaggctgaaacaggagaatggcgtgaacctgggaggcagagctcgcagtgagccaagatcgcaccactgcactgcagcctgcacgacagagcgagactccgtctcaaaaaaaaccaaaagacccagcaacccgagtcatatcctgatgatatccatgctcctcagtcacgcatcccgtggtgcaggggctgaccccaagaggagctgctgcccccagagggtggggagccgaggcagggcctgggtcagacttaccaggctatgctcccagcccagccctcactagggacccccgagtgcatctctctcctctccaggcctctgtttctccatctgtgcaaccacagtgttggacatggtagtcccaagtgtctgctcgtaactttgccctctctgtgcccccaggtcagggctgcgataagacccggtcacgggtgaccctgcaggagtggaatgaccctcttgaccatgaccttgaggcccagctcatctgccggcacctgctgggcgtggaggccatgctgtggtgagtcccacccaggagagcctgtgcagcctggggaacgtgccaggcttggggctgctccatggtagctggtcacctctcttgtaattggccacatctggggagggcaccaggagtacctctctgcagcacgaggccagctcaggttaggacagaggccccaagggtcagggccatgcctgcttcatgccatgtgctgactgagcccaccttgcaacctgcccagctgttcccctgggcccttggagtacaggcacagcctcagccaattccctgagttccctggccatcttgcttcgtccagtggttctcagccttggcagaagcttaggaaaaacaggatgcttttaaagatcagctttaaaaaacaggatgccaaggccccatcactcagggtctctggagatgtggctggtgccaggatacttcaaggtgatccagtgcttcccatgcgctcagcttcccctgagccaactggagctggagctcattcatctgtcacacccctacactctgcaaggaggaagggtgtggtcctccccattcctcatcgactcttccagggtttatctctgccctcaaaacccacgaaggtggggtgcggtgactcatgcctgttatcccagctctttgggaggtggaggcaggcagattgttggagatcaggagtttgaaaccagcctggccaacatggcaaaacctcttctctactaaaaatacaaaaattaactgatgtggtggagggtgcctgtaatcccagctactcaggaggctgaggcaggagaattgcttgaacccaggaggtggaagttgcagtgagcttagattgcgccactgcactccagcctggaccgcagaacaagactctgtctcaaaaacaaaacaaaacaaatgcaaagggccttctggtcaacccacagtgggatggggaggaggaggagacacggggagggagaaggtggaaggtgccctggtgggccttggcccagcctcttcacagactgaaagagtcagccctgccatcacccccagagccacctctagcccaggaggcccatgctgggccacccccagccttcccacaaggacaccagaaggtctggcagggagagtggaggctgctggccctcactctgacctcccccacagggagaggcaccgggagctgagcgggggcgcagaggcaggcacggtgcccacgagccctggcaaaggtaggtcttgcccacctgggacttgctcgtggcccgagccccctccagagatgcacgggtggggtcctcgggctcactcccaggggccctcacggctgcctctgcccacagtccccgaggactcattggcccggctgctccgggtgctgcaggacctccgcgaggcccatagctccagcccggccggctccccaccctcagagcccaactgcctcctggagctgcagacgtgaggcccgccctacgctccccttgctgagtcccctgccaagcgctcggagcccccccaggacactctgcaccccctcaccccggtcctcctcattagggtgcagggcctaggtctcttccaggtgggggaggggggagagtcaggaataaggggatccccagaagtgcagagctgagcaggcttgggcctgtcatggctggccggaagtgtccccagctccctacagacgctgtagccatcactgcctctccagggaccctcctctcctgcccaggacagacccagccagaaccactgctaggatgggccgcacccaggggtctggcctccagggacctagagaatgggagggagaacggggccccaggagacccggccgccaccccacccgctacccttgggtgccacagggctgtgctgttgccaacagtaaacctgctcttactgtccaggctctggggtcttgtgatgagggtctggggagaaagtgggcccggggggaccccggaggctgtcggtggatgtgccgatgatggggctgacagtatgggctctgggcatccctgttcccccctctttcttccccccactcttctggggtcgggggttcctttcccttcccagttgctgtccctgggtcccctctttcatgtcccacaggccacagagcccagtgtgtccaaccagctgttctctcctcaaagcagcccccaagcaagtcccttctctagggtgtccctgaggacagcacagaggcgggactcagagaccccattcctcttcacgcagcccttaccccaagccctctagctgtgtggctggcagtgttggccacgtaggggctcccatccccccaccattgtgtcacatgggctgccaggctcagctcccagctgcgtccacagtgacctggatcagggtggggacaaggactggaccctccttctccagaaggccttcagctcttgccttgccatgcagtcacctccttccccctctgaccccagatcccaaaggtgcaccgttgccccagcccctttctggccccatggggtttctctgatgccttcatcatagaggcccggggctggtccgatggttggcaaaacttgactccggcccagtccccactcttggggacttagaacccctgctgtcctgggatctggcctgcctttctttggtcagtccctgtggtcccccaccagctccccctcccatagggctgcccaccaagccctgcccccagcccaagaggagcccccactgcctgcggggcagtgatgtctggccaccggctcacaccaatgacttggtcctggggtggcagaagcagcaggtgacaggagcagggcccctgtccctctcttctggccctgtggtacccaggccacacgttgtgcccgctcttggggctgaccggctgtagggaccaccagccgctgctactgtgggccgccccggggcagggtgggcagggcttttgtgggttatgaggacacagaagtccctgaggcccccagacctggctcagccaacctccttcctcccccggttgccccccactctaaagcctcctccctcccagcgtccactggctccaggctcctcacaacagcagctcatagacacggggcgtctccaggtggtcccagccctccagatgtttctagctctccaggtgggcgctgttttcacgtctgcctgcatccattcattccttcattcctcacctttatcctgttatctctatttttttaagctaccaggaaggaaagggaagaagagatcacgaaactgggacccccagaagggaggagtgggctttgaacttagacatctacctcagagctcaaataggttgtttaaaatcacattcaattttcagatgaaggggaactttatagtttttttttttttttttttttttgagacagagtctcactgtgttgcccaggctggagtgcaaatggcttgatcttggttcactgcaacctctgcctcccaggttcaagcaattctcttgcctcagcctcccgagtagctgggactaaaggcgtgtgccaccatgcccagctaattcttgtatttttagtagagacggagtttctccatgttggccagactggtctcgaactcctgacctcaggtgatctgaccgccttggcctccgaaagtgctgagattacagttgcgagccactgtgcgtggccagaactttataataagagacttgaagctgggtgtgacggtgcacacctctagtcccagctactcgggaggccaagacagaaggatcaccttgaggccaggagtttaaggccagcctgggcaacatagcaaaacctagtccctaaaattaaaaaaaaaaaaaaaaaaaaaggaaaataaaggagacttgaaatttttgaactaaatagtggtgatggctacacattgtgaatgtaattaacaccactgagttaaacacttaaaatggttaaaatggcaaattgtatgttatacctattttactacaataaaaagtataaaaaagagaagatatttaggtgacttacagcaaccaattgcaacaaaacaaaatgttaagaaatgatctttttatgaggcaattggaaatttgaacactgatcaactataggatgattggaattattaatttttaaaggtgtgataagatactgcacttggctgggcacagtggcacatgcctgtaatcccagctacttggcaggctgaggtgggagaatcgcttgagctcaggagttcgagaccagcctgggcaacgtggcgaaatccccgtctttacaaaaacaaacaaacaaacaaaaaagatattgcagttgtgttgtaagcgtccttatctttcagagctacatagtggaatgtttatggaatatttaggataaatgatataggcatttgggatttgctgcaaaatgacccagaggcaggggtcagggggagaggtagagatgagacaagaggtagaggggagaggtagaggtagccacgagctgataattacagacaagagatgcggagtatgtgggggctcattatcctgcatagtctatctttgtatatctttgaacttttcaagaataaaaaagcttaaaaagtatacatggcctggtcctaccagagactcacccaatgccagcctccagccagggagagccaagtttgcattttcacacgcatctcacactcctctgcactctcaacttggagcgctccaaacagggaaaccccaagccttgctggcttctgccaaccccctgagcagaagcatgggtccccctgatcaccacctcaccaccctcatcctgatctcactgtacacagcaagcaaaccccagtgtaattaacaagactgaggatacctgtaatcccagcactttgggaggctgaggccggcagatcacatgagttcaggagttcgagaccagcctggcaaaccatggccaacatggcaaagcccagtctctactaaaaacacagaaattagccaggtgcggtggtgcgcatctgtaacccctactacttaggagaattggattgaacccgggaggcggaggttgcagtgaactgagatcgcaccactgcactccagcctgggtgacagagtgattctccatctcaaaaaaaaaaaaaaaaacaacaagacagaggagaggaggggcagagtcttccagaacatctttttttttttttttttttgagacagagtcttactgtgttgcccaggctggagtggagtggagtgatcataactcactgcggcttccaactcctgagttcaagtgatcctcctgcctcagccttctgagtagctgggactacaggtgtgcaccatcatgcccagctaattttttgtacttttggtagagacagggtctcaccatgttgtccaggttggtctccaactcctgggctcaagccatcctcccacttcagcctcccagagcgctgggattacaggaccaccgcacccagccacgaacacattctcaacgctcatccaagcaagctctgagcctcccctttgtccttagcctaggacagcccccagtgtcccagggctcagacatgacccctgacctggctgcccagctaggttctggggcagggccagtggggcatcctgtataccttggctcttccaggaatagtagggaaaggcaaagagggctgggcacggtggctcatgcctgtaatcccagtactttgggaggccaaggtgggtggatcacctgaggtcaggagttagagaccagcctggtcaacatggtgaaaccctgtctctactaaaaatacaaaaattagctggacatggtggcaggtgcctgtaatcactacttgagaggctgaggcaggagaattgcttgaatttaggaggtggaggttgcagtgagccgagatcgggccactccactccagcctgggtgacagagagacgctgtctcaaaaaaaaaaataaaaaggtcaaggggaaattaagattcaaggggcctgccctctccctcttccctatggcttttggggccataccaaccttttctcctggctgtggccttggtggataccatttcccccataggcagcccaccagcccgaccccttagagcttcatagcctgctgtgctctgccgtcctatgggcaactaagcctttttgctggattgggtccctcccctcccttcctggcccaaggccctgggtccagggacagctgagcctgatgcagcccctaccccaccccaaatcaccagcacctggttctctggctacttccttaggggtacacaaaaatcctcatgcctgttggatctccttgggtttcaaggaccttctctctaggaagtatggcctccagggtctcctgagcctaaacgcaggcagtgaaagggaaggagccgtggacttattagggctacaagtggaaacctggatcttgagtgcacacacacagccacccttctggcagcatccaccatgcctctcctcccctccccagatcctgaccactgcagctggaagggaagttaacatcagtgcatgcctggcactgccccctggttctcctctcaggccctgagcagagctgcccatagtccatcatgccacattgtttctggaggccacctggaaaagcgagaggagcaggagagggtataccttccatctgtcccacaagtctataccaccccctcccagagacttcaagccctggcattcaactggggcctggggcaggttcccagatgttttgtttgtttgtttttgtttttgtttttgtttgagatggaatctggttctgtcgcccaggctggagggcagtggtgtgatctcaactcactgcaacctccacctcccaggttgaggcgattctcctgcctcagcctcctgagtagctgggatttacaggcacatgccaccactcccagctaatattttgtctttttttttttttagcagagatggggtttcatggagtttcaccatattggccatgctggtcttaaactcctgacctaagatgatccttctgcctcagcctcccaaagtgctgagattacaggtgtaagccaccgcacctggcctatttttttttttttttttttgagacagagtctccctctgttacccaagctggaggatagtggaacattctcggctcactgcaacctctgccttccaagttcaagtgattatcccacctcagcctcccgaatagctggaattacaggtgcccatcaccacgcctgactaatttttgtattttttagtagagatggggtttcaccatgttggccaggctggttttgaactcctgaactcaagtgatccgcccacctcatcctcacaaagtgctaggattacaggcatgagctaccatgcccagtcccagatgtcttgaggatctgattggacccccaggcagatatctgcagcccagggggctggtgttcaccacagggcagggcaggcaccctgggaaggcttgactggacccagctcacccagtacctgtttctctcaacctcttaggaaatctgaacaggtatgcagcaaccacctccggacaaggccttttcaaagtataattttcaaacatgtaaaaaagatgactcatatatatcatgaacacatatcaaagatctactacgcatcaattcagaaaaagagtgggaactctgattcatggatcattctgagaagctgggtacatgtatttcatgtatttgtaagcattcttttgttttgttttgtttttgagacagggtctcgctctgccacccaggctggaatgcagtggtgcagtcatagctcattgtagcctgttcctcctgggctcaagcaatcctattgcttcagcctcccaagtaaatgcaactacaggcacacactactatgccctgctgattttttaaatttttagtagagacaagatcttgctatattgcccaggctagtttcaaactcctgggctcaggcaatcctccctcagcctccctaagtgctgggactacagatgtgagccactgtgcctggcccgtaaacactttaagagaagaatgctagaataagattgtgatattacatacgaaagtggttaattaatgaagttttcttttcttttttttttttttgttttttgttttttttgagacggaggctcattctgtcgtccaggctggagtacagtggtgcgatcttggctcactgcaacttctgcctcccgggttcaagcaattctcctgcctcagcctcccgagtagctgggattacaggtgctcaccgccacacccagctaatttttgtatttttagtagagatggggtttcaccatgttggccaggatcgtctccatctcctgacctcgtgatctgcccacctcggcctcccaaagtgctgggattacaggtgtgagccactgcgcccggccatgaagttttcttttctacgggatagacacccacaagctaataagtaacttcagtgtctgggctgtaaccaaacaataaatagcaaagtcaacacaggcacaacccccaagattaggatcagcaaacgttttctgtaaaggaccagacatgcagctctcagtcacaaccaccatgccatgtagcatgaaagcaaccacagccaatgtgtaacaaattagcatggtatgtgtggccaggggcagggctcacgcctataatcccagcactttgagaggccaaggcgagaagattgcttgaggtcaggagttcgagaccagcctaggcaataaagttggaccccatcgctacaaaaagtaaaaagaaaaaaactagcagggcgccgtggtgcctgcctgtggtcccaggtactggggaggctgaggcaggaggactgcttgagcccaggagcttgaagctgcagtgagctatgatctcaccactgcactgcagcctagacaacagagccgagatcatgccactgcactccagcctgggcgacagaacgagatgctatctcaaaaaaaaaaagaaaaaaagagaaaaaaagaaaaaaagtgataagggtaataattccctgaggatataacctccacacttctgggtcaccctggaaaagcttgggtcaaggatgctacccttggctaggaacagtggctcacgcctgtaatcccagtactttgggaggctgagtgcagtggcgcaatctcggctcactgcaacctccgcctcccgggttcaagcaattctcctgcctctgcctcccaagtagctgggattacaggtgcccaccaccacgcccgactaattttttgtatttttagtagagatgaggtttcaccatgttggccaggctggtctctaactcctgacctcaagtgatccgcctgcctcagcctctcaaagtgccaaagtgctaggattacaggcgtgagtcaccgcacccagcctacccatatcactttctgttcctcagcagttaaagaggtgcccacccacttcgcctggaaagtcagattctgtgtggggctgttgcagaatgagatttcctcctgccagggtgcaagaactcaagtaggaaatgggccttcccagaaggggtagatgctccagatttcctcagcactgtagctgctgctcagccttggagaaacagctgccaaactgttagggccctggtagctcctctgagaccccagtccctgttgcttcaaacagcttaaggagggcctttatgctaaactgtagtctgcaaagcaaccaaaccaaaaagggtaagatacacttatccacctgtagggtccagccccacagggtcggtgggtttctctccatgtgcagagacgagagagcgcagaaataaagacacaagacaaagagataaaagacagctgggctggggggaccactaccaccaagacgcagagaccagtagtggccctgaatgccaggctgcgctgatatttattggatacaagacaaaggggcaggataaggagagtgagccatctccaatgataggtaaggccacatgggtcacgtgtccactggacaggggcccttccctgcctggcaaccgaggcagagagagagaggagaaagagagagagacagcttacaccattatttctgcttatcagagacttttagtactttcactaatttgctactgctatctagaaggcagagccaggtgtacaggatggaacatgaaggtggactaggagcgtgaccactgaagcacagcatcacagggagacggttatgcctccgggtaactgtgggcgggcctgtggaggagtagagtcttctctaaactcccccggggaaagggagactccctttcccggtctgctaagtagcgggtgtttttccttgacactaacgctaccgctagaccacggtgggcttggcaacaggtgtcttcccagatgctggcgttaccgctagaccaaggagccctctggtggccctgtccgggcataacagaaagcttgcactcttgtcttctggtcactcctcactgtcccctcagctcccatctctgtatggcctggtttttcctaggttatgattgtagagcgaggattattataatattggaataaagaataattactacaaactaatgattaatgattcatatataatcatatctaagatctatatctagtataactattcttattttatatattttattatactggaacagctcgtgccctcggtctcttgcctcggcacctgggtggcttgctgcccacatccaccaagtgcactttgggaggctgaggctggaggactgctggaggccaggagttcaataccagcctgggcaacatagggagaccccccccccccccaccatctccaaaaataaaaaaaaaaatagctaggtgtgatggcacatgcctgcagtcctaactatcgggaggctgaggcaggaggatctctggagcccaggagttggaggctgcatgatggcgccactgtacttcagcctgggcgacagagggagacgctgtctctaaataataataataatatataaaaagatatacgtaaccctacgggctcccctctcactgctgtatacacctgcctccttctgcatccctcacccaattgtccctcccggctaacaagccagctgcaccccccaggcagaggtcctgcagcagtcggtataggcagcctgggcagggggcttgtcatagatctgcctctggccacgagatcttctgctccccctacggctgctagccgcagctgtccactaccccttagctttattctctctattcaggggcctcctgctgcgcactccaagggggtgcacgtttccaaagagagatcaagggacatggggaaaggagaaaggtacagcttgaggttcccgctcagccaggactaggaggcctctgtaggggcagaacagccctgccggcgctctgagcatgcgccgaaaagcgcccacggggaaagccatgccggggtgcggggtggggggggggggggggcggtgcgctcgccactgacgcatgcgcagagacacgggtctctctcggggttttctcgcgcctgcgcaagatcctccagcccaacagggggcgatgagccgatccttgagcgggtttgccccgccggagtaatccggaagaggcctcttattagggctctggtggcgacggtggcggacacttggggtctggacgcaacggcggcgggagcatgaacgcccctccagccttcgagtcgttcttgctcttcgagggcgagaagtaagtgacgccggctgcggagggccgaggtgcgcgggcctgcggctgtcgcattctggggtgtctcctgcccatctcttgccccgggagggaaacagcttttgcttgatcgtctgctcggcctgtgtcagccaaatttaaggccgttccgcgaggtagtcgcgagtgtgttcattttgcagatgaggacactgaggcttggaggcgagcaagttgccccggacacaggaagtggcggcgcttgtattcgaacccagagtgtctggttctccatagcccttttcctctgggatctggcagcttcctctttttgtctctcgcccgccacctccatccttcatcccctccgatctgtcagcggcctgcgtaaaagccgcagtggctcctcgctgcctgcgggttgaaggagtccaggggagagagcttgggctcactctgtatctgtgggcgagtggcttgatctgtcggaggccctgtttcctcatctggaaaatgagtttattactgtacttaaacgtgggattcaggccgggtgcggtggctgacacctgtaatcccagcactttgggagcccgaggcgggcggatcacctgaggtcgggagttcaagaccagcctgaccaacatggagaaaccctgtctctactaaaaatacaaaattagccgggcgtggtggcagatgcctgtaatcccagctactcagaaggctgaggcaggagaatcacttgaacctgggaagtggaggttgaggtgagcggagatcacgccattgcactccagcctgagcaacaagagcgaaactccgtttcaaaaaacaaaaaaagggatctggccaggtgcgctggttcacgcttttaatcccggcactttgggaggctcaggtgggaggattgcttgagctgagcacaggagttccaggctgcagtgagctttgatcgcatcactgcactccagcctgggcgactgagcgcaatcctgtctcaaaataaaaagaattggatgaggtaatttgtgtaaaactcttggtccaaagctcagcccctggaaaacctcataaccattggctcttattgttcccaggttggcgtgcagaggtcttggtctggcccttgccagccacttcggcctcctttagagcagactctcaactggggaccatttggccccacaagggacatgtggcaataactgagaacattattggttgtcaaagggggttatgactggaatctactgggtagaggtcagggatgctgctgatgtcctacaatgtccacaagacagcaccccacaggaaagaatgatgcaccctaaaatgtgtgtggtgtcagcctggagaaaccaccttagaaaaaaggatattcaacttgatacctctcacctgtgtgagattttactgtcagcatagttgccctcaaattgagtgtgcattagaatcacctgaagggcttcttaaaccatagatccctgtgccccacctccagagttctgggtccagtaagtgggaggtgggagactcttaacaagttcccaggggattctgatgctgcttatctgggaactgcactctgaaaaccccttgtcttggaaaatccaatggaagtttcctgtctttgtccatctaagcgttcactaaaatcatagtaaatgccagagcatgacttgttttggggaatggatggaaggactgcacactccagccagggggatggttgctttcggaactcacagggtcaggaccagcctcaccgtgtaacttcagggcctctcttcttcaccttttggttgcttcattatgttggcgtaagaaaaaaggaacaggccaggcatgatgacgtgcgcctgtggtcccagctactcaggaggctaaggcgggaggatcactttagccgaggagttggaggctgcagtgagctatgatcacaccactgcactccagcctgggcagcaggagagatcatttgaggcgaggagttcaagaccagcgaggacaacaaggcaaaaccccatctctaccggaaaaaaaaaaggaggaattcagaaaaggccaaggttccatagacctggggtagcaggaagtaggggaccgggttggcgaggcctttatgggatgaagagattgagaaaggccttcaggaagaggcagagcttatttttcctttttatttttatatttttgatacagagttccactctgttgcccaggctggagtgcagtggcatgatctcggctcactgcaactccacctcccaggttcaggtgattctcctgcctcagtctcccaagtagctaggactacaggcatggatcaccacactcagctaatttttttttttctttgtatttttagtacagacagggttttgccatgttgggcaggctggtcttgaacttctgacctcaggtcatccatccacctcagcctcccaaagtgttgggattacagacacgagccaccacacctggccgggaggagacagggtttagactcagggtgggaggaggggagcccctgctcctcatctgtggcctctgtgtcttcctaggatcaccattaacaaggacaccaaggtacccaatgcctgtttattcaccatgaacaaagaagaccacacactgggaaacatcattaaatcgtaagtttcccgtcacaggctctcaggggctatgtttaatgggcccctctgggcaaagcacaagttccaagtcttcctcagtctctctgagctacaggaagtccttctgggggcttgctctgggcattttataatcttggtacctttttatcaaaatgacaccgacctccatgtcttcacataagccacacttctgtagtggcttattctgtagatgaacacgtgcatttacattcactgtttgtttgtttgtttgtttttgagatggaattttctctctttgttgcccaggctggagtgcaatggcacgatcttgctcactgcaacctccgcctcccgggttcaagtgattctcctgcttcagccttcctagtagctgggactacaggcccacgccacgatgcccggctaatttttgtgtttttagtagagacggggattcactgtgttggccaggctggtctcgaactcctgacctcaggtgatccacctgccggcttcccaaagtgctgggattagagacgtgagctactgcgcccggccttacattcaccattaagtgcacattctgtaaatgaacgagcagcagattccagcacagtaggcctgtttgttattcatgctgagggacattgaaaagcagtcaagattagctcccctcatgccacccgtttgtgacctcagtgccacctgggcctctaagatgtatgtggcagagactagaacttctatttaacaagagaaagaagtaggagttctgtggctatccagtgcccctaatgggcaccctgcattggttggtgggtgtttgtttataccagtgaggtcctagccaggtggttggtcagggatgtgtctgccattctggcccagggacttgccattttactgtagacttggctcttccctctggtgccaccagcgaagccaggcttttccccgtctgtgagtggggtctgcccagtctcctggtgtggtcaggcacggggatggaggaatcctgcccggagcccagccagtggggcacccaagctctcctggggccctgctgctcccctgctcttgagctcagtgttgccacctatgcacgggtcccccttgtggtgctcacgccactctgagccgctctagaggagacccctgcgtgcccagggcacagtacgcgacgcagccccatggcacaaggcccagtgtgcgtggccagccgctggcggctgtgctgctcggagggcagagttccatttgcattccactcactgccgggagctggccctcctctcctgctgcctttatttgctgcgtggaggctctttgtgggctttttgggtctgagtcactgaacactggctggacattcactgcctgggagatctctttgccagcactgcggtgcccggtgggaagaggttgctggaatctctttccagctcgggtccttcaacctcatggatgcagagtgtgtgtgtgtggcggggatagggtgtccccacatgaggtatcttcctaaaagtgttactttccttgaagactttttgggaaaaaagttgggggggatttttggctagaaagtctgttcttctgctctgagctggctgctacccagcttgggcgtgaggctagaccctcacactgtctgtccctggacctcagacaactcctaaaagacccgcaagtgctatttgctggctacaaagtcccccaccccttggagcacaagatcatcatccgagtgcagaccacgccggactacagcccccaggaagcctttaccaacgccatcaccgacctcatcagcgagctgtccctgctggaggagcgcttccgggtgagggcagggcctggaggggcagacggggtgggctggacactggcccgtgtgcccaggcctgggacagccctggcctgtttcttcggaggtcctcagggagaggcggcggtgatggaagaacagggacttccaccacaggctccaggacatgtggactgaggggctgtggagtctgggcctgtggctcccgtctgccccatgggacttctgtagtgctgcagggtccctcgggtgctgtgggccagatccgggcggggacctactgtcctttgggggtgctcttctacgtcccttgtcggtgattggcaaggcctggtccttccaggcctctgggaggcagctcaccccagggtggcccacacctgttcctagcagggcgcctgggaatctagaacagtttagaggggaaagagccacagcaaagaaaagccgaggcagggtgatcacgaggtcaggagttcaagaccagcctggcaaacatggtgaagccctgtttctactaaaaatacaaaaattagctaggcatggtggcatgtgctgtagtcccagctactcgggaggctgaggcaggagaatcgcttgaacccgggaggcggaggttgcagtgagccgagattgtgccactgcactccagcctaggtaacagagcaggactccatctcagtcaatcaatcaatcaatcaatctcagcggttgaactacccttgacatggttcagctctgtatccacacccaaatgtcatgtcaaattgtaattcccagtgttgtgggagggacctggtgggaggtgattggctcatgggggccgacttcccccttgctgttctcgtgatattgagtgagcgcttgtgggatctggttgtttaaaagcgtgcagccctcccacttcactctctctgtctctcctgctccaacatggccagacgtgcctgcttccccttcgccttctgccgtgattgtcagtttcctgaggcctccccagccacgcttcctgtacagcctgcagaactgtgagtcaattaaacctcttttcttcataaattacccagtttctcatagttctttatagcagtgtgaaaacagactaatggacccttctggttgaaggaatgcagccattctgcttgtttgactatgtcctttctattcatctctatttcctgggaggtgtttatccaagtgcaataggaggtattggtgaccgcacagtcccctcagtgttctgctagtaaatagttgaaggttgatcattgatcttctgcgttttcagtctggcatggaaaagcccctgtgcaactggtaaagatatcaataagcaccaggaggtatctaaatccaccaggagccataggcatcacgttgacgtccatttaccagtcttccctggcaagattcttctgaattgtgctgccttggccaaaagaggtatgggaggggctgggcgcagtggcttgtgcctgtaatcccaacattttgggaaaccaattcaggtggatcgttagaggtcaggggttcaagaccatcctggccaacatggtgacatcccatctctactaaaaatacaataagttagctgggtttggtgttgggtgcctgtaatcccagctactcgagaggctgaggcaggataatcgcttgaacctgggaggaggaggtggcagtgagctgagatcgtgccattgcactccagcgtgggcaacaagagtgaaacgtcgtctcaaaaaataaaaaaaaagtccgggcttgatggctcacacctgtaatcccagcactttgggaggccaagacgggtggatcacgaggtcaggagttcaagaccagcctggcctagatggtgaaaccctgtctctactaaaactacaaatattagctgggcatggtggcatgcacctgtaatctcagctactcagaagtctgatgcaggagaattgctaaaacccaggagggagaggttgcagtgagccgagattgcaccactgcactctagcctgggcgacagagcaagactccgtctcgaaagaaagaaagaaagaaaggaaattccccagggaagtacctcggctgatttcataaacaggtaccgaaggaagcagaggcatgtggaggacttccccacctcatgcagctatttgggccgtggcgtctgaaatttattatttcagagtcacccctttgatgaccttggcagtgaactgcagtcatctgtttaggcctttccatggcccacgtcaatgccgttatttctgtttgttgcacatttgatttccttgttgttggcatttagaaggccccctgcttcccagatcacaccacgggcatggaccacagagattgcatcttgtgagtctgtagaaatggtcaaggccttgtcctctcttaggtccagagctcaggtgaatgcagattttcccggccatctgtgctgaagtccctgtggggaggctcctggctggtttcctgtaggtagacagctacacgtcctgcccttcattggcttcttttcatgaagctcctgccatctacaaaacatgtctcccttcttgaatcacatctctgttattgaaactctagaagtcaaccgggcatggtggctatgcctataatcccagcattttgggatgccaaggcgggtggatcacctgaggtcaggagttcaagaccagcctggccaacatggcgaaaccccgtctctaatacaaatacaaaaattagccaagcatggtggtcactgtactccagcctgggtgacagagcaagactccgtctcaaaaaaaaaaaaaaaaaaaaaagagaaagaaagtatcatgcttttctgcattctgtgaattgttttagtgagttatcgaacttgagggcatggtgggaacctccaaatttgcagccagttggtgagaagtacatgtggtctgaggacacccaagcctgcaggtgtgtctaaagcgagggcagcctagtggggactggtggccttaacctgtggcatttgaggtaacatcagggagttgacatcagaattgcatcacataggctgggcgcggtggctcacgcctgtaatcctagcactttgggaggccaaggcgggcagatcacgaggtcaggagatcgagaccatcctggctaagacagtgaaatcccgtctctactaaaaattcaaaaaattagccaggcatggtggcgggcgcctgtagtcccagctactcgggaggctgaggcaggagaatggcgtgaacccaggaggcagagcttgcattgagccaagatcacgccaccgcactccagcctgggtgacagagcgagactccatccccccgaaaaaaaaaagacaaaaaaaaaattgtgtcacacaggccagatgcagtggctcatgcttataatcccagcaatttgaaaggcaaggtaagaggatcgcttgagcttgagtctgaggccgcagtgagctatgaccacaccactgcaccccagtctgggtgacagcgcaagaccccaactccaaaaaggaaaaagaaaaatcacaaggaattgcatgtcagagtgcctgtctttcacagctttaactgctgcaggaacgaactttttttttttttttttttttgagagggtgtgaggagacacaatctctgctagtgattctcctgcctcagcctcccaaatagctgggattataggcgtgcaccaccacgcctgcctaatttttgtatttttagtagagacagggtttcaccatgttggccaggctggtctcaaactcctgctgggatcatgggcgtgagccaccacgcccggccacctttagagttttcttaccacctggttttcctctctcaatatctttctctcatttcctgccttaaaactctagcttggcatctgggcgcagtagctcatgcctgtaatcccagcactttgggaggccgaggtaggtggatcacttgaagtcaggagttcgagaccagcctggccaacatggtgaaaccttgtctctactatttttacaaaagttagtcggacgtacagacgggtgcctgtagtcccagctacttgggaggctgaggcaggagaatttgtttgaacccagaggtgaaagttgcagggagccgaggttgtgccactgcactccagcctgggagacagagcaagactctgtctccaaaacaaacaaacaaacaaaaaaaccctgtagcttgggatcagccttctcttctattgtttttctttaaaaaataaaaattaaaaatggatgtagatgctatgttgctgaggctggcctcaaactcctggcctcaggtgatcctcccgccatgacctccaaaaccacagggattgtaggtgtgagcactgcacccagccttatgtttttttctacataaaaaacagcacaggattatcttccagagctaataaatatgttcaaataaccacaaccccattaaggaaaaatatcactgggcagcaaataatcaatccagaccaatatgatcacaattgctgtgaaggtgagaaaagttcatttttattatgtttccccaagagacgcactctattgttctcttgaaaacacacagctcatgtcctcctttagaacacacatcctctttaaagtaacatacaaacatgccaaaacaaggtaaaaaattacatctgaattctcacatttcaaatatatatgaaatatcaaataaaaatttatttttacaagaatttaggggaactactacatagctataaatgtaatatatatgttaactaagtatcatagataaaaaccatgctcccttcagcagcacgtgtaataatagatgcaaagattgaaaggtaaaagatttaggatgaaaagaatcctctcttaaaaaggaaaacaaaattatatgtatgtgtatataacagttataatatccatcacacagctttatagaaacagcatctattcaaaaataccagtatttccaaaatatttaaaataatatttaaagtaataataatatttaaataaataaatatatttaataaatatttaaataaataaaataatatttaaataattctatacccatgtttttcaaaataaaccaataaaatagatagtatatattagacgtgttagtatatatatctgaggcatgttaaaaatcacaactgaattctcacaattcagtcacaaacctaaacagcaaataaaaatttctatcaccagaattatgtttttttctggtggggaactaccaatagctataaatagaagagattattatggaagtatcatagataaaaagagtgctcgcttcaggagcacatgtaataatacagaaacaaatttaaagataataaaatatttaggataaaaagaattgtctcttaaaaatgaaaagaaaattagctttatgtatatataacaactataactctcatcaaaaaactacaggaacagcatgttttcaaaagtacaacaatttccaaactatttgaaataaatctatgaataattcaatggccgacattttccatacaaaccaataaaatgcagagtgtgcatgaagctatctgttacaatctgtggcactgatatttcacaaaagaattctgtcccaatctgagcccctgcattgtgccttcaaatcctcctggactgcaagtccgtaagaaacaggacctccaggttccgccccagggaggttggaattcagcaatataaaaagggtggtggtgccgcaggaaagggtggaactggaaacgctcctggtttcttacttttctccaaggactcctagaaggaccccacccccctccccccacccctgctcctaggaggacaacgtgatcactgtattcagctccatcaagaatggtccaggttcttctagatgatctgcacaaatggttcctctcctcctgcctggtgtctgccattagcattggaataaagttcctgctgaaaatccacatctcccctgggtccggtgttctggaagcgagagagacaatgtcacacttcaaggaggcagctctctagacaggaaagttattcacgtcccatgtcaattgagaaatgcaattttatctgctgcctttcattctataccctgcttctgaaccatcgtgttcaactgtgaaactcacactttggtgaccacgactccaaaactcacttaatacacccaaggtcagccccagtgatctgcttcatagcaaggactttgggtgggtctgcccagggagtagggcaccctcagagaatgtggctttggactttatcacagctggggccttttgtgtcacttaagatctaaacttgtaaccatgctagatgtgtttctaatgtgacaacatcacgaaccacgagtccagaagcctaatccttaatcctacctcctcatgatgaagtctcatgctctgtgctcaacgtggttagctgcacaagatgtaaaccaaagcttcactgaaccctcgacccaaatcagtaactcaagtgcgtcaatcataatgaacctccccaaactcagtttttatgattatttttgaggcagggtctcactctgtcgcccagactggagtgcagtggcaggatcagggctccgtgcggccccgaccttccaggctccagcgatcctcccgcctcagcctcctgagtagttgggagtagagatgcgtcccacatcgcctggctaatttttgtatttttgtggagaggggatctcgccacgttgcccaggcttgaagccagatcaagcaattgggttcttcggatttccgaaatagaccccaatattctgcctttaccccagaggatgcagatgtaccttctctcaggccgatgacctcaggcctccccggtccctggagctctaggaaaggtgagcgcgatctcgcgcccacacccagtgctctgggtcataagcctggatctggaaaaacaaacgccctttgagaagacggggactcgccaggatatccctctctcccctcatccagcctccagcccacccaattcctccccacctcctccacctccccaggccccactcacctcctccaactcctctggggaaacccaagccctgcagctcatggaacagaagaagtggaaccgtcgtttctggaacaggactatctgagagcggttcttcctggccctcgagttcatggaacggtataactggaaccgacgcttacggagcaagggtatgcgagagcggttcttcctggccctcgggttcgtggaacggtataactggaaccgacgcttacggagcaagggtatgcgagagcggttcttcctggccctcgggttcatggaatggcctaactggaaccaacgcttacggagcaagggtatgcgagagcggttcttcctatacaggaagtggaagatgttttgtttggagtcctcgtcgtcctcctccatgtcattggccaggtagctgaggacagaaatcaggttgctgctcaggggcaccaccaggagaggcctccggctgaggtcagcttcccagagaggaaggtaagggaccgtccctagctcaggactggcacccaccctgcagagagccatgccttcctcaggagggctctgctggacagagacctgatcaagggcgtctcccactccttcaggatggagacaaaaacccaactggtggccgagagtggtggcttacgcctggaatcccagcacactgggaggccaaagcaggaggatcacttgaggccaggagtttgagacgggcctgggcaacatagcaagaccctcgtctctattaaaaatgtaagaaatatgccagacgcggtggctcatgcctgtaatcccagcactttagaaggctgaagcaggtggatcgcttgagaccaggagttggagaccagcctggtcaacacggagaaaccccatctctactaaaaatacaaaaatgagcctggtgcggtggcacacccgttaggcctagctactcaggaggctgaagcacaagaattgtgtgaacccaggaggcggaggttgcagtgagttgagattgggccactccattccagcctgagaggcagaacaagactctgtctcaataaacaaacaaacaaacaaactgtccaggtgtggtggcacagccctgtagtcggagctaataaagaagctgaggtgggaggatcgcttgagcccaggatatggaggctgcggtgagctatgatctcaccactgcactccagcttgggggacagggcaagtctgtctcaaaaaaataaaagaaattgaatacattgatattttgccaggaccctgccttctacaggcatctagtctaatgggactgggagtaatcaaggcagatgacctaatcccagtgtccaggatgtaactagagagctacgggcatgcagaagttggaagatgagggaaggcatcacagaggctgtggggtgaactgatttcaaggaatgggtccttcccttcagagccacatgtgtgcgggacacccagacagaaaacacaaacacaaagtcgagtggagggcatttggaaggagcagtgaagccgagccaggaaataccaagatggcgagccagtgtgcttgtagagattgtagagagggtagaattgacactgtggaccctggcctcgatagagaaaggcatcagctaaggaagttgttcaggtgggcagtgaggttgtcgtgctttggaaagatgttcaggctgcactaggaagccccctggcttggggagagactccaggagaccccagcggggagcatttgacagtaaattcgagtgatgcgagggggacctgaactgtggcctctgtcatgggaacccagaggaggtcgatggcatttgtggttgatgtgggaaggagagagagagaagaaccagaaacgtctgcttgctggaggaagcggcatgtccgctcctccactccttttcttttccccttaggagcggtttatggttccttttgttttattcttttatttgtacactggcattggagtttgtttttttggctttttttttttttttttttttgagaaaaagtctcactctgtcacccaggctggagtacagtggctcgaccttagcttactgcaacctccacctcctgggttcaaagggttctcttgcctcagcctcccgagtagctgggattacagatgcacaccaccacgcccagctaatttttctatttttagtagagacgggatttggccatgttggccaggctggtctcgaactgctgacctcaggtgatccgcctgcctcggcctcccaaagtgctgggattacaggcgtattccactgtgcccagcctgagtttctgtttagaaacaacagtctatgatagtataatcctctcttttttgtacacagagtaaagaggacaaataggtgaaagaataaatgaaaggctggaatcccacttcccccgctgtcccagggcattggatattgacggataggaggcagcaaaccactcacagagccaggaagaaatgaatgcgttggtattgccaggaggggaagccggcccggctgaaatacgctatgaccatagccaggagatactgatggagagaaaggaacacagagagggagaggtcacatcttggaagaggaagattgtggagagggggaatgagggtctggggaggggctgcccatcagagaagggacctcagtgttggggtgactactcatttggaaattgcgggatggaggggtattcgaaggtcggatgcaaatccgagaagccagaggaagggttttgggtgatgctcccaggatggtgggctccgatgggatctttggagggggtgtgtctaggtcggctggtgtcaggagggtcttttgtgtgccaggcagagaactgtcccgaagagctgagagtagaggggccaggagcttcagggctgcggccagactgtggcccagagctcagatcccaaaggacccataggagaggcaggggccactcattcactctgcaagagaccagcagaatcctgagggagatgctgacaaatcataaaaagaccaagaatagccgggagtggcggctcaagcctgtgatcccagtactttttgagaggtggagacaggaggatcatgtgagcccaacagttcgagaacaacctgggcaacatagtgagaccctgtttctacaaacatttcaaaaattagttgagcatggtggcatgtgcctagtcccagctcctcaggaggctgaggaaagaagattgcttgagcccaggaattagaggctgcaatgagctatgatcatgccactgcactccatcctggggagcagagctagactctgtctcacaaaaaaaaaatttgtgggtgccaagactcaagaccatgggagctggtcgggcacagtggctgacgtctataatctcagcactttgggaggccaaggcgggtggatcgcctgaggtcaggtgttcaggaccaacctggccaacatggcaaaaccccgtttctactaaaaacacaaaaattagccaggcgtggtggttcatgtctgtaatcccagctgcttggaggctgaggcaggagaatcgcttgaacccaggaggcatcggctgcagtgagtcaagatcgagacactgccctccagcctgggcaacagagcaagactctgtctcacacacacacacacacacacacacacacaaaaaaaaaaaaaaaaaaaaagactgtaggagcatctggtgggaggtggtggagggagaactgtgggtttggaagctgcgccctccccccagccatgcgttggaacaggaacagttacatggagaacaaccttaccttgtccgacaccctcagatctttgtcccaggccaagaatcttttaatgacaggatcctctgtgattagagagcagatgtcagtgtgagaagcaggacagggtttccgtgggagcagcagggcagggaggagaagtgtgcctcccggggggaagtctcaggattgtggccgcgggtgaggtggatgggagaggggagaatgactttcactgggcaagggagagaggctcctgctctgagactcccctgagaagaggccgaaggaggccctgggtgtgagaatctacaggatgtagagctgggaatcagccaggaccccctccagcagacacggagggaccactgcagagtcataaaggaattcccatcatttcctcatgagacagtcacatcagggtgtgaccatggccttggtatcccccactatggatggagacacttaggtttagaaaagtcagtaagagacattaagtttcagagggcacagctgaaaccactttctttgtttattgattttgtttttctttatttgatttttatttttatttatttattaatttattttgagacagagtcttgctctgtgggccaggctggaatgcagtggcctgatcttggctcactgcaacctctgcctcccgggtttaagcgattctcctgtctcagcctcccgagtagctgggattacatgcatgagctactgtgcccagccttggtttttcttttgagacagggttttgctctgtcacccaggctggagtgcagtggtgtagtcatagctcactgcagcctcaaagtcctgagttcaagcaatcctcttgcctcagcctcccaacgtgctgggatctcaggcgggagccactgcgcctggcccgaaaccaagctttcttatcccaagcgctgacctttatcaagttgacctaatcctttatcatctcctaagtgtccctcatgagtgatcacttcacattcctcccacatggagagctcacccactggggcctatttttcccattggaaaagtgtggttattggaagtttcctgtttttggaaagaacaggattggaggtgctctctggggtgtcctcctaccaagcagcctgttgaaggcctcgtggtgctcagggagcacgagcgacactcgccgtcgcttcagcttcatcttgaggccacacagcatctccgccacccagatctcctcaggctcaggggcgagcaccttccgtggctcctcctccaacgactcctcagattcgtcccaccactccatcttccttttccagcaaaaggacctatgcggggggctgggatctaccccaggggctgagtaaagaaaccaggccacggtgtaatgcttctgcagttgatcacactagagcccgacccaaaaccccaaaccactctccatcctccccagcctcgcagactgctggcttctccaagccatctttccttctgtctgtctcctctgctgagctgcatgtgccgctccttctcctccccattctcccgtttttctgtcctcagaacacttcctcatatccttccctggtccctggctctctgagtcccttttttttttttttttttttgttgttgttgttgttgttgagaaacagtcttgctttgtggcctaggctggagtgtagtggtgcgatcttggctcactgcaacctctgcctcccgggttccagtgattctcctgcctaagcctcccaagtagctgggattacaggtgcccaccagaacgcccagctcatttttgtgcttctagaagagacagggtttcaccatgttggccaggctggtctccaactcctggcctcaagtgatctgcctgcctggcctcccaaagtgctgggattacaggtgtgagccactgcaccctgcctcagtacctccattcttcccacacaccctcctcatgtgctccttcctgacttctgggcccttccttccttctttttttttttttttttttttttttgagacagcgtctcactctctcacccagaatggaatgcagtggcgctatcttggctcaaagcaacctcttccacctgggttcaagcgattatcctgtctcagcctcctgagtagctgggataacaggcatgcctggctaatttttgtattgttagtataaatgaggtttcgctatattggtctggttggtctcgaacaactgacctcaagtgatccacccatctcagcctcccaaagtaatgggattacaggcatgagctaccacacctggccttcgtttttcttttgacacagggttttgctctgtcacccaggctggagtgcagtggtgcagtcatagctcagtgcagcctcaaagtcctgagttcaagcaatcctcttgcctcagcctcccaacgtgctagcatctcaggcgtgagccactgcacctggcccgaaaccaagctttctcatcccaagcgccaacctttatcaagtctagcctagtcttctatcgtctcctaagtgtccctcatgagtgatcacttctgagtcctcctgcgtggagagctcagccactgggggcgtatctttcccattggaaaagtgtggttattggaagtttcctctttttagaaagaacaggattggaggtgctctctggggtatcctcctaccaagctgactgttgaagtccttgtggtgctcagggaggatgggtgacactcgctgttgcttcagcttcatcttgagcccacacagcatctccactacccaggtctcctcaggctcaggggcgagctccttctccggctcctcctcagattcatctgaccactccctcttccttttccagccaagggacctacatggggggctgggatctaccccaggggctgagtaaagaaaccaggccaccgtgtaatgcttctgcatctgatcaccttagaccccgaccccaaaccccaaaccactctccatcctccccagacttgcagactgctggcttctctaagccatctttctgattttctcctctgctcaaccccatgtgccgctccttcccctccccattcttctctctctctgtcctccgaacactgcttcatgtccttccctggtccctggctctctgagtccctccttttttgttttgttttgttttgacacagaatcttgctttgtcacccaggctggagtgtagtggtgcaatctcagctcactgcaacatccatctcctggattccatttattcttctgcctcagcctctcaggtagctgggattacaggtgcctgccataatgcccagctcaattttgtacttttagtagagacagggtttcaccatgttggccaggctggtctcaaactcctggcctcaagtgatccgcctgccttggcctcccaaagttctgggcttacaggtgtgagccactgcacccagcctgaatttctccattcttcccacacaccctcctcaggttctccttcctgaccgctgacccttcttttcttttttttttttttttggagtgcagtagtgtgctctcagctcactgcaacctcttcctcccagtctcaagtgattctcctgtctcagcctcctgagtagctgggattacaggtgtgcaccactacaacttggctaatttttatacttttagtagagatggggtttcaccatattggccaggctggccttgatctcctgacctcaggtgatccgcccgcctcggcctcccaaagtgctggggttacaggcgtgagccaccgcacccggcccccttccttcgtcttagtcaatcctatcccacctcttcttccaccagtcccctcacctgatggtcccaacatttcatcatccaccacctcctggagggggtaccccgaggtgctccgctggggactctgctcattctgggggtgcggttgacggctggtcgtgatctttcccgtaatctgtcccctcttacggaacctagtctccgttctgtccatggccttcttctggacacttctaggatccagaagagtatgttatcaattctcaagcctaggagaagtcaggagtagagaacagctctgagaagatactgttgtccaactgatctccaggcaccacggagtccggtccctccaatcaggaaggtcggaatctctgatgtcatcgttcatgccaacctggcaaccagtttgaaaaaaaacacatgtaactgccaggctgatctcttgtcctggagatcctgggtgaatggtatctcctgccactgtcccaacctcagaccattgtccaaaagcatcttcagggactccacatccctctattccctgtcccagcagaggctgtgtcctctccactcaaagcctgaagcatgttggggtctcttcgtctctgtacatgcccatttcagagtccagtctggtgggagagggaacagagtgggaaagaaaactagggtaagcagaaacgatgaaaccttataagagtgagagtatcatgtacaagagtgagattatcatgtacaagagtgagattatcacgtacaagagatcccaggaatactgacttgatgaaaaagtcacatcagagcactcagtttggcagagcttttctgccgaatgtttactcacattcactgtccgagattctatactgggggtacacacgtcctctgccctaaggcaattttgagtccaagagacattttgaggcctaaaaatcataggaaactgcccctgagctcacacatatttccaatggtgtccccagtttcagggaatccatggattacctaagccagcccctccagttcggctaagaaactctagtctatatatcaagttttgtatcatatgtattgctctgaactcagaaatttcccttccatttatggattctatgaataaaatatcacatgtacaaaaagactaagtcaaaaaatttcagctgtgcacagtggctcatgcttgtaatcccagcactttgggtggccaagggaggaagattgcctgaggccagcagttcgagaccagtataggcaacatagcaagagcccatctctaaaaaaacaaaaccaaaccaaattagccaggtgtggtggctggcacctgtgttccaactacttgggagactcatgtgacaggaagatcacttgagcccaggagttagaagctgcagtgagccgtgatcttgccactgcactccagtctgggcaacacagcaagatactgtgtcaaaaaaagtttttttgataaaaaataaaagagttacatgacattcagagaccatccaaaaaacctgtgggttcccggctgggctcagtggctcatgcctgtaatcccagcactttgggaggccaaagtgggtggatcacttgaggtcaggagtttgagaccagcctggacaacatggtgaaaccccatctctactaaaaatacaaaaaattagccaggcatggtggtggatgcctgtaatcgcagctactcaggagagggcgctggagaatcacttgaactcatggtgcgcaggttgcagggagccaagatggcaccattgtgctccagcctgggcaacgagagcaaaactccatctcaaaaaaaataaagaacctgcgagtgagttcccacacgttttcctaatgggctgctgctttcctaggagtctctcgctcatagaaaaggcacaaactgaaagaggaagcagatcccattgctgtggaagtcccattgttaggaagctctgcttttctggagttcaaattcgcattcatgacgctttaaaccgtcagagctgggtgggtcctcctacaacaaaatagtttgctctctctctcctagttaacaggctttcaaatattagaagatcaatgttctgaccccattaaaatttctcttttgtggaatgaaaagctctgatttaacccatcttcaaggctggtttgatggaggaataggggctgagtcacctgcatttcccctccctgcacaaagtcctgggcccagatctggggtctgtctctgctgagggtggggtgaaccaggaagcacctccttcttcatctccttgatgaatgggtataatggttgccatggaactggggcttgtttgatgacctggggctgggtgggcctctgagagcctttatagctgattgccttttgggagaggggaggtgggagccccaccctgtctcatgagtcaccccaaaggtgcatgggcaggcaggtgctggggaatcggctactccccagagcttggcgtggccatccctgtggcccctctgggagtctggagcccattccctcacactggtactcactgcagctggggacatctgcactaggaagacaggacacggcatggaagctggcctctgcccagaagccatgacattctggtcaccagcctgatgctataaaacgagtgtcacggccgggcatggtggttcacacctgtaatcccagcactttaggaggccaaggcgggtggatcatgaggtctggagttcgagaccagcctggccaacatggcgaaatcccgtctctactaaaaataagaacattagccaggtgtggtggcacatacctgtagtcccagctcctctggaggctgaggcaggagaatcacttaaacccaggaggcggagattgcagtgagccgagaccacggcattggactccaggctgggcaacagagcacgactccatctcaaaaacaaacaaaaaaaaagagtgtcacctggggctacttggccagacacagagagcaaggagacatccctattatctgtcaaaaataattgttggggctgagcacagtggctcatgcctataatctcagcactttgggaggtcggggcaggaggacttgaggcctagagtttgagaccagcctgggcaacatagcgagcaccccatctccagaaaaaatttaaaaattggctgggcgcagtggctcatgcctgtaatcccagcactttgggaggccgagggggatggatcatttgaggtcaggagtttgagaccagcctggccaacgtggtgaaaccccatctctactaaaaatacaaaaattagccgggcatggtggtgggcacctgtaatcctagctacttgggaggctgaggcaggagaatcgcttgaacccaggaggcggtggttgtagtgagctaggatcatgccattgcagtccagcctggacagcaaagctagactccatctcaaaaaagaaaagaaaaagtaaaaaatttaaaaattagatgggcatggtgacatgtgcctgtaatcccaggtactaaggaagctgaggtaggaggatgacttgagcctaggagttcgaggctgcagtgagctctgatcgcaccactgcactccagcctgagtgacacagcaagaccctgcttcaaaaaaaaaaaaaaaattactggacacaattattgtgccagacccctaggttaacagtgaggattcagtgggagaactaaacagataaacagatccagtccccgccctcagagagttacagtttaatgggaaaaagagacattcaccaaagaaggacacagcccactagtgagttacactcgagagggatcatttacagaacaaagcagattataaaaatacagcgattggctgggtgcagtggctcacgcctgtaatcccagcactttgggaggcagaggcgggcggatgacttgaggtcaggagttctcaaccagcctggccaaaatggtgaaaccccatctctactaaaaacacaaaaattaaccaggcgtggtggtgggcacctgtaatcctagctacttgggaggctgaggcaggagaatgaatcgcttgaacccaggaggtggaggttgcagtgagccgagaccacaccattgcactctagcctgggcaacaagagcaaaactccgtctcaaaataaatacatacatacatacatgcatacatacatacagggattaaaatagtctagtactgacacctgaacagacagattgatccaagaaatgaaacagaaattccagaagttgacctgaacacacacacacacacacacacacacacacacacacacacacacacacgaaggcgtgaaagactccatgaccctcaaggtataagatgcatttttttttttttttgagacagggtctcactctgtcacccagactggatgcagtggtgcactatcccagctcagttctaccctccatccccccaacctcccccaaccaccctgagctcaagcaattctcatgcctcaaccctcagcctcatgagtaactgggactacaggcgtgcaccaccatgcgcagctaattttttgtatttttagtagagatgggtctaaccatattgcctaggctggtctcgaactcctgagctcaagcgatcctcttgcctcaacctcccaaagtgctgggattacagctgtgagccaccgcacccggccgcattcttctaaatcacagtacatctggttcccagtgcccaggctctcagggcagagggtccagtgtgatcactttgcatggcctctctcccctcctgagcttgtgccagggccccagggctgacctggagaaggaaaatggcagagggtgaagatggggtgtctggtttggggaccatcctggccccccttgtcactgttggcatctcttctgcacagtggcattgctgggaggtgcttactgtgcctattcaaggggctggcagccgcagcctcactgcagatcagggacttggcttcccggttgaccacaggtccaagaacctgcagggtccagcctcccccccatccccagtcttccccaccctggcccggccctccaggtgcagaaacatgcaggcccctctccaggactgtgggaggagtgtgtccctcagactggcctgtgtcctggctcctcttaccacctcttccagaggttgtcacctgcagctgccccaggataaaggcaaggccagagaggactcctgaactcctgtgtgcctggggtggcaggggcaaacatagccaactggtggcctgagcagggccatggtgaggacacccttggtggcttgtcccacatcaagctgggaggtgacactgaggatgcattagtctgcagcgtatgataaaaacggcatttcaggccaggcgtggtggctcatgcctgtcaccccagcaccttgggaggccgaggtgggcagatcatatgaggtcaggactttgagaccagcctggccaacatggtgaaaactcatctgtactaaaaaaacaaaaattatgtgggttggtggtgtgcgcctgtaatcccagctacttgggaggctgaggcaggagaatcacttgaacctgggaggcggaggctacaacgagccgaaattgcaccactgcactccagcctgactccgtctcaaaaaaaaaaaaaaaaaaaaaaaggcatttcagttcaaatagggaaaggatacatctttctttctttctttctttctttctttctttctttctttctttctttctttctttctttcctttctttgtttttctttcctcccttcctttatctctctctctctctctctttctctctttctttttgagatggagtttcactctcgctgcccaggctggagtacaatggtgcgatctcggcttattattattctccatgttggtcaggctggtcttgaactcccaacctcaggtgatccgcctgcgttggcctcccaaagtgctggtgtgagccactgcacccggcttaggatgcatttttcaatattttagtgtttgaataacgggctaacttgagaaaaaaataatttgaatcacacatcacaccaaaaatctaggtggattttaacactttcaaaaattattattagtttagagacagggtctcactccgtcgcctaggctggagtgcagtggtatgatcatggttcactgcaaccttaaactcctggcctcatatgatcctccggcctcagcctctcaaagtactggaactacaaacatgcaccaccacgcccagcctaggtgggtttttaaaatccattcaagggcgggtgcagtggctcacacctgtaatcccagcattttgggaagccaaggtgggaggatcacttgagcccaggagttcgagaccagcctgggcaacatagtgagaccacatctctacaaaaaatttaaaaatgagccaggcatggtggtgcacacctgtagtctctgctattcaggaggctgaggcgggatcattgtttgagcccaggagacagattgcagtgagctatgatggcaccactgcatggcagcctgggtgacaaagggagactcagtctcaaaaaaaaaaaaaaaaaggtgacaggctgggtgcagtggctcacacctgtaatcccagcactttgggaggccgaggcaggtggatcacctgaagtcaggagtttgagaccagcctggccaacatggtgaaaccctgtctttactaaaaatactaaagttggccaggcgtggtggcgtgtgcccgtaatctcagctacctgggaggttgagacaggagaatcactggaaccccagaggcagaagctgcagtgagctgagattgtgccactgcactccagcctgggtgacagaccgagactccaactcaaattaattaattaaattttaaaaaaaaaggcaaagagggagtcgtgcctgggtacaggagacctggggttgggtcccagccctgccgctgacctgccctgggactgcaggaagtttctttccctgccgggccccaggtttcttctcatccgtataatgagattagtcataacacctgccctgcccatctctggggacccaaggagaggatgagtcgatgagcaagaacgtattttcaaaaaggggaaacaagcccttcccacattctgatgtctgctttcaactgccttttgaaagggagtgagagagggaaaaattgtattatacaggccggacatggtggctcacacctggaatcccagaacttcaggaggctgaggtgggaggatcacttgagcccaggaatttgagaccagcctgggcaacacagtaagactctctttctacaaaaagttaaaaaattagctgggcagccggacacagtggctcacacctgtaatttcagcactttgggaggccgaggcaggcagatcacatgaggtcaggggttcaagaccagcctggtcaacatggcgaaccccgtctctaccaaaaatacaaaaattaaccaggagtggttgtgcacgccagtaatcccagctacgcagcaggctgaggcaggagaatcacttgaacccaggaggtggaggttgcagtgagccgagatcgtgccattgcactccagcctgaacgacagtgagactctgtctcaaaacaaacaaaaaattagctgggcatggtgccttatacctgcagtctcagctactagggaggctgaagtgggaggatcacttgatcctgggaggtcaaggctgcagtgagctatgatcacaccactgcactccagcctgggcaacagagtgagacccgtctcaaaaaataataataatataataaataaaagagaccccagagagcttccttgacccttccaccatatgaggacacagctagaaggcactctctacgaaccaaaaacagacaccaactcttcctagtttttgtttttgttcttgttcttttttttttaagatggagtctcattttgtcacccaggctggagtgcagtggtgtgatctcggctcactacggcctccccatcctgggtccaagcgattcttgtgcctcaacctcccgagtagctggaattacaggcgcgtgccaccacgactggctaattttgtatctttagtagagacggggtttttccacattggtcaggctggtctcaaacttctgacctcagatgatccgcccaccttggccacccaaagtgctgggattacaggtgtgaaccactgcgcccggccagaagtttttcttgtgagttgaggtgaacatctgaatcccctcggggaactgaggtatgtccctttcagggaatggcccctcttcatccaaaaatggggcccacaggtgataatctgttgggtttcccagaatgaagacatctggcataattagagcgtgaaggtcaggctgacgggggctagtaccccgatccaggggagaggtggtcacaggtctcgctccacctgccctgagccccatctcatctttttttttttttttttttttttttgagggggagtttcactcttgttgcccaggctggaatgcagtgatgcgatctcggctcattgcaacctctgcctcctgggttcaagtggttctcctgcctcagcctcctgcgtagctggagttacaagaacaagccaccacgtccagctaattttgtatttttagtagagatggggtttcgccgtgttggccaggctggtctcgaactcctgacctcaggtgatccacccacctcggcctctcaaaatgctgggattacaggcgtgagccaccatgcctggacttccatctggtaattttctgtctcgaggaggaactaccctgccctgacagaggctggggatgaaggaggaagagacagccggtcccctggataaagaggtgagggggtgtgaagtctgggagcccccctcccagggccaggctacagggggacagcagggctataccaggagtgccacagagcttgcatttgggtgtgatctaatccccagtcctgccctgacctcctgggtgctcttagccaagccctcttccctgtgtgtgctagaaagtgagggtcactcccttccacacaagggagtgaattgagggtggggatctgagctctctgagcagcaaagctggacacaggccaaattgccaatcctgagtccctctgtcatgtggacctgggaagctt

SEQ ID NO: 30 - DNA, 1247 bp, Homo sapiens
  Genbank Accession No. AF040639
ggaattcgccgcccgctccgagcctggtgcttccgaccgctgcgcgcggctcctgggctgtcacagtctcccgttgccgccgtcatgtcccggcagctgtcgcgggcccggccagccacggtgctgggcgccatggagatggggcgccgcatggacgcgcccaccagcgccgcagtcacgcgcgccttcctggagcgcggccacaccgagatagacacggccttcgtgtacagcgacggccagtccgagaccatccttggcggcctggggctccggctgggcggcagcgactgcagagtgaaaattgataccaaggccattccactgtttgggaactccctgaagcctgacagtctccggttccagctggagacgtcactgaagcggctgcagtgtccccgagtggacctcttctacctgcatatgccagaccacagcaccccggtggaagagacactgcgtgcctgccaccagctgcaccaggagggcaagttcatggagcttggcctctccaactatgcagcctgggaagtggccgagatctgtaccctctgcaagagcaacggctggatcctgcccactgtgtaccagggcatgtacaatgccatcacccggcaggtggaaacggagctcttcccctgcctcaggcactttggactgaggttctatgccttcaaccctctggctgggggcctgctgaccggcaagtacaagtatgaggacaaggatgggaaacagcctgtgggccgcttctttgggaatacctgggcagagatgtacaggaatcgctactggaaggagcaccactttgagggcattgccctggtggagaaggccctgcaggccgcgtatggcgccagcgcccccagcatgacctcggccaccctccggtggatgtaccaccactcacagctgcagggtgcccacggggacgcggtcatcctgggcatgtccagcctggagcagctggagcagaacttggcagcggcagaggaagggcccctggagccggctgtcgtggacgcctttaatcaagcctggcatttggttgctcacgaatgtcccaactacttccgctaggcccatcgtttctcaggctgcccaaggctcttctgtaacatcttttgttactcacattttctttaatttagaactgcctcagtaaattcttagggatggaagtntttggacaaaaacctaatagaagagtcaccaccaaatgaagaataaaactcccaaaaaaaaaa

SEQ ID NO: 31 - DNA, 2518 bp, Homo sapiens
  Genbank Accession No. L76465
ccagcagtggctgcaccatgcacgtgaacggcaaagtggcgctggtgaccggcgcggctcagggcataggcagagcctttgcagaggcgctgctgcttaagggcgccaaggtagcgctggtggattggaatcttgaagcaggtgtacagtgtaaagctgccctgcatgagcaatttgaacctcagaagactctgttcatccagtgcgatgtggctgaccagcaacaactgagagacacttttagaaaagttgtagaccactttggaagactggacattttggtcaataatgctggagtgaataatgagaaaaactgggaaaaaactctgcaaattaatttggtttctgttatcagtggaacctatcttggtttggattacatgagtaagcaaaatggaggtgaaggcggcatcattatcaatatgtcatctttagcaggactcatgcccgttgcacagcagccggtttattgtgcttcaaagcatggcatagttggattcacacgctcagcagcgttggctgctaatcttatgaacagtggtgtgagactgaatgccatttgtccaggctttgttaacacagccatccttgaatcaattgaaaaagaagaaaacatgggacaatatatagaatataaggatcatatcaaggatatgattaaatactatggaattttggacccaccattgattgccaatggattgataacactcattgaagatgatgctttaaatggtgctattatgaagatcacaacttctaagggaattcattttcaagactatgatacaactccatttcaagcaaaaacccaatgaacagcttatgtgttagccatagctgaaaataagcacaaatagcttatattcagatcctatcttcatttgaatatagcttttaaatgaaatgttacagtttgaagttttccttcatgcacttggtgataaacgttttctaaatttttagttaagtatatggataaaaagttatgaactattaaaaatgtgatgtggaccaaaggctaggttgtaatcttgatagtctaaaaaatgatcaaaacaaatgattttcaaggaatattcaatattctgcctttcagaaagtgtatttatatctgtgcttcataaatattaatgttcttcagaacatcattttaaaggagatacttgaattgttatttaaatcaaaccagatgtaaaacactcacatacaagttcatactttaaaagaggaaagctacttaacaatgacaaatatttcacaataataatttttacttatataccatctttcaactgaacatttcagttcttccaagagcttcttagagtagtatattttgggggcagtcaaggaataaactacagtgtaaacatatcccagatgaaaactgctgtatggaaaaatgacagaaagtaactgattgacactgttgattcacagttcagcctcctatctgggaaagacatttctttcctctgctcactttaagaacttttaccgactccaaaaatctcaggaattaaacttttaacagttacagcaataaagaatagttagtactccaaaaatattatatttaagatgctcaacaagaaaaaaatgcaaatgtaatatttttttcaaattacttctttattgacttgtccaaatttcaaaagtgcctacccttcaataaaacttttttattctgatctccataaattacttagtcttctatgtatagctatcaaggaaataaaaccaattttgccacagccacaactgtaaatgtttttgtacccatgctgaaactcataacaacacagacataaaaatagctgtgaggttttgctttttttgttgtcagctatcttaagaatcattaaatacacctgctttgggtaaaactctttgcaagcagtaattaacactagtaacagtgaaagcacaagatttccaaatcagtcgttttctcaaaaaaatatcgtataagtgactcatcctgtctgctaactccagacctcccagcttgaagccaaatctttccatgtgagattgatatggatttcctagaagtactggaatgttgtcatatcttgccctattttaattctgctatagaaaacaattgccttcacttttaaggagtaatttgaatattaataactctggtctagattttcatataatgtattaaagacaaagtagtgaacatcaatgaacatctgatagagataaactgtaatcaggcataagcttgtttgtatgttctggcagtgactaatcagtaaatgatgtcggtttgcccagtatcacttatcttctgtatttttcctctgtcgtgtaaatagtataaccttttcatttatggacaattttttggactagtagccttcaatatacattctgctttgaattaattttttcaaatcaataaattatgtagacatttaaaatcaaatatcaagtagaattgaaaaatgtgagttacataagttaaaaacttactttaaatcttaccttctataggtagctc

SEQ ID NO: 32 - DNA, 1959 bp, Homo sapiens
  Genbank Accession No. Z84717
gccactgagcctggctgtggggggagggggggtggtctttcaagctctcccaagccttactggtccctgtgctgcagcctggggggccacgaagaggcactggtgctcggcttgtccacggccagggctcagggcagaatggagcagcacgcttgttccctgctgggttgtcaggtgtggagagggtgctcctggggttgtggtggcctggggcactcacggccccatcccccagatgccccgtggtgtgggcactgccaggccctggcccccgagtacagcaaggcagctgccgtgctcgcggccgagtcaatggtggtcacgctggccaaggtggatgggcccgcgcagcgcgagctggctgaggagtttggtgtgacggagtaccctacgctcaagttcttccgcaatgggaaccgcacgcacccggaggagtacacaggaccacgggacgctgagggcattgccgagtggctgcgacggcgggtggggcccagtgccatgcggctggaggacgaggcggccgcccaggcgctgatcggtggccgggacctagtggtcattggcttcttccaggacctgcaggacgaggacgtggccaccttcttggccttggcccaggacgccctggacatgacctttggcctcacagaccggccgcggctctttcagcagtttggcctcaccaaggacactgtggttctcttcaagaaggtaggtcaggcccaggtgtgggttggggtccggctgcagcgcccgctaacccacctgctgctgtccagtttgatgaggggcnggcagacttccccgtggacgaggagcttggcctggacctgggggatctgtcgcgcttcctggtcacacacagcatgcgcctggtcacggagttcaacagccaggtgcgtaggctgcagtgcctggnctgggggtgtcccagggtagagtcgtccagggatgggggtgcctaggctgggggtcctgtggagtcatgagcaccctccctactgtagacgtctgccaagatcttcgcggccaggtcctcaaccacctgctgctgtttgtcaaccagacgctggctgcgcaccgggagctcctagcggctttggggaggcagctccccgcttccggggcaggtactggggggctgggggaaaggggcagcgggagagtggccggtgccagcatggactccctgccacaggtgctgttcgtggtggtggacgtggcggccgacaatgagcacgtgctgcagtactttggactcaaggctgaggcagcccccactctgcgcttggtcaaccttgaaaccactaagaagtatgcgcctgtggatgggggccctgtcaccgcagcgtccatcactgctttctgccatgcagtcctcaacggccaagtcaagccctatctcctgagccaggagataccccctgattgggatcagcggccagttaagaccctcgtgggcaagaattttgagcaggtggcttttgacgaaaccaagaatgtgtttgtcaagttctatgccccgtggtgcacccactgcaaggagatggcccctgcctgggaggcattggctgagaagtaccaagaccacgaggacatcatcattgctgagctggatgccacagccaacgagctggatgccttcgctgtgcacggcttccctactctcaagtacttcccagcagggccaggtcggaaggtgattgaatacaaaagcaccagggacctggagactttctccaagttcctggacaacgggggcgtgctgcccacggaggagcccccggaggagccagcagccccgttcccggagccaccggccaactccactatggggtccaaggaggaactgtagctgcccccgtgtcacccccgccatcactgctggacaggagccacccccttgggtaccagagggagctgtgcattgtgaataaagagtgagcttgggaattc

SEQ ID NO: 33 - DNA, 1922 bp, Homo sapiens
  Genbank Accession No. J03910
gagacctgggcgttggtgaaggcgaggggaaagccagaatactggggtggcggacgccaaggggcgggtgtagcaggcaacctcagggaagctgggaaggcctagcagggtgagagaacccgcacacggggggcacagcgcctccctcgcgagccggtgcgaaaggggccgcctgcggtgtgcgcccagctgcgctcaagggaccttgcacttggcccatctcctgcgcacagcccaggccgggaccgcgggcggtgcggactcagcgggctgggtgcaagggcggggcggggcgtctgcgcccggccccgtctcctgactataaaagcagccgctggctgttgggctccactccgccttccacgtgcacccactgcctcttcccttctcgcttgggaactctagtctcgcctcgggttgcaatggaccccaactgctcctgtgccgctggtaagggacgcccgggttctgtgccttggaatgccaaattcccagacaccatagagagtgtccctgggtttgaggaggtcgtattttgctatcagaggtaaggggactcctttattggtccagtgctttcctgttggccaagctcctgagggcattttcctcctccctgttcctctatgtcagagttgagggtcctgaggctcaaggctgtcctgctcatgtcagagttgagggtcctgaggctcaaggctgtcctgcctcacgtcacctagttggtcacagggctgctggctgagccccaattctctaacctgactctgagctaccggattggataggagacattggataggagggacattgcctcttccaagttcaggacagaaagtcgaagtcttcctaggccgtgatctgcagggactttcctttggagtagaaataggagggtgcttggttttcccagcatgaatggagaggacatggggcttctcttcctcgtctctgagtgggaaaggagctctgacggctggctctggcacagagaagggggaagtggacactcattgacccactgctgtaccttctgcatctcactcaccgttcactggctttttctcttctagcaggtgtctcctgcacctgcgccagctcctgcaagtgcaaagagtgcaaatgcacctcctgcaagaagagtgagtgcggggccatctccaggaatctggggctgtggctaaggttgggagggaacccaaggctgtccctgagtgcctgcttctggggaaccggccttcctttgtccctgtaggttgtcacgcctgtctagtcttctgcactttccaaggcttatgtgaggtggggcagctttctcaaaggaagacccattccaatgtccaccagttgtctcctgacaaaaaccatgccatcatgaactaagggtcctctggggctggagggatggagacaggcctctgttggggcagggagttctatgatcgagtctgctctgacctctcaatctcctttcctccccaaggctgctgctcctgctgccctgtgggctgtgccaagtgtgcccaaggctgcatctgcaaaggggcatcggagaagtgcagctgctgcgcctgatgtcgggacagccctgctcccaagtacaaatagagtgacccgtaaaatctaggattttttgttttttgctacaatcttgacccctttgctacattcccttttttctgtgaaatatgtgaataataattaaacacttagacttgattcccgttctggttcctgttgtgtttttggaatgagggactggggtgggagattgaactgggagttcacactgggctctggacggaaatgtgagtgctaaacaagctgagcgccttcaggcagccccgttacttctctgacctccttcctctgtaaaaggcacctgcaccgtgccggatgatatggggatggggacatacgcg

(19) United States
(12) Patent Application Publication
(10) Pub. No.: US 2006/0183186 A1
(43) Pub. Date: Aug. 17, 2006

(54) Gene expression profiles in stomach cancer

(76) Inventors: Sang Seok Koh, Daejeon (KR); Qing Liu, Gaithersburg, MD (US); Hyun-Ho Chung, San Diego, CA (US); Wen Zeng, Gaithersburg, MD (US); Bogman Lee, Daejeon (KR); Subrahmanyam Yeramilli, Clarksville, MD (US); Si Young Song, Madu (KR)

Correspondence Address: MORGAN LEWIS & BOCKIUS LLP, 1111 PENNSYLVANIA AVENUE NW, WASHINGTON, DC, 20004, US

(21) Appl. No.: 10/499,698

(22) PCT Filed: Dec. 20, 2002
(86) PCT No.: PCT/US02/40717

§ 371 Date: Jul. 12, 2005

Related U.S. Application Data
(60) Provisional application No. 60/341,816, filed on Dec. 21, 2001.
(60) Provisional application No. 60/343,191, filed on Dec. 31, 2001.

(51) Int. Cl. C12P 21/06

(52) U.S. Cl. 435/69.1

(57) ABSTRACT

The present invention results from the examination of tissue from stomach tumors to identify genes that are differentially expressed between cancerous and normal tissue. The invention includes diagnostic, monitoring, drug design and therapeutic methods using these genes, as well as solid supports comprising oligonucleotide arrays that are complementary to or hybridize to the differentially expressed genes.

### RELATED APPLICATIONS

This application claims the benefit of U.S. Provisional Applications 60/341,816 and 60/343,191, both of which are herein incorporated by reference in their entirety.

### FIELD OF THE INVENTION

The invention relates generally to the changes in gene expression in stomach tissue from patients with gastric cancer. The invention specifically relates to a set of human genes that are differentially expressed in cancerous stomach tissue compared to non-cancerous stomach tissue.

### BACKGROUND OF THE INVENTION

Stomach Cancer

In the United States, approximately 24,000 new cases of stomach cancer, or gastric cancer, are diagnosed every year. Although the incidence of stomach cancer has declined significantly in the last 60 years, it is still a serious disease caused by factors that remain elusive. Under similar circumstances, some people develop stomach cancer and others do not.

Stomach cancer usually occurs in people over the age of 55 and is twice as common in men as in women. This type of cancer is not one of the major ones in the United States, but it is much more prevalent in Japan, Korea, Latin America and parts of Eastern Europe, where people eat more foods that are preserved by drying, pickling, smoking or salting. Conversely, consuming fresh fruits and vegetables may protect against this disease.

Stomach cancer can develop in any part of the stomach and spread throughout the stomach and/or to other organs. The cancer may also grow along the stomach wall and spread to the esophagus or small intestine. If the cancer grows through the stomach wall, it can extend to nearby lymph nodes, the liver and the pancreas and the colon. Stomach cancer can spread even farther, to the ovaries, lungs and distant lymph nodes. When stomach cancer metastasizes to another part of the body, these tumor cells are of the same type as those in the original tumor. In other words, metastasized cells in the liver are still stomach tumor cells. Such tumor cells that spread to an ovary, establishing one or more ovarian tumors, are known as Krukenberg tumors and are composed of transformed stomach cells, not ovarian cells.

Because the symptoms of stomach cancer are non-specific, this cancer is difficult to detect in its early stages. Symptoms include indigestion, heartburn, abdominal pain, nausea and vomiting, diarrhea or constipation, loss of appetite, weakness and fatigue, and bleeding which is detected by blood in the stool or by the affected person vomiting blood. Diagnosis is usually performed by x-rays of the upper gastrointestinal tract and esophagus, the x-rays taken after the patient has consumed a liquid barium tracer. Endoscopy of the stomach and esophagus, with a gastroscope, can also be performed. If abnormal tissue is found, it can be biopsied through the gastroscope. Should the biopsy specimen show cancerous cells, surrounding lymph nodes are then biopsied, and surrounding organs, such as the liver and pancreas, are examined via CT scan to determine the extent or stage of the disease. Treatment methods for stomach cancer are similar to those employed in other types of cancer-removal of the affected organ (partial or total gastrectomy), possibly with removal of nearby lymph nodes as well, chemotherapy, radiation therapy and immunotherapy (stimulating immune system components that attack cancer cells) (http://cancemet.nci.nih.gov/cancertypes.html). As early stomach cancer causes few symptoms, diagnosis is not usually made before the advanced stages of the disease, where treatments are less effective.

Molecular Changes in Stomach Cancer

Little is known about the molecular changes in stomach cells associated with the development and progression of stomach cancer. Accordingly, there exists a need for the investigation of the changes in global gene expression levels, as well as the need for the identification of new molecular markers associated with the development and progression of stomach cancer. Furthermore, if intervention is expected to be successful in halting or slowing the progression of stomach cancer, means of accurately assessing the early manifestations of this disease need to be established. One way to accurately assess the early manifestations of stomach cancer is to identify markers which are uniquely associated with disease progression (see for example Kim et al., Oncogene 20:4568-4575, 2001). Likewise, the development of therapeutics to prevent or stop the progression of stomach cancer relies on the identification of genes responsible for cancerous transformation and growth in the stomach.

To date, researchers have been able to identify a few genetic alterations believed to underlie tumor development. These genetic alterations include amplification of oncogenes and mutations that result in the loss of tumor suppressor genes. Tumor suppressor genes are genes that, in their wild-type alleles, express proteins that suppress abnormal cellular proliferation. When the gene coding for a tumor suppressor protein is mutated or deleted, the resulting mutant protein or the complete lack of tumor suppressor protein expression may fail to correctly regulate cellular proliferation, and abnormal proliferation may take place, particularly if there is already existing damage to the cellular regulatory mechanism. A number of well-studied human tumors and tumor cell lines have missing or non-functional tumor suppressor genes. Examples of tumor suppressor genes include, but are not limited to, the retinoblastoma susceptibility gene or RB gene, the p53 gene, the deletion in colon carcinoma (DCC) gene and the neurofibromatosis type 1 (NF-1) tumor suppressor gene (Weinberg, Science 254:1138-1146, 1991). Loss of function or inactivation of tumor suppressor genes may play a central role in the initiation and/or progression of a significant number of human cancers.

Classification of heterogeneous populations of tumor types is a daunting task; yet, initial studies utilizing gene expression patterns to identify subtypes of cancer produced rather intriguing results (see Perou et al., Proc Natl Acad Sci USA 96:9212-9217, 1999; Golub et al., Science 286:531-537, 1999; Alizadeh et al., Nature 403:503-511, 2000; Alon et al. Proc Natl Acad Sci USA 96:6745-6750, 1999; and Bittner et al., Nature 406:536-540, 2000). Molecular classification of B-cell lymphoma by gene expression profiling elucidated clinically distinct diffuse large-B-cell lymphoma subgroups (see Alizadeh supra). Stratification of patients based on their distinctive gene expression profiles may allow researchers to precisely group similar patient populations for evaluating chemotherapeutic agents. The more homogenous population of patients decreases the variability of patient-to-patient responses leading to the development of agents capable of eradicating specific subtypes of cancers previously unknown using standard classification techniques.

The utilization of gene expression profiles to classify tumors, to identify drug targets, to identify diagnostic markers and/or to gain further insights into the consequences of chemotherapeutic treatments could facilitate the design of more efficacious patient-specific stratagems for treating a variety of cancers. In breast cancer, studies utilizing limited numbers of genes (8,102 genes) have classified tumors into subtypes based on gene expression profiles, and this study indicated a diversity of molecular phenotypes associated with breast tumors (Perou et al., Nature 406:747-752, 2000). The advent of cDNA and oligonucleotide arrays has enabled researchers to map tissue-specific expression levels for thousands of genes (Alon et al., Proc Natl Acad Sci USA 96:6745-6750, 1999; Iyer et al., Science 283:83-87, 1999; Khan et al., Cancer Res 58:5009-5013, 1998; Lee et al., Science 285:1390-1393, 1999; Wang et al., Gene 229:101-108, 1999; Whitney et al., Ann Neurol 46:425-428, 1999). The study by Martin et al. (Cancer Res 60:2232-2238, 2000) used a custom microarray composed of 124 genes discovered by differential display associated with either normal breast epithelial cells or from the MDA-MB-435 malignant breast tumor cell line. Using the custom microarray, researchers examined the relationship between expression patterns discovered by clustering a number of genes with clinical stages of breast cancer indicating that gene expression patterns were capable of grouping breast tumors into distinct categories (Martin et al., supra).

Although these studies have demonstrated that expression profiling may be used to produce improvements in diagnosis of human diseases such as cancer, as well as in the development of improved therapeutic strategies, further studies are needed. Accordingly, there remains a need in the art for materials and methods that permit a more accurate diagnosis of stomach cancer. In addition, there remains a need in the art for methods to treat and methods to identify agents that can effectively treat this disease. The present invention meets these and other needs.

### SUMMARY OF THE INVENTION

The present invention is based on the discovery of the genes and their expression profiles associated with various types and stages of stomach cancer.

The invention includes methods of diagnosing stomach cancer in a patient, comprising the step of detecting the level of expression in a tissue sample of one or more genes from Table 1; wherein differential expression of the genes in Table 1 is indicative of stomach cancer. The invention also includes methods of detecting the progression of stomach cancer. For instance, methods of the invention include detecting the progression of stomach cancer in a patient, comprising the step of detecting the level of expression in a tissue sample of one or more genes from Table 1; wherein differential expression of the genes in Table 1 is indicative of stomach cancer progression. In some preferred embodiments, PCA analysis based on all or a portion of the group of genes identified in Table 1 may be used to differentiate between the different stages of stomach cancer, such as in the metastasis of the disease to healthy regions of the stomach and to other organs. In some preferred embodiments, one or more genes may be selected from Table 1.

In some aspects, the present invention provides a method of monitoring the treatment of a patient with stomach cancer, comprising administering a pharmaceutical composition to the patient, preparing a gene expression profile from a cell or tissue sample from the patient and comparing the patient gene expression profile to a gene expression from a cell population comprising normal stomach cells, or to a gene expression profile from a cell population comprising diseased stomach cells, or to both. In some preferred embodiments, the gene profile will include the expression level of one or more genes in Table 1.

Another aspect of the present invention includes a method of treating a patient with stomach cancer, comprising administering to the patient a pharmaceutical composition, wherein the composition alters the expression of at least one gene in Table 1, preparing a gene expression profile from a cell or tissue sample from the patient comprising diseased cells and comparing the patient expression profile to a gene expression profile from an untreated cell population comprising stomach tumor cells.

In another aspect, the present invention provides a method of detecting the progression of carcinogenesis in a patient, comprising detecting the level of expression in a tissue sample of one or more genes from Table 1; wherein differential expression of the genes in Table 1 is indicative of stomach carcinogenesis.

The invention further includes methods of screening for an agent capable of modulating the onset or progression of stomach cancer, comprising the steps of exposing a cell to the agent; and detecting the expression level of one or more genes from Table 1. In some preferred embodiments, one or more genes may be selected from a group consisting of those listed in Table 1. In some preferred methods, it may be desirable to detect all or nearly all of the genes in the table.

The invention further includes compositions comprising at least two oligonucleotides, wherein each of the oligonucleotides comprises a sequence that specifically hybridizes to a gene in Table 1, as well as solid supports comprising at least two probes, wherein each of the probes comprises a sequence that specifically hybridizes to a gene in Table 1. In some preferred embodiments, one or more genes may be selected from a group consisting of those listed in Table 1.

The invention further includes computer systems comprising a database containing information identifying the expression level in stomach tissue of a set of genes comprising at least two genes in Table 1 and a user interface to view the information. In some preferred embodiments, one or more genes may be selected from a group consisting of those listed in Table 1. The database may further include sequence information for the genes, information identifying the expression level for the set of genes in normal stomach tissue and in cancerous stomach tissue and may contain links to external databases such as GenBank.

Lastly, the invention includes methods of using the databases, such as methods of using the disclosed computer systems to present information identifying the expression level in a tissue or cell of at least one gene in Table. 1, comprising the step of comparing the expression level of at least one gene in Table 1 in the tissue or cell to the level of expression of the gene in the database. In some preferred embodiments, one or more genes may be selected from a group consisting of those listed in Table 1.

### DETAILED DESCRIPTION OF THE PREFERRED EMBODIMENTS

Many biological functions are accomplished by altering the expression of various genes through transcriptional (e.g., through control of initiation, provision of RNA precursors, RNA processing, etc.) and/or translational control. For example, fundamental biological processes such as cell cycle, cell differentiation and cell death, are often characterized by the variations in the expression levels of groups of genes.

Changes in gene expression also are associated with pathogenesis. For example, the lack of sufficient expression of functional tumor suppressor genes and/or the over expression of oncogene/protooncogenes could lead to tumorgenesis or hyperplastic growth of cells (Marshall, Cell 64:313-326, 1991; Weinberg, Science, 254:1138-1146, 1991). Thus, changes in the expression levels of particular genes (e.g., oncogenes or tumor suppressors) serve as signposts for the presence and progression of various diseases.

Monitoring changes in gene expression may also provide certain advantages during drug screening and development. Often drugs are pre-screened for the ability to interact with a major target without regard to other effects the drugs have on cells. Often such other effects cause toxicity in the whole animal, which prevent the development and use of the potential drug.

Applicants have examined samples from normal stomach tissue and from cancerous stomach tissue to identify global changes in gene expression between tumor biopsies and normal tissue. These global changes in gene expression, also referred to as expression profiles, provide useful markers for diagnostic uses as well as markers that can be used to monitor disease states, disease progression, drug toxicity, drug efficacy and drug metabolism.

The gene expression profiles described herein were derived from normal and disease state stomach samples from five Korean patients between the ages of 47 and 68. The disease state associated with each sample is indicated in Table 2.

The present invention provides compositions and methods to detect the level of expression of genes that may be differentially expressed dependent upon the state of the cell, i.e., normal versus cancerous. These expression profiles of genes provide molecular tools for evaluating toxicity, drug efficacy, drug metabolism, development, and disease monitoring. Changes in the expression profile from a baseline profile can be used as an indication of such effects. Those skilled in the art can use any of a variety of known techniques to evaluate the expression of one or more of the genes and/or gene fragments identified in the instant application in order to observe changes in the expression profile in a tissue or sample of interest.

Definitions

In the description that follows, numerous terms and phrases known to those skilled in the art are used. In the interest of clarity and consistency of interpretation, the definitions of certain terms and phrases are provided.

As used herein, the phrase “detecting the level of expression” includes methods that quantify expression levels as well as methods that determine whether a gene of interest is expressed at all. Thus, an assay which provides a yes or no result without necessarily providing quantification of an amount of expression is an assay that requires “detecting the level of expression” as that phrase is used herein.

As used herein, oligonucleotide sequences that are complementary to one or more of the genes described herein, refers to oligonucleotides that are capable of hybridizing under stringent conditions to at least part of the nucleotide sequence of said genes. Such hybridizable oligonucleotides will typically exhibit at least about 75% sequence identity at the nucleotide level to said genes, preferably about 80% or 85% sequence identity or more preferably about 90% or 95% or more nucleotide sequence identity to said genes.

“Bind(s) substantially” refers to complementary hybridization between a probe nucleic acid and a target nucleic acid and embraces minor mismatches that can be accommodated by reducing the stringency of the hybridization media to achieve the desired detection of the target polynucleotide sequence.

The terms “background” or “background signal intensity” refer to hybridization signals resulting from non-specific binding, or other interactions, between the labeled target nucleic acids and components of the oligonucleotide array (e.g., the oligonucleotide probes, control probes, the array substrate, etc.). Background signals may also be produced by intrinsic fluorescence of the array components themselves. A single background signal can be calculated for the entire array, or a different background signal may be calculated for each target nucleic acid. In a preferred embodiment, background is calculated as the average hybridization signal intensity for the lowest 5% to 10% of the probes in the array, or, where a different background signal is calculated for each target gene, for the lowest 5% to 10% of the probes for each gene. Of course, one of skill in the art will appreciate that where the probes to a particular gene hybridize well and thus appear to be specifically binding to a target sequence, they should not be used in a background signal calculation. Alternatively, background may be calculated as the average hybridization signal intensity produced by hybridization to probes that are not complementary to any sequence found in the sample (e.g., probes directed to nucleic acids of the opposite sense or to genes not found in the sample such as bacterial genes where the sample is mammalian nucleic acids). Background can also be calculated as the average signal intensity produced by regions of the array that lack any probes at all.

The phrase “hybridizing specifically to” refers to the binding, duplexing or hybridizing of a molecule substantially to or only to a particular nucleotide sequence or sequences under stringent conditions when that sequence is present in a complex mixture (e.g., total cellular) DNA or RNA.

Assays and methods of the invention may utilize available formats to simultaneously screen at least about 100, preferably about 1000, more preferably about 10,000 and most preferably about 1,000,000 or more different nucleic acid hybridizations.

The terms “mismatch control” or “mismatch probe” refer to a probe whose sequence is deliberately selected not to be perfectly complementary to a particular target sequence. For each mismatch (MM) control in a high-density array there typically exists a corresponding perfect match (PM) probe that is perfectly complementary to the same particular target sequence. The mismatch may comprise one or more bases that are not complementary to the corresponding bases of the target sequence.

While the mismatch(s) may be located anywhere in the mismatch probe, terminal mismatches are less desirable as a terminal mismatch is less likely to prevent hybridization of the target sequence. In a particularly preferred embodiment, the mismatch is located at or near the center of the probe such that the mismatch is most likely to destabilize the duplex with the target sequence under the test hybridization conditions.

The term “perfect match probe” refers to a probe that has a sequence that is perfectly complementary to a particular target sequence. The test probe is typically perfectly complementary to a portion (subsequence) of the target sequence. The perfect match (PM) probe can be a “test probe”, a “normalization control” probe, an expression level control probe and the like. A perfect match control or perfect match probe is, however, distinguished from a “mismatch control” or “mismatch probe.”

As used herein a “probe” is defined as a nucleic acid, preferably an oligonucleotide, capable of binding to a target nucleic acid of complementary sequence through one or more types of chemical bonds, usually through complementary base pairing, usually through hydrogen bond formation. As used herein, a probe may include natural (i.e., A, G, U, C or T) or modified bases (7-deazaguanosine, inosine, etc.). In addition, the bases in probes may be joined by a linkage other than a phosphodiesteir bond, so long as it does not interfere with hybridization. Thus, probes may be peptide nucleic acids in which the constituent bases are joined by peptide bonds rather than phosphodiester linkages.

The term “stringent conditions” refers to conditions under which a probe will hybridize to its target subsequence, but with only insubstantial hybridization to other sequences or to other sequences such that the difference may be identified. Stringent conditions are sequence-dependent and will be different in different circumstances. Longer sequences hybridize specifically at higher temperatures. Generally, stringent conditions are selected to be about 5° C. lower than the thermal melting point (Tm) for the specific sequence at a defined ionic strength and pH.

Typically, stringent conditions will be those in which the salt concentration is at least about 0.01 to 1.0 M sodium ion concentration (or other salts) at pH 7.0 to 8.3 and the temperature is at least about 30° C. for short probes (e.g., 10 to 50 nucleotide). Stringent conditions may also be achieved with the addition of destabilizing agents such as formamide.

The “percentage of sequence identity” or “sequence identity” is determined by comparing two optimally aligned sequences or subsequences over a comparison window or span, wherein the portion of the polynucleotide sequence in the comparison window may optionally comprise additions or deletions (i.e., gaps) as compared to the reference sequence (which does not comprise additions or deletions) for optimal alignment of the two sequences. The percentage is calculated by determining the number of positions at which the identical subunit (e.g., nucleic acid base or amino acid residue) occurs in both sequences to yield the number of matched positions, dividing the number of matched positions by the total number of positions in the window of comparison and multiplying the result by 100 to yield the percentage of sequence identity. Percentage sequence identity when calculated using the programs GAP or BESTFIT (see below) is calculated using default gap weights.

Homology or identity may be determined by BLAST (Basic Local Alignment Search Tool) analysis using the algorithm employed by the programs blastp, blastn, blastx, tblastn and tblastx (Karlin et al., Proc Natl Acad Sci USA 87:2264-2268, 1990 and Altschul, J Mol Evol 36:290-300, 1993, fully incorporated by reference) which are tailored for sequence similarity searching. The approach used by the BLAST program is to first consider similar segments between a query sequence and a database sequence, then to evaluate the statistical significance of all matches that are identified and finally to summarize only those matches which satisfy a preselected threshold of significance. For a discussion of basic issues in similarity searching of sequence databases, see Altschul et al., (Nature Genet 6:119-129, 1994) which is fully incorporated by reference. The search parameters for histogram, descriptions, alignments, expect (i.e., the statistical significance threshold for reporting matches against database sequences), cutoff, matrix and filter are at the default settings. The default scoring matrix used by blastp, blastx, tblastn, and tblastx is the BLOSUM62 matrix (Henikoff et al., Proc Natl Acad Sci USA 89:10915-10919, 1992, fully incorporated by reference). Four blastn parameters were adjusted as follows: Q=10 (gap creation penalty); R=10 (gap extension penalty); wink=1 (generates word hits at every wink^{th} position along the query); and gapw=16 (sets the window width within which gapped alignments are generated). The equivalent Blastp parameter settings were Q=9; R=2; wink=1; and gapw=32. A Bestfit comparison between sequences, available in the GCG package version 10.0, uses DNA parameters GAP=50 (gap creation penalty) and LEN=3 (gap extension penalty) and the equivalent settings in protein comparisons are GAP=8 and LEN=2.

Uses of Differentially Expressed Genes

The present invention identifies those genes that are differentially expressed between normal stomach tissue and cancerous stomach tissue. One of skill in the art can select one or more of the genes identified as being differentially expressed in Table 1 and use the information and methods provided herein to interrogate or test a particular sample. For a particular interrogation of two conditions or sources, it may be desirable to select those genes which display a great deal of difference in the expression pattern between the two conditions or sources. In other instances, it may be appropriate to select genes whose expression changes only slightly between the two conditions. A difference of at least about two-fold may be desirable, but a three-fold, five-fold or ten-fold difference may be preferred in some instances. Interrogations of the genes or proteins can be performed to yield different information.

Diagnostic Uses for the Stomach Cancer Markers

As described herein, the genes and gene expression information provided in Table 1 may be used as diagnostic markers for the prediction or identification of a disease state of stomach tissue. For instance, a stomach tissue sample or other sample from a patient may be assayed by any of the methods known to those skilled in the art, and the expression levels from one or more genes from Table 1 may be compared to the expression levels found in normal stomach tissue, cancerous stomach tissue or both. Expression profiles generated from the tissue or other samples that substantially resemble an expression profile from normal or diseased stomach tissue may be used, for instance, to aid in disease diagnosis. Comparison of the expression data, as well as available sequence or other information may be done by researcher or diagnostician or may be done with the aid of a computer and databases as described herein.

Use of the Stomach Cancer Markers for Monitoring Disease Progression

Molecular expression markers for stomach cancer can be used to confirm the type and progression of disease made on the basis of morphological criteria. For example, normal stomach tissue can be distinguished from cancerous stomach tissue based on the level and type of genes expressed in a tissue sample. In some situations, identifications of cell type or source is ambiguous based on classical criteria. In these situations, the molecular expression markers of the present invention are useful for identifying the region of the stomach from which a sample came, as well as whether or not normal levels of gene expression have been altered (signs of metabolic disturbances).

In addition, progression of the carcinoma to new areas of the stomach or to other organs can be monitored by following the expression patterns of the involved genes using the molecular expression markers of the present invention. Monitoring of the efficacy of certain drug regimens can also be accomplished by following the expression patterns of the molecular expression markers.

As described above, the genes and gene expression information provided in Table 1 may also be used as markers for the direct monitoring of disease progression, for instance, the development of stomach cancer. A stomach tissue sample or other sample from a patient may be assayed by any of the methods known to those of skill in the art, and the expression levels in the sample from a gene or genes from Table 1 may be compared to the expression levels found in normal stomach tissue, tissue from a gastric carcinoma or both. Comparison of the expression data, as well as available sequence or other information may be done by a researcher or diagnostician or may be done with the aid of a computer and databases as described herein.

Use of the Stomach Cancer Markers for Drug Screening

According to the present invention, potential drugs can be screened to determine if application of the drug alters the expression of one or more of the genes identified herein. This may be useful, for example, in determining whether a particular drug is effective in treating a particular patient with stomach cancer. In the case where a gene's expression is affected by the potential drug such that its level of expression returns to normal, the drug is indicated in the treatment of stomach cancer. Similarly, a drug which causes expression of a gene which is not normally expressed by healthy stomach cells may be contra-indicated in the treatment of stomach cancer.

According to the present invention, the genes identified in Table 1 may also be used as markers to evaluate the effects of a candidate drug or agent on a cell, particularly a cell undergoing malignant transformation, for instance, a stomach cancer cell or tissue sample. A candidate drug or agent can be screened for the ability to stimulate the transcription or expression of a given marker or markers (drug targets) or to down-regulate or inhibit the transcription or expression of a marker or markers. According to the present invention, one can also compare the specificity of a drug's effects by looking at the number of markers affected by the drug and comparing them to the number of markers affected by a different drug. A more specific drug will affect fewer transcriptional targets. Similar sets of markers identified for two drugs indicates a similarity of effects.

Assays to monitor the expression of a marker or markers as defined in Table 1 may utilize any available means of monitoring for changes in the expression level of the nucleic acids of the invention. As used herein, an agent is said to modulate the expression of a nucleic acid of the invention if it is capable of up- or down-regulating expression of the nucleic acid in a cell.

Agents that are assayed in the above methods can be randomly selected or rationally selected or designed. As used herein, an agent is said to be randomly selected when the agent is chosen randomly without considering the specific sequences involved in the association of a protein of the invention alone or with its associated substrates, binding partners, etc. An example of randomly selected agents is the use a chemical library or a peptide combinatorial library, or a growth broth of an organism.

As used herein, an agent is said to be rationally selected or designed when the agent is chosen on a nonrandom basis which takes into account the sequence of the target site and/or its conformation in connection with the agents action. Agents can be selected or designed by utilizing the peptide sequences that make up these sites. For example, a rationally selected peptide agent can be a peptide whose amino acid sequence is identical to or a derivative of any functional consensus site.

The agents of the present invention can be, as examples, peptides, small chemical molecules, vitamin derivatives, as well as carbohydrates, lipids, oligonucleotides and covalent and non-covalent combinations thereof. Dominant negative proteins, DNA encoding these proteins, antibodies to these proteins, peptide fragments of these proteins or mimics of these proteins may be introduced into cells to affect function. “Mimic” as used herein refers to the modification of a region or several regions of a peptide molecule to provide a structure chemically different from the parent peptide but topographically and functionally similar to the parent peptide (see Grant, in Molecular Biology and Biotechnology, Meyers (ed.), VCH Publishers, 1995). A skilled artisan can readily S recognize that there is no limit as to the structural nature of the agents of the present invention.

Use of the Stomach Cancer Markers as Therapeutic Agents

Agents that up- or down-regulate or modulate the expression of the nucleic acid molecules of Table 1, or at least one activity of a protein encoded by the nucleic acid molecules of Table 1, such as agonists or antagonists, may be used to modulate biological and pathologic processes associated with the function and activity of the proteins encoded by these nucleic acid molecules. The agents can be the nucleic acid molecules of Table 1 themselves, the encoded proteins, or portions of these molecules, such as all or part of the open reading frames of these nucleic acid molecules.

Anti-sense oligonucleotide molecules derived from the nucleic acid sequences of Table 1 may also be used to down-regulate the expression of one or more of the genes in Table 1 that are expressed at elevated levels in stomach cancer, the use of antisense gene therapy being an example. Down-regulation of expression of one or more of the genes of Table 1 is accomplished by administering an effective amount of antisense oligonucleotides. These antisense molecules can be fashioned from the DNA sequences of these genes or sequences containing various mutations, deletions, insertions or spliced variants. Isolated RNA or DNA sequences derived from these genes may also be used therapeutically in gene therapy. These agents may be used to induce gene expression in stomach cancers associated with an absence of or considerably decreased expression of one or more of the proteins encoded by genes in Table 1.

As used herein, a subject can be any mammal, so long as the mammal is in need of modulation of a pathological or biological process mediated by a gene of the invention. The term “mammal” is defined as an individual belonging to the class Mammalia. The invention is particularly useful in the treatment of human subjects.

Pathological processes refer to a category of biological processes which produce a deleterious effect. For example, expression of a gene of the invention may be associated with hyperplasia in the stomach, in particular malignant hyperplasia. As used herein, an agent is said to modulate a pathological process when the agent reduces the degree or severity of the process. For instance, stomach cancer may be prevented or disease progression modulated by the administration of agents which up- or down-regulate or modulate in some way the expression or at least one activity of a gene of the invention.

The agents of the present invention can be provided alone, or in combination with other agents that modulate a particular pathological process. For example, an agent of the present invention can be administered in combination with other known drugs. As used herein, two agents are said to be administered in combination when the two agents are administered simultaneously or are administered independently in a fashion such that the agents will act at the same time.

The agents of the present invention can be administered via parenteral, subcutaneous, intravenous, intramuscular, intraperitoneal, transdermal, or buccal routes. Alternatively, or concurrently, administration may be by the oral route. The dosage administered will be dependent upon the age, health, and weight of the recipient, kind of concurrent treatment, if any, frequency of treatment, and the nature of the effect desired.

The present invention further provides compositions containing one or more agents which modulate expression or at least one activity of a protein of the invention. While individual needs vary, determination of optimal ranges of effective amounts of each component is within the skill of the art. Typical dosages comprise 0.1 to 100 μg/kg body wt. The preferred dosages comprise 0.1 to 10 μg/kg body wt. The most preferred dosages comprise 0.1 to 1 μg/kg body wt.

In addition to the pharmacologically active agent, the compositions of the present invention may contain suitable pharmaceutically acceptable carriers comprising excipients and auxiliaries which facilitate processing of the active compounds into preparations which can be used pharmaceutically for delivery to the site of action. Suitable formulations for parenteral administration include aqueous solutions of the active compounds in water-soluble form, for example, water-soluble salts. In addition, suspensions of the active compounds as appropriate oily-injection suspensions may be administered. Suitable lipophilic solvents or vehicles include fatty oils, e.g., sesame oil, or synthetic fatty acid esters, e.g., ethyl oleate or triglycerides. Aqueous injection suspensions may contain substances which increase the viscosity of the suspension include, for example, sodium carboxymethyl cellulose, sorbitol, and/or dextran. Optionally, the suspension may also contain stabilizers. Liposomes can also be used to encapsulate the agent for delivery into the cell.

The pharmaceutical formulation for systemic administration according to the invention may be formulated for enteral, parenteral or topical administration. Indeed, all three types of formulations may be used simultaneously to achieve systemic administration of the active ingredient.

Suitable formulations for oral administration include hard or soft gelatin capsules, pills, tablets, including coated tablets, elixirs, suspensions, syrups or inhalations and controlled release forms thereof.

In practicing the methods of this invention, the compounds of this invention may be used alone or in combination, or in combination with other therapeutic or diagnostic agents. In certain preferred embodiments, the compounds of this invention may be coadministered along with other compounds typically prescribed for these conditions according to generally accepted medical practice. The compounds of this invention can be utilized in vivo, ordinarily in mammals, such as humans, rats, mice, dogs, cats, sheep, horses, cattle and pigs, or in vitro.

Assay Formats

The genes identified as being differentially expressed in stomach cancer may be used in a variety of nucleic acid detection assays to detect or quantify the expression level of a gene or multiple genes in a given sample. For example, traditional Northern blotting, nuclease protection, RT-PCR and differential display methods may be used for detecting gene expression levels. In methods where small numbers of genes are assayed, such as 5-50 genes, high-throughput PCR may be used.

The protein products of the genes identified herein can also be assayed to determine the amount of expression. Methods for assaying for a protein include Western blot, immunoprecipitation and radioimmunoassay. In some methods, it is preferable to assay the mRNA as an indication of expression. Methods for assaying for mRNA include Northern blots, slot blots, dot blots, and hybridization to an ordered array of oligonucleotides. Any method for specifically and quantitatively measuring a specific protein or mRNA or DNA product can be used. However, methods and assays of the invention are most efficiently designed with array or chip hybridization-based methods for detecting the expression of a large number of genes.

Any hybridization assay format may be used, including solution-based and solid support-based assay formats. A preferred solid support is a high density array also known as a DNA chip or a gene chip. One variation of the DNA chip contains hundreds of thousands of discrete microscopic channels that pass completely through it. Probe molecules are attached to the inner surface of these channels, and molecules from the samples to be tested flow throughout the channels, coming into close proximity with the probes for hybridization. In one assay format, gene chips containing probes to at least two genes from Table 1 may be used to directly monitor or detect changes in gene expression in the treated or exposed cell as described herein.

The genes of the present invention may be assayed in any convenient sample form. For example, samples may be assayed in the form of mRNA or reverse transcribed mRNA. Samples may be cloned or not, and the samples or individual genes may be amplified or not. The cloning itself does not appear to bias the representation of genes within a population. However, it may be preferable to use polyA+RNA as a source, as it can be used with less processing steps. In some embodiments, it may be preferable to assay the protein or peptide expressed by the gene.

The sequences of the expression marker genes of Table 1 are available in the public databases. Table 1 provides the Accession number, Sequence Number ID and name for each of the sequences. The sequences of the genes in GenBank are herein expressly incorporated by reference in their entirety (see www.ncbi.nim.nih.gov).

Additional assay formats may be used to monitor the ability of the agent to modulate the expression of a gene identified in Table 1. For instance, as described above, mRNA expression may be monitored directly by hybridization of probes to the nucleic acids of the invention. Cell lines are exposed to an agent to be tested under appropriate conditions and time and total RNA or mRNA is isolated by standard procedures such those disclosed in Sambrook et al., Molecular Cloning—A Laboratory Manual, Third ed., Cold Spring Harbor Laboratory Press, Cold Spring Harbor, New York, 2001. In some embodiments, it may be desirable to amplify one or more of the RNA molecules isolated prior to application of the RNA to the gene chip. Using techniques well known in the art, the RNA may be reverse transcribed and amplified in the form of DNA or may be reverse transcribed into DNA and the DNA used as a template for transcription to generate recombinant RNA. Any method that results in the production of a sufficient quantity of nucleic acid to be hybridized effectively to the gene chip may be used.

In another format, cell lines that contain reporter gene fusions between the open reading frame and/or the 3′ or 5′ regulatory regions of a gene in Table 1 and any assayable fusion partner may be prepared. Numerous assayable fusion partners are known and readily available including the firefly luciferase gene and the gene encoding chloramphenicol acetyltransferase (Alam et al., Anal Biochem 188:245-254, 1990). Cell lines containing the reporter gene fusions are then exposed to the agent to be tested under appropriate conditions and time. Differential expression of the reporter gene between samples exposed to the agent and control samples identifies agents which modulate the expression of the nucleic acid.

In another assay format, cells or cell lines are first identified which express one or more of the gene products of the invention physiologically. Cells and/or cell lines so identified would preferably comprise the necessary cellular machinery to ensure that the transcriptional and/or translational apparatus of the cells would faithfully mimic the response of normal or cancerous stomach tissue to an exogenous agent. Such machinery would likely include appropriate surface transduction mechanisms and/or cytosolic factors. Such cell lines may be, but are not required to be, derived from stomach tissue. The cells and/or cell lines may then be contacted with an agent and the expression of one or more of the genes of interest may then be assayed. The genes may be assayed at the mRNA level and/or at the protein level.

In some embodiments, such cells or cell lines may be transduced or transfected with an expression vehicle (e.g., a plasmid or viral vector) containing an expression construct comprising an operable 5′-promoter containing end of a gene of interest identified in Table 1 fused to one or more nucleic acid sequences encoding one or more antigenic fragments. The construct may comprise all or a portion of the coding sequence of the gene of interest which may be positioned 5′- or 3′- to a sequence encoding an antigenic fragment. The coding sequence of the gene of interest may be translated or un-translated after transcription of the gene fusion. At least one antigenic fragment may be translated. The antigenic fragments are selected so that the fragments are under the transcriptional control of the promoter of the gene of interest and are expressed in a fashion substantially similar to the expression pattern of the gene of interest. The antigenic fragments may be expressed as polypeptides whose molecular weight can be distinguished from the naturally occurring polypeptides.

In some embodiments, gene products of the invention may further comprise an immunologically distinct tag. Such a process is well known in the art (see Sambrook et al., supra). Cells or cell lines transduced or transfected as outlined above are then contacted with agents under appropriate conditions; for example, the agent comprises a pharmaceutically acceptable excipient and is contacted with cells comprised in an aqueous physiological buffer such as phosphate buffered saline (PBS) at physiological pH, Eagles balanced salt solution (BSS) at physiological pH, PBS or BSS comprising serum or conditioned media comprising PBS or BSS and serum incubated at 37° C. Said conditions may be modulated as deemed necessary by one of skill in the art. Subsequent to contacting the cells with the agent, said cells will be disrupted and the polypeptides of the lysate are fractionated such that a polypeptide fraction is pooled and contacted with an antibody to be further processed by immunological assay (e.g., ELISA, immunoprecipitation or Western blot). The pool of proteins isolated from the “agent-contacted” sample will be compared with a control sample where only the excipient is contacted with the cells and an increase or decrease in the immunologically generated signal from the “agent-contacted” sample compared to the control will be used to distinguish the effectiveness of the agent.

Another embodiment of the present invention provides methods for identifying agents that modulate the levels, concentration or at least one activity of a protein(s) encoded by the genes in Table 1. Such methods or assays may utilize any means of monitoring or detecting the desired activity.

In one format, the relative amounts of a protein of the invention produced in a cell population that has been exposed to the agent to be tested may be compared to the amount produced in an un-exposed control cell population. In this format, probes such as specific antibodies are used to monitor the differential expression of the protein in the different cell populations. Cell lines or populations are exposed to the agent to be tested under appropriate conditions and time. Cellular lysates may be prepared from the exposed cell line or population and a control, unexposed cell line or population. The cellular lysates are then analyzed with the probe, such as a specific antibody.

Probe Design

Probes based on the sequences of the genes described herein may be prepared by any commonly available method. Oligonucleotide probes for assaying the tissue or cell sample are preferably of sufficient length to specifically hybridize only to appropriate, complementary genes or transcripts. Typically the oligonucleotide probes will be at least 10, 12, 14, 16, 18, 20 or 25 nucleotides in length. In some cases longer probes of at least 30, 40, or 50 nucleotides will be desirable.

One of skill in the art will appreciate that an enormous number of array designs are suitable for the practice of this invention. The high density array will typically include a number of probes that specifically hybridize to the sequences of interest. See WO 99/32660 for methods of producing probes for a given gene or genes. In addition, in a preferred embodiment, the array will include one or more control probes.

High density array chips of the invention include “test probes.” Test probes may be oligonucleotides that range from about 5 to about 500 or about 5 to about 50 nucleotides, more preferably from about 10 to about 40 nucleotides and most preferably from about 15 to about 40 nucleotides in length. In other particularly preferred embodiments, the probes are about 20 or 25 nucleotides in length. In another preferred embodiment, test probes are double or single strand DNA sequences. DNA sequences may be isolated or cloned from natural sources or amplified from natural sources using natural nucleic acid as templates. These probes have sequences complementary to particular subsequences of the genes whose expression they are designed to detect. Thus, the test probes are capable of specifically hybridizing to the target nucleic acid they are to detect.

In addition to test probes that bind the target nucleic acid(s) of interest, the high density array can contain a number of control probes. The control probes fall into three categories referred to herein as (1) normalization controls; (2) expression level controls; and (3) mismatch controls.

Normalization controls are oligonucleotide or other nucleic acid probes that are complementary to labeled reference oligonucleotides or other nucleic acid sequences that are added to the nucleic acid sample. The signals obtained from the normalization controls after hybridization provide a control for variations in hybridization conditions, label intensity, “reading” efficiency and other factors that may cause the signal of a perfect hybridization to vary between arrays. In a preferred embodiment, signals (e.g., fluorescence intensity) read from all other probes in the array are divided by the signal (e.g., fluorescence intensity) from the control probes thereby normalizing the measurements.

Virtually any probe may serve as a normalization control. However, it is recognized that hybridization efficiency varies with base composition and probe length. Preferred normalization probes are selected to reflect the average length of the other probes present in the array, however, they can be selected to cover a range of lengths. The normalization control(s) can also be selected to reflect the (average) base composition of the other probes in the array, however in a preferred embodiment, only one or a few probes are used and they are selected such that they hybridize well (i.e., no secondary structure) and do not match any target-specific probes.

Expression level controls are probes that hybridize specifically with constitutively expressed genes in the biological sample. Virtually any constitutively expressed gene provides a suitable target for expression level controls. Typical expression level control probes have sequences complementary to subsequences of constitutively expressed “housekeeping genes” including, but not limited to the β-actin gene, the transferrin receptor gene, the GAPDH gene, and the like.

Mismatch controls may also be provided for the probes to the target genes, for expression level controls or for normalization controls. Mismatch controls are oligonucleotide probes or other nucleic acid probes identical to their corresponding test or control probes except for the presence of one or more mismatched bases. A mismatched base is a base selected so that it is not complementary to the corresponding base in the target sequence to which the probe would otherwise specifically hybridize. One or more mismatches are selected such that under appropriate hybridization conditions (e.g., stringent conditions) the test or control probe would be expected to hybridize with its target sequence, but the mismatch probe would not hybridize (or would hybridize to a significantly lesser extent). Preferred mismatch probes contain a central mismatch. Thus, for example, where a probe is a twenty-mer, a corresponding mismatch probe may have the identical sequence except for a single base mismatch (e.g., substituting a G, a C or a T for an A) at any of positions 6 through 14 (the central mismatch).

Mismatch probes thus provide a control for non-specific binding or cross hybridization to a nucleic acid in the sample other than the target to which the probe is directed. Mismatch probes also indicate whether a hybridization is specific or not. For example, if the target is present the perfect match probes should be consistently brighter than the mismatch probes. In addition, if all central mismatches are present, the mismatch probes can be used to detect a mutation. The difference in intensity between the perfect match and the mismatch probe (I(PM)-I(MM)) provides a good measure of the concentration of the hybridized material.

Nucleic Acid Samples

As is apparent to one of ordinary skill in the art, nucleic acid samples used in the methods and assays of the invention may be prepared by any available method or process. Methods of isolating total mRNA are also well known to those of skill in the art. For example, methods of isolation and purification of nucleic acids are described in detail in Chapter 3 of Laboratory Techniques in Biochemistry and Molecular Biology Vol. 24, Hybridization With Nucleic Acid Probes: Theory and Nucleic Acid Probes, P. Tijssen (ed.) Elsevier Press, New York, 1993. Such samples include RNA samples, but also include cDNA synthesized from a mRNA sample isolated from a cell or tissue of interest. Such samples also include DNA amplified from the cDNA, and an RNA transcribed from the amplified DNA. One of skill in the art would appreciate that it may be desirable to inhibit or destroy RNase present in homogenates before homogenates can be used.

Biological samples may be of any biological tissue or fluid or cells from any organism as well as cells raised in vitro, such as cell lines and tissue culture cells. Frequently the sample will be a “clinical sample” which is a sample derived from a patient. Typical clinical samples include, but are not limited to, stomach tissue biopsy, sputum, blood, blood-cells (e.g., white cells), tissue or fine needle biopsy samples, urine, peritoneal fluid, and pleural fluid, or cells therefrom. Biological samples may also include sections of tissues, such as frozen sections or formalin fixed sections taken for histological purposes.

Solid Supports

Solid supports containing oligonucleotide probes for differentially expressed genes can be any solid or semisolid support material known to those skilled in the art. Suitable examples include, but are not limited to, membranes, filters, tissue culture dishes, polyvinyl chloride dishes, beads, test strips, silicon or glass based chips and the like. Suitable glass wafers and hybridization methods are widely available, for example, those disclosed by Beattie (WO 95/11755). Any solid surface to which oligonucleotides can be bound, either directly or indirectly, either covalently or non-covalently, can be used. In some embodiments, it may be desirable to attach some oligonucleotides covalently and others non-covalently to the same solid support.

A preferred solid support is a high density array or DNA chip. These contain a particular oligonucleotide probe in a predetermined location on the array. Each predetermined location may contain more than one molecule of the probe, but each molecule within the predetermined location has an identical sequence. Such predetermined locations are termed features. There may be, for example, from 2, 10, 100, 1000 to 10,000, 100,000 or 400,000 of such features on a single solid support. The solid support, or the area within which the probes are attached may be on the order of a square centimeter.

Oligonucleotide probe arrays for expression monitoring can be made and used according to any techniques known in the art (see for example, Lockhart et al., Nat Biotechnol 14:1675-1680, 1996; McGall et al, Proc Nat Acad Sci USA 93: 13555-13460, 1996). Such probe arrays may contain at least two or more oligonucleotides that are complementary to or hybridize to two or more of the genes described herein. Such arrays my also contain oligonucleotides that are complementary or hybridize to at least 3, 4, 5, 6, 7, 8, 9, 10, 20, 30, 50, 70 or more the genes described herein.

Methods of forming high density arrays of oligonucleotides with a minimal number of synthetic steps are known. The oligonucleotide analogue array can be synthesized on a solid substrate by a variety of methods, including, but not limited to, light-directed chemical coupling, and mechanically directed coupling (see Pirrung et al., (1992) U.S. Pat. No. 5,143, 854; Fodor et al., (1998) U.S. Pat. No. 5,800,992; Chee et al., (1998) U.S. Pat. No. 5,837,832).

In brief, the light-directed combinatorial synthesis of oligonucleotide arrays on a glass surface proceeds using automated phosphoramidite chemistry and chip masking techniques. In one specific implementation, a glass surface is derivatized with a silane reagent containing a functional group, e.g., a hydroxyl or amine group blocked by a photolabile protecting group. Photolysis through a photolithogaphic mask is used selectively to expose functional groups which are then ready to react with incoming 5′ photoprotected nucleoside phosphoramidites. The phosphoramidites react only with those sites which are illuminated (and thus exposed by removal of the photolabile blocking group). Thus, the phosphoramidites only add to those areas selectively exposed from the. preceding step. These steps are repeated until the desired array of sequences have been synthesized on the solid surface. Combinatorial synthesis of different oligonucleotide analogues at different locations on the array is determined by the pattern of illumination during synthesis and the order of addition of coupling reagents.

In addition to the foregoing, additional methods which can be used to generate an array of oligonucleotides on a single substrate are described in Fodor et al. WO 93/09668. High density nucleic acid arrays can also be fabricated by depositing pre-made or natural nucleic acids in predetermined positions. Synthesized or natural nucleic acids are deposited on specific locations of a substrate by light directed targeting and oligonucleotide directed targeting. Another embodiment uses a dispenser that moves from region to region to deposit nucleic acids in specific spots.

Hybridization

Nucleic acid hybridization simply involves contacting a probe and target nucleic acid under conditions where the probe and its complementary target can form stable hybrid duplexes through complementary base pairing (see Lockhart et al., (1999) WO 99/32660). The nucleic acids that do not form hybrid duplexes are then washed away leaving the hybridized nucleic acids to be detected, typically through detection of an attached detectable label. It is generally recognized that nucleic acids are denatured by increasing the temperature or decreasing the salt concentration of the buffer containing the nucleic acids. Under low stringency conditions (e.g., low temperature and/or high salt) hybrid duplexes (e.g., DNA-DNA, RNA-RNA or RNA-DNA) will form even where the annealed sequences are not perfectly complementary. Thus, specificity of hybridization is reduced at lower stringency. Conversely, at higher stringency (e.g., higher temperature or lower salt) successful hybridization requires fewer mismatches. One of skill in the art will appreciate that hybridization conditions may be selected to provide any degree of stringency. In a preferred embodiment, hybridization is performed at low stringency, in this case in 6× SSPE-T at 37° C. (0.005% Triton x-100) to ensure hybridization and then subsequent washes are performed at higher stringency (e.g., 1× SSPE-T at 37° C.) to eliminate mismatched hybrid duplexes. Successive washes may be performed at increasingly higher stringency (e.g., down to as low as 0.25× SSPET at 37° C. to 50° C.) until a desired level of hybridization specificity is obtained. Stringency can also be increased by addition of agents such as formamide. Hybridization specificity may be evaluated by comparison of hybridization to the test probes with hybridization to the various controls that can be present (e.g., expression level control, normalization control, mismatch controls, etc.).

In general, there is a tradeoff between hybridization specificity (stringency) and signal intensity. Thus, in a preferred embodiment, the wash is performed at the highest stringency that produces consistent results and that provides a signal intensity greater than approximately 10% of the background intensity. Thus, in a preferred embodiment, the hybridized array may be washed at successively higher stringency solutions and read between each wash. Analysis of the data sets thus produced will reveal a wash stringency above which the hybridization pattern is not appreciably altered and which provides adequate signal for the particular oligonucleotide probes of interest.

Signal Detection

The hybridized nucleic acids are typically detected by detecting one or more labels attached to the sample nucleic acids. The labels may be incorporated by any of a number of means well known to those of skill in the art (see Lockhart et al., (1999) WO 99/32660).

Databases

The present invention includes relational databases containing sequence information, for instance for one or more of the genes of Table 1, as well as gene expression information in various stomach tissue samples. Databases may also contain information associated with a given sequence or tissue sample such as descriptive information about the gene associated with the sequence information, descriptive information concerning the clinical status of the tissue sample, or information concerning the patient from which the sample was derived. The database may be designed to include different parts, for instance a sequence database and a gene expression database. The databases of the invention may be stored on any available computer-readable medium. Methods for the configuration and construction of such databases are widely available, for instance, see Akerblom et al., (U.S. Pat. No. 5,953,727), which is specifically incorporated herein by reference in its entirety.

The databases of the invention may be linked to an outside or external database. In a preferred embodiment, as described in Table 1, the external database is GenBank and the associated databases maintained by the National Center for Biotechnology Information or NCBI (http://www.ncbi.nlm.nih.gov/Entrez/). Other external databases that may be used in the invention include those provided by Chemical Abstracts Service (http://stnweb.cas.org/) or Incyte Genomics (http://www.incyte.com/sequence/index.shtml).

Any appropriate computer platform may be used to perform the necessary comparisons between sequence information, gene expression information and any other information in the database or provided as an input. For example, a large number of computer workstations are available from a variety of manufacturers, such has those available from Silicon Graphics. Client-server environments, database servers and networks are also widely available and appropriate platforms for the databases of the invention.

The databases of the invention may be used to produce, among other things, electronic Northern blots (E-Northerns) to allow the user to determine the cell type or tissue in which a given gene is expressed and to allow determination of the abundance or expression level of a given gene in a particular tissue or cell. The E-northern analysis can be used as a tool to discover tissue specific candidate therapeutic targets that are not over-expressed in tissues such as the liver, kidney, or heart. These tissue types often lead to detrimental side effects once drugs are developed and a first-pass screen to eliminate these targets early in the target discovery and validation process would be beneficial.

The databases of the invention may also be used to present information identifying the expression level in a tissue or cell of a set of genes comprising at least one gene in Table 1, comprising the step of comparing the expression level of at least one gene in Table 1 in the tissue to the level of expression of the gene in the database. Such methods may be used to predict the physiological state of a given tissue by comparing the level of expression of a gene or genes in Table 1 from a sample to the expression levels found in tissue from normal stomach tissue, tissue from stomach tumors or both. Such methods may also be used in the drug or agent screening assays as described herein.

Without further description, it is believed that one of ordinary skill in the art can, using the preceding description and the following illustrative examples, make and utilize the compounds of the present invention and practice the claimed methods. The preceding working examples therefore, are illustrative only and should not be construed as limiting in any way the scope of the invention.

### EXAMPLES

### Example 1

### Preparation of Stomach Cancer Profiles

Tissue Sample Acquisition and Preparation

The patient tissue samples were derived from five Korean patients, four men and one woman, aged 47 to 68, who had been diagnosed with advanced gastric cancer (AGC). For each patient, tissue was obtained from two areas of the stomach to produce a set of biopsy samples. Tissue was removed from a gastric tumor and from the non-cancerous surrounding area composed of normal stomach tissue (NOR).

Histological analysis of each of the tissue samples was performed and samples were segregated into either normal (NOR) or cancerous (AGC) categories.

With minor modifications, the sample preparation protocol followed the Affymetrix GeneChip Expression Analysis Manual. Frozen tissue was first ground to powder using the Spex Certiprep 6800 Freezer Mill. Total RNA was then extracted using Trizol (Life Technologies). The total RNA yield for each sample (average tissue weight of 300 mg) was 200-500 μg. Next, mRNA was isolated using the Oligotex mRNA Midi kit (Qiagen). Since the mRNA was eluted in a final volume of 400 μl, an ethanol precipitation step was required to bring the concentration to 1 μg/μl. Using 1-5 μg of mRNA, double stranded cDNA was created using the SuperScript Choice system (Gibco-BRL). First strand cDNA synthesis was primed with a T7-(dT₂₄) oligonucleotide. The cDNA was then phenol-chloroform extracted and ethanol precipitated to a final concentration of 1 μg/μl.

From 2 μg of cDNA, cRNA was synthesized according to standard procedures. To biotin label the cRNA, nucleotides Bio-11-CTP and Bio-16-UTP (Enzo Diagnostics) were added to the reaction. After a 37° C. incubation for six hours, the labeled cRNA was cleaned up according to the Rneasy Mini kit protocol (Qiagen). The cRNA was then fragmented (5×fragmentation buffer: 200 mM Tris-Acetate (pH 8.1), 500 mM KOAc, 150 mM MgOAc) for thirty-five minutes at 94° C.

55 μg of fragmented cRNA was hybridized on the human and the Human Genome U95 set of arrays for twenty-four hours at 60 rpm in a 45° C. hybridization oven. The chips were washed and stained with Streptavidin Phycoerythrin (SAPE) (Molecular Probes) in Affymetrix fluidics stations. To amplify staining, SAPE solution was added twice with an anti-streptavidin biotinylated antibody (Vector Laboratories) staining step in between. Hybridization to the probe arrays was detected by fluorometric scanning (Hewlett Packard Gene Array Scanner). Following hybridization and scanning, the microarray images were analyzed for quality control, looking for major chip defects or abnormalities in hybridization signal. After all chips passed QC, the data was analyzed using Affymetrix GeneChip software (v3.0), and Experimental Data Mining Tool (EDMT) software (v1.0).

Gene Expression Analysis

All samples were prepared as described and hybridized onto the Affymetrix Human Genome U95 array. Each chip contains 16-20 oligonucleotide probe pairs per gene or cDNA clone. These probe pairs include perfectly matched sets and mismatched sets, both of which are necessary for the calculation of the average difference. The average difference is a measure of the intensity difference for each probe pair, calculated by subtracting the intensity of the mismatch from the intensity of the perfect match. This takes into consideration variability in hybridization among probe pairs and other hybridization artifacts that could affect the fluorescence intensities. Using the average difference value that has been calculated, an absolute call for each gene is made.

The absolute call of present, absent or marginal is used to generate a Gene Signature, a tool used to identify those genes that are commonly present or commonly absent in a given sample set, according to the absolute call.

The Gene Signature Curve is a graphic view of the number of genes consistently present in a given set of samples as the sample size increases, taking into account the genes commonly expressed among a particular set of samples, and discounting those genes whose expression is variable among those samples. The curve is also indicative of the number of samples necessary to generate an accurate Gene Signature. As the sample number increases, the number of genes common to the sample set decreases. The curve is generated using the positive Gene Signatures of the samples in question, determined by adding one sample at a time to the Gene Signature, beginning with the sample with the smallest number of present genes and adding samples in ascending order. The curve displays the sample size required for the most consistency and the least amount of expression variability from sample to sample. The point where this curve begins to level off represents the minimum number of samples required for the Gene Signature. Graphed on the x-axis is the number of samples in the set, and on the y-axis is the number of genes in the positive Gene Signature. As a general rule, the acceptable percent of variability in the number of positive genes between two sample sets should be less than 5%.

For the purposes of this study, the following statistical methods were used for the data analysis. A gene set consists of genes that have a certain percentage of present calls in at least one group of samples. These genes are analyzed, and others are excluded. For example, a gene having 40% present calls (2 out of 5 samples) in at least in one sample group, cancerous stomach cells or non-cancerous stomach cells, is included in the analysis if 40% is above the lower limit for percent present calls. Also, the genes are divided into two groups depending on their expression values across samples. For the genes in the high expression group, the average difference value is transformed to log scale before the analysis. For the genes in the low expression group, the original values are used in the analysis. An Analysis of Variance (ANOVA) method is used for data analysis (Steel et al., Principles and Procedures of Statistics: A Biometrical Approach, Third Ed., McGraw-Hill, 1997). Prior to the final analysis, a leave-one-out approach is used for outlier detection. One sample is left out of the ANOVA analysis to see whether omitting a specific sample from the analysis has any significant effect on the final result. If so, that particular sample is excluded from the final analysis. After outlier detection, the final analysis produces a list of genes that are differentially expressed with a p-value ≦0.001 as determined by the contrast from the ANOVA.

Differentially expressed genes were discovered by comparing biopsy samples from different regions of the same stomach in patients with advanced stomach cancer (advanced gastric cancer). Gene expression levels in a patient's stomach tumor cells were compared to those in the patient's normal stomach cells (AGC samples vs. NOR samples). Genes which showed no difference in expression level between a diseased state and the normal control were not included in Table 1. Table 1 (33 genes) lists the genes that were found to be differentially expressed when the level in cancerous stomach cells was compared to the level in normal stomach cells.

Fold Change analysis

The data was first filtered to exclude all genes that showed no expression in any of the samples. The ratio (cancerous/normal) was calculated by comparing the mean expression value for each gene in a cancerous sample set against the mean expression value of that gene in the normal tissue sample set. Genes were included in the analysis if they had a fold change ≧1.9 in either direction, and a p-value <0.00097 as determined by an Analysis of Variance Test (ANOVA). Out of the ˜60,000 genes surveyed by the Human Genome U95 set, 33 genes were present in the overall fold change analysis. In Table 1, numbers representing a comparison, or fold change, between the level of expression of a gene in disease state versus normal biopsy samples can be positive or negative. Positive values indicate a higher expression level in the tumor sample compared to the control (up-regulation), while negative values indicate a lower expression level in the tumor sample (down-regulation).

Expression Profiles of Genes Differentially Expressed in Stomach Cancer

Using the above described methods, genes that were predominantly over-expressed in stomach cancer, or predominantly under-expressed in stomach cancer, were identified. Genes with consistent differential expression patterns provide potential targets for broad range diagnostics and therapeutics.

Table 1 lists the set of genes determined to be differentially expressed in cancerous stomach tissue compared to normal stomach tissue, with the fold change value for each gene. This set of genes, along with the relative expression levels of each gene, creates a profile for the disease examined. A profile produced by a subset of these genes can also be of diagnostic or prognostic value, if examination of a patient's stomach biopsy sample shows up-regulation and/or down-regulation of a subset of the genes in Table 1. Likewise, in addition to their diagnostic and monitoring uses, a subset of the genes of Table 1 can be used to screen therapeutic agents or used in pharmaceutical compositions as therapeutic agents.

These genes or subsets of the genes of Table 1 confirm an overall stomach cancer gene expression profile. The genes in Table 1 may be used alone, or in combination with the methods, compositions, databases and computer systems of the invention.

Although the present invention has been described in detail with reference to examples above, it is understood that various modifications can be made without departing from the spirit of the invention. Accordingly, the invention is limited only by the following claims. All cited patents and publications referred to in this application are herein incorporated by reference in their entirety.

TABLE 1
Genes Differentially Expressed in Stomach Cancer

| Fragment | | Accession | | |
|---|---|---|---|---|
| Name | Seq. ID | Number | UniGene ID | Gene Symbol |
| 89128_at | 1 | AI968491 | Hs.236516 | MINCLE |
| 80846_at | 2 | AI198352 | Hs.7165 | ZNF259 |
| 51709_at | 3 | A1925439 | Hs.194694 | MAP3K6 |
| 34345_at | 4 | AF026031 | Hs.31334 | TOM |
| 57342_at | 5 | AA524258 | Hs.279862 | TOK-1 |
| 40076_at | 6 | AF004430 | Hs.154718 | TPD52L2 |
| 45723_at | 7 | AI805297 | Hs.24135 | DKFZp761C241 |
| 32849_at | 8 | D80000 | Hs.211602 | SMC1L1 |
| 43809_at | 9 | AI984100 | Hs.178761 | POH1 |
| 40036_at | 10 | AF035940 | Hs.57904 | MAGOH |
| 65586_at | 11 | AI951998 | Hs.65588 | DAZAP1 |
| 38964_r_at | 12 | U12707 | Hs.2157 | WAS |
| 37700_at | 13 | X92106 | Hs.78943 | BLMH |
| 36968_s_at | 14 | AL050353 | Hs.274170 | OIP2 |
| 31879_at | 15 | U69127 | Hs.153636 | FUBP3 |
| 37766_s_at | 16 | AF035309 | Hs.79387 | PSMC5 |
| 33150_at | 17 | AI126004 | Hs.322901 | SAS10 |
| 49015_at | 18 | AA149864 | Hs.22393 | DENR |
| 37490_at | 19 | L27213 | Hs.1176 | SLC4A3 |
| 34845_at | 20 | AL035398 | Hs.4877 | CGI-51 |
| 37189_at | 21 | AL023553 | Hs.75835 | PMM1 |
| 34986_at | 22 | AF030455 | Hs.116651 | EVA1 |
| 912_s_at | 23 | M21056 | Hs.992 | PLA2G1B |
| 56491_at | 24 | AL079368 | Hs.44017 | SIR2L |
| 41071_at | 25 | X57655 | Hs.98243 | SPINK2 |
| 37874_at | 26 | Z47553 | Hs.14286 | FMO5 |
| 34506_at | 27 | M13928 | Hs.1227 | ALAD |
| 47490_at | 28 | AL123839 | Hs.132957 | LOC64102 |
| 32177_s_at | 29 | AC004084 | Hs.184367 | GAPL |
| 35925_at | 30 | AF040639 | Hs.284236 | AKR7A3 |
| 32570_at | 31 | L76465 | Hs.77348 | HPGD |
| 34070_s_at | 32 | Z84717 | Hs.306284 | PDI |
| 926_at | 33 | J03910 | Hs.334409 | MT1G |

| Fragment | | | | |
|---|---|---|---|---|
| Name | Description | | | |
| 89128_at | C-type (calcium dependent, carbohydrate-recognition | | | |
| | domain) lectin, superfamily member 9 | | | |
| 80846_at | zinc finger protein 259 | | | |
| 51709_at | mitogen-activated protein kinase kinase kinase 6 | | | |
| 34345_at | putative mitochondrial outer membrane protein import | | | |
| | receptor | | | |
| 57342_at | cdk inhibitor p21 binding protein | | | |
| 40076_at | tumor protein D52-like 2 | | | |
| 45723_at | transmembrane protein vezatin; hypothetical protein | | | |
| | DKFZp761C241 | | | |
| 32849_at | SMC1 (structural maintenance of chromosomes 1, yeast)- | | | |
| | like 1 | | | |
| 43809_at | 26S proteasome-associated pad1 homolog | | | |
| 40036_at | mago-nashi (Drosophila) homolog, proliferafion-associated | | | |
| 65586_at | DAZ associated protein 1 | | | |
| 38964_r_at | Wiskott-Aldrich syndrome (eczema-thrombocytopenia) | | | |
| 37700_at | bleomycin hydrolase | | | |
| 36968_s_at | Opa-interacdng protein 2 | | | |
| 31879_at | far upstream element (FUSE) binding protein 3 | | | |
| 37766_s_at | protease (prosome, macropain) 26S subunit, ATPase | | | |
| | 5,proteasome (prosome, macropain) 26S subunit, ATPase, 5 | | | |
| 33150_at | disrupter of silencing 10 | | | |
| 49015_at | density-regulated protein | | | |
| 37490_at | solute carrier family 4, anion exchanger, member 3 | | | |
| 34845_at | CGI-51 protein | | | |
| 37189_at | phosphomannomutase 1 | | | |
| 34986_at | epithelial V-like antigen 1 | | | |
| 912_s_at | phospholipase A2, group IB (pancreas) | | | |
| 56491_at | sirtuin (silent mating type information regulation 2, | | | |
| | S. cerevisiae, homolog) 2 | | | |
| 41071_at | serine protease inhibitor, Kazal type, 2 (acrosin- | | | |
| | trypsin inhibitor) | | | |
| 37874_at | flavin containing monooxygenase 5 | | | |
| 34506_at | aminolevulinate, delta-, dehydratase | | | |
| 47490_at | tenomodulin protein | | | |
| 32177_s_at | GTPase activating protein-like | | | |
| 35925_at | aldo-keto reductase family 7, member A3 (aflatoxin | | | |
| | aldehyde reductase) | | | |
| 32570_at | hydroxyprostaglandin dehydrogenase 15-(NAD) | | | |
| 34070_s_at | protein disulfide isomerase | | | |
| 926_at | metallothionein 1G | | | |
| Fragment | | | Mean- | |
| Name | AGC/NOR | P-value | Normals | Mean-AGC |
| 89128_at | 7.65 | 0.000198 | 39.95 | 305.49 |
| 80846_at | 6.78 | 0.000506 | 27.64 | 187.33 |
| 51709_at | 6.43 | 0.000577 | 20 | 128.69 |
| 34345_at | 3.77 | 0.000591 | 44.96 | 169.39 |
| 57342_at | 3.41 | 0.000375 | 28.73 | 98.11 |
| 40076_at | 3.03 | 0.000486 | 92.39 | 279.87 |
| 45723_at | 2.92 | 0.000064 | 24.63 | 71.87 |
| 32849_at | 2.84 | 0.000775 | 33.5 | 95.24 |
| 43809_at | 2.64 | 0.00065 | 239.5 | 632.92 |
| 40036_at | 2.50 | 0.000579 | 22.25 | 55.64 |
| 65586_at | 2.45 | 0.000854 | 89.46 | 219.17 |
| 38964_r_at | 2.41 | 0.000717 | 20 | 48.2 |
| 37700_at | 2.33 | 0.000087 | 37.66 | 87.74 |
| 36968_s_at | 2.22 | 0.000836 | 40.98 | 90.92 |
| 31879_at | 2.18 | 0.000029 | 35.44 | 77.41 |
| 37766_s_at | 2.18 | 0.00031 | 199.93 | 435.96 |
| 33150_at | 2.11 | 0.000238 | 45.87 | 96.67 |
| 49015_at | 2.10 | 0.000511 | 424.76 | 893.38 |
| 37490_at | −1.85 | 0.000932 | 176.36 | 95.17 |
| 34845_at | −1.96 | 0.000969 | 316.63 | 161.32 |
| 37189_at | −2.54 | 0.000229 | 359.08 | 141.13 |
| 34986_at | −2.58 | 0.000147 | 62.22 | 24.08 |
| 912_s_at | −2.91 | 0.000877 | 64.97 | 22.31 |
| 56491_at | −2.92 | 0.00066 | 380.98 | 130.31 |
| 41071_at | −3.19 | 0.000528 | 84.55 | 26.51 |
| 37874_at | −3.62 | 0.000424 | 113.64 | 31.35 |
| 34506_at | −3.72 | 0.000335 | 99.7 | 26.79 |
| 47490_at | −3.72 | 0.000877 | 109.5 | 29.41 |
| 32177_s_at | −4.10 | 0.00098 | 158.6 | 38.72 |
| 35925_at | −5.21 | 0.000349 | 268.55 | 51.52 |
| 32570_at | −7.29 | 0.000098 | 601.07 | 82.43 |
| 34070_s_at | −7.86 | 0.000805 | 191.26 | 24.33 |
| 926_at | −16.09 | 0.000467 | 2347.13 | 145.83 |

TABLE 2
Patient Information

| | Donor | Donor | Donor Age | Date of | Organ/ | Tissue | Normal or | Specimen |
|---|---|---|---|---|---|---|---|---|
| Sample ID | Gender | Race | at Excision | Collection | Fluid | Site | Diseased | Diagnosis |
| YUMC-009-01 | Male | Korean | 54 | Jan. 16, 2001 | Stomach | antrum | Normal | NL, stomach |
| YUMC-009-02 | Male | Korean | 54 | Jan. 16, 2001 | Stomach | antrum | Malignant | adenoca, moderate(AGC) |
| YUMC-048-01 | Female | Korean | 65 | Jan. 10, 2001 | Stomach | body, cardia | Normal | normal, stomach |
| YUMC-048-02 | Female | Korean | 65 | Jan. 10, 2001 | Stomach | body, cardia | Malignant | advanced gastric cancer |
| YUMC-050-01 | Male | Korean | 62 | Apr. 23, 2001 | Stomach | UB | Normal | normal, stomach |
| YUMC-050-02 | Male | Korean | 62 | Apr. 23, 2001 | Stomach | UB | Malignant | advanced gastric cancer |
| YUMC-053-01 | Male | Korean | 68 | Mar. 30, 2001 | Stomach | body | Normal | normal, stomach |
| YUMC-053-02 | Male | Korean | 68 | Mar. 30, 2001 | Stomach | body | Malignant | advanced gastric cancer |
| YUMC-057-01 | Male | Korean | 47 | Mar. 7, 2001 | Stomach | body | Normal | normal, stomach |
| YUMC-057-02 | Male | Korean | 47 | Mar. 7, 2001 | Stomach | body | Malignant | advanced gastric cancer |

1. A method of diagnosing stomach cancer in a patient, comprising:
(a) detecting the level of expression in a tissue sample of one or more genes from Table 1; wherein differential expression of the genes in Table 1 is indicative of stomach cancer.

2. A method of detecting the progression of stomach cancer in a patient, comprising:
(a) detecting the level of expression in a tissue sample of one or more genes from Table 1; wherein differential expression of the genes in Table 1 is indicative of stomach cancer progression.

3. A method of monitoring the treatment of a patient with stomach cancer, comprising:
(a) administering a pharmaceutical composition to the patient;
(b) preparing a gene expression profile of one or more of the genes in Table 1 from a cell or tissue sample from the patient; and
(c) comparing the patient gene expression profile to a gene expression profile from a cell population selected from the group consisting of normal stomach cells and cancerous stomach cells.

4. A method of treating a patient with stomach cancer, comprising:
(a) administering to the patient a pharmaceutical composition;
(b) preparing a gene expression profile of one or more of the genes in Table 1 from a cell or tissue sample from the patient; and
(c) comparing the patient expression profile to a gene expression profile selected from the group consisting of normal stomach cells and cancerous stomach cells.

5. A method of typing stomach disease in a patient, comprising:
(a) detecting the level of expression in a tissue sample of one or more genes from Table 1; wherein differential expression of the genes in Table 1 is indicative that the stomach disease is stomach cancer.

6. A method of screening for an agent capable of modulating the onset or progression of stomach cancer, comprising:
(a) preparing a first gene expression profile of a cell population comprising cancerous stomach cells, wherein the expression profile comprises the expression level of one or more genes from Table 1;
(b) exposing the cell population to the agent;
(c) preparing second gene expression profile of the agent-exposed cell population; and
(d) comparing the first and second gene expression profiles.

7. A composition comprising at least two oligonucleotides, wherein each of the oligonucleotides comprises a sequence that specifically hybridizes to a gene in Table 1.

8. A composition according to claim 7, wherein the composition comprises at least 3 oligonucleotides.

9. A composition according to claim 7, wherein the composition comprises at least 5 oligonucleotides.

10. A composition according to claim 7, wherein the composition comprises at least 7 oligonucleotides.

11. A composition according to claim 7, wherein the composition comprises at least 10 oligonucleotides.

12. A composition according to any one of claims 7, wherein the oligonucleotides are attached to a solid support.

13. A composition according to claim 12, wherein the solid support is selected from a group consisting of a membrane, a glass support, a filter, a tissue culture dish, a polymeric material, a bead and a silica support.

14. A solid support comprising at least two oligonucleotides, wherein each of the oligonucleotides comprises a sequence that specifically hybridizes to a gene in Table 1.

15. A solid support according to claim 14, wherein the oligonucleotides are covalently attached to the solid support.

16. A solid support according to claim 14, wherein the oligonucleotides are non-covalently attached to the solid support.

17. A solid support according to claim 14, wherein the support comprises at least about 10 different oligonucleotides in discrete locations per square centimeter.

18. A solid support according to claim 14, wherein the support comprises at least about 100 different oligonucleotides in discrete locations per square centimeter.

19. A solid support according to claim 14, wherein the support comprises at least about 1000 different oligonucleotides in discrete locations per square centimeter.

20. A solid support according to claim 14, wherein the support comprises at least about 10,000 different oligonucleotides in discrete locations per square centimeter.

21. A computer system comprising:
(a) a database containing information identifying the expression level in stomach tissue of a set of genes comprising at least one gene in Table 1; and
(b) a user interface to view the information.

22. A computer system of claim 21, wherein the database further comprises sequence information for the genes.

23. A computer system of claim 21, wherein the database further comprises information identifying the expression level for the genes in normal stomach tissue.

24. A computer system of claim 21, wherein the database further comprises information identifying the expression level for the genes in tissue from a stomach tumor.

25. A computer system of any of claims 21-24, further comprising records including descriptive information from an external database, which information correlates said genes to records in the external database.

26. A computer system of claim 25, wherein the external database is GenBank.

27. A method of using a computer system of any one of claims 21-24 to present information identifying the expression level in a tissue or cell of at least one gene in Table 1, comprising:
(a) comparing the expression level of at least one gene in Table 1 in the tissue or cell to the level of expression of the gene in the database.

28. A method of claim 27, wherein the expression level of at least two genes are compared.

29. A method of claim 27, wherein the expression level of at least five genes are compared.

30. A method of claim 27, wherein the expression level of at least ten genes are compared.

31. A method of claim 27, further comprising displaying the level of expression of at least one gene in the tissue or cell sample compared to the expression level in stomach cancer.

32. A therapeutic agent for slowing or halting the progression of stomach cancer, wherein the agent is selected from the group consisting of the genes in Table 1, functional fragments of the genes in Table 1, proteins encoded by the genes in Table 1 and functional fragments of said proteins.

33. A method of treating a patient with stomach cancer, comprising:
(a) administering to a patient with stomach cancer a pharmaceutical composition comprising all or a portion of at least one gene in Table 1, or a protein encoded therein.
